(19) United States
(12) Patent Application Publication
(10) Pub. No.: US 2021/0077632 A1
(43) Pub. Date: Mar. 18, 2021

(54) Formulations of Benzazepine Conjugates and Uses Thereof

(71) Applicant: Silverback Therapeutics, Inc., Seattle, WA (US)

(72) Inventors: Sean Wesley Smith, Seattle, WA (US); Sateesh Natarajan, Redmond, WA (US)

(73) Assignee: Silverback Therapeutics, Inc., Seattle, WA (US)

(21) Appl. No.: 16/993,539

(22) Filed: Aug. 14, 2020

Related U.S. Application Data
(60) Provisional application No. 62/887,335, filed on Aug. 15, 2019.

(51) Int. Cl. A61K 47/68; A61K 47/54; A61K 31/55; A61K 39/395; A61K 47/12; A61K 47/22; A61K 47/26; A61K 47/40; A61K 47/36; A61K 9/08

(52) U.S. Cl. CPC A61K 47/6803; A61K 47/6851; A61K 47/545; A61K 31/55; A61K 39/39558; A61K 9/08; A61K 47/22; A61K 47/26; A61K 47/40; A61K 47/36; A61K 47/12

(57) ABSTRACT

Aqueous formulations comprising conjugates that comprise a compound comprising benzazepine or a benzazepine-like structure conjugated to a polypeptide are provided, as well as methods of treatment using the aqueous formulations. Lyophilized compositions comprising the conjugates that comprise a compound comprising benzazepine or a benzazepine-like structure conjugated to a polypeptide are also provided.

### CROSS-REFERENCE TO RELATED APPLICATIONS

This application claims the benefit of priority of U.S. Provisional Application No. 62/887,335, filed Aug. 15, 2019, which is incorporated by reference herein in its entirety for any purpose.

### SEQUENCE LISTING

The present application is filed with a Sequence Listing in electronic format. The Sequence Listing is provided as a file entitled “2020-08-12_01230-0008-00US_Seq_List_ST25.txt” created on Aug. 12, 2020, which is 65,536 bytes in size. The information in the electronic format of the sequence listing is incorporated herein by reference in its entirety.

### FIELD

The present application relates to formulations of benzazepine and benzazepine-like conjugates. In some embodiments, the benzazepine and benzazepine-like conjugates are immune-stimulatory conjugates comprising a benzazepine compound and a polypeptide, such as an antibody.

### BACKGROUND

One of the leading causes of death in the United States is cancer. Conventional methods of cancer treatment, like chemotherapy, surgery, or radiation therapy, tend to be highly toxic and/or nonspecific to a cancer, resulting in limited efficacy and harmful side effects. The immune system has the potential to be a powerful, specific tool in fighting cancers. This observation has led to the development of immunotherapeutics as drug candidates for clinical trials. Immunotherapeutics can act by boosting a specific immune response and have the potential to be a powerful anti-cancer treatment. Such immunotherapeutics may comprise benzazepine compounds, which in some instances, act as TLR8 agonists.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

In some embodiments, an aqueous formulation is provided, comprising a conjugate comprising a compound linked to a polypeptide, wherein the compound comprises the structure:

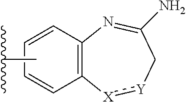

wherein [symbol] is a double bond or a single bond;

wherein when [symbol] is a double bond, X and Y are each CH; and

when [symbol] is a single bond, one of X and Y is CH₂ and the other is CH₂, O, or NH; and

the structure is optionally substituted at any position other than the —NH₂;
wherein the pH of the formulation ranges from about 4.5 to about 5.2. In certain embodiments, the pH of the formulation ranges from 4.4 to 5.4. In further embodiments, the pH of the formulation is about 4.9 or is 4.9.

In some embodiments, a lyophilized formulation is provided, comprising a conjugate comprising a compound linked to a polypeptide, wherein the compound comprises the structure:

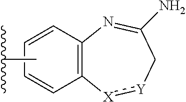

wherein [symbol] is a double bond or a single bond;
wherein when [symbol] is a double bond, X and Y are each CH; and
when is a single bond, one of X and Y is CH₂ and the other is CH₂, O, or NH; and

the structure is optionally substituted at any position other than the —NH₂;

wherein upon reconstitution of the lyophilized composition in water to form an aqueous formulation, the pH of the aqueous formulation ranges from about 4.5 to about 5.2. In certain embodiments, the pH of the formulation ranges from 4.4 to 5.4. In further embodiments, the pH of the formulation is about 4.9 or is 4.9.

Methods of controlling hydrolysis of a compound conjugated to a polypeptide in an aqueous formulation are provided, wherein the compound comprises the structure:

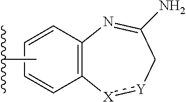

wherein [symbol] is a double bond or a single bond;

wherein when [symbol] is a double bond, X and Y are each CH; and

when [symbol] is a single bond, one of X and Y is CH₂ and the other is CH₂, O, or NH; and

the structure is optionally substituted at any position other than the —NH₂;
comprising formulating the conjugate to form an aqueous formulation, wherein the pH of the aqueous formulation ranges from about 4.5 to about 5.2. In certain embodiments, the pH of the formulation ranges from 4.4 to 5.4. In further embodiments, the pH of the formulation is about 4.9 or is 4.9.

Methods of treating a disease or disorder in a subject are also provided, comprising administering to the subject a therapeutically effective amount of an aqueous formulation provided herein. In some embodiments, the disease or disorder is cancer, fibrosis, or an infectious disease.

### BRIEF DESCRIPTION OF THE DRAWINGS

Certain features of the disclosure are set forth with particularity in the appended claims. A better understanding of the features and advantages of the present disclosure will be obtained by reference to the following detailed description that sets forth illustrative aspects, in which the principles of the disclosure are utilized, and the accompanying drawings of which:

FIG. 1 shows conjugate stability at 2-8° C., 25° C., and 40° C. in formulations 1-5 from Table 1.

FIG. 2 shows the hydrophobic interaction chromatography (HIC) profiles of formulation 1 from Table 1 following 2 week incubation at 2-8° C., 25° C., and 40° C.

FIG. 3 shows the HIC profiles for formulations 1 and 3 from Table 1 at time zero and after storage at 25° C. for 2 weeks.

FIG. 4 shows the HIC profiles for formulations 11 and 24 from Table 1, which have conjugate concentrations of 10 mg/ml and 80 mg/ml, respectively, at time 0 and following a 1 week incubation at 25° C.

FIG. 5 shows the HIC profiles for formulations 3 and 24 from Table 1, which are pH 6.5 and 4.5, respectively, and have conjugate concentrations of 10 mg/ml and 80 mg/ml, respectively, at time 0 and following a 2 week incubation at 25° C.

FIGS. 6A and 6B show the measure of drug to antibody ratio (DAR) by HIC (FIG. 6A) and free linker-payload (% wt/wt) by RP-HPLC (FIG. 6B) for formulation 24 from Table 1, following a 2 week incubation at 25° C. The payload is the benzazepine compound, which is conjugated to a HER2 antibody as described in Example 1. Dotted horizontal lines represent the analytical variability window expected for the assay and the dashed line represents the center point.

FIG. 7 shows RP-HPLC traces of digested antibody conjugates comprising a benzazepine compound (top), a lactam compound (bottom), and a sample of a benzazepine conjugate that has been incubated under stress conditions (i.e., at 40° C. in PBS (neutral pH) for 3 days).

### DETAILED DESCRIPTION

The present disclosure provides conjugates comprising benzazepine compounds stably conjugated to polypeptides. While such linked benzazepine compounds are stably attached to the protein (e.g., an antibody), such compounds may undergo a chemical transformation (e.g., deaminate) when in an aqueous formulation at neutral pH, particularly when stored under stress conditions (e.g., at a temperature 25° C. or higher). The inventors surprisingly discovered that formulating the conjugates at a pH from about 4.5 to about 5.2 reduces the chemical transformation. Accordingly, in various embodiments herein, aqueous formulations of conjugates comprising a benzazepine, or a benzazepine-like compound, linked to a polypeptide (such as an antibody) are provided, wherein the aqueous formulations have a pH from about 4.5 to about 5.2 or have a pH from 4.4 to 5.4 or have a pH of about 4.9.

Additional aspects and advantages of the present disclosure will become apparent to those skilled in this art from the following detailed description, wherein illustrative aspects of the present disclosure are shown and described. As will be appreciated, the present disclosure is capable of other and different aspects, and its several details are capable of modifications in various respects, all without departing from the disclosure. Accordingly, the descriptions are to be regarded as illustrative in nature, and not as restrictive.

### Definitions

As used herein, a “tumor associated antigen” or “tumor antigen” refers to an antigen present on a cancer cell that can be recognized by an antibody and is preferentially present on a cancer cell as compared to normal (non-cancerous) cells.

As used herein, the term “antibody” refers to an immunoglobulin molecule that specifically binds to, or is immunologically reactive toward, a specific antigen. The portion of the antibody that binds a specific antigen may be referred to as an “antigen binding domain.” The term antibody can include, for example, polyclonal, monoclonal, genetically engineered, and antigen binding fragments thereof. An antibody can be, for example, murine, chimeric, humanized, a heteroconjugate, bispecific, diabody, triabody, or tetrabody. An antigen binding fragment can include, for example, a Fab′, F(ab′)₂, Fab, Fv, rIgG, scFv, hcAbs (heavy chain antibodies), a single domain antibody, V_{HH}, V_{NAR}, sdAbs, or nanobody.

As used herein, “recognize” refers to the specific association or specific binding between an antigen binding domain and an antigen. Specific association or specific binding does not require that the antigen binding domain does not associate with or bind to any other antigen, but rather that it preferentially associates with or binds to the antigen, as compared to association with or binding to an unrelated antigen.

As used herein, an “Fc domain” refers to a domain from an Fc portion of an antibody that can specifically bind to an Fc receptor, such as a Fcgamma receptor or an FcRn receptor.

As used herein, “recognize” refers to the specific association or specific binding between an antigen binding domain and an antigen. Specific association or specific binding does not require that the antigen binding domain does not associate with or bind to any other antigen, but rather that it preferentially associates with or binds to the antigen, as compared to association with or binding to an unrelated antigen.

As used herein, a “myeloid cell” refers to a dendritic cell, a macrophage, a monocyte, a neutrophil, a myeloid derived suppressor cell (MDSC).

As used herein, an “antigen presenting cell” or “APC” refers to a cell that can present antigen to a T-, or B-cell, in a productive manner leading to activation and/or expansion of T-, or B-cell clones specific for said antigen. Nonlimiting exemplary APCs include dendritic cells, macrophages, monocytes, and B cells. In some embodiments, an antigen presenting cell is a dendritic cell, a macrophage, or a monocyte.

As used herein, an “immune stimulatory compound” is a compound that activates or stimulates an immune cell, such as a myeloid cell or an APC.

As used herein, a “myeloid cell agonist” refers to a compound that activates or stimulates an immune response by a myeloid cell.

As used herein, the term “B-cell depleting agent” refers to an agent that, when administered to a subject, causes a reduction in the number of B cells in the subject. In some embodiments, a B-cell depleting agent binds a B cell surface molecule, such as, for example, CD20, CD22, or CD19. In some embodiments, a B-cell depleting agent inhibits a B cell survival factor, such as, for example, BLyS or APRIL. B-cell depleting agents include, but are not limited to, anti-CD20 antibodies, anti-CD19 antibodies, anti-CD22 antibodies, anti-BLyS antibodies, TACI-Ig, BR3-Fc, and anti-BR3 antibodies. Nonlimiting exemplary B-cell depleting agents include rituximab, ocrelizumab, ofatumumab, epratuzumab, MEDI-51 (anti-CD19 antibody), belimumab, BR3-Fc, AMG-623, and atacicept.

As used herein, the term “conjugate” refers to a polypeptide attached to at least one compound, optionally via a linker(s). In some embodiments, the polypeptide is an antibody.

As used herein, an “immune-stimulatory conjugate” refers to a conjugate that activates or stimulates the immune system or a portion thereof, as determined by an in vitro or in vivo assay.

As used herein, an “immune cell” refers to a T cell, B cell, NK cell, NKT cell, or an antigen presenting cell. In some embodiments, an immune cell is a T cell, B cell, NK cell, or NKT cell. In some embodiments, an immune cell is an antigen presenting cell. In some embodiments, an immune cell is not an antigen presenting cell.

The terms “salt” or “pharmaceutically acceptable salt” refer to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine.

In some embodiments, the pharmaceutically acceptable base addition salt is chosen from ammonium, potassium, sodium, calcium, and magnesium salts.

The term “C_{x-y}” when used in conjunction with a chemical moiety, such as alkyl, alkenyl, or alkynyl is meant to include groups that contain from x to y carbons in the chain. For example, the term “C₁₋₆alkyl” refers to substituted or unsubstituted saturated hydrocarbon groups, including straight-chain alkyl and branched-chain alkyl groups that contain from 1 to 6 carbons.

The term —C_{x-y}alkylene- refers to a substituted or unsubstituted alkylene chain with from x to y carbons in the alkylene chain. For example —C₁₋₆alkylene- may be selected from methylene, ethylene, propylene, butylene, pentylene, and hexylene, any one of which is optionally substituted.

The terms “C_{x-y}alkenyl” and “C_{x-y}alkynyl” refer to substituted or unsubstituted unsaturated aliphatic groups analogous in length and possible substitution to the alkyls described above, but that contain at least one double or triple bond, respectively. The term —C_{x-y}alkenylene- refers to a substituted or unsubstituted alkenylene chain with from x to y carbons in the alkenylene chain. For example, —C₂₋₆alkenylene- may be selected from ethenylene, propenylene, butenylene, pentenylene, and hexenylene, any one of which is optionally substituted. An alkenylene chain may have one double bond or more than one double bond in the alkenylene chain. The term —C_{x-y}alkynylene- refers to a substituted or unsubstituted alkynylene chain with from x to y carbons in the alkenylene chain. For example, —C₂₋₆alkenylene- may be selected from ethynylene, propynylene, butynylene, pentynylene, and hexynylene, any one of which is optionally substituted. An alkynylene chain may have one triple bond or more than one triple bond in the alkynylene chain.

“Alkylene” refers to a divalent hydrocarbon chain linking the rest of the molecule to a radical group, consisting solely of carbon and hydrogen, containing no unsaturation, and preferably having from one to twelve carbon atoms, for example, methylene, ethylene, propylene, butylene, and the like. The alkylene chain is attached to the rest of the molecule through a single bond and to the radical group through a single bond. The points of attachment of the alkylene chain to the rest of the molecule and to the radical group are through the terminal carbons respectively. In other embodiments, an alkylene comprises one to five carbon atoms (i.e., C₁-C₅ alkylene). In other embodiments, an alkylene comprises one to four carbon atoms (i.e., C₁-C₄ alkylene). In other embodiments, an alkylene comprises one to three carbon atoms (i.e., C₁-C₃ alkylene). In other embodiments, an alkylene comprises one to two carbon atoms (i.e., C₁-C₂ alkylene). In other embodiments, an alkylene comprises one carbon atom (i.e., C₁ alkylene). In other embodiments, an alkylene comprises five to eight carbon atoms (i.e., C₅-C₈ alkylene). In other embodiments, an alkylene comprises two to five carbon atoms (i.e., C₂-C₅ alkylene). In other embodiments, an alkylene comprises three to five carbon atoms (i.e., C₃-C₅ alkylene). Unless stated otherwise specifically in the specification, an alkylene chain is optionally substituted by one or more substituents such as those substituents described herein.

“Alkenylene” refers to a divalent hydrocarbon chain linking the rest of the molecule to a radical group, consisting solely of carbon and hydrogen, containing at least one carbon-carbon double bond, and preferably having from two to twelve carbon atoms. The alkenylene chain is attached to the rest of the molecule through a single bond and to the radical group through a single bond. The points of attachment of the alkenylene chain to the rest of the molecule and to the radical group are through the terminal carbons respectively. In other embodiments, an alkenylene comprises two to five carbon atoms (i.e., C₂-C₅ alkenylene). In other embodiments, an alkenylene comprises two to four carbon atoms (i.e., C₂-C₄ alkenylene). In other embodiments, an alkenylene comprises two to three carbon atoms (i.e., C₂-C₃ alkenylene). In other embodiments, an alkenylene comprises two carbon atom (i.e., C₂ alkenylene). In other embodiments, an alkenylene comprises five to eight carbon atoms (i.e., C₅-C₈ alkenylene). In other embodiments, an alkenylene comprises three to five carbon atoms (i.e., C₃-C₅ alkenylene). Unless stated otherwise specifically in the specification, an alkenylene chain is optionally substituted by one or more substituents such as those substituents described herein.

“Alkynylene” refers to a divalent hydrocarbon chain linking the rest of the molecule to a radical group, consisting solely of carbon and hydrogen, containing at least one carbon-carbon triple bond, and preferably having from two to twelve carbon atoms. The alkynylene chain is attached to the rest of the molecule through a single bond and to the radical group through a single bond. The points of attachment of the alkynylene chain to the rest of the molecule and to the radical group are through the terminal carbons respectively. In other embodiments, an alkynylene comprises two to five carbon atoms (i.e., C₂-C₅ alkynylene). In other embodiments, an alkynylene comprises two to four carbon atoms (i.e., C₂-C₄ alkynylene). In other embodiments, an alkynylene comprises two to three carbon atoms (i.e., C₂-C₃ alkynylene). In other embodiments, an alkynylene comprises two carbon atom (i.e., C₂ alkynylene). In other embodiments, an alkynylene comprises five to eight carbon atoms (i.e., C₅-C₈ alkynylene). In other embodiments, an alkynylene comprises three to five carbon atoms (i.e., C₃-C₅ alkynylene). Unless stated otherwise specifically in the specification, an alkynylene chain is optionally substituted by one or more substituents such as those substituents described herein.

“Heteroalkylene” refers to a divalent hydrocarbon chain including at least one heteroatom in the chain, containing no unsaturation, and preferably having from one to twelve carbon atoms and from one to 6 heteroatoms, e.g., —O—, —NH—, —S—. The heteroalkylene chain is attached to the rest of the molecule through a single bond and to the radical group through a single bond. The points of attachment of the heteroalkylene chain to the rest of the molecule and to the radical group are through the terminal atoms of the chain. In other embodiments, a heteroalkylene comprises one to five carbon atoms and from one to three heteroatoms. In other embodiments, a heteroalkylene comprises one to four carbon atoms and from one to three heteroatoms. In other embodiments, a heteroalkylene comprises one to three carbon atoms and from one to two heteroatoms. In other embodiments, a heteroalkylene comprises one to two carbon atoms and from one to two heteroatoms. In other embodiments, a heteroalkylene comprises one carbon atom and from one to two heteroatoms. In other embodiments, a heteroalkylene comprises five to eight carbon atoms and from one to four heteroatoms. In other embodiments, a heteroalkylene comprises two to five carbon atoms and from one to three heteroatoms. In other embodiments, a heteroalkylene comprises three to five carbon atoms and from one to three heteroatoms. Unless stated otherwise specifically in the specification, a heteroalkylene chain is optionally substituted by one or more substituents such as those substituents described herein.

The term “carbocycle” as used herein refers to a saturated, unsaturated or aromatic ring in which each atom of the ring is carbon. Carbocycle includes 3- to 10-membered monocyclic rings, 6- to 12-membered bicyclic rings, and 6- to 12-membered bridged rings. Each ring of a bicyclic carbocycle may be selected from saturated, unsaturated, and aromatic rings. In an exemplary embodiment, an aromatic ring, e.g., phenyl, may be fused to a saturated or unsaturated ring, e.g., cyclohexane, cyclopentane, or cyclohexene. A bicyclic carbocycle includes any combination of saturated, unsaturated and aromatic bicyclic rings, as valence permits. A bicyclic carbocycle includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. Exemplary carbocycles include cyclopentyl, cyclohexyl, cyclohexenyl, adamantyl, phenyl, indanyl, and naphthyl. The term “unsaturated carbocycle” refers to carbocycles with at least one degree of unsaturation and excluding aromatic carbocycles. Examples of unsaturated carbocycles include cyclohexadiene, cyclohexene, and cyclopentene.

The term “heterocycle” as used herein refers to a saturated, unsaturated or aromatic ring comprising one or more heteroatoms. Exemplary heteroatoms include N, O, Si, P, B, and S atoms. Heterocycles include 3- to 10-membered monocyclic rings, 6- to 12-membered bicyclic rings, and 6- to 12-membered bridged rings. A bicyclic heterocycle includes any combination of saturated, unsaturated and aromatic bicyclic rings, as valence permits. In an exemplary embodiment, an aromatic ring, e.g., pyridyl, may be fused to a saturated or unsaturated ring, e.g., cyclohexane, cyclopentane, morpholine, piperidine or cyclohexene. A bicyclic heterocycle includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. The term “unsaturated heterocycle” refers to heterocycles with at least one degree of unsaturation and excluding aromatic heterocycles. Examples of unsaturated heterocycles include dihydropyrrole, dihydrofuran, oxazoline, pyrazoline, and dihydropyridine.

The term “heteroaryl” includes aromatic single ring structures, preferably 5- to 7-membered rings, more preferably 5- to 6-membered rings, whose ring structures include at least one heteroatom, preferably one to four heteroatoms, more preferably one or two heteroatoms. The term “heteroaryl” also includes polycyclic ring systems having two or more rings in which two or more carbons are common to two adjoining rings wherein at least one of the rings is heteroaromatic, e.g., the other rings can be aromatic or non-aromatic carbocyclic, or heterocyclic. Heteroaryl groups include, for example, pyrrole, furan, thiophene, imidazole, oxazole, thiazole, pyrazole, pyridine, pyrazine, pyridazine, and pyrimidine, and the like.

The term “substituted” refers to moieties having substituents replacing a hydrogen on one or more carbons or substitutable heteroatoms, e.g., —NH—, of the structure. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)R^{a} (where t is 1 or 2), —R—S(O)ₜR^{a} (where t is 1 or 2), and —R—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R—N(R^{a})₂, —R^{b}—C(O)R^{a}, —R^{b}—C(O)OR^{a}, —R—C(O)N(R^{a})₂, —R^{b}—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R¹⁰—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)R^{a} (where t is 1 or 2), —R—S(O)OR^{a} (where t is 1 or 2) and —R—S(O)ₜN(R^{a})₂ (where t is 1 or 2); and wherein each R^{b} is independently selected from a direct bond or a straight or branched alkylene, alkenylene, or alkynylene chain, and each R is a straight or branched alkylene, alkenylene or alkynylene chain.

It will be understood by those skilled in the art that substituents can themselves be substituted, if appropriate. Unless specifically stated as “unsubstituted,” references to chemical moieties herein are understood to include substituted variants. For example, reference to a “heteroaryl” group or moiety implicitly includes both substituted and unsubstituted variants.

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, chemical entities described herein are intended to include all Z-, E- and tautomeric forms as well.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

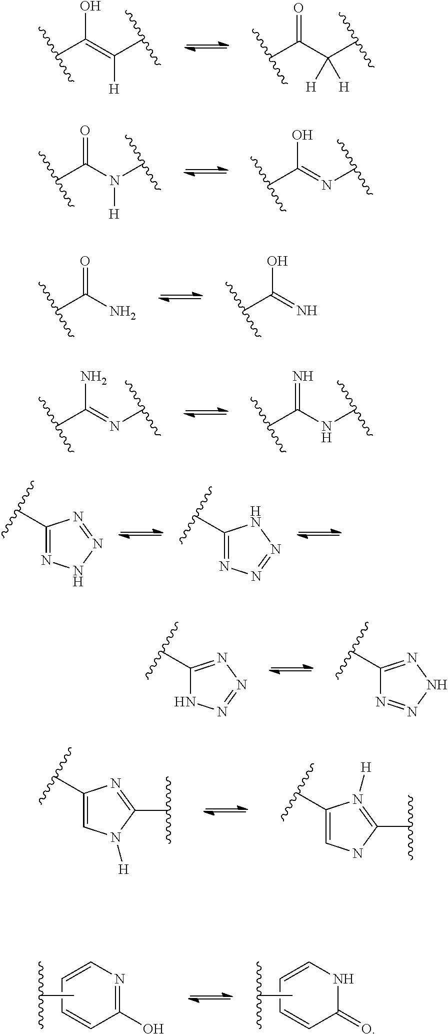

Where structures include a bond crossed by a wavy line, e.g.,

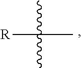

the wavy line indicates the bond is covalently attached to at least one additional moiety. In some aspects, conjugates described herein comprise a benzazepine or benzazepine-like compound linked to a polypeptide, and the benzazepine or benzazepine-like compound structure is shown as a substituent with a pendant wavy line/bond indicating the benzazepine or benzazepine-like compound is connected directly or indirectly to a polypeptide via the indicated bond.

The phrases “intravenous administration” and “administered intravenously” as used herein refer to injection or infusion of a conjugate into a vein of a subject.

The phrases “intravenous slow infusion” and “IV slow infusion” as used here refer to an intravenous infusion that results in a Tmax of 4 hours or more.

The phrases “subcutaneous administration”, “subcutaneously administering” and the like refer to administration of a conjugate into the subcutis of a subject. For clarity, a subcutaneous administration is distinct from an intratumoral injection into a tumor or cancerous lesion located in the subcuta.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “targeting moiety” refers to a structure that has a selective affinity for a target molecule relative to other non-target molecules. A targeting moiety binds to a target molecule. A targeting moiety may be a polypeptide, such as, for example, an antibody, a peptide, a ligand, a receptor, or a binding portion thereof. The target biological molecule may be a biological receptor or other structure of a cell such as a tumor antigen. A targeting moiety is often specific for a particular cell surface antigen, so as to target an immune-stimulatory compound to a target cell or disease site.

The term “about” as used herein in the context of a number refers to a range centered on that number and spanning 10% less than that number and 10% more than that number. The term “about” used in the context of a range refers to an extended range spanning 10% less than that the lowest number listed in the range and 10% more than the greatest number listed in the range.

The phrase “at least one of” when followed by a list of items or elements refers to an open ended set of one or more of the elements in the list, which may but does not necessarily include more than one of the elements.

### Exemplary Polypeptides

In various embodiments, a conjugate comprises a benzazepine or benzazepine-like compound linked to a polypeptide. Nonlimiting exemplary polypeptides that may be included in the conjugates include antibodies, fusion proteins, peptides, and the like. In some embodiments, the polypeptide is a receptor or receptor extracellular domain, a cytokine (such as an immunocytokine), or a ligand. In some embodiments, the polypeptide is a fusion protein comprising, for example, a receptor extracellular domain fused to an Fc domain. In some embodiments, the polypeptide is a non-antibody molecule that specifically binds to an antigen, including, but not limited to, a DARPin, an affimer, an avimer, a knottin, a monobody, lipocalin, an anticalin, ‘T-body’, an affibody, a peptibody, an affinity clamp, or peptide. In some embodiments, the polypeptide is a bicyclic peptide (e.g., a Bicycle®), as described in Published International Application No. WO 2014/140342, WO 2013/050615, WO 2013/050616, and WO 2013/050617 (the binding polypeptides of which are incorporated by reference herein).

In some embodiments, a conjugate as described herein comprises an antibody. In some such embodiments, the antibody comprises one or more antigen binding domains and an Fc domain, wherein each antigen binding domain specifically binds to an antigen. An antibody can have, for example, a first antigen binding domain that specifically binds to a first antigen, a second antigen binding domain that specifically binds to a second antigen, and an Fc domain. In various embodiments, an antibody can include two antigen binding domains, in which each antigen binding domain recognizes the same epitope on the antigen. An antibody can include two antigen binding domains in which each antigen binding domain recognizes a different epitope of the same antigen. An antibody can include two antigen binding domains in which each antigen binding domain recognizes different antigens. In various embodiments, an antibody has one antigen binding domain. In various embodiments, an antigen binding domain may comprise, for example, a heavy chain variable domain (VH) and a light chain variable domain (VL), or in the case of a heavy-chain only antibody, a V_{HH}.

Nonlimiting exemplary antigens that may be bound by a polypeptide, such as an antibody, include CD5, CD25, CD37, CD33, CD45, BCMA, CS-1, PD-L1, B7-H3, B7-DC (PD-L2), HLD-DR, carcinoembryonic antigen (CEA), TAG-72, EpCAM, MUC1, folate-binding protein (FOLR1), A33, G250 (carbonic anhydrase IX), prostate-specific membrane antigen (PSMA), GD2, GD3, GM2, Ley, CA-125, CA19-9 (MUC1 sLe(a)), epidermal growth factor, HER2, IL-2 receptor, EGFRvIII (de2-7 EGFR), fibroblast activation protein (FAP), a tenascin, a metalloproteinase, endosialin, avB3, LMP2, EphA2, PAP, AFP, ALK, polysialic acid, TRP-2, fucosyl GM1, mesothelin (MSLN), PSCA, sLe(a), GM3, BORIS, Tn, TF, GloboH, STn, CSPG4, AKAP-4, SSX2, Legumain, Tie 2, Tim 3, VEGFR2, PDGFR-B, ROR2, TRAIL1, MUC16, EGFR, CMET, HER3, MUC1, MUC15, CA6, NAPI2B, TROP2, CLDN18.2, RON, LY6E, FRAlpha, DLL3, PTK7, LIV1, ROR1, CLDN6, GPC3, ADAM12, LRRC15, CDH6, TMEFF2, TMEM238, GPNMB, ALPPL2, UPK1B, UPK2, LAMP-1, LY6K, EphB2, STEAP, ENPP3, CDH3, Nectin4, LYPD3, EFNA4, GPA33, SLITRK6, and HAVCR1.

In certain embodiments, a polypeptide, such as an antibody, specifically binds to a non-proteinaceous or glycoantigen, such as GD2, GD3, GM2, Ley, polysialic acid, fucosyl GM1, GM3, Tn, STn, sLe(animal), or GloboH.

In certain embodiments, a polypeptide, such as an antibody, specifically binds to a solid tumor antigen. In some embodiments, the solid tumor antigen is preferentially present on sarcoma or carcinoma cell(s). In some embodiments, the solid tumor antigen is preferentially present on a sarcoma cell(s). In some embodiments, the solid tumor antigen is preferentially present on a carcinoma cell(s).

In some embodiments, the solid tumor antigen is present on cells of a brain, breast, lung, liver, kidney, pancreatic, colorectal, ovarian, head and neck, bone, skin, mesothelioma, bladder, esophageal, stomach (gastric), prostate, thyroid, uterine or cervical/endometrial cancer.

In some embodiments, the solid tumor antigen is an antigen present on breast cancer, such as HER2, TROP2, LIV-1, CDH3 (p-cadherin), MUC1, Sialo-epitope CA6, PTK7, GPNMB, LAMP-1, LRRC15, ADAM12, EPHA2, TNC, LYPD3, EFNA4 and CLDN6. In certain embodiments, the breast cancer antigen is HER2.

In some embodiments, the solid tumor antigen is an antigen present on brain cancer, such as EGFRvIII, TNC and DLL-3.

In some embodiments, the solid tumor antigen is an antigen present on lung cancer, such as mesothelin, HER2, EGFR, PD-L1, MSLN, LY6K, CD56, PTK7, FOLR1, DLL3, SLC34A2, CECAM5, MUC16, LRRC15, ADAM12, EGFRvIII, LYPD3, EFNA4 and MUC1.

In certain embodiments, the lung cancer antigen is HER2.

In some embodiments, the solid tumor antigen is an antigen present on liver cancer, such as GPC3, EPCAM, CECAM5.

In some embodiments, the solid tumor antigen is an antigen present on kidney cancer, such as HAVCR1, ENPP3, CDH6, CD70, and cMET.

In some embodiments, the solid tumor antigen is an antigen present on pancreatic cancer, such as PTK7, MUC16, MSLN, LRRC15, ADAM12, EFNA4, MUC5A and MUC1. In certain embodiments, the pancreatic cancer antigen is LRRC15.

In some embodiments, the solid tumor antigen is an antigen present on colorectal cancer, such as EPHB2, TMEM238, CECAM5, LRRC15, ADAM12, EFNA4 and GPA33. In certain embodiments, the colorectal cancer antigen is HER2.

In some embodiments, the solid tumor antigen is an antigen present on ovarian cancer, such as MUC16, MUC1, MSLN, FOLR1, sTN, VTCN1, HER2, PTK7, FAP, TMEM238, LRRC15, CLDN6, SLC34A2 and EFNA4. In certain embodiments, the ovarian cancer antigen is HER2.

In some embodiments, the solid tumor antigen is an antigen present on head and neck cancer, such as LY6K, PTK7, LRRC15, ADAM12, LYPD3, EFNA4 and TNC.

In some embodiments, the solid tumor antigen is an antigen present on bone cancer, such as EPHA2, LRRC15, ADAM12, GPNMB, TP-3 and CD248.

In some embodiments, the solid tumor antigen is an antigen present on mesothelioma, such as MSLN.

In some embodiments, the solid tumor antigen is an antigen present on bladder cancer, such as LY6K, PTK7, UPK1B, UPK2, TNC, Nectin4, SLITRK6, LYPD3, EFNA4 and HER2.

In certain embodiments, the bladder cancer antigen is Nectin4. In certain other embodiments, the bladder cancer antigen is HER2.

In some embodiments, the solid tumor antigen is an antigen present on esophageal or stomach (gastric) cancer, such as HER2, EPHB2, TMEM238, CECAM5 and EFNA4. In certain embodiments, the esophogeal cancer antigen is HER2. In certain other embodiments, the gastric cancer antigen is HER2.

In some embodiments, the solid tumor antigen is an antigen present on prostate cancer, such as PSMA, FOLH1, PTK7, STEAP, TMEFF2 (TENB2), OR51E2, SLC30A4 and EFNA4.

In certain embodiments, the prostate cancer antigen is PSMA.

In some embodiments, the solid tumor antigen is an antigen present on thyroid cancer, such as PTK7.

In some embodiments, the solid tumor antigen is an antigen present on uterine cancer, such as present on uterine cancer such as LY6K, PTK7, EPHB2, FOLR1, ALPPL2, MUC16 and EFNA4.

In some embodiments, the solid tumor antigen is an antigen present on cervical/endometrial cancer, such as LY6K, PTK7, MUC16, LYPD3, EFNA4 and MUC1.

In some embodiments, the solid tumor antigen is an antigen present on a sarcoma, such as LRRC15.

In some embodiments, the tumor antigen is HER2. In some aspects, the HER2 antigen is expressed for example, on an ovarian, bladder, esophageal, stomach, or breast cancer cell.

In some aspects, the antigen is a liver cell antigen. In some aspects, the liver cell antigen is expressed on a canalicular cell, Kupffer cell, hepatocyte, or any combination thereof. In some aspects, the liver cell antigen is a hepatocyte antigen. In some aspects, the liver cell antigen is selected from the group consisting of ASGR1 (asialoglycoprotein receptor 1), ASGR2 (asialoglycoprotein receptor 2), TRF2, UGT1A1, SLC22A7, SLC13A5, SLC22A1, and C9. In some aspects, the liver cell antigen is selected from the group consisting of ASGR1, ASGR2, and TRF2. In certain embodiments, the liver cell antigen is ASGR1. In some aspects, the liver cell antigen is expressed on a liver cell infected with a virus selected from the group consisting of HBV and HCV. In certain embodiments, the liver cell antigen is ASGR1 and the liver cell is infected with HBV.

In some aspects, the antigen is a viral antigen from a virus selected from the group consisting of HBV and HCV. In some aspects, the viral antigen is an HBV antigen. In some aspects, the viral antigen is HBsAg, HBcAg, or HBeAg. In some aspects, the viral antigen is HBsAg.

In some embodiments, an antibody comprises an antigen binding domain and an Fc domain. In some embodiments, an antibody comprises two light chain polypeptides (light chains) and two heavy chain polypeptides (heavy chains), held together covalently by disulfide linkages. The heavy chain typically comprises a heavy chain variable region (VH) and a heavy chain constant region. The heavy chain constant region comprises three domains, CH1, CH2, and CH3. An Fc domain typically comprises heavy chain CH2 and CH3 domains. The light chain typically comprises a light chain variable region (VL) and a light chain constant region. The antigen-recognition regions of the antibody variable domains typically comprise six complementarity determining regions (CDRs), or hypervariable regions, that lie within the framework of the heavy chain variable region and light chain variable region at the N-terminal ends of the two heavy and two light chains. The constant domains provide the general framework of the antibody and may not be involved directly in binding the antibody to an antigen, but can be involved in various effector functions, such as participation of the antibody in antibody-dependent cellular cytotoxicity (ADCC).

An antibody can be any class, e.g., IgA, IgD, IgE, IgG, and IgM. Certain classes can be further divided into isotypes, e.g., IgG1, IgG2, IgG3, IgG4, IgA1, and IgA2. The heavy-chain constant regions that correspond to the different classes of immunoglobulins can be α, δ, ε, γ, and μ, respectively. The light chains can be either kappa (or κ) or lambda (or λ).

In some embodiments an antigen binding domain comprises a light chain complementary determining region 1 (LCDR1), a light chain complementary determining region 2 (LCDR2), a light chain complementary determining region 3 (LCDR3), a heavy chain complementary determining region 1 (HCDR1), a heavy chain complementary determining region 2 (HCDR2), and a heavy chain complementary determining region 3 (HCDR3). In some embodiments, an antibody may be a heavy-chain only antibody, in which case the antigen binding domain comprises HCDR1, HCDR2, and HCDR3, and the antibody lacks a light chain. Unless stated otherwise, the CDRs described herein can be defined according to the IMGT (the international ImMunoGeneTics information) system.

An antibody can be chimeric or humanized. Chimeric and humanized forms of non-human (e.g., murine) antibodies can be intact (full length) chimeric immunoglobulins, immunoglobulin chains or antigen binding fragments thereof (such as Fv, Fab, Fab′, F(ab′)₂ or other target-binding subdomains of antibodies), which can contain sequences derived from non-human immunoglobulin. In general, the humanized antibody can comprise substantially all of at least one, and typically two, variable domains, in which all or substantially all of the CDR regions correspond to those of a non-human immunoglobulin and all or substantially all of the framework (FR) regions are those of a human immunoglobulin sequence. A humanized antibody can also comprise at least a portion of an immunoglobulin constant region (Fc), an Fc domain, typically that of a human immunoglobulin sequence.

An antibody described herein can be a human antibody. As used herein, “human antibodies” can include antibodies having, for example, the amino acid sequence of a human immunoglobulin and include antibodies isolated from human immunoglobulin libraries or from animals transgenic for one or more human immunoglobulins and that typically do not express endogenous immunoglobulins. Human antibodies can be produced using transgenic mice which are incapable of expressing functional endogenous immunoglobulins, but which can express human immunoglobulin genes. Completely human antibodies that recognize a selected epitope can be generated using guided selection. In this approach, a selected non-human monoclonal antibody, e.g., a mouse antibody, is used to guide the selection of a completely human antibody recognizing the same epitope

An antibody described herein can be a bispecific antibody or a dual variable domain antibody (DVD). Bispecific and DVD antibodies are monoclonal, often human or humanized, antibodies that have binding specificities for at least two different antigens.

An antibody described herein can be derivatized or otherwise modified. For example, derivatized antibodies can be modified by glycosylation, acetylation, pegylation, phosphorylation, amidation, derivatization by known protecting/blocking groups, proteolytic cleavage, or the like.

An antibody described herein can specifically bind to a cancer antigen. An antibody can specifically bind to a solid tumor antigen.

In some embodiments, the antibody may comprise the CDRs (such as LCDR1, LCDR2, LCDR3, HCDR1, HCDR2 and HCDR3, according to the IMGT system), the variable regions, or the entire heavy and light chains of an antibody selected from trastuzumab, cetuximab, panitumumab, ofatumumab, belimumab, ipilimumab, pertuzumab, tremelimumab, nivolumab, pembrolizumab, atezolizumab, MDX-1105 (WO 2007/005874), dacetuzumab, urelumab, MPDL3280A, lambrolizumab, blinatumomab, nimotuzumab, zalutumumab, onartuzumab, patritumab, clivatuzumab, sofituzumab, edrecolomab, adecatumumab, anetumab, huDS6, lifastuzumab, sacituzumab, PR1A3, humanized PR1A3, humanized Ab2-3, claudiximab, AMG595, ABT806, sibrotuzumab, DS-8895a variant 1, DS-8895a variant 2, MEDI-547, narnatumab, RG7841, farletuzumab, mirvetuximab, J591 variant 1, J591 variant 2, rovalpituzumab, PF-06647020, ladiratuzumab, cirmtuzumab, ladiratuzumab, huLiv1-14 (WO 2012078688), Liv1-1.7A4 (US 2011/0117013), huLiv1-22 (WO 2012078688), 4H11 (US 2013/0171152), 4H5 (US 2013/0171152), glembatumumab, oportuzumab, enfortumab, depatuxizumab, the antibody of ASG-15ME, huM25 (WO2017/095808A1), and codrituzumab.

In some embodiments, an antibody specifically binds to a breast cancer antigen. In some such embodiments, the antibody may comprise the CDRs (such as LCDR1, LCDR2, LCDR3, HCDR1, HCDR2 and HCDR3, according to the IMGT system), the variable regions, or the entire heavy and light chains of an antibody selected from trastuzumab, pertuzumab, sacituzumab, ladiratuzumab, huLiv1-14 (WO 2012078688), Liv1-1.7A4 (US 2011/0117013), huLiv1-22 (WO 2012078688), huDS6, glembatumumab, PF-0664720, MEDI-547, DS-8895a variant 1, and DS-08895a variant 2.

In some embodiments, an antibody specifically binds to an antigen present on brain cancer. In some such embodiments, the antibody may comprise the CDRs (such as LCDR1, LCDR2, LCDR3, HCDR1, HCDR2 and HCDR3, according to the IMGT system), the variable regions, or the entire heavy and light chains of an antibody selected from AMG595, ABT806, rovalpituzumab or depatuxizumab.

In some embodiments, an antibody specifically binds to an antigen present on lung cancer. In some such embodiments, the antibody may comprise the CDRs (such as LCDR1, LCDR2, LCDR3, HCDR1, HCDR2 and HCDR3, according to the IMGT system), the variable regions, or the entire heavy and light chains of an antibody selected from panitumumab, cetuximab, pembrolizumab, nivolumab, atezolizumab, and nimotuzumab, lifastuzumab, anetumab, PF-0664720, farletuzumab, rovalpituzumab, lifastuzumab, sofituzumab, huDS6, ABT806, AMG595, and huM25 (WO 2017/095808A1).

In some embodiments, an antibody specifically binds to an antigen present on liver cancer. In some such embodiments, the antibody may comprise the CDRs (such as LCDR1, LCDR2, LCDR3, HCDR1, HCDR2 and HCDR3, according to the IMGT system), the variable regions, or the entire heavy and light chains of an antibody selected from codrituzumab, oportuzumab, and humanized PR1A3.

In some embodiments, an antibody specifically binds to an antigen present on kidney cancer. In some such embodiments, the antibody may comprise the CDRs (such as LCDR1, LCDR2, LCDR3, HCDR1, HCDR2 and HCDR3, according to the IMGT system), the variable regions, or the entire heavy and light chains of an antibody selected from AGS-16M8F, AGS-16C3, the antibody of CDX-014, and onartuzumab.

In some embodiments, an antibody specifically binds to an antigen present on pancreatic cancer. In some such embodiments, the antibody may comprise the CDRs (such as LCDR1, LCDR2, LCDR3, HCDR1, HCDR2 and HCDR3, according to the IMGT system), the variable regions, or the entire heavy and light chains of an antibody selected from PF-0664720, clivatuzumab, 4H11(US 2013/0171152), 4H5 (US 2013/0171152), anetumumab, huDS6, sofituzumab, huM25 (WO 2017/095808A1), and RG7841.

In some embodiments, an antibody specifically binds to an antigen present on colorectal cancer. In some such embodiments, the antibody may comprise the CDRs (such as LCDR1, LCDR2, LCDR3, HCDR1, HCDR2 and HCDR3, according to the IMGT system), the variable regions, or the entire heavy and light chains of an antibody selected from huM25 (WO 2017/095808A1), PR1A3, humanized PR1A3, pantumumab, cetuximab, nimotuzumab, and zalutumumab.

In some embodiments, an antibody specifically binds to an antigen present on ovarian cancer. In some such embodiments, the antibody may comprise the CDRs (such as LCDR1, LCDR2, LCDR3, HCDR1, HCDR2 and HCDR3, according to the IMGT system), the variable regions, or the entire heavy and light chains of an antibody selected from sofituzumab, 4H11(US 2013/0171152, 4H5 (US 2013/0171152), huDS6, farletuzumab, anetumab, trastuzumab, pertuzumab, PF-0664720, sibrotuzumab, huM25 (WO 2017/095808), and lifastuzumab.

In some embodiments, an antibody specifically binds to an antigen present on head and neck cancer. In some such embodiments, the antibody may comprise the CDRs (such as LCDR1, LCDR2, LCDR3, HCDR1, HCDR2 and HCDR3, according to the IMGT system), the variable regions, or the entire heavy and light chains of an antibody selected from cetuximab, panitumumab, nimtuzumab, PF-0664720, pantumumab, cetuximab, nimotuzumab, and zalutumumab.

In some embodiments, an antibody specifically binds to an antigen present on bone cancer. In some such embodiments, the antibody may comprise the CDRs (such as LCDR1, LCDR2, LCDR3, HCDR1, HCDR2 and HCDR3, according to the IMGT system), the variable regions, or the entire heavy and light chains of an antibody selected from huM25 (WO2017/095808A1), DS-8895a variant 1, DS-8895a variant 2, and glembatumab.

In some embodiments, an antibody specifically binds to an antigen present on skin cancer.

In some embodiments, an antibody specifically binds to an antigen present on mesothelioma.

In some embodiments, an antibody specifically binds to an antigen present on cervical/endometrial cancer. In some such embodiments, the antibody may comprise the CDRs (such as LCDR1, LCDR2, LCDR3, HCDR1, HCDR2 and HCDR3, according to the IMGT system), the variable regions, or the entire heavy and light chains of an antibody selected from PF-0664720, anetumumab, 4H11(US 2013/0171152), 4H5 (US 2013/0171152), huDS6, and sofituzumab.

In some embodiments, an antibody specifically binds to an antigen present on bladder cancer. In some such embodiments, the antibody may comprise the CDRs (such as LCDR1, LCDR2, LCDR3, HCDR1, HCDR2 and HCDR3, according to the IMGT system), the variable regions, or the entire heavy and light chains of an antibody selected from enfortumab, trastuzumab, pertuzumab and SLITRK6.

In some embodiments, an antibody specifically binds to an antigen present on stomach cancer. In some such embodiments, the antibody may comprise the CDRs (such as LCDR1, LCDR2, LCDR3, HCDR1, HCDR2 and HCDR3, according to the IMGT system), the variable regions, or the entire heavy and light chains of an antibody selected from sofituzumab, anetumab, pertuzumab, trastuzumab, and humanized PR1A3.

In some embodiments, an antibody specifically binds to an antigen present on prostate cancer. In some such embodiments, the antibody may comprise the CDRs (such as LCDR1, LCDR2, LCDR3, HCDR1, HCDR2 and HCDR3, according to the IMGT system), the variable regions, or the entire heavy and light chains of an antibody selected from mirvetuximab, J591 variant 1, and J591 variant 2.

In some embodiments, an antibody specifically binds to an antigen present on thyroid cancer.

In some embodiments, an antibody specifically binds to an antigen present on uterine cancer. In some such embodiments, the antibody may comprise the CDRs (such as LCDR1, LCDR2, LCDR3, HCDR1, HCDR2 and HCDR3, according to the IMGT system), the variable regions, or the entire heavy and light chains of an antibody selected from PF-0664720, farletuzumab, sofituzumab, 4H11(US 2013/0171152, and 4H5 (US 2013/0171152).

In some embodiments, an antibody specifically binds to an antigen present on a sarcoma.

In some embodiments, an antibody specifically binds to an antigen present on a liver cell and the subject has a viral infection (e.g., HBV or HCV). The antibody can be, for example, an antibody that binds to ASGR1 or ASGR2.

### Exemplary Fc Domains

A polypeptide, such as a fusion protein or an antibody, may comprise an Fe domain. An Fe domain is a structure that can bind to one or more Fc receptors (FcRs). In various embodiments, an Fc domain is from an IgG antibody, such as an IgG1, IgG2, or IgG4 antibody. An Fc domain typically comprises C_{H}2 and C_{H}3 domains of a heavy chain constant region, but may comprise more or less of the heavy chain constant region as well.

An Fc domain can be a domain of an antibody that can bind to an FcR(s). FcRs are organized into classes (e.g., gamma (γ), alpha (α) and epsilon (ε)) based on the class of antibody that the FcR recognizes. The FcαR class binds to IgA and includes several isoforms, FcαRI (CD89) and FcαμR. The FcγR class binds to IgG and includes several isoforms, FcγRI (CD64), FcγRIIA (CD32a), FcγRIIB (CD32b), FcγRIIIA (CD16a), and FcγRIIIB (CD16b). An FcγRIIIA (CD16a) can be an FcγRIIIA (CD16a) F158 variant or a V158 variant. FcRs also can be FcRn receptors.

Each FcγR isoform can differ in binding affinity to the Fc domain of the IgG antibody. For example, FcγRI can bind to IgG with greater affinity than FcγRII or FcγRIII. The affinity of a particular FcγR isoform to an IgG can be controlled, in part, by a glycan (e.g., oligosaccharide) at position CH2 84.4 of the IgG antibody. For example, fucose containing CH2 84.4 glycans can reduce IgG affinity for FcγRIIIA. In addition, G0 glucans can have increased affinity for FcγRIIIA due to the lack of galactose and terminal GlcNAc moiety.

Binding of an Fc domain to an FcR can enhance an immune response. FcR-mediated signaling that can result from an Fc domain binding to an FcR and can lead to the maturation of immune cells. FcR-mediated signaling that can result from an Fc domain binding to an FcR can lead to the maturation of dendritic cells (DCs). FcR-mediated signaling that can result from an Fc domain binding to an FcR can lead to antibody dependent cellular cytotoxicity. FcR-mediated signaling that can result from an Fc domain binding to an FcR can lead to more efficient immune cell antigen uptake and processing. FcR-mediated signaling that can result from an Fc domain binding to an FcR can promote the expansion and activation of T cells. FcR-mediated signaling that can result from an Fc domain binding to an FcR can promote the expansion and activation of CD8+ T cells. FcR-mediated signaling that can result from an Fc domain binding to an FcR can influence immune cell regulation of T cell responses. FcR-mediated signaling that can result from an Fc domain binding to an FcR can influence immune cell regulation of T cell responses. FcR-mediated signaling that can result from an Fc domain binding to an FcR can influence dendritic cell regulation of T cell responses. FcR-mediated signaling that can result from an Fc domain binding to an FcR can influence functional polarization of T cells (e.g., polarization can be toward a TH1 cell response).

An Fe domain can be modified, such as by a modification of the amino acid sequence, to alter the recognition of an FcR for the Fc domain. Such modification(s) may still allow for FcR-mediated signaling, depending on the modification. A modification can be a substitution of an amino acid at a residue of an Fc domain for a different amino acid at that residue. A modification can be an insertion or deletion of an amino acid at a residue of an Fc domain. A modification can permit binding of an FcR to a site on the Fc domain to which the that the FcR may not otherwise bind. A modification can increase binding affinity of an FcR to the Fc domain. A modification can decrease binding affinity of an FcR to the Fc domain.

An Fc domain can be a variant of a naturally occurring Fc domain (e.g., a wild type Fc domain) and can comprise at least one amino acid change as compared to the sequence of a wild-type Fc domain. An amino acid change in an Fc domain can allow the antibody or conjugate to bind to at least one Fc receptor with greater affinity compared to a wild-type Fc domain. An amino acid change in an Fc domain can allow the antibody or conjugate to bind to at least one Fc receptor with lessor affinity compared to a wild-type Fc domain.

In some embodiments, an Fc domain exhibits increased binding affinity to one or more Fc receptors. In some embodiments, an Fc domain exhibits increased binding affinity to one or more Fcgamma receptors. In some embodiments, an Fc domain exhibits increased binding affinity to FcRn receptors. In some embodiments, an Fc domain exhibits increased binding affinity to Fcgamma and FcRn receptors. In other embodiments, an Fc domain exhibits the same or substantially similar binding affinity to Fcgamma and/or FcRn receptors as compared to a wild-type Fc domain from an IgG antibody (e.g., IgG1 antibody).

In some embodiments, an Fc domain exhibits decreased binding affinity to one or more Fc receptors. In some embodiments, an Fc domain exhibits decreased binding affinity to one or more Fcgamma receptors. In some embodiments, an Fc domain exhibits decreased binding affinity to FcRn receptors. In some embodiments, an Fc domain exhibits decreased binding affinity to Fcgamma and FcRn receptors. In some embodiments, an Fc domain is an Fc null domain. In some embodiments, an Fc domain exhibits decreased binding affinity to FcRn receptors, but exhibits the same or increased binding affinity to one or more Fcgamma receptors as compared to a wildtype Fc domain. In some embodiments, an Fc domain exhibits increased binding affinity to FcRn receptors, but exhibits the same or decreased binding affinity to one or more Fcgamma receptors.

An Fc domain may have one or more, two or more, three or more, or four or more amino acid substitutions that decrease binding of the Fc domain to an Fc receptor. In certain embodiments, an Fc domain has decreased binding affinity for one or more of FcγRI (CD64), FcγRIIA (CD32), FcγRIIIA (CD16a), FcγRIIIB (CD16b), or any combination thereof. In order to decrease binding affinity of an Fc domain to an Fc receptor, the Fc domain may comprise one or more amino acid substitutions that reduces the binding affinity of the Fc domain to an Fc receptor. In other embodiments, an Fc domain exhibits the same or substantially similar binding affinity to one or more of FcγRI (CD64), FcγRIIA (CD32), FcγRIIIA (CD16a), FcγRIIIB (CD16b), or any combination thereof as compared to a wild-type Fc domain from an IgG antibody (e.g., IgG1 antibody). In some embodiments, an Fc domain can comprise a sequence of an IgG isoform that has been modified from the wild-type IgG sequence. In some embodiments, the Fc domain can comprise a sequence of the IgG1 isoform that has been modified from the wild-type IgG1 sequence. In some embodiments, the modification comprises substitution of one or more amino acids that reduce binding affinity of an IgG Fc domain to all Fc receptors.

A modification can be substitution of E233, L234 and L235, such as E233P/L234V/L235A or E233P/L234V/L235A/AG236, according to the EU index of Kabat. A modification can be a substitution of P238, such as P238A, according to the EU index of Kabat. A modification can be a substitution of D265, such as D265A, according to the EU index of Kabat. A modification can be a substitution of N297, such as N297A, according to the EU index of Kabat. A modification can be a substitution of A327, such as A327Q, according to the EU index of Kabat. A modification can be a substitution of P329, such as P239A, according to the EU index of Kabat.

In some embodiments, an IgG Fc domain comprises at least one amino acid substitution that reduces its binding affinity to FcγR1, as compared to a wild-type or reference IgG Fc domain. A modification can comprise a substitution at F241, such as F241A, according to the EU index of Kabat. A modification can comprise a substitution at F243, such as F243A, according to the EU index of Kabat. A modification can comprise a substitution at V264, such as V264A, according to the EU index of Kabat. A modification can comprise a substitution at D265, such as D265A according to the EU index of Kabat.

In some embodiments, an IgG Fc domain comprises at least one amino acid substitution that increases its binding affinity to FcγR1, as compared to a wild-type or reference IgG Fc domain. A modification can comprise a substitution at A327 and P329, such as A327Q/P329A, according to the EU index of Kabat.

In some embodiments, the modification comprises substitution of one or more amino acids that reduce binding affinity of an IgG Fc domain to FcγRII and FcγRIIIA receptors. A modification can be a substitution of D270, such as D270A, according to the EU index of Kabat. A modification can be a substitution of Q295, such as Q295A, according to the EU index of Kabat. A modification can be a substitution of A327, such as A237S, according to the EU index of Kabat.

In some embodiments, the modification comprises substitution of one or more amino acids that increases binding affinity of an IgG Fc domain to FcγRII and FcγRIIIA receptors. A modification can be a substitution of T256, such as T256A, according to the EU index of Kabat. A modification can be a substitution of K290, such as K290A, according to the EU index of Kabat.

In some embodiments, the modification comprises substitution of one or more amino acids that increases binding affinity of an IgG Fc domain to FcγRII receptor. A modification can be a substitution of R255, such as R255A, according to the EU index of Kabat. A modification can be a substitution of E258, such as E258A, according to the EU index of Kabat. A modification can be a substitution of S267, such as S267A, according to the EU index of Kabat. A modification can be a substitution of E272, such as E272A, according to the EU index of Kabat. A modification can be a substitution of N276, such as N276A, according to the EU index of Kabat. A modification can be a substitution of D280, such as D280A, according to the EU index of Kabat. A modification can be a substitution of H285, such as H285A, according to the EU index of Kabat. A modification can be a substitution of N286, such as N286A, according to the EU index of Kabat. A modification can be a substitution of T307, such as T307A, according to the EU index of Kabat. A modification can be a substitution of L309, such as L309A, according to the EU index of Kabat. A modification can be a substitution of N315, such as N315A, according to the EU index of Kabat. A modification can be a substitution of K326, such as K326A, according to the EU index of Kabat. A modification can be a substitution of P331, such as P331A, according to the EU index of Kabat. A modification can be a substitution of S337, such as S337A, according to the EU index of Kabat. A modification can be a substitution of A378, such as A378A, according to the EU index of Kabat. A modification can be a substitution of E430, such as E430, according to the EU index of Kabat.

In some embodiments, the modification comprises substitution of one or more amino acids that increases binding affinity of an IgG Fc domain to FcγRII receptor and reduces the binding affinity to FcγRIIIA receptor. A modification can be a substitution of H268, such as H268A, according to the EU index of Kabat. A modification can be a substitution of R301, such as R301A, according to the EU index of Kabat. A modification can be a substitution of K322, such as K322A, according to the EU index of Kabat.

In some embodiments, the modification comprises substitution of one or more amino acids that decreases binding affinity of an IgG Fc domain to FcγRII receptor but does not affect the binding affinity to FcγRIIIA receptor. A modification can be a substitution of R292, such as R292A, according to the EU index of Kabat. A modification can be a substitution of K414, such as K414A, according to the EU index of Kabat.

In some embodiments, the modification comprises substitution of one or more amino acids that decreases binding affinity of an IgG Fc domain to FcγRII receptor and increases the binding affinity to FcγRIIIA receptor. A modification can be a substitution of S298, such as S298A, according to the EU index of Kabat. A modification can be substitution of S239,1332 and A330, such as S239D/I332E/A330L. A modification can be substitution of S239 and 1332, such as S239D/I332E.

In some embodiments, the modification comprises substitution of one or more amino acids that decreases binding affinity of an IgG Fc domain to FcγRIIIA receptor. A modification can be substitution of F241 and F243, such as F241S/F243S or F241I/F243I, according to the EU index of Kabat.

In some embodiments, the modification comprises substitution of one or more amino acids that decreases binding affinity of an IgG Fc domain to FcγRIIIA receptor and does not affect the binding affinity to FcγRII receptor. A modification can be a substitution of S239, such as S239A, according to the EU index of Kabat. A modification can be a substitution of E269, such as E269A, according to the EU index of Kabat. A modification can be a substitution of E293, such as E293A, according to the EU index of Kabat. A modification can be a substitution of Y296, such as Y296F, according to the EU index of Kabat. A modification can be a substitution of V303, such as V303A, according to the EU index of Kabat. A modification can be a substitution of A327, such as A327G, according to the EU index of Kabat. A modification can be a substitution of K338, such as K338A, according to the EU index of Kabat. A modification can be a substitution of D376, such as D376A, according to the EU index of Kabat.

In some embodiments, the modification comprises substitution of one or more amino acids that increases binding affinity of an IgG Fc domain to FcγRIIIA receptor and does not affect the binding affinity to FcγRII receptor. A modification can be a substitution of E333, such as E333A, according to the EU index of Kabat. A modification can be a substitution of K334, such as K334A, according to the EU index of Kabat. A modification can be a substitution of A339, such as A339T, according to the EU index of Kabat. A modification can be substitution of S239 and 1332, such as S239D/I332E.

In some embodiments, the modification comprises substitution of one or more amino acids that increases binding affinity of an IgG Fc domain to FcγRIIIA receptor. A modification can be substitution of L235, F243, R292, Y300 and P396, such as L235V/F243L/R292P/Y300L/P396L (IgG1VLPLL) according to the EU index of Kabat. A modification can be substitution of S298, E333 and K334, such as S298A/E333A/K334A, according to the EU index of Kabat. A modification can be substitution of K246, such as K246F, according to the EU index of Kabat.

Other substitutions in an IgG Fc domain that affect its interaction with one or more Fcγ receptors are disclosed in U.S. Pat. Nos. 7,317,091 and 8,969,526 (the disclosures of which are incorporated by reference herein).

In some embodiments, an IgG Fc domain comprises at least one amino acid substitution that reduces the binding affinity to FcRn, as compared to a wild-type or reference IgG Fc domain. A modification can comprise a substitution at H435, such as H435A according to the EU index of Kabat. A modification can comprise a substitution at 1253, such as I253A according to the EU index of Kabat. A modification can comprise a substitution at H310, such as H310A according to the EU index of Kabat. A modification can comprise substitutions at 1253, H310 and H435, such as I253A/H310A/H435A according to the EU index of Kabat.

A modification can comprise a substitution of one amino acid residue that increases the binding affinity of an IgG Fc domain for FcRn, relative to a wildtype or reference IgG Fc domain. A modification can comprise a substitution at V308, such as V308P according to the EU index of Kabat. A modification can comprise a substitution at M428, such as M428L according to the EU index of Kabat. A modification can comprise a substitution at N434, such as N434A according to the EU index of Kabat or N434H according to the EU index of Kabat. A modification can comprise substitutions at T250 and M428, such as T250Q and M428L according to the EU index of Kabat. A modification can comprise substitutions at M428 and N434, such as M428L and N434S, N434A or N434H according to the EU index of Kabat. A modification can comprise substitutions at M252, S254 and T256, such as M252Y/S254T/T256E according to the EU index of Kabat. A modification can be a substitution of one or more amino acids selected from P257L, P257N, P257I, V279E, V279Q, V279Y, A281S, E283F, V284E, L306Y, T307V, V308F, Q311V, D376V, and N434H. Other substitutions in an IgG Fc domain that affect its interaction with FcRn are disclosed in U.S. Pat. No. 9,803,023 (the disclosure of which is incorporated by reference herein).

In some embodiments, an antibody is a human IgG2 antibody, including an IgG2 Fc region. In some embodiments, the heavy chain of the human IgG2 antibody can be mutated at cysteines as positions 127, 232, or 233. In some embodiments, the light chain of a human IgG2 antibody can be mutated at a cysteine at position 214. The mutations in the heavy and light chains of the human IgG2 antibody can be from a cysteine residue to a serine residue.

### Exemplary Conjugates and Benzazepine Compounds

In some aspects, the conjugates described herein comprise benzazepine or benzazepine-like compounds, such as benzazepine immune stimulatory compounds, which can be attached via a linker(s) to form immune-stimulatory conjugates. A conjugate can include one or more benzazepine or benzazepine-like compounds, typically from about 1 to about 10 compounds per polypeptide, such as per antibody. In some embodiments, the average drug loading (e.g., drug-to-antibody ratio or DAR) of the conjugate is from about 2 to about 8, or 1 to about 3, or about 3 to about 5.

In some embodiments, an immune stimulatory compound activates human immune cells, including but not limited to dendritic cells, macrophages, monocytes, myeloid-derived suppressor cells, NK cells, B cells, T cells, or tumor cells, or a combination thereof. In some embodiments, an immune-stimulatory compound is a myeloid cell agonist. A myeloid cell agonist is a compound that activates or stimulates an immune response by a myeloid cell. For example, a myeloid cell agonist can stimulate an immune response by causing the release of cytokines by myeloid cells, which results in the activation of immune cells. The stimulation of an immune response by a myeloid cell agonist can be measured in vitro by co-culturing immune cells (e.g., peripheral blood mononuclear cells (PBMCs)) with cells targeted by the conjugate and measuring cytokine release, chemokine release, proliferation of immune cells, upregulation of immune cell activation markers, and/or ADCC. ADCC can be measured by determining the percentage of remaining target cells in the co-culture after administration of the conjugate with the target cells and PBMCs.

Conjugates generally comprise a benzazepine or benzazepine-like compound, such as an immune-stimulatory compound, covalently bound to a polypeptide, such as a targeting moiety or antibody that localizes the conjugate to a target tissue, cell population or cell. The targeting moiety can comprise all or part of an antibody variable domain, although alternate targeting moieties are also contemplated. The polypeptide is covalently attached to each compound, either directly or through a linker that tethers the compound to the polypeptide. Antibodies listed herein as well as antibodies to antigens or epitiopes thereof listed herein or otherwise known to one of skill in the art are consistent with the conjugates as disclosed herein.

The immune-stimulatory conjugates as described herein can activate, stimulate or augment an immune response against cell of a disease of condition. The activation, stimulation or augmentation of an immune response by an immune-stimulatory conjugate, such as a myeloid cell agonist, can be measured in vitro by co-culturing immune cells (e.g., myeloid cells) with cells targeted by the conjugate and measuring cytokine release, chemokine release, proliferation of immune cells, upregulation of immune cell activation markers, and/or ADCC. ADCC can be measured by determining the percentage of remaining target cells in the co-culture after administration of the conjugate with the target cells, myeloid cells, and other immune cells. In some embodiments, an immune-stimulatory conjugate can activate or stimulate immune cell activity, as determined by in vitro assay, such as a cytokine release assay, by detection of activation markers (e.g., MHC class II markers) or other assays known in the art. In some embodiments, an immune-stimulatory conjugate has an EC50 of 100 nM or less, as determine by cytokine release assay. In some embodiments, an immune-stimulatory conjugate has an EC50 of 50 nM or less, as determine by cytokine release assay. In some embodiments, an immune-stimulatory conjugate has an EC50 of 10 nM or less, as determine by cytokine release assay. In some embodiments, an immune-stimulatory conjugate has an EC50 of 1 mM or less.

In general, an immune stimulatory compound acts on toll like receptors (TLRs), nucleotide-oligomerization domain-like receptors (NOD), RIG-I-Like receptors (RLR), c-type lectin receptors (CLR), or cytosolic DNA Sensors (CDS), or a combination thereof. In some embodiments, an immune stimulatory compound comprises a ligand of one or more TLRs selected from the group consisting of: TLR2, TLR3, TLR4, TLR5, TLR7, TLR8, TLR7/TLR8, TLR9, and TLR10.

In some embodiments, an immune-stimulatory compound is a myeloid cell agonist. In certain embodiments the myeloid cell agonist is a TLR8 agonist. In certain embodiments, the TLR8 agonist is a benzazepine or benzazepine-like compound. Examples of TLR8 agonists include motolimod, VTX-763, VTX-1463, and the compounds disclosed in WO 2017216054 (Roche), WO 2017190669 (Shanghai De Novo Pharmatech), WO 2017202704 (Roche), WO2017202703 (Roche), WO 2017/046112 (Roche), WO 2016/096778 (Roche), US 20080234251 (Array Biopharma), US 20080306050 (Array Biopharma), US 20100029585 (Ventirx Pharma), US 20110092485 (Ventirx Pharma), US 20110118235 (Ventirx Pharma), US 20120082658 (Ventirx Pharma), US 20120219615 (Ventirx Pharma), US 20140066432 (Ventirx Pharma), US 20140088085 (Ventirx Pharma), and US 2019/0016808 (Birdie Biopharmaceuticals). In some embodiments, a TLR8 agonist has an EC50 value of about 500 nM or less by PBMC assay measuring TNFalpha production. In some embodiments, a TLR8 agonist has an EC50 value of about 100 nM or less by PBMC assay measuring TNFalpha production. In some embodiments, a TLR8 agonist has an EC50 value of about 50 nM or less by PBMC assay measuring TNFalpha production. In some embodiments, a TLR8 agonist has an EC50 value of about 10 nM or less by PBMC assay measuring TNFalpha production.

The aqueous formulations and lyophilized compositions described herein comprise a conjugate comprising a compound linked to a polypeptide, wherein the compound comprises the structure:

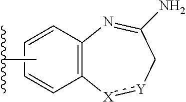

wherein [symbol] is a double bond or a single bond;

wherein when [symbol] is a double bond, X and Y are each CH; and

when [symbol] is a single bond, one of X and Y is CH₂ and the other is CH₂, O, or NH; and

the structure is optionally substituted at any position other than the —NH₂;
wherein the pH of the formulation ranges from about 4.5 to about 5.2. In certain embodiments, the pH of the formulation ranges from 4.4 to 5.4. In further embodiments, the pH of the formulation is about 4.9 or is 4.9. In certain embodiments, the polypeptide is an antibody.

In some aspects, the compound comprises the structure:

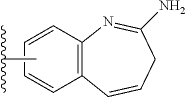

wherein the structure is optionally substituted at any position other than the —NH₂ position.

In some aspects, the aqueous formulations and lyophilized compositions comprise a conjugate comprising a compound linked to a polypeptide, wherein the compound comprises a structure selected from:

a 3H-benzo[b]azepin-2-amine structure:

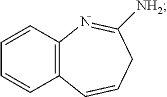 [SMILES: NC1=Nc2ccccc2C=CC1]

a 4,5-dihydro-3H-benzo[b]azepin-2-amine:

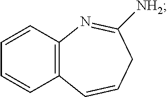 [SMILES: NC1=Nc2ccccc2C=CC1]

a 2,3-dihydrobenzo[b][1,4]oxazepin-4-amine structure:

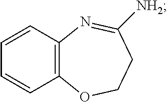 [SMILES: NC1=Nc2ccccc2OCC1]

a 3,5-dihydrobenzo[e][1,4]oxazepin-2-amine structure:

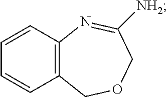 [SMILES: NC1=Nc2ccccc2COC1]

a 2,3-dihydro-1H-benzo[b][1,4]diazepin-4-amine:

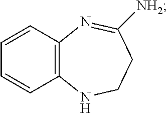 [SMILES: NC1=Nc2ccccc2NCC1]

and a 4,5-dihydro-3H-benzo[e][1,4]diazepin-2-amine:

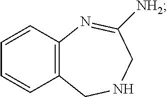 [SMILES: NC1=Nc2ccccc2CNC1]

wherein each structure is optionally substituted at any position other than the 2-amino position.

In some aspects, the aqueous formulations and lyophilized compositions described herein comprise a conjugate comprising a compound linked to a polypeptide, wherein the compound comprises a benzazepine of Formula (XI-A):

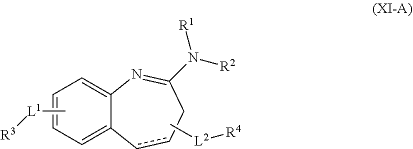

or a pharmaceutically acceptable salt thereof, wherein: [symbol]
represents an optional double bond;
L¹ is selected from —X¹—, —X²—C₁₋₆ alkylene-X²—C₁₋₆ alkylene-, —X²—C₂₋₆ alkenylene-X²—, and —X²—C₂₋₆ alkynylene-X²—, each of which is optionally substituted on alkylene, alkenylene or alkynylene with one or more R¹²;
L² is selected from —X²—, —X²—C₁₋₆ alkylene-X²—, —X²—C₂₋₆ alkenylene-X²—, and —X²—C₂₋₆ alkynylene-X²—, each of which is optionally substituted on alkylene, alkenylene or alkynylene with one or more R¹²;
X¹ is selected from —C(O)—, —C(O)N(R¹⁰)—*, —S—*, —N(R¹⁰)—*, —C(O)O—*, —OC(O)—*, —OC(O)O—*, —C(O)N(R¹⁰)C(O)—*, —C(O)N(R¹⁰)C(O)N(R¹⁰)*, —N(R¹⁰)C(O)—*, —CR¹⁰₂N(R¹⁰)C(O)—*, —N(R¹⁰)C(O)N(R¹⁰)—*, —N(R¹⁰)C(O)O—*, —OC(O)N(R¹⁰)—*, —C(NR¹⁰)—*, —N(R¹⁰)C(NR¹⁰)—*, —C(NR¹⁰)N(R¹⁰)—*, —N(R¹⁰)C(NR¹⁰)N(R¹⁰)—*, —S(O)₂—*, —OS(O)—*, —S(O)O—*, —S(O), —OS(O)₂—*, —S(O)₂O*, —N(R¹⁰)S(O)₂—*, —S(O)₂N(R¹⁰)—*, —N(R¹⁰)S(O)—*, —S(O)N(R¹⁰)—*, —N(R¹⁰)S(O)₂N(R¹⁰)—*, and —N(R¹⁰)S(O)N(R¹⁰)—*, wherein * represents where X¹ is bound to R³;
X² is independently selected at each occurrence from —O—, —S—, —N(R¹⁰)—, —C(O)—, —C(O)O—, —OC(O)—, —OC(O)O—, —C(O)N(R¹⁰)—, —C(O)N(R¹⁰)C(O)—, —C(O)N(R¹⁰)C(O)N(R¹⁰)—*, —N(R¹⁰)C(O)—, —N(R¹⁰)C(O)N(R¹⁰)—, —N(R¹⁰)C(O)O—, —OC(O)N(R¹⁰)—, —C(NR¹⁰)—, —N(R¹⁰)C(NR¹⁰)—, —C(NR¹⁰)N(R¹⁰)—, —N(R¹⁰)C(NR¹⁰)N(R¹⁰)—, —S(O)₂—, —OS(O)—, —S(O)O—, —S(O), —OS(O)₂—, —S(O)₂O, —N(R¹⁰)S(O)₂—, —S(O)₂N(R¹⁰)—, —N(R¹⁰)S(O)—, —S(O)N(R¹⁰)—, —N(R¹⁰)S(O)₂N(R¹⁰)—, and —N(R¹⁰)S(O)N(R¹⁰)—;
R¹ and R² are each hydrogen;
R³ is selected from optionally substituted C₃₋₁₂ carbocycle, and optionally substituted 3- to 12-membered heterocycle, wherein substituents on R³ are independently selected at each occurrence from: —C(O)NHNH₂, —C(O)NH—C₁₋₃ alkylene-NH(R¹⁰), —C₁₋₃ alkylene-NHC(O)OR¹¹, —C₁₋₃alkylene-NHC(O)—C₁₋₃alkylene-R¹⁰, halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰), —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and C₃₋₁₂ carbocycle, and 3- to 12-membered heterocycle, wherein each C₃₋₁₂ carbocycle, and 3- to 12-membered heterocycle in R³ is optionally substituted with one or more substituents independently selected from R¹², halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl;
R⁴ is selected from: —OR¹⁰, —N(R¹⁰)₂, —C(O)N(R¹⁰)₂, —C(O)R¹⁰, —C(O)OR¹⁰, —S(O)R¹⁰, and —S(O)₂R¹⁰; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and C₃₋₁₂ carbocycle, and 3- to 12-membered heterocycle, wherein each C₃₋₁₂ carbocycle, and 3- to 12-membered heterocycle in R⁴ is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl;
R¹⁰ is independently selected at each occurrence from: hydrogen, —NH₂, —C(O)OCH₂C₆H₅; and C₁₋₁₀ alkyl, C₂₋₁₀ alkenyl, C₂₋₁₀ alkynyl, C₃₋₁₂ carbocycle, and 3- to 12-membered heterocycle, each of which is optionally substituted with one or more substituents independently selected from halogen, —CN, —NO₂, —NH₂, ═O, ═S, —C(O)OCH₂C₆H, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and haloalkyl;
R¹¹ is selected from C₃₋₁₂ carbocycle and 3- to 12-membered heterocycle, each of which is optionally substituted with one or more substituents independently selected from R¹²;
R¹² is independently selected at each occurrence from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), and —CN; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), —CN, C₃₋₁₀ carbocycle and 3- to 10-membered heterocycle; and C₃₋₁₀ carbocycle and 3- to 10-membered heterocycle, wherein each C₃₋₁₀ carbocycle and 3- to 10-membered heterocycle in R¹² is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), —CN, C₁₋₆ alkyl, C₂₋₆ alkenyl, and C₂₋₆ alkynyl; and
wherein any substitutable carbon on the benzazepine core is optionally substituted by a substituent independently selected from R¹² or two substituents on a single carbon atom combine to form a 3- to 7-membered carbocycle.

In some aspects, the structure of Formula (XI-A) is a structure of Formula (XI-B):

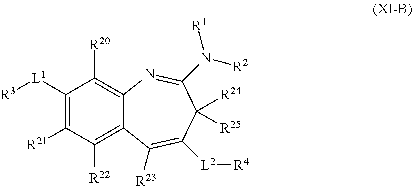

or a pharmaceutically acceptable salt thereof, wherein:
R²⁰, R²¹, R²², and R²³ are independently selected from hydrogen, halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl; and
R²⁴ and R²⁵ are independently selected from hydrogen, halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl; or R²⁴ and R²⁵ taken together form an optionally substituted saturated C₃₋₇ carbocycle.

In conjugates of the present disclosure, a structure of Formula (XI-A) or (XI-B) is connected to the rest of the conjugate via a covalent bond to a substitutable nitrogen atom, oxygen atom, or sulfur atom. In some embodiments, the rest of the conjugate is connected at R³ of Formula (XI-A) or (XI-B).

In some embodiments, R²⁰, R²¹, R²², and R²³ are independently selected from hydrogen, halogen, —OH, —OR¹⁰, —NO₂, —CN, and C₁₋₁₀ alkyl. In some embodiments, R²⁰, R²¹, R²², and R²³ are each hydrogen. In certain embodiments, R²¹ is halogen. In certain embodiments, R²¹ is hydrogen. In certain embodiments, R²¹ is —OR¹⁰. In some embodiments, R²¹ is —OCH₃.

In some embodiments, R²⁴ and R²⁵ are independently selected from hydrogen, halogen, —OH, —NO₂, —CN, and C₁₋₁₀ alkyl, or R²⁴ and R²⁵ taken together form an optionally substituted saturated C₃₋₇ carbocycle. In certain embodiments, R²⁴ and R²⁵ are each hydrogen. In other embodiments, R²⁴ and R²⁵ taken together form an optionally substituted saturated C₃₋₅ carbocycle, wherein substituents are selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN; and C₁₋₁₀ alkyl, C₂₋₁₀ alkenyl, C₂₋₁₀ alkynyl, each of which is independently optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle.

In some embodiments, L¹ is selected from —N(R¹⁰)C(O)—*, —S(O)₂N(R¹⁰)—*, —CR¹⁰₂N(R¹⁰)C (O)—* and —X²—C₁₋₆ alkylene-X²—C₁₋₆ alkylene-. In some embodiments, L¹ is selected from —N(R¹⁰)C(O)—*. In certain embodiments, R¹⁰ of —N(R¹⁰)C(O)—* is selected from hydrogen and C₁₋₆ alkyl. For example, L¹ may be —NHC(O)—*. In some embodiments, L¹ is selected from —S(O)₂N(R¹⁰)—*. In certain embodiments, R¹⁰ of —S(O)₂N(R¹⁰)—* is selected from hydrogen and C₁₋₆ alkyl. For example, L¹ is —S(O)₂NH—*. In some embodiments, L¹ is —CR¹⁰₂N(R¹⁰)C(O)—*. In certain embodiments, L¹ is selected from —CH₂N(H)C(O)—* and —CH(CH₃)N(H)C(O)—*. In some embodiments, L¹ is selected from —C(O)N(R¹⁰)—*. In certain embodiments, R¹⁰ of —C(O)N(R¹⁰)—* is selected from hydrogen and C₁₋₆ alkyl. For example, L¹ may be —C(O)NH—*.

In some embodiments, R³ is selected from optionally substituted C₃₋₁₂ carbocycle, and optionally substituted 3- to 12-membered heterocycle, wherein substituents on R³ are independently selected at each occurrence from: halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and C₃₋₁₂ carbocycle, and 3- to 12-membered heterocycle, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl. In certain embodiments, R³ is selected from optionally substituted C₃₋₁₂ carbocycle, and optionally substituted 3- to 12-membered heterocycle, wherein substituents on R³ are independently selected at each occurrence from: halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle.

In some embodiments, R³ is selected from an optionally substituted aryl and an optionally substituted heteroaryl. In some embodiments, R³ is an optionally substituted heteroaryl. R³ may be an optionally substituted heteroaryl substituted with one or more substituents independently selected from halogen, —OR¹⁰, —SR¹⁰, —N(R¹⁰)₂, —C(O)R¹⁰, —C(O)OR¹⁰, —OC(O)R¹⁰, —NO₂, ═O, ═S, —CN, C₁₋₆ alkyl, C₂₋₆ alkenyl, and C₂₋₆ alkynyl. In certain embodiments, R³ is selected from an optionally substituted 6-membered heteroaryl. For example, R³ may be an optionally substituted pyridine. In some embodiments, R³ is an optionally substituted aryl. In certain embodiments, R³ is an optionally substituted aryl substituted with one or more substituents independently selected from halogen, —OR¹⁰, —SR¹⁰, —N(R¹⁰)₂, —C(O)R¹⁰, —C(O)OR¹⁰, —OC(O)R¹⁰, —NO₂, ═O, ═S, —CN, C₁₋₆ alkyl, C₂₋₆ alkenyl, and C₂₋₆ alkynyl. R³ may be an optionally substituted phenyl. In certain embodiments, R³ is selected from pyridine, phenyl, tetrahydronaphthalene, tetrahydroquinoline, tetrahydroisoquinoline, indane, cyclopropylbenzene, cyclopentapyridine, and dihydrobenzoxaborole, any one of which is optionally substituted.

In some embodiments, R³ is selected from an optionally substituted fused 5-5, fused 5-6, and fused 6-6 bicyclic heterocycle. In certain embodiments, R³ is an optionally substituted fused 5-5, fused 5-6, and fused 6-6 bicyclic heterocycle with one or more substituents independently selected from —C(O)OR¹⁰, —N(R¹⁰)₂, —OR¹⁰, and optionally substituted C₁₋₁₀ alkyl. In certain embodiments, R³ is an optionally substituted fused 5-5, fused 5-6, and fused 6-6 bicyclic heterocycle substituted with —C(O)OR¹⁰. In certain embodiments, R³ is an optionally substituted fused 6-6 bicyclic heterocycle. For example, the fused 6-6 bicyclic heterocycle may be an optionally substituted pyridine-piperidine. In some embodiments, L¹ is bound to a carbon atom of the pyridine of the fused pyridine-piperidine. R³ may be an optionally substituted tetrahydronaphthyridine.

In some embodiments, R³ is an optionally substituted bicyclic carbocycle. In certain embodiments, R³ is an optionally substituted 8- to 12-membered bicyclic carbocycle. R³ may be an optionally substituted 8- to 12-membered bicyclic carbocycle substituted with one or more substituents independently selected from halogen, —OR¹⁰, —SR¹⁰, —N(R¹⁰)₂, —C(O)R¹⁰, —C(O)OR¹⁰, —OC(O)R¹⁰, —NO₂, ═O, ═S, —CN, C₁₋₆ alkyl, C₂₋₆ alkenyl, and C₂₋₆ alkynyl. In certain embodiments, R⁵ is an optionally substituted 8- to 12-membered bicyclic carbocycle substituted with one or more substituents independently selected from —OR¹⁰, —N(R¹⁰)₂, and ═O. In some embodiments, R³ is an optionally substituted indane, and optionally substituted tetrahydronaphthalene.

In some embodiments, R³ is an optionally substituted unsaturated C₄₋₈ carbocycle. In certain embodiments, R³ is an optionally substituted unsaturated C₄₋₆ carbocycle. In certain embodiments, R³ is an optionally substituted unsaturated C₄₋₆ carbocycle with one or more substituents independently selected from optionally substituted C₃₋₁₂ carbocycle, and optionally substituted 3- to 12-membered heterocycle. R³ may be an optionally substituted unsaturated C₄₋₆ carbocycle with one or more substituents independently selected from optionally substituted phenyl, optionally substituted 3- to 12-heterocycle, optionally substituted C₁₋₁₀ alkyl, optionally substituted C₂₋₁₀ alkenyl, and halogen.

In some embodiments, R³ is selected from a 5- and 6-membered heteroaryl substituted with one or more substituents independently selected from R¹². In certain embodiments, R³ is selected from 5- and 6-membered heteroaryl substituted with one or more substituents independently selected from —C(O)CH₃, —C₁₋₃alkylene-NHC(O)OR¹⁰, —C₁₋₃alkylene-NHC(O)R¹⁰, —C₁₋₃alkylene-NHC(O)NHR¹⁰, and —C₁₋₃alkylene-NHC(O)—C₁₋₃alkylene-(R¹⁰); and 3- to 12-membered heterocycle, which is optionally substituted with one or more substituents selected from —OH, —N(R¹⁰)₂, —NHC(O)(R¹⁰)—NHC(O)O(R¹⁰), —NHC(O)N(R¹⁰)₂, —C(O)R¹⁰, —C(O)N(R¹⁰)₂, —C(O)₂R¹⁰, and —C₁₋₃alkylene-(R¹⁰), and R³ is optionally further substituted with one or more additional substituents independently selected from R¹². R³ may be selected from substituted pyridine, pyrazine, pyrimidine, pyridazine, furan, pyran, oxazole, thiazole, imidazole, pyrazole, oxadiazole, oxathiazole, and triazole, and R³ is optionally further substituted with one or more additional substituents independently selected from R¹². In some embodiments, R³ is substituted pyridine and R³ is optionally further substituted with one or more additional substituents independently selected from R¹². R³ may be represented as follows:

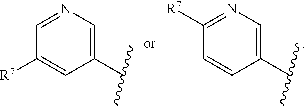

In some embodiments, R³ is substituted pyridine, and is substituted with —C₁₋₃alkylene-NHC(O)—C₁₋₃alkylene-R¹⁰ or —C₁alkylene-NHC(O)—C₁alkylene-NH₂.

R³ may be selected from:

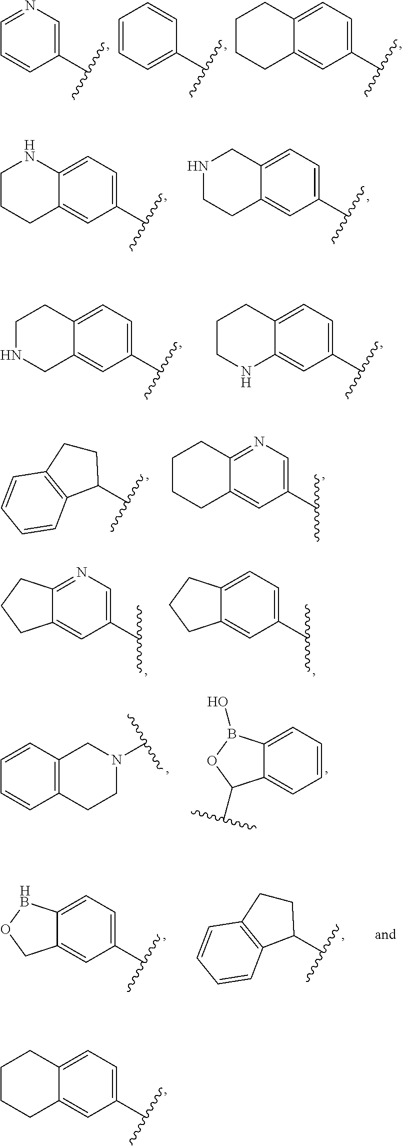

any one of which is optionally substituted. In some aspects, R³ may be selected from:

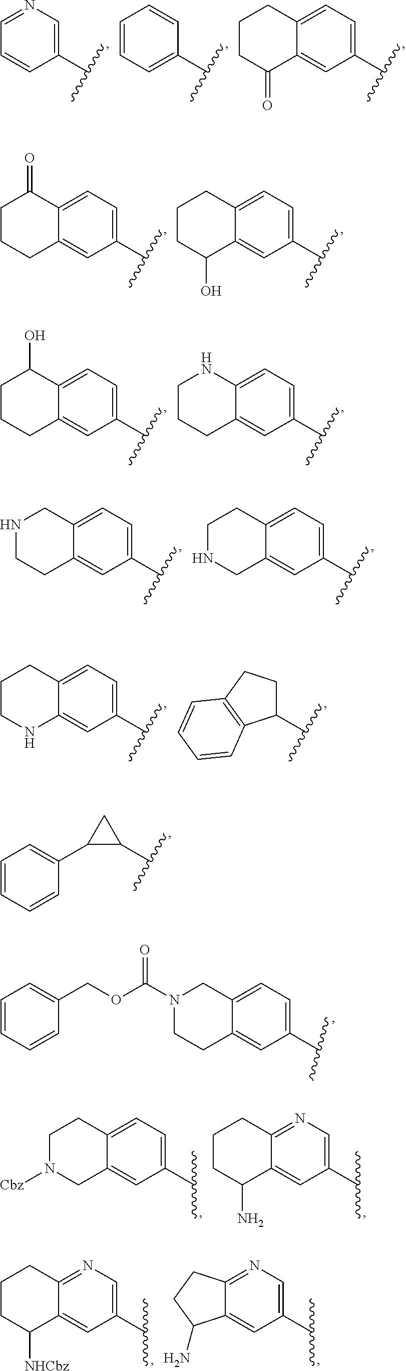

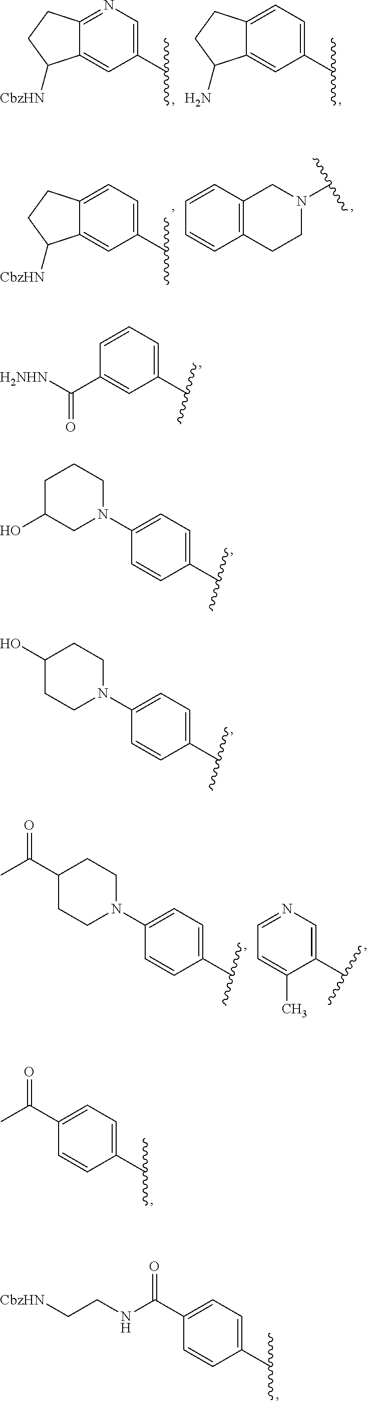

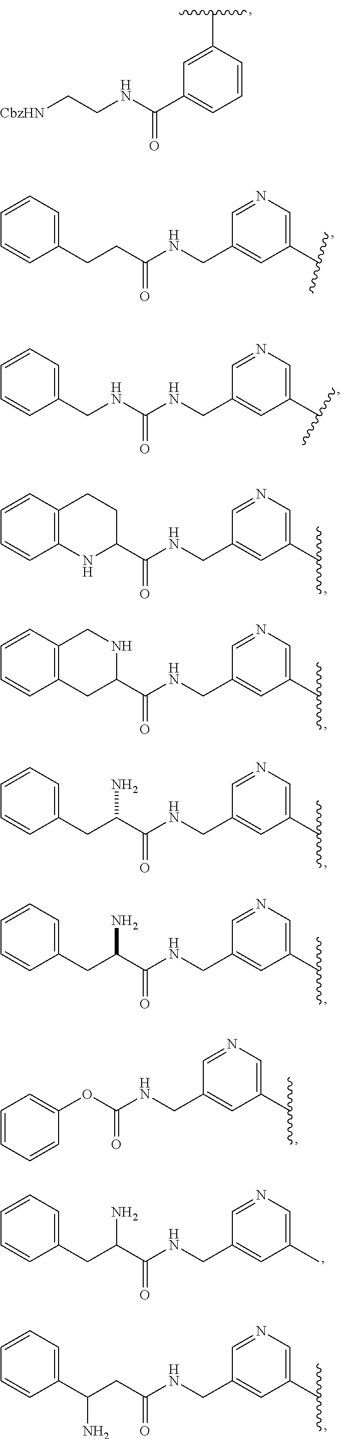

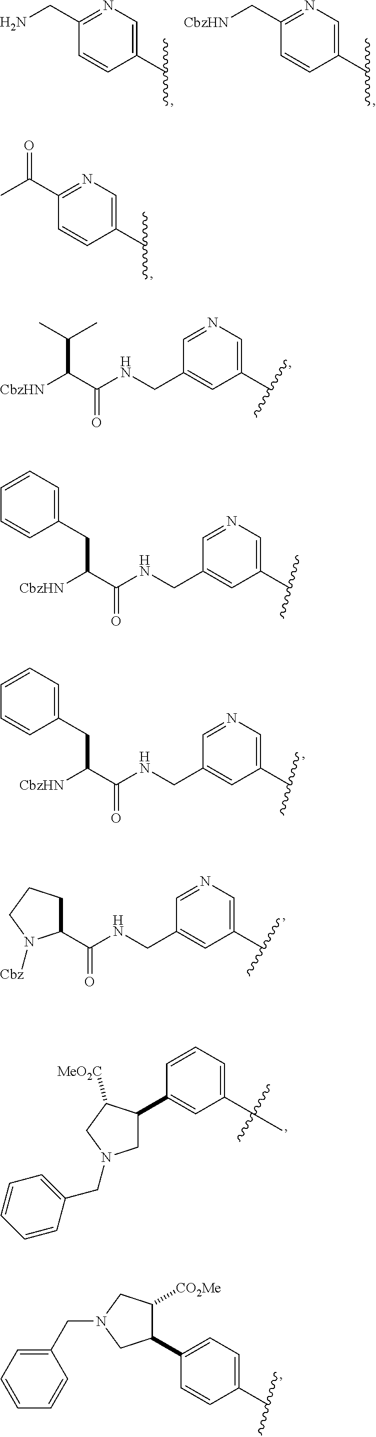

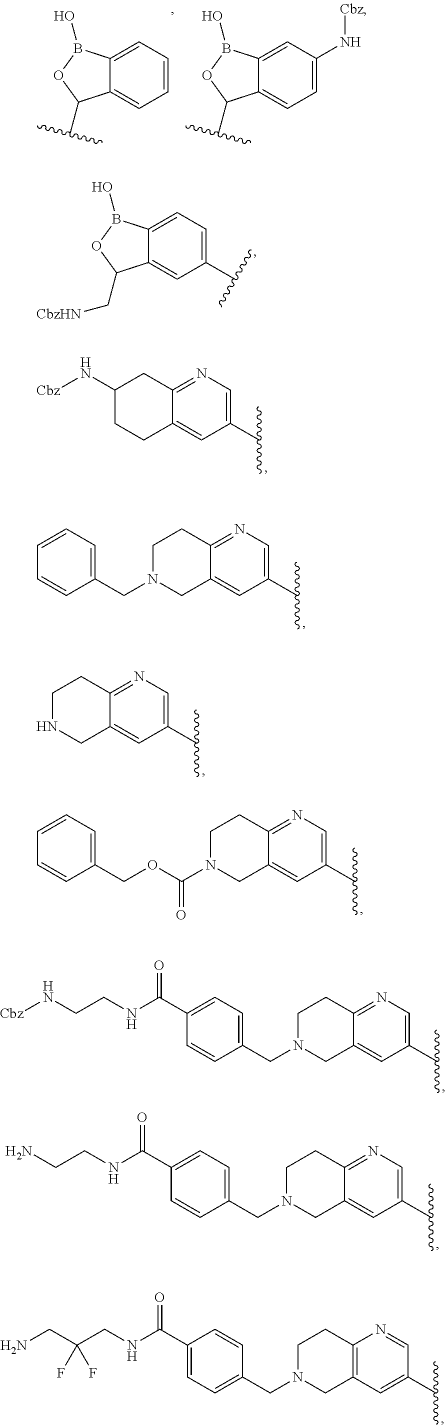

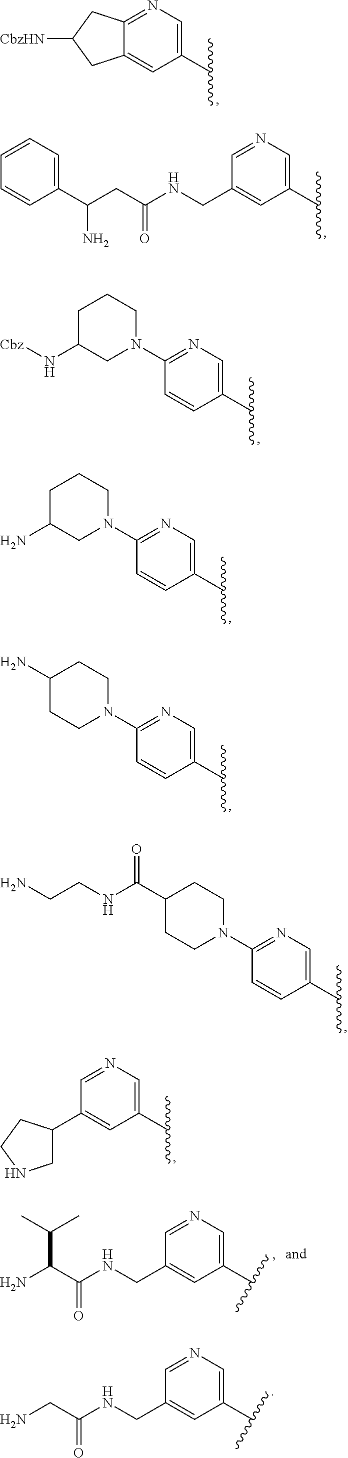

In some embodiments, when R³ is substituted, substituents on R³ are independently selected at each occurrence from: halogen, —OR, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and C₃₋₁₂ carbocycle, and 3- to 12-membered heterocycle, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR, —OC(O)R¹⁰, —NO₂, ═O, ═S, ═N(R¹⁰), —CN, C₁₋₆ alkyl, C₂₋₆ alkenyl, and C₂₋₆ alkynyl. In certain embodiments, the substituents on R³ are independently selected at each occurrence from: halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle. In certain embodiments, the substituents on R³ are independently selected at each occurrence from: halogen, —OR¹⁰, —SR¹⁰, —C(O)N(R¹⁰)₂, —N(R¹⁰)C(O)R¹⁰, —N(R¹⁰)₂, —C(O)R¹⁰, —C(O)OR¹⁰, —OC(O)R¹⁰, —NO₂, ═O, and —CN; and C₁₋₁₀ alkyl optionally substituted with one or more substituents independently selected from halogen, —OR¹⁰, —SR¹⁰, —N(R¹⁰)₂, —C(O)R¹⁰, —C(O)OR¹⁰, —NO₂, ═O, and —CN. In some embodiments, R³ is not substituted.

In some embodiments, L² is selected from —C(O)—, and —C(O)NR¹⁰—. In certain embodiments, L² is —C(O)—. In certain embodiments, L² is selected from —C(O)NR¹⁰—. R¹⁰ of —C(O)NR¹⁰— may be selected from hydrogen and C₁₋₆ alkyl. For example, L² may be —C(O)NH—.

In some embodiments, R⁴ is selected from: —OR¹⁰, —N(R¹⁰)₂, —C(O)N(R¹⁰)₂, —C(O)R¹⁰, —C(O)OR¹⁰, —S(O)R¹⁰, and —S(O)₂R¹⁰; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and C₃₋₁₂ carbocycle, and 3- to 12-membered heterocycle, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰), —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl.

In some embodiments, R⁴ is selected from: —OR¹⁰, —N(R¹⁰)₂, —C(O)N(R¹⁰)₂, —C(O)R¹⁰, —C(O)OR¹⁰, —S(O)R¹⁰, and —S(O)₂R¹⁰; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle. In some embodiments, R⁴ is selected from: —OR¹⁰, and —N(R¹⁰)₂; and C₁₋₁₀ alkyl, C₂₋₁₀ alkenyl, C₂₋₁₀ alkynyl, C₃₋₁₂ carbocycle, and 3- to 12-membered heterocycle, each of which is optionally substituted with one or more substituents independently selected from halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl. In certain embodiments, R⁴ is —N(R¹⁰)₂. R¹⁰ of —N(R¹⁰)₂ may be independently selected at each occurrence from optionally substituted C₁₋₆ alkyl. In certain embodiments, R¹⁰ of —N(R¹⁰)₂ is independently selected at each occurrence from methyl, ethyl, propyl, and butyl, any one of which is optionally substituted. For example, R⁴ may be

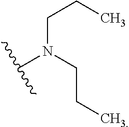 [SMILES: *N(CCC)CCC]

In certain embodiments, L²-R⁴ is

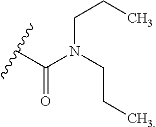 [SMILES: *C(=O)N(CCC)CCC]

In some embodiments, L¹¹ is —C(O)N(R¹⁰)—*. In some embodiments, R¹⁰ of —C(O)N(R¹⁰)—* is hydrogen or C₁₋₆ alkyl. For example, L¹¹ may be —C(O)NH—*.

In some embodiments, R¹² is independently selected at each occurrence from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), and —CN; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), —CN, C₃₋₁₀ carbocycle and 3- to 10-membered heterocycle; and C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), —CN, C₁₋₆ alkyl, C₂₋₆ alkenyl, C₂₋₆ alkynyl. In some embodiments, R¹² is independently selected at each occurrence from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), and —CN; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), —CN, C₃₋₁₀ carbocycle and 3- to 10-membered heterocycle.

In some aspects, the compound comprises a structure of Formula (XIV):

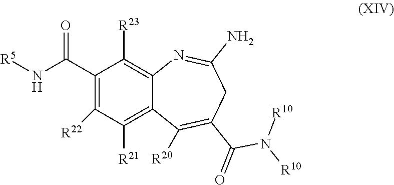

or a pharmaceutically acceptable salt thereof,
wherein:
R¹⁰ is independently selected at each occurrence from hydrogen, —NH₂, —C(O)OCH₂C₆H₅; and C₁₋₁₀ alkyl, C₂₋₁₀ alkenyl, C₂₋₁₀ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle, and haloalkyl;
R²⁰, R²¹, R²², and R²³ are independently selected from hydrogen, halogen, —OR¹⁰, —SR¹⁰, —N(R¹⁰)₂, —S(O)R¹⁰, —S(O)₂R¹, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl; and
R⁵ is a C₃₋₁₂ carbocycle or C₃₋₁₂ membered heterocycle (preferably a fused 5-5, fused 5-6, or fused 6-6 bicyclic heterocycle); wherein R⁵ is optionally substituted and wherein substituents are independently selected at each occurrence from: halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and C₃₋₁₂ carbocycle, and 3- to 12-membered heterocycle, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl.

Structures of Formula (XIV) include:

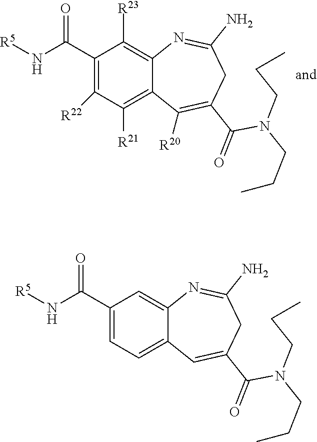

or a pharmaceutically acceptable salt thereof.

In some embodiments, R⁵ is selected from an optionally substituted fused 5-5, fused 5-6, and fused 6-6 bicyclic heterocycle. In certain embodiments, R⁵ is an optionally substituted fused 5-5, fused 5-6, and fused 6-6 bicyclic heterocycle with one or more substituents independently selected from —C(O)OR¹⁰, —N(R¹⁰)₂, —OR¹⁰, and optionally substituted C₁₋₁₀ alkyl. In certain embodiments, R⁵ is an optionally substituted fused 5-5, fused 5-6, and fused 6-6 bicyclic heterocycle substituted with —C(O)OR¹⁰. In certain embodiments, R⁵ is an optionally substituted fused 6-6 bicyclic heterocycle. For example, the fused 6-6 bicyclic heterocycle may be an optionally substituted pyridine-piperidine. In some embodiments, L¹⁰ is bound to a carbon atom of the pyridine of the fused pyridine-piperidine. In certain embodiments, R⁵ is selected from tetrahydroquinoline, tetrahydroisoquinoline, tetrahydronaphthyridine, cyclopentapyridine, and dihydrobenzoxaborole, any one of which is optionally substituted. R⁵ may be an optionally substituted tetrahydronaphthyridine. In some embodiments, R⁵ is:

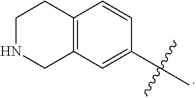 [SMILES: *c1ccc2c(c1)CNCC2]

In some preferred aspects, the compound is attached to a linker through R⁵ or a substituent on R⁵. In some aspects, attachment of the linker of R⁵ is at the position marked with the asterisk:

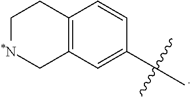

In some embodiments, the compound comprises a structure selected from:

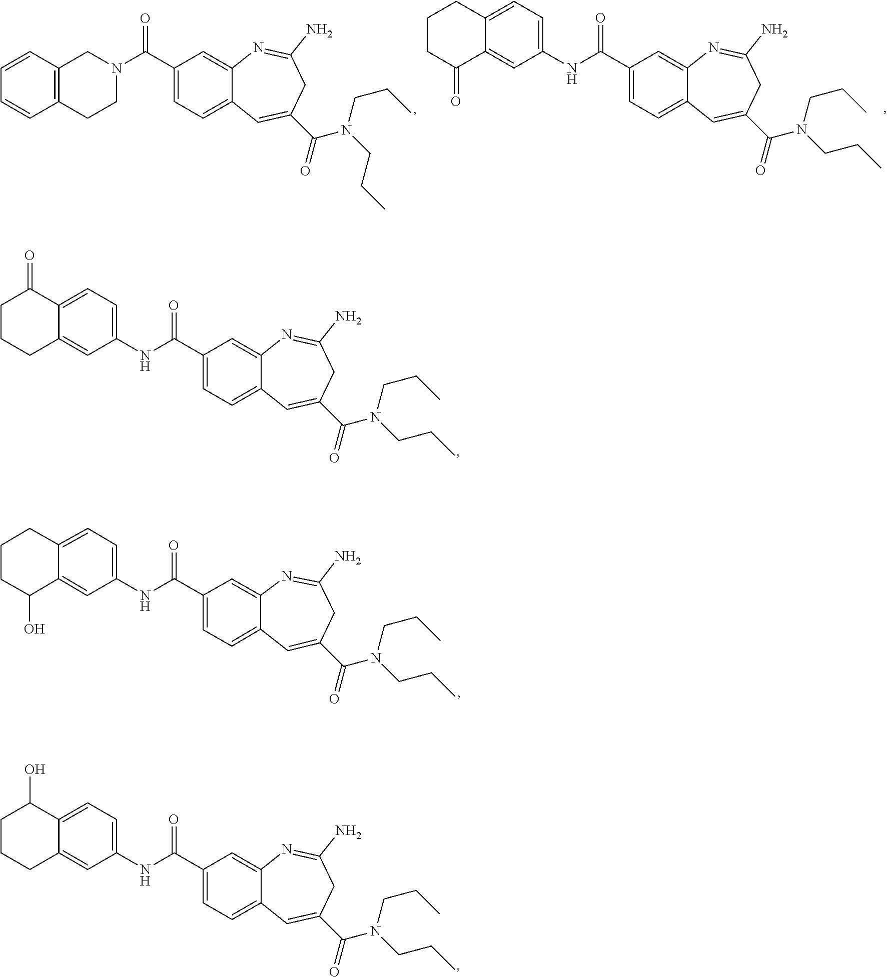 [SMILES: CCCN(CCC)C(=O)C1=Cc2ccc(C(=O)N3CCc4ccccc4C3)cc2N=C(N)C1 ; CCCN(CCC)C(=O)C1=Cc2ccc(C(=O)Nc3ccc4c(c3)C(=O)CCC4)cc2N=C(N)C1 ; CCCN(CCC)C(=O)C1=Cc2ccc(C(=O)Nc3ccc4c(c3)CCCC4=O)cc2N=C(N)C1 ; CCCN(CCC)C(=O)C1=Cc2ccc(C(=O)Nc3ccc4c(c3)C(O)CCC4)cc2N=C(N)C1 ; CCCN(CCC)C(=O)C1=Cc2ccc(C(=O)Nc3ccc4c(c3)CCCC4O)cc2N=C(N)C1]

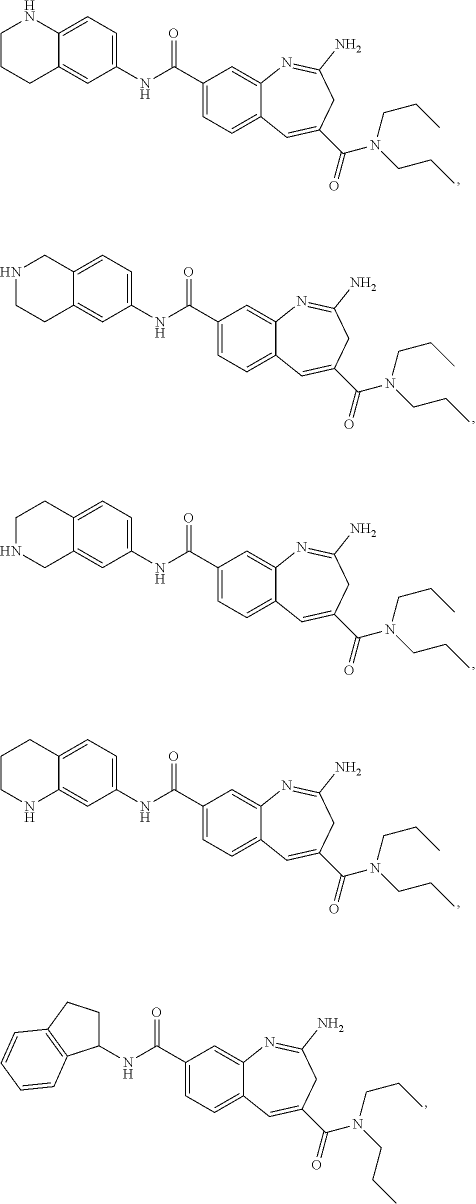 [SMILES: CCCN(CCC)C(=O)C1=Cc2ccc(C(=O)Nc3ccc4c(c3)CCCN4)cc2N=C(N)C1 ; CCCN(CCC)C(=O)C1=Cc2ccc(C(=O)Nc3ccc4c(c3)CCNC4)cc2N=C(N)C1 ; CCCN(CCC)C(=O)C1=Cc2ccc(C(=O)Nc3ccc4c(c3)CNCC4)cc2N=C(N)C1 ; CCCN(CCC)C(=O)C1=Cc2ccc(C(=O)Nc3ccc4c(c3)NCCC4)cc2N=C(N)C1 ; CCCN(CCC)C(=O)C1=Cc2ccc(C(=O)NC3CCc4ccccc43)cc2N=C(N)C1]

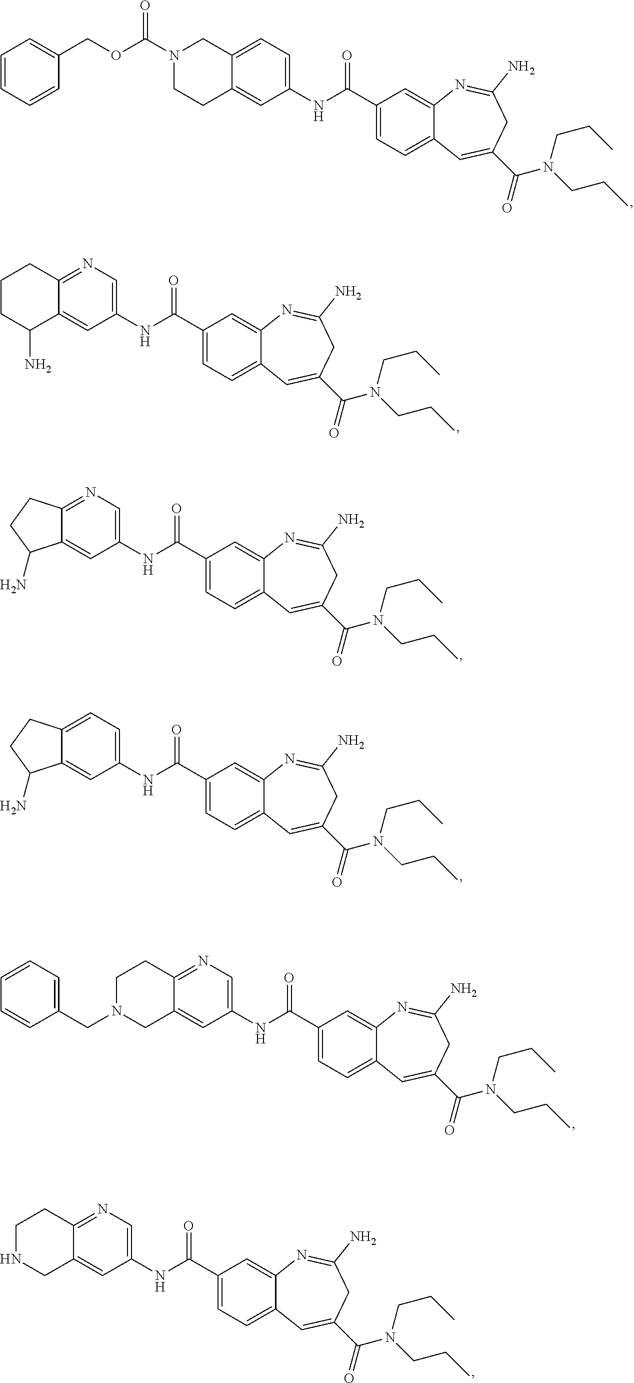 [SMILES: CCCN(CCC)C(=O)C1=Cc2ccc(C(=O)Nc3ccc4c(c3)CCN(C(=O)OCc3ccccc3)C4)cc2N=C(N)C1 ; CCCN(CCC)C(=O)C1=Cc2ccc(C(=O)Nc3cnc4c(c3)C(N)CCC4)cc2N=C(N)C1 ; CCCN(CCC)C(=O)C1=Cc2ccc(C(=O)Nc3cnc4c(c3)C(N)CC4)cc2N=C(N)C1 ; CCCN(CCC)C(=O)C1=Cc2ccc(C(=O)Nc3ccc4c(c3)C(N)CC4)cc2N=C(N)C1 ; CCCN(CCC)C(=O)C1=Cc2ccc(C(=O)Nc3cnc4c(c3)CN(Cc3ccccc3)CC4)cc2N=C(N)C1 ; CCCN(CCC)C(=O)C1=Cc2ccc(C(=O)Nc3cnc4c(c3)CNCC4)cc2N=C(N)C1]

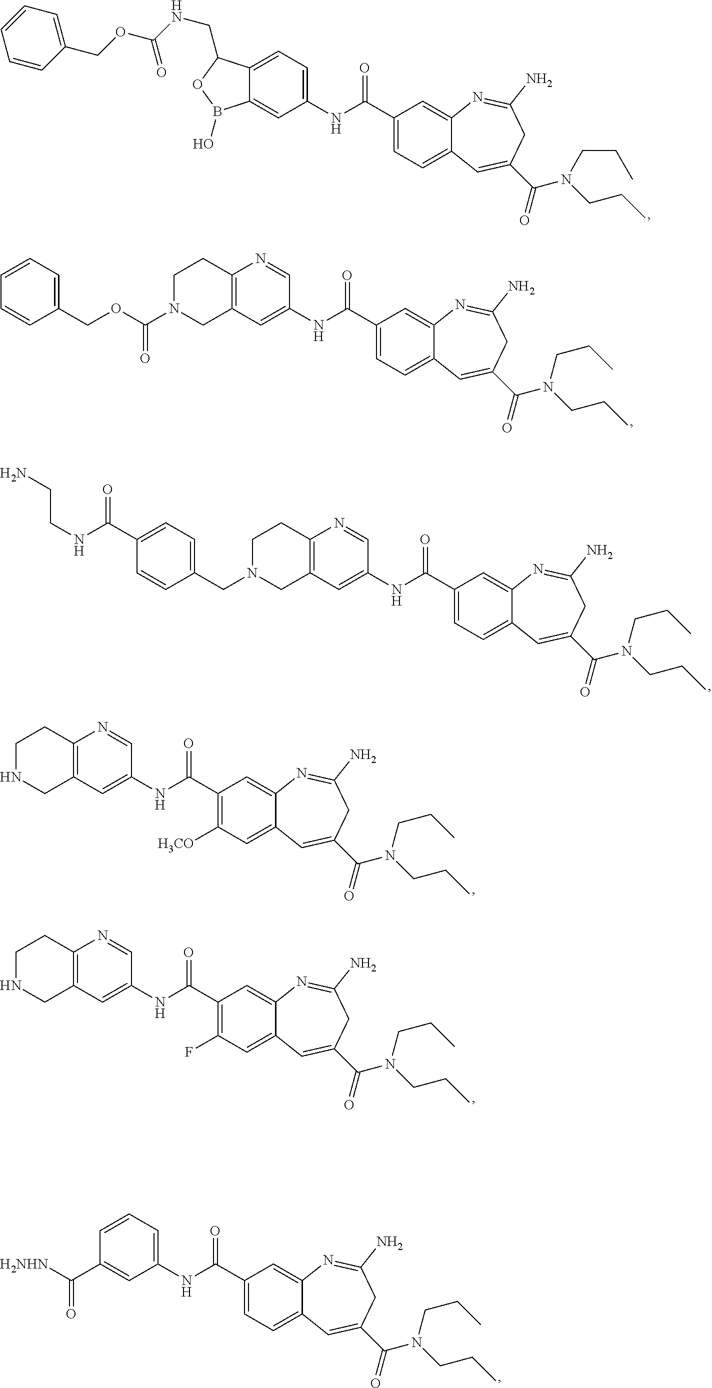

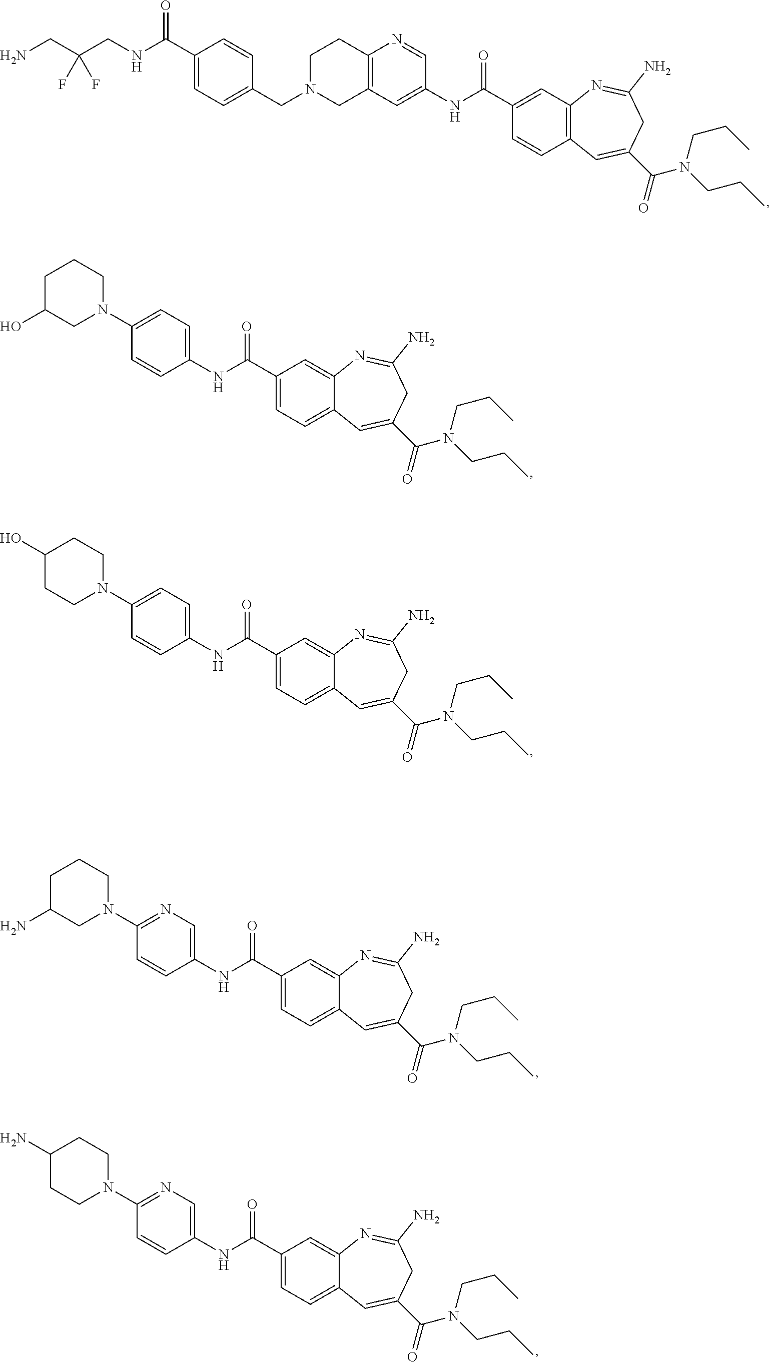 [SMILES: CCCN(CCC)C(=O)C1=Cc2ccc(C(=O)Nc3cnc4c(c3)CN(Cc3ccc(C(=O)NCC(F)(F)CN)cc3)CC4)cc2N=C(N)C1 ; CCCN(CCC)C(=O)C1=Cc2ccc(C(=O)Nc3ccc(N4CCCC(O)C4)cc3)cc2N=C(N)C1 ; CCCN(CCC)C(=O)C1=Cc2ccc(C(=O)Nc3ccc(N4CCC(O)CC4)cc3)cc2N=C(N)C1 ; CCCN(CCC)C(=O)C1=Cc2ccc(C(=O)Nc3ccc(N4CCCC(N)C4)nc3)cc2N=C(N)C1 ; CCCN(CCC)C(=O)C1=Cc2ccc(C(=O)Nc3ccc(N4CCC(N)CC4)nc3)cc2N=C(N)C1]

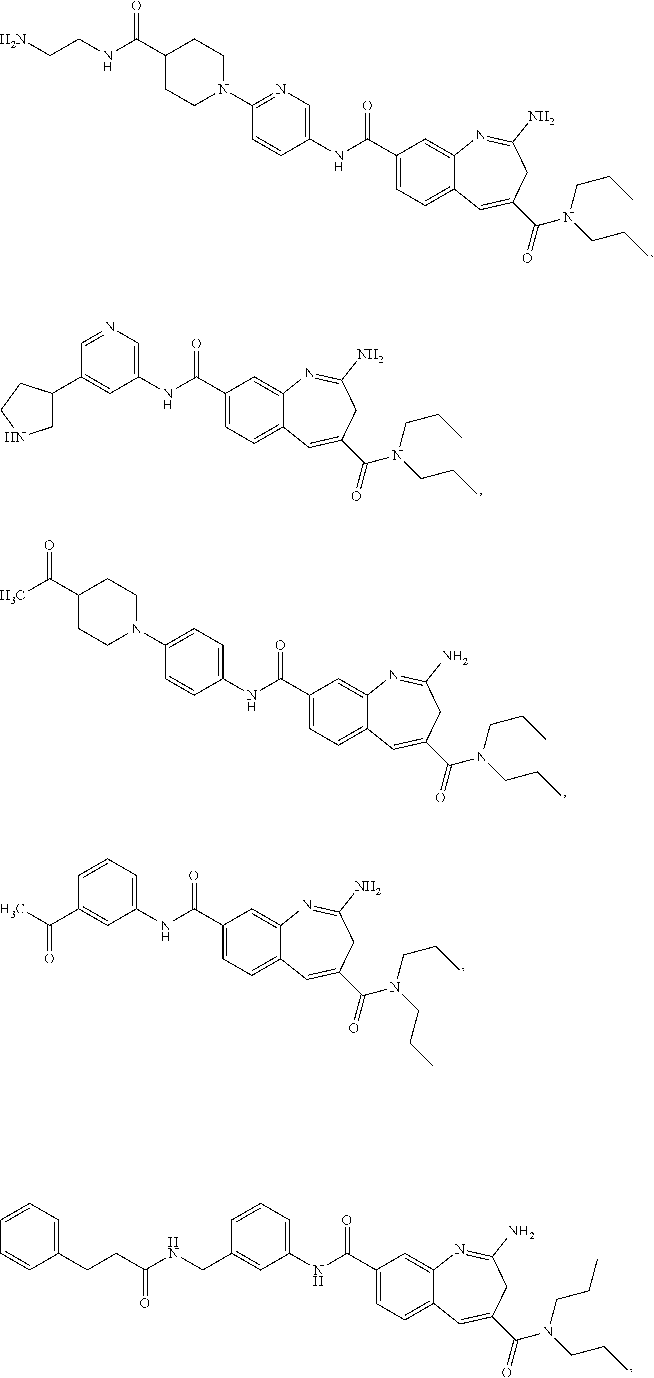

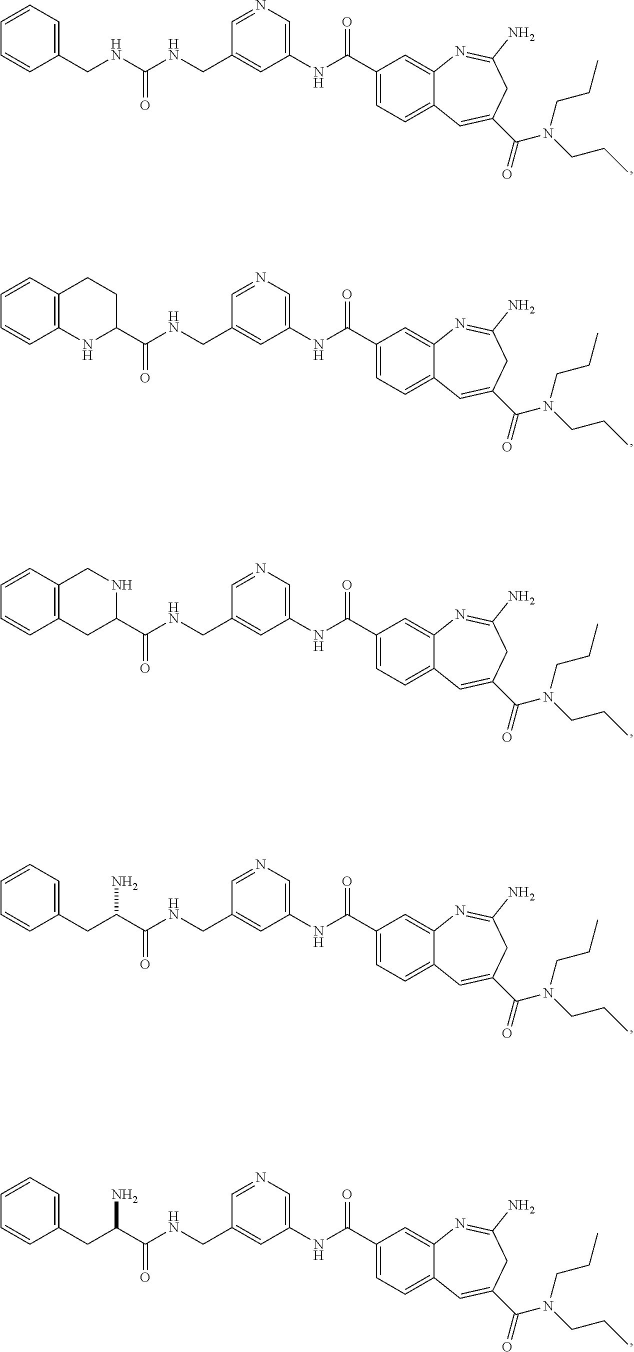 [SMILES: CCCN(CCC)C(=O)C1=Cc2ccc(C(=O)Nc3cncc(CNC(=O)NCc4ccccc4)c3)cc2N=C(N)C1 ; CCCN(CCC)C(=O)C1=Cc2ccc(C(=O)Nc3cncc(CNC(=O)C4CCc5ccccc5N4)c3)cc2N=C(N)C1 ; CCCN(CCC)C(=O)C1=Cc2ccc(C(=O)Nc3cncc(CNC(=O)C4Cc5ccccc5CN4)c3)cc2N=C(N)C1 ; CCCN(CCC)C(=O)C1=Cc2ccc(C(=O)Nc3cncc(CNC(=O)[C@@H](N)Cc4ccccc4)c3)cc2N=C(N)C1 ; CCCN(CCC)C(=O)C1=Cc2ccc(C(=O)Nc3cncc(CNC(=O)[C@H](N)Cc4ccccc4)c3)cc2N=C(N)C1]

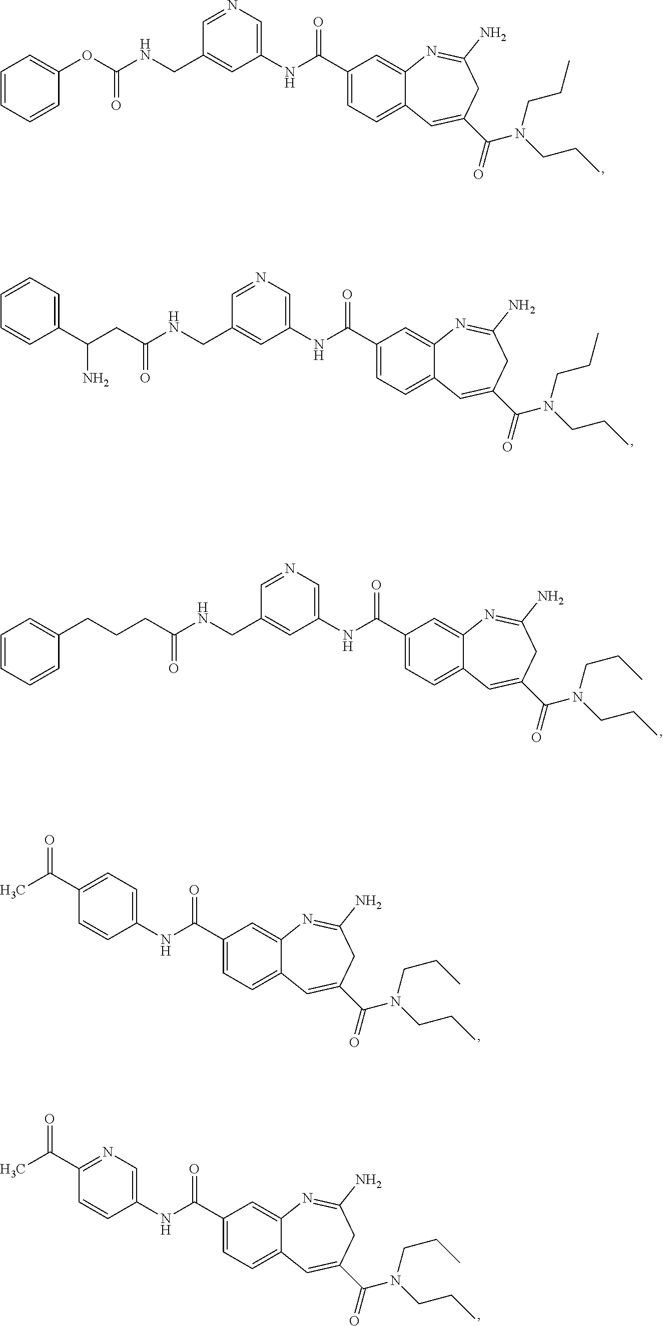

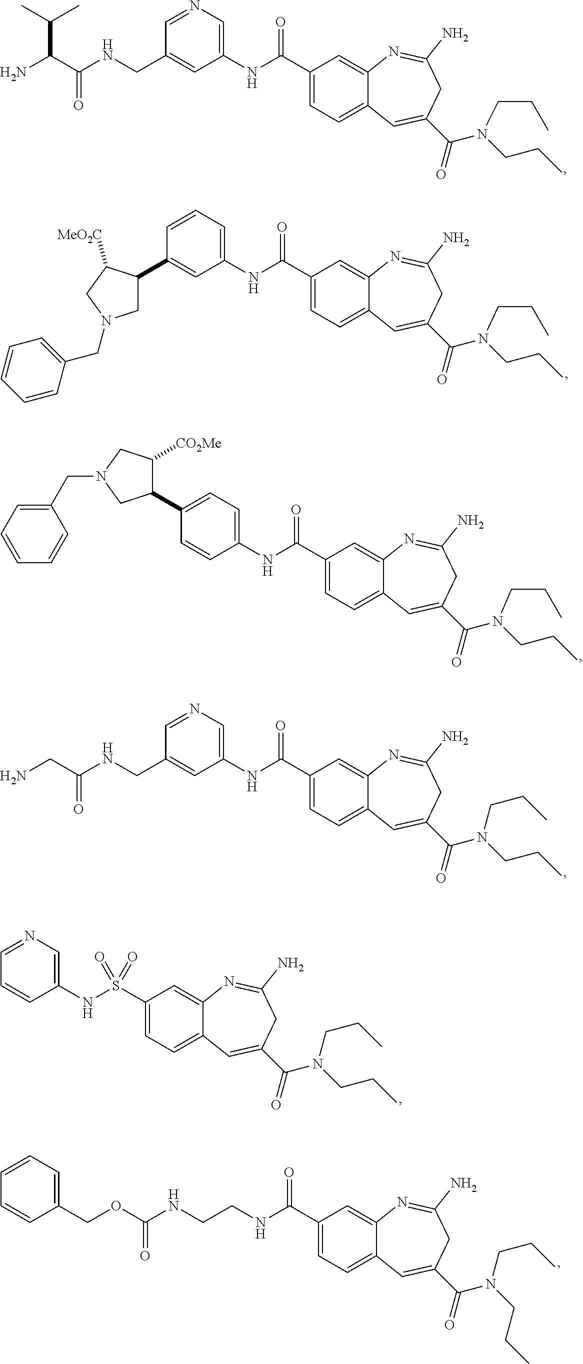

and salts thereof, including pharmaceutically acceptable salts thereof.

In some aspects, the conjugate is represented by Formula (I):

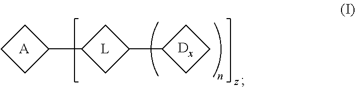

wherein:
A is a polypeptide;
L is a linker;
Dₓ is a benzazepine compound;
n is selected from 1 to 20; and
z is selected from 1 to 20.

In some aspects of Formula (I), n is 1.

In Formula (I), the drug loading is represented by z, the number of compound molecules per polypeptide, or the number of immune-stimulatory compounds per antibody, depending on the particular conjugate. Depending on the context, z can represent the average number of compound molecules per conjugate, also referred to the average drug loading. z can range from 1 to 20, from 1-50 or from 1-100. In some conjugates, z is preferably from 1 to 8. In some preferred embodiments, when z represents the average drug loading, z ranges from about 2 to about 5. In some embodiments, z is about 2, about 3, about 4, or about 5. The average number of compounds per conjugate (e.g., drug-antibody ratio, DAR) may be characterized by conventional means such as mass spectroscopy, liquid chromatography/mass spectrometry (LC/MS), HIC, ELISA assay, and HPLC. In some aspects, z is from 1 to 8. In some aspects, n is 1 and z is from 1 to 8.

In some aspects, L is a cleavable linker. In some aspects, L is a non-cleavable linker.

In some aspects of Formula (I), Dₓ is a structure of Formula (XI-A), (XI-B), or (XIV).

In some aspects of Formula (I), L and Dₓ together are a compound of Formula IVB:

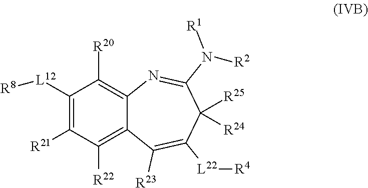

or a pharmaceutically acceptable salt thereof, wherein:
L¹² is selected from —X³—, —X³—C₁₋₆ alkylene-X³—, —X³—C₂₋₆ alkenylene-X³—, and —X³—C₂₋₆ alkynylene-X³—, each of which is optionally substituted on alkylene, alkenylene, or alkynylene with one or more substituents independently selected from R¹²;
L²² is independently selected from —X⁴—, —X⁴—C₁₋₆ alkylene-X⁴—, —X⁴—C₂₋₆ alkenylene-X⁴—, and —X⁴—C₂₋₆ alkynylene-X⁴—, each of which is optionally substituted on alkylene, alkenylene, or alkynylene with one or more substituents independently selected from R¹⁰;
X³ and X⁴ are independently selected at each occurrence from a bond, —O—, —S—, —N(R¹⁰)—, —C(O)—, —C(O)O—, —OC(O)—, —OC(O)O—, —C(O)N(R¹⁰)—, —C(O)N(R¹⁰)C(O)—, —C(O)N(R¹⁰)C(O)N(R¹⁰)—, —N(R¹⁰)C(O)—, —N(R¹⁰)C(O)N(R¹⁰)—, —N(R¹⁰)C(O)O—, —OC(O)N(R¹⁰)—, —C(NR¹⁰)—, —N(R¹⁰)C(NR¹⁰)—, —C(NR¹⁰)N(R¹⁰)—, —N(R¹⁰)C(NR¹⁰)N(R¹⁰)—, —S(O)₂—, —OS(O)—, —S(O)O—, —S(O)—, —OS(O)₂—, —S(O)₂O—, —N(R¹⁰)S(O)₂—, —S(O)₂N(R¹⁰)—, —N(R¹⁰)S(O)—, —S(O)N(R¹⁰)—, —N(R¹⁰)S(O)₂N(R¹⁰)—, and —N(R¹⁰)S(O)N(R¹⁰)—;
R¹ and R² are each hydrogen;
R⁴ and R⁸ are independently selected from: —OR¹⁰, —N(R¹⁰)₂, —C(O)N(R¹⁰)₂, —C(O)R¹⁰, —C(O)OR¹⁰, —S(O)R¹⁰, and —S(O)₂R¹⁰; C₁₋₁₀ alkyl, C₂₋₁₀ alkenyl, C₂₋₁₀ alkynyl, each of which is optionally bound to L³ and each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and C₃₋₁₂ carbocycle and 3- to 12-membered heterocycle, wherein each C₃₋₁₂ carbocycle and 3- to 12-membered heterocycle in R⁴ and R⁸ is optionally bound to L³ and each C₃₋₁₂ carbocycle and 3- to 12-membered heterocycle in R⁴ and R⁸ is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl;
R¹⁰ is independently selected at each occurrence from L³, hydrogen, —NH₂, —C(O)OCH₂C₆H₅; and C₁₋₁₀ alkyl, C₂₋₁₀ alkenyl, C₂₋₁₀ alkynyl, C₃₋₁₂ carbocycle, and 3- to 12-membered heterocycle, each of which is optionally substituted with one or more substituents independently selected from halogen, —CN, —NO₂, —NH₂, ═O, ═S, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and haloalkyl;
L³ is a linker moiety, wherein there is at least one occurrence of L³;
wherein any substitutable carbon on the benzazepine core is optionally substituted by a substituent independently selected from R¹² or two substituents on a single carbon atom combine to form a 3- to 7-membered carbocycle;
R¹² is independently selected at each occurrence from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), and —CN; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰), —NO₂, ═O, ═S, ═N(R¹⁰), —CN, C₃₋₁₀ carbocycle and 3- to 10-membered heterocycle; and C₃₋₁₀ carbocycle and 3- to 10-membered heterocycle, wherein each C₃₋₁₀ carbocycle and 3- to 10-membered heterocycle in R¹² is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰), —NO₂, ═O, ═S, ═N(R¹⁰), —CN, C₁₋₆ alkyl, C₂₋₆ alkenyl, C₂₋₆ alkynyl; and
R²⁰, R²¹, R²², and R²³ are independently selected from hydrogen, halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl; and
R²⁴ and R²⁵ are independently selected from hydrogen, halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl; or R²⁴ and R²⁵ taken together form an optionally substituted saturated C₃₋₇ carbocycle.

In some aspects of Formula (I), L and Dₓ together are a compound of Formula (IVC):

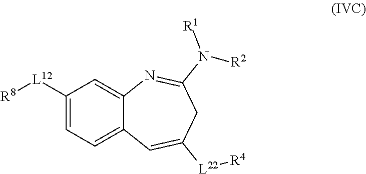

or a pharmaceutically acceptable salt thereof,
wherein:
R¹ and R² are each hydrogen;

### L²² is —C(O)—;

R⁴ is —N(R¹⁰)₂;
R¹⁰ is independently selected at each occurrence from hydrogen, —NH₂, —C(O)OCH₂C₆H₅; and C₁₋₁₀ alkyl, C₂₋₁₀ alkenyl, C₂₋₁₀ alkynyl, C₃₋₁₂ carbocycle, and 3- to 12-membered heterocycle, each of which is optionally substituted with one or more substituents independently selected from halogen, —CN, —NO₂, —NH₂, ═O, ═S, —C(O)OCH₂C₆H, —NHC(O)OCH₂C₆H, C₁₋₁₀ alkyl, C₂₋₁₀ alkenyl, C₂₋₁₀ alkynyl, C₃₋₁₂ carbocycle, 3- to 12-membered heterocycle, and haloalkyl;
L¹² is —C(O)N(R¹⁰)—*, wherein * represents where L¹² is bound to R⁸;
R⁸ is an optionally substituted fused 5-5, fused 5-6, or fused 6-6 bicyclic heterocycle bound to linker moiety L³,
and wherein optional substituents are independently selected at each occurrence from:
   halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN;
   C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and
   C₃₋₁₂ carbocycle and 3- to 12-membered heterocycle, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl.

In some aspects of Formula (IVB) and Formula (IVC), L¹² is —C(O)N(R¹⁰)—. In some embodiments, R¹⁰ of —C(O)N(R¹⁰)— is selected from hydrogen, C₁₋₆ alkyl, and L³. For example, L¹² may be —C(O)NH—.

In some embodiments, R⁸ is an optionally substituted 5- or 6-membered heteroaryl. R⁸ may be an optionally substituted 5- or 6-membered heteroaryl, bound to L³. In some embodiments, R⁸ is an optionally substituted pyridine, bound to L³.

In some embodiments, L²² is selected from —C(O)—, and —C(O)NR¹⁰—. In certain embodiments, L²² is —C(O)—. In certain embodiments, L²² is —C(O)NR¹⁰—. R¹⁰ of —C(O)NR¹⁰— may be selected from hydrogen, C₁₋₆ alkyl, and -L³. For example, L²² may be —C(O)NH—.

In some embodiments, R⁴ is selected from: —OR¹⁰, and —N(R¹⁰)₂; and C₁₋₁₀ alkyl, C₂₋₁₀ alkenyl, C₂₋₁₀ alkynyl, C₃₋₁₂ carbocycle, 3- to 12-membered heterocycle, aryl, and heteroaryl, each of which is optionally substituted with one or more substituents independently selected from halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl and each of which is further optionally bound to L³. In some embodiments, R⁴ is —N(R¹⁰)₂ and R¹⁰ of —N(R¹⁰)₂ is selected from L³ and hydrogen, and wherein at least one R¹⁰ of —N(R¹⁰)₂ is L³. In some aspects, R⁴ is —N(C₁₋₄ alkyl)₂ and L¹² is —C(O)N(H)—*. In some aspects of Formula (IVB) and (IVC), R⁴ is

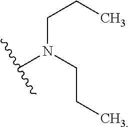 [SMILES: *N(CCC)CCC]

In certain embodiments: R¹⁰ of —N(R¹⁰)₂ is independently selected at each occurrence from methyl, ethyl, propyl, and butyl, any one of which is optionally substituted. In certain embodiments, R¹⁰ of —C(O)N(R¹⁰)—* is hydrogen.

In some embodiments, L³ is a noncleavable linker. In some embodiments, L³ is a cleavable linker. L³ may be cleavable by a lysosomal enzyme. In some embodiments, the compound is covalently attached to a polypeptide, such as an antibody. In some embodiments, the compound is covalently attached to a polypeptide, optionally through the linker. In some embodiments, the polypeptide is a targeting moiety or antibody that specifically binds to a tumor antigen.

In some embodiments, L³ is represented by the formula:

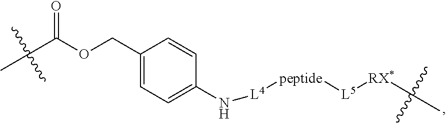

wherein
L⁴ represents the C-terminal of the peptide;
L⁵ is selected from a bond, alkylene and heteroalkylene,
wherein L⁵ is optionally substituted with one or more groups independently selected from R³²;
RX* comprises a bond, a succinimide moiety, or a hydrolyzed succinimide moiety bound to a residue of a polypeptide, such as an antibody,
   wherein

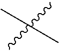

on RX* represents the point of attachment to the residue of the polypeptide; and,
R³² is independently selected at each occurrence from halogen, —OH, —CN, —O-alkyl, —SH, ═O, ═S, —NH₂, —NO₂; and C₁₋₁₀ alkyl, C₂₋₁₀ alkenyl, C₂₋₁₀ alkynyl, each of which is optionally substituted with one or more substituents independently selected from halogen, —OH, —CN, —O-alkyl, —SH, ═O, ═S, —NH₂, —NO₂. In some embodiments, the peptide of L³ comprises Val-Cit or Val-Ala or Glu-Val-Cit.

In some aspects of Formula (I), L and Dₓ together have a structure selected from:

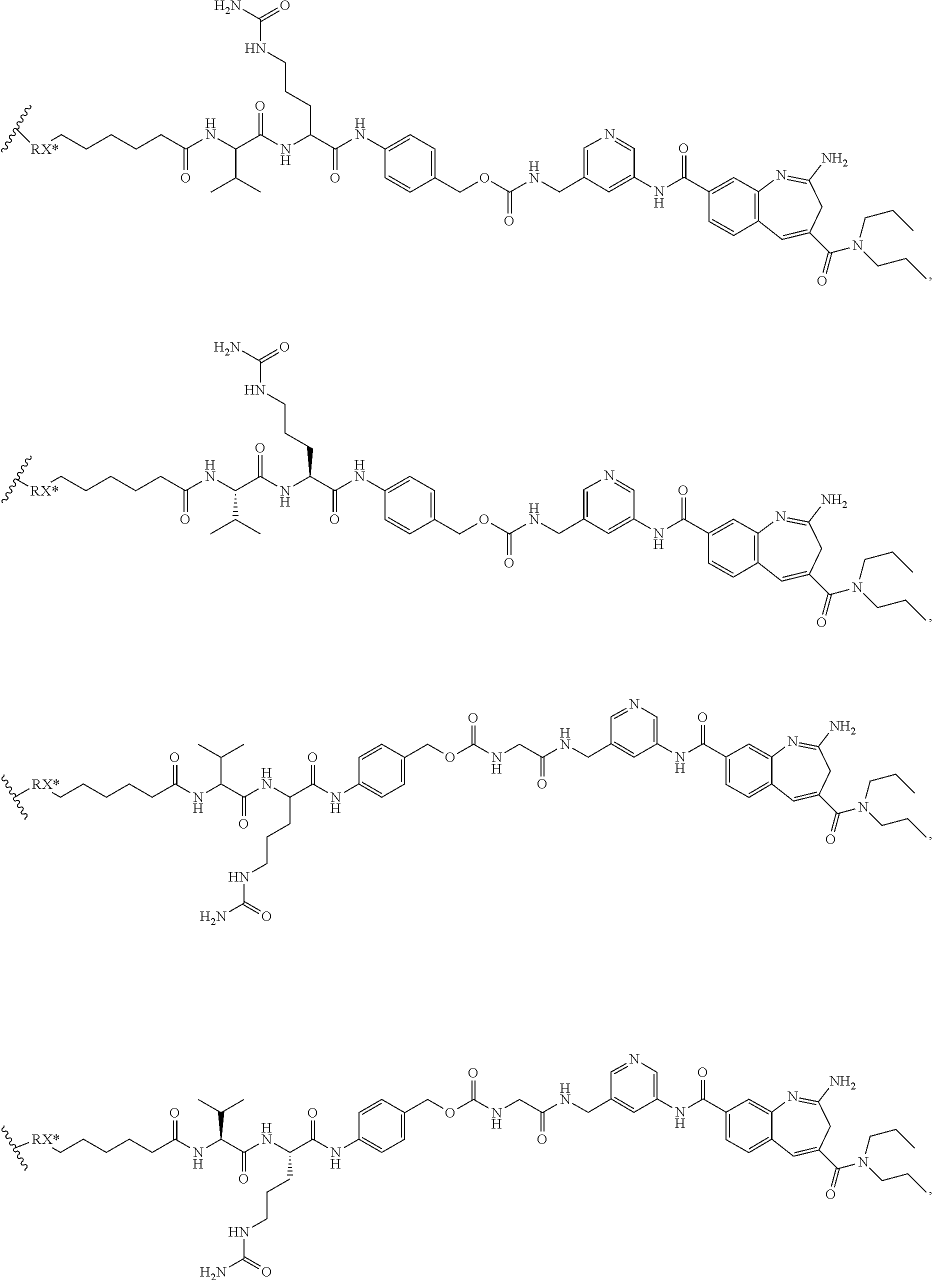

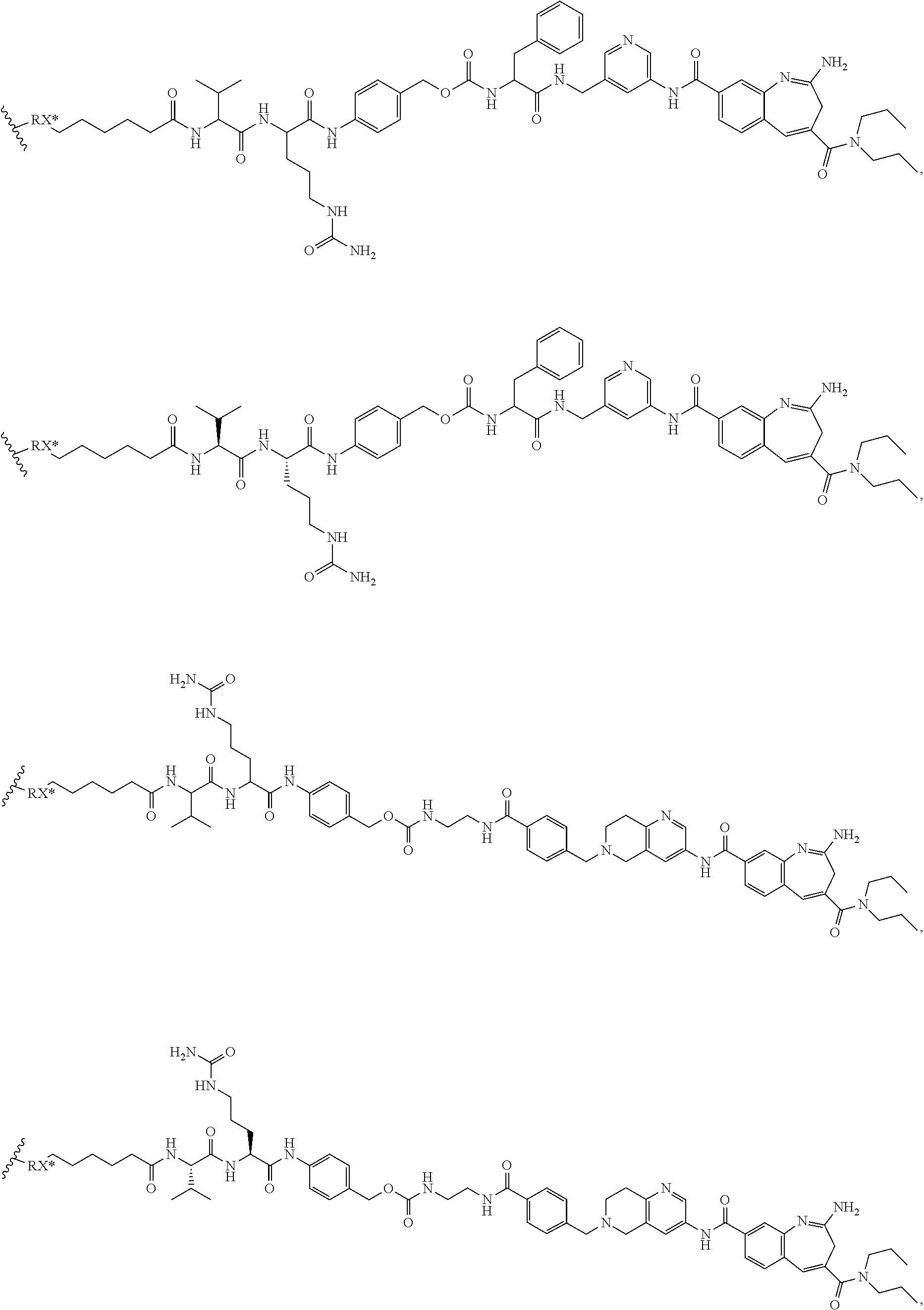

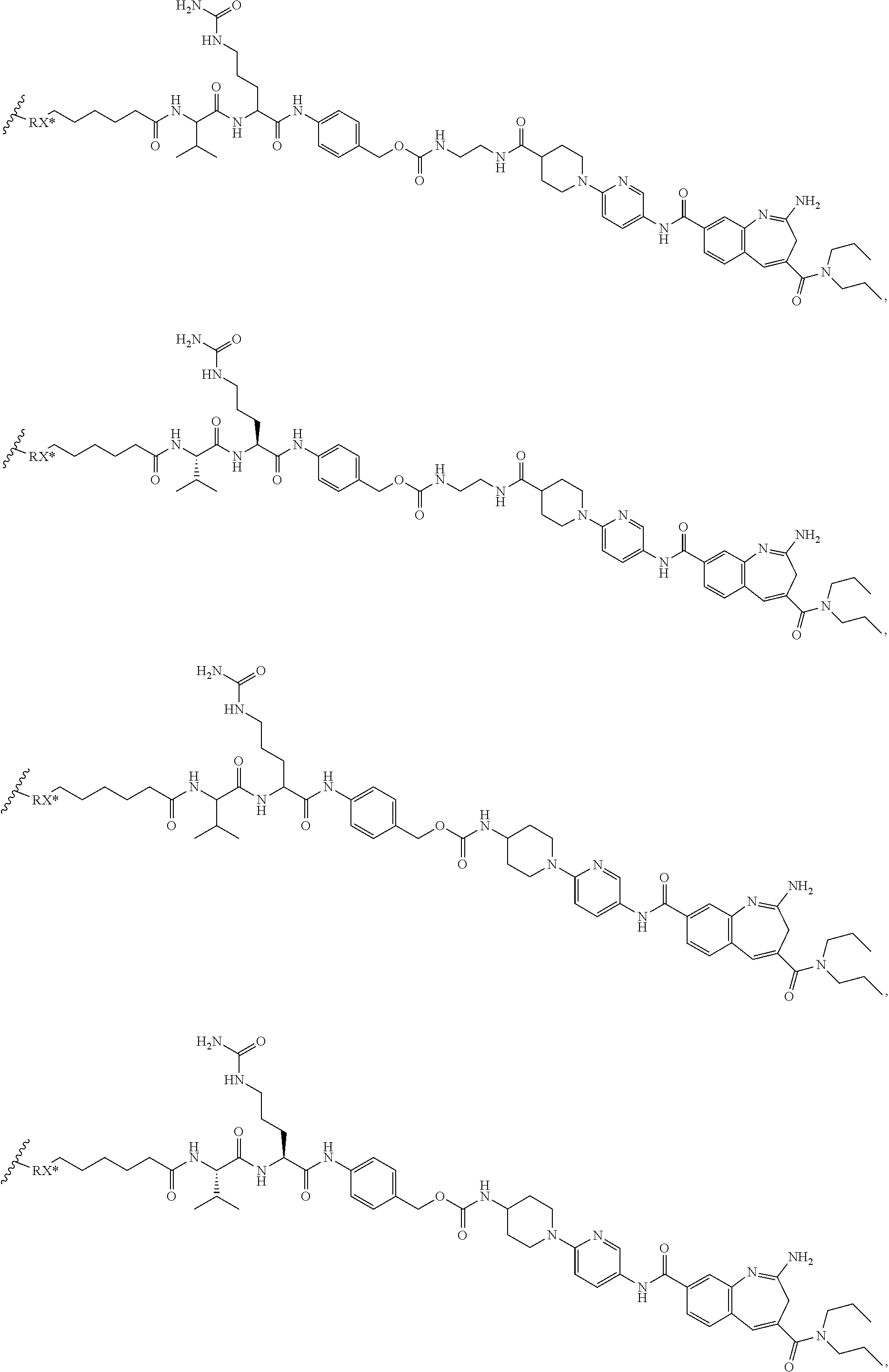

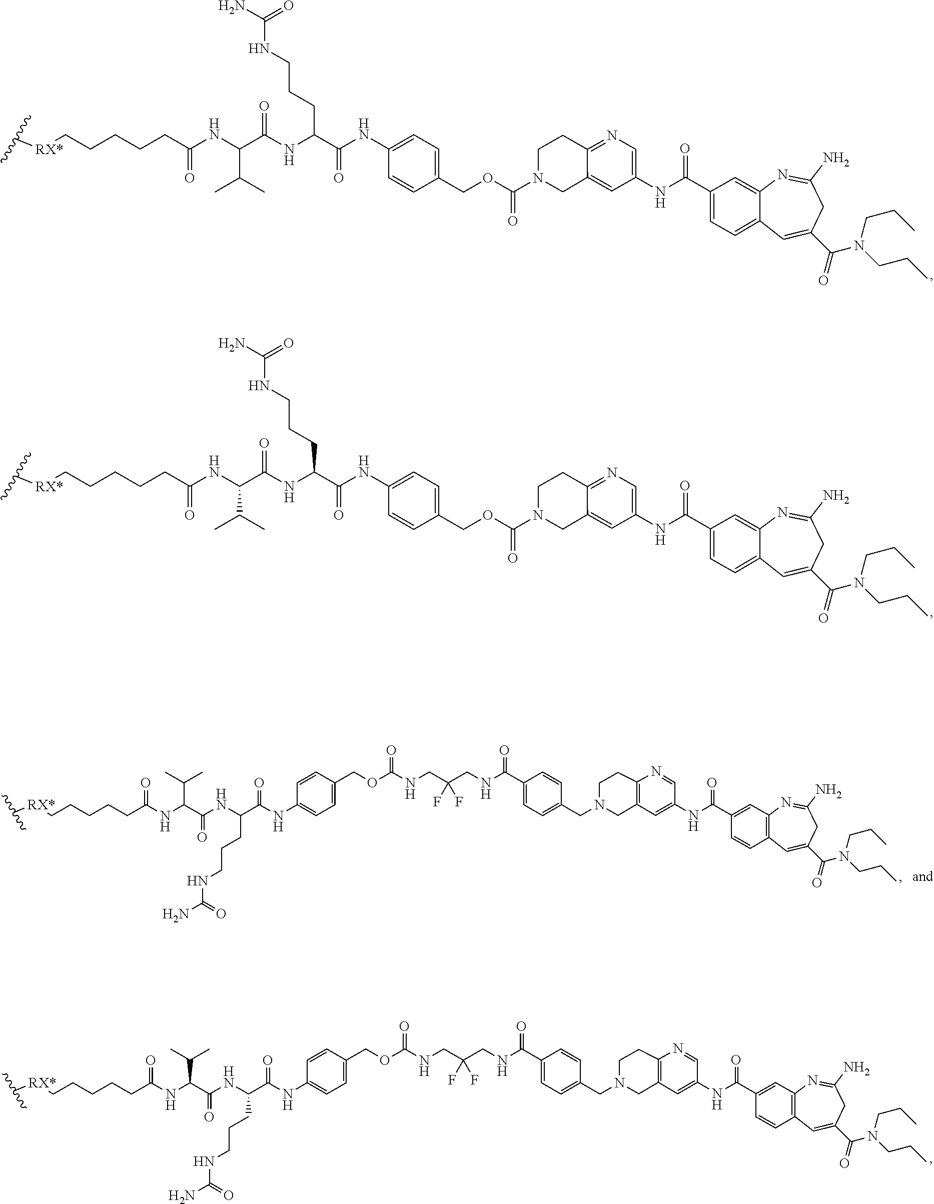

wherein the RX* is a bond, a succinimide moiety, or a hydrolyzed succinimide moiety bound to a residue of a polypeptide, such as an antibody,
wherein

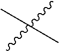

on RX* represents the point of attachment to the residue of the polypeptide.

In some embodiments, L³ is represented by the formula:

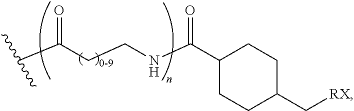

wherein RX comprises a reactive moiety, and n=0-9. In some embodiments, RX comprises a leaving group. In some embodiments, RX comprises a maleimide. In some embodiments, L³ is represented as follows:

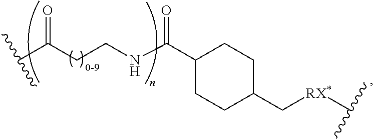

wherein RX* comprises a bond, a succinimide moiety, or a hydrolyzed succinimide moiety bound to a residue of a polypeptide, such as an anibody, wherein

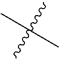

on RX*represents the point of attachment to the residue of the polypeptide, and n=0-9.

In some aspects, the compound comprises a structure selected from:

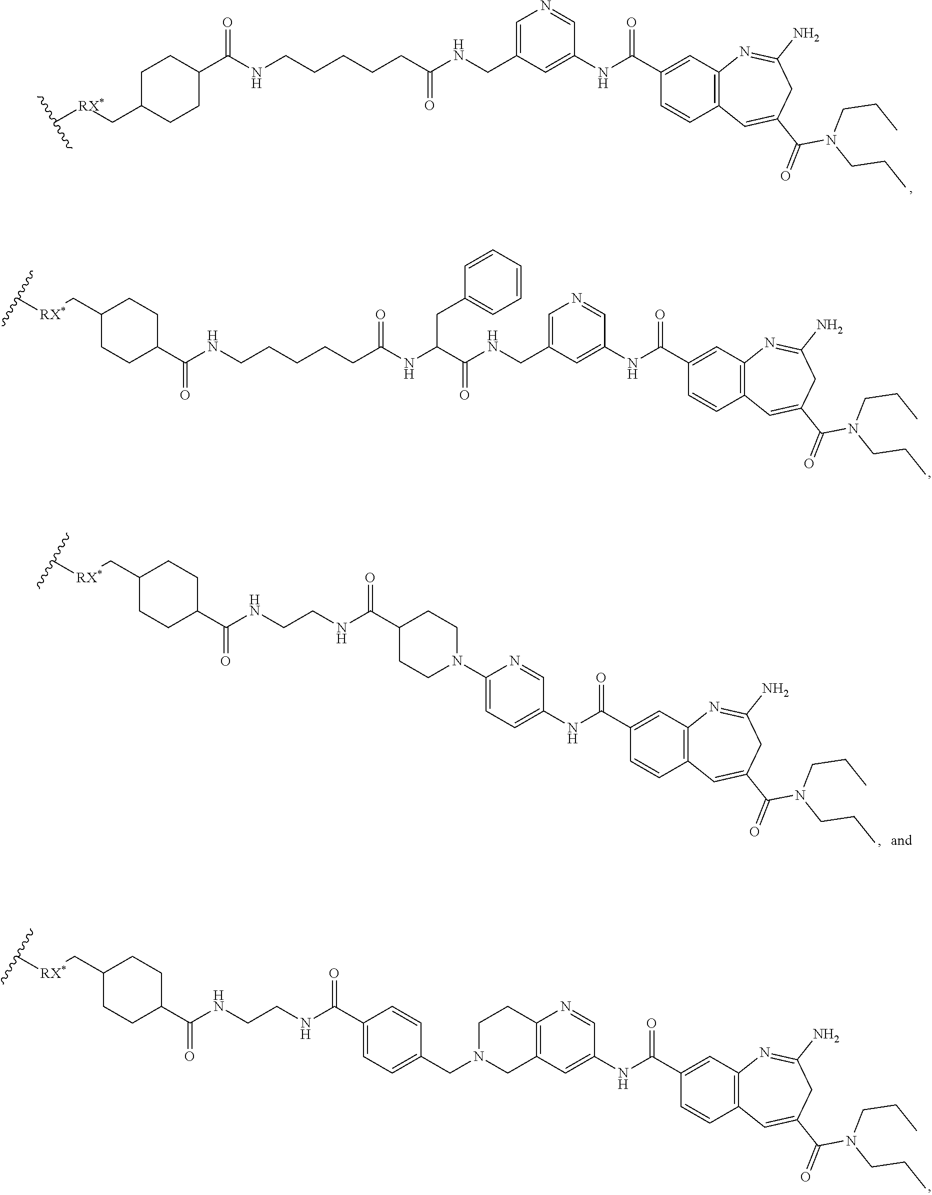

and a salt of any one thereof, wherein the RX* comprises a bond, a succinimide moiety, or a hydrolyzed succinimide moiety bound to a residue of a polypeptide, such as an antibody, wherein

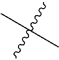

on RX* represents the point of attachment to the residue of polypeptide.

In some embodiments, RX* comprises a succinamide moiety and is bound to a cysteine residue of a polypeptide, such as an antibody. In some embodiments, RX* comprises a hydrolyzed succinamide moiety and is bound to a cysteine residue of a polypeptide.

In some aspects, the aqueous formulations and lyophilized compositions described herein comprise a conjugate comprising a compound linked to a polypeptide, wherein the compound comprises a benzazepine-4-carboxamide compound. In some aspects, the benzazepine-4-carboxamide compound has the structure of Formula X-1:

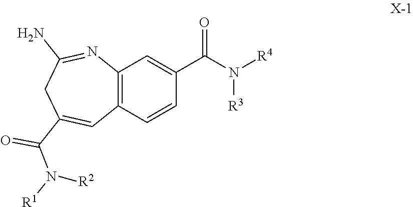

wherein:
R¹ is C₃₋₇alkyl;
R² is C₃₋₇alkyl or C₃₋₇cycloalkyl-C₁₋₇alkyl;
R³ is hydrogen;
R⁴ is selected from the group consisting of
C₁₋₇alkyl, said C₁₋₇alkyl being unsubstituted or substituted by one or two groups selected from the group consisting of phenyl and heteroaryl, said heteraryl being an aromatic 5- or 6-membered ring which comprises one, two, or three atoms selected from nitrogen, oxygen, and/or sulfur;
C₃₋₇cycloalkyl, said C₃₋₇cycloalkyl being unsubstituted or substituted by phenyl or phenylamino-C₁₋₄alkyl, and
heterocyclyl, said heterocyclyl being a saturated 3- to 7-membered ring containing one heteroatom selected from N and O and being unsubstituted or substituted by phenyl.
   Structures of Formula X-1 are described, for example, in PCT Publication No. WO2017/202703.

In some aspects, the aqueous formulations and lyophilized compositions described herein comprise a conjugate comprising a compound linked to a polypeptide, wherein the compound comprises a benzazepine-dicarboxamide compound. In some aspects, the benzazepine-dicarboxamide compound has the structure of Formula X-2:

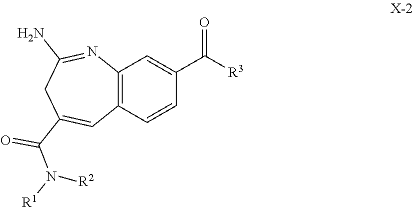

wherein:
R¹ is C₃₋₇alkyl;
R² is C₃₋₇alkyl or C₃₋₇cycloalkyl-C₁₋₇alkyl;
R³ is a heterocycle selected from

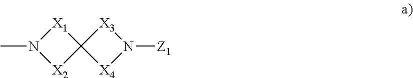

wherein
X₁ is (CH₂)ₘ wherein m is 1 or 2;
X₂ is (CH₂)ₙ wherein n is 1 or 2;
X₃ is (CH₂)ₒ wherein o is 1 or 2;
X₄ is (CH₂)ₚ wherein p is 1 or 2; and
Z₁ is phenyl, wherein phenyl is unsubstituted or substituted by one or two groups selected from the group consisting of C₁₋₇alkyl, halogen, halogen-C₁₋₇alkyl, C₁₋₇alkoxy, hydroxy-C₁₋₇alkyl, amino-C₁₋₇alkyl, C₁₋₇alkyl-amino-C₁₋₇alkyl, and di-C₁₋₇alkyl-amino-C₁₋₇alkyl; or

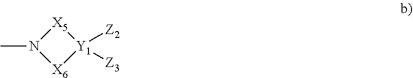

wherein
X₅ is (CH₂)_{q} wherein q is 1 or 2;
X₆ is (CH₂)ᵣ wherein r is 1 or 2;
Y₁ is a carbon or nitrogen atom;
Z₂ is hydrogen; and
Z₃ is selected from the group consisting of hydrogen, C₁₋₇alkoxy, C₂₋₇alkenyloxy, phenyl, phenyl-C₁₋₇alkyl, phenyl-C₁₋₇alkyloxy, phenyl-C₁₋₇alkylamino, phenylamino-C₁₋₇alkyl, phenylamino, wherein phenyl is unsubstituted or substituted by one or two groups selected from the group consisting of C₁₋₇alkyl, halogen, halogen-C₁₋₇alkyl, C₁₋₇alkoxy, hydroxy-C₁₋₇alkyl, amino-C₁₋₇alkyl, C₁₋₇alkyl-amino-C₁₋₇alkyl, and di-C₁₋₇alkyl-amino-C₁₋₇alkyl; or

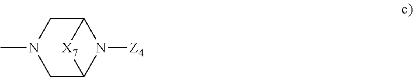

wherein
X₇ is (CH₂)ₛ wherein s is 1 or 2; and
Z₄ is phenyl, wherein phenyl is unsubstituted or substituted by one or two groups selected from the group consisting of C₁₋₇alkyl, halogen, halogen-C₁₋₇alkyl, C₁₋₇alkoxy, hydroxy-C₁₋₇alkyl, amino-C₁₋₇alkyl, C₁₋₇alkyl-amino-C₁₋₇alkyl, and di-C₁₋₇alkyl-amino-C₁₋₇alkyl; or

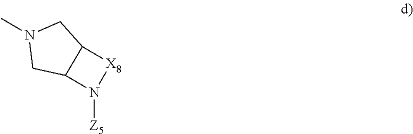

wherein
X₈ is (CH₂)ₜ wherein t is 1 or 2; and
Z₅ is phenyl, wherein phenyl is unsubstituted or substituted by one or two groups selected from the group consisting of C₁₋₇alkyl, halogen, halogen-C₁₋₇alkyl, C₁₋₇alkoxy, hydroxy-C₁₋₇alkyl, amino-C₁₋₇alkyl, C₁₋₇alkyl-amino-C₁₋₇alkyl, and di-C₁₋₇alkyl-amino-C₁₋₇alkyl.
   Compounds of Formula X-2 are described, for example, in PCT Publication No. WO2017/202704.

In some aspects, the aqueous formulations and lyophilized compositions described herein comprise a conjugate comprising a compound linked to a polypeptide, wherein the compound comprises a benzazepine sulfonamide compound. In some aspects, the benzazepine sulfonamide compound has the structure of Formula X-3:

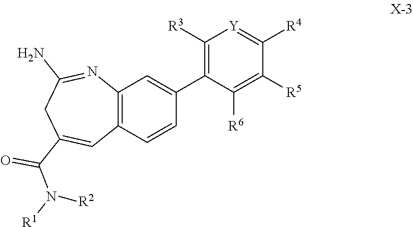

wherein
R¹ and R² are the same or different and are selected from the group consisting of C₁₋₇alkyl, hydroxy-C₂₋₇alkyl, amino-C₂₋₇alkyl, C₂₋₇alkenyl, and C₃₋₇alkynyl;
R³ is hydrogen or C₁₋₇alkyl;
R⁶ is hydrogen or C₁₋₇alkyl;
one of R⁴ and R⁵ is selected from the group consisting of hydrogen, C₁₋₇alkyl, halogen-C₁₋₇alkyl, and C₁₋₇alkoxy,
and the other one of R⁴ and R⁵ is

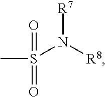

wherein R⁷ and R⁸ are the same or different and are selected from the group consisting of hydrogen, C₁₋₇alkyl, halogen-C₁₋₇alkyl, hydroxy-C₁₋₇alkyl, hydroxy-C₁₋₇alkoxy-C₁₋₇alkyl, amino-C₁₋₇alkyl, C₁₋₇alkyl-amino-C₁₋₇alkyl, amino-C₁₋₇alkoxy-C₁₋₇alkyl, C₁₋₇alkyl-amino-C₁₋₇alkoxy-C₁₋₇alkyl, amino-C₁₋₇alkyl-carbonyl, and C₁₋₇alkyl-xamino-C₁₋₇alkyl-carbonyl; or
R⁷ and R⁸ together with the nitrogen atom they are attached to form a 4- to 6-membered heterocycle which is unsubstituted or substituted with a group selected from the group consisting of amino, C₁₋₇alkyl-amino, hydroxy, and hydroxy-C₁₋₇alkyl, and which may contain an additional N—R¹⁰ group, wherein R¹⁰ is selected from the group consisting of hydrogen, amino-C₁₋₇alkyl, and C₁₋₇alkyl-amino-C₁₋₇alkyl; and
Y is N or CR⁹;
   wherein R⁹ is selected from the group consisting of hydrogen, C₁₋₇alkyl, and halogen-C₁₋₇alkyl.
      Compounds of Formula X-3 are described, for example, in PCT Publication No. WO 2016/096778.

In some aspects, the aqueous formulations and lyophilized compositions described herein comprise a conjugate comprising a compound linked to a polypeptide, wherein the compound comprises a dihydropyrimidinyl benzazepine carboxamide compound. In some aspects, the dihydropyrimidinyl benzazepine carboxamide compound has the structure of Formula X-4:

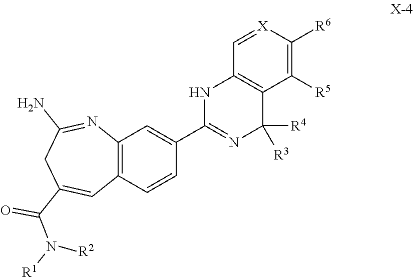

wherein
R¹ is C₃₋₇alkyl;
R² is C₃₋₇alkyl or C₃₋₇cycloalkyl-C₁₋₇alkyl;
R³ is hydrogen or C₁₋₇alkyl;
R⁴ is hydrogen or C₁₋₇alkyl;
R⁵ is selected from the group consisting of hydrogen, halogen, C₁₋₇alkyl, and C₁₋₇alkoxy;
R⁶ is selected from the group consisting of hydrogen, halogen, C₁₋₇alkyl, and C₁₋₇alkoxy; and
X is N or CR⁷, wherein R⁷ is selected from the group consisting of hydrogen, halogen, C₁₋₇alkyl, and C₁₋₇alkoxy.
   Compounds of Formula X-4 are described, for example, in PCT Publication No. WO 2017/216054.

In some aspects, the aqueous formulations and lyophilized compositions described herein comprise a conjugate comprising a compound linked to a polypeptide, wherein the compound comprises a sulfinylphenyl or sulfonimidoylphenyl benzazepine compound. In some aspects, the sulfinylphenyl or sulfonimidoylphenyl benzazepine compound has the structure of Formula X-5:

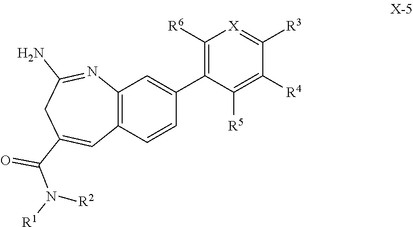

wherein
X is CR⁷ or N;
R¹ is C₃₋₇alkyl or C₃₋₇cycloalkyl;
R² is selected from the group consisting of C₃₋₇alkyl, hydroxy-C₁₋₇alkyl, C₃₋₇-alkynyl, amino-C₁₋₇alkoxy-C₁₋₇alkoxy-C₁₋₇alkyl, halogen-C₁₋₇alkyl, and C₃₋₇cycloalkyl-C₁₋₇alkyl; one of R³ and R⁴ is

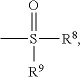

and the other one of R³ and R⁴ is selected from the group consisting of hydrogen, C₁₋₇alkyl, and halogen;
R⁵, R⁶, and R⁷ are independently from each other selected from hydrogen, C₁₋₇alkyl, and halogen;
R⁸ is C₁₋₇alkyl; and
R⁹ is absent or is ═N—R¹⁰, wherein R¹⁰ is selected from the group consisting of hydrogen, C₁₋₇alkyl, halogen-C₁₋₇alkyl, hydroxy-C₁₋₇alkyl, and hydroxy-C₁₋₇alkoxy-C₁₋₇alkyl.
   Compounds of Formula X-5 are described, for example, in PCT Publication No. WO 2017/046112.

In some aspects, the aqueous formulations and lyophilized compositions described herein comprise a conjugate comprising a compound linked to a polypeptide, wherein the compound comprises a TLR modulator compound that has the structure of Formula X-6:

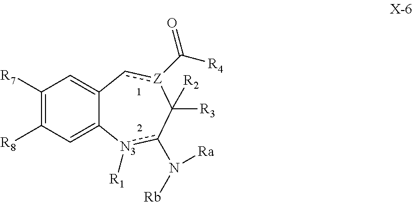

wherein
[symbol] (1) is a double bond or a single bond;
[symbol] (2) is double bond and R₁ is absent;
R² and R³ are independently selected from H and lower alkyl, or R² and R³ are connected to form a saturated carbocycle having from 3 to 7 ring members;
one of R₇ and R₈ is —NR_{f}R_{g},

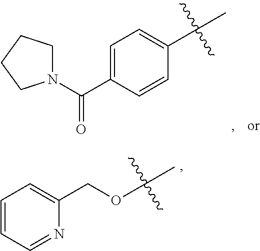 [SMILES: *c1ccc(C(=O)N2CCCC2)cc1 ; *OCc1ccccn1]

and the other is hydrogen;
where R_{f} and R_{g} are lower alkyl or R_{f} and R_{g} together with the nitrogen to which they are attached form a saturated heterocyclic ring having 4 to 6 ring members;
R⁴ is —NR_{c}R_{d} or —OR₁₀;
   R_{c} and R_{d} are lower alkyl, where the alkyl is optionally substituted with one or more —OH;
   R₁₀ is alkyl, where the alkyl is optionally substituted with one or more —OH;
Z is C and [symbol] (1) is a double bond, or Z is N and [symbol] (1) is a single bond;

R^{a} and R^{b} are each H.

In some aspects, the aqueous formulations and lyophilized compositions described herein comprise a conjugate comprising a compound linked to a polypeptide, wherein the compound comprises a TLR modulator compound that has the structure of Formula X-7:

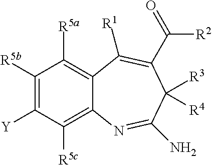

wherein
Y is CF₂CF₃, CF₂CF₇R⁶, or an aryl or heteroaryl ring, wherein said aryl and heteroaryl rings are substituted with one or more groups independently selected from alkenyl, alkynyl, Br, CN, OH, NR⁶R⁷, C(═O)R⁸, NR⁶SO₂R⁷, (C₁-C₆ alkyl)amino, R⁶OC(═O)CH═CH₂—, SR⁶ and SO₂R⁶, and wherein the aryl and heteroaryl rings are optionally further substituted with one or more groups independently selected from F, Cl, CF₃, CF₃O—, HCF₂O—, alkyl, heteroalkyl and ArO—;
R¹, R³ and R⁴ are independently selected from H, alkyl, alkenyl, alkynyl, heteroalkyl, cycloalkyl, cycloalkenyl, heterocycloalkyl, aryl and heteroaryl, wherein the alkyl, alkenyl, alkynyl, heteroalkyl, cycloalkyl, cycloalkenyl, heterocycloalkyl, aryl and heteroaryl are optionally substituted with one or more groups independently selected from alkyl, alkenyl, alkynyl, F, Cl, Br, I, CN, OR⁶, NR⁶R⁷, C(═O)R⁶, C(═O)OR⁶, OC(═O)R⁶, C(═O)NR⁶R⁷, (C₁-C₆ alkyl)amino, CH₃OCH₂O—, R⁶OC(O)CH═CH₂—, NR⁶SO₂R⁷, SR⁶ and SO₂R⁶,
or R³ and R⁴ together with the atom to which they are attached form a saturated or partially unsaturated carbocyclic ring, wherein the carbocyclic ring is optionally substituted with one or more groups independently selected from alkyl, alkenyl, alkynyl, F, Cl, Br, I, CN, OR⁶, NR⁶R⁷, C(═O)R⁶, C(═O)OR⁶, OC(═O)R⁶, C(═O)NR⁶R⁷, (C₁-C₆ alkyl)amino, CH₃OCH₂O, R⁶OC(═O)CH═CH₂—, NR⁶SO₂R⁷, SR⁶ and SO₂R⁶;
R² and R⁸ are independently selected from H, OR⁶, NR⁶R⁷, alkyl, alkenyl, alkynyl, heteroalkyl, cycloalkyl, cycloalkenyl, heterocycloalkyl, aryl and heteroaryl, wherein the alkyl, alkenyl, alkynyl, heteroalkyl, cycloalkyl, cycloalkenyl, heterocycloalkyl, aryl and heteroaryl are optionally substituted with one or more groups independently selected from alkyl, alkenyl, alkynyl, F, Cl, Br, I, CN, OR⁶, NR⁶R⁷, C(═O)R⁶, C(═O)OR⁶, OC(═O)R⁶, C(O)NR⁶R⁷, (C₁-C₆ alkyl)amino, CH₃OCH₂O—, R⁶OC(═O)CH═CH₂—, NR⁶SO₂R⁷, SR⁶ and SO₂R⁶;
R^{5a}, R^{5b}, and R^{5c} are independently H, F, Cl, Br, I, OMe, CH₃, CH₂F, CHF₂ or CF₃; and
R⁶ and R⁷ are independently selected from H, alkyl, alkenyl, alkynyl, heteroalkyl, cycloalkyl, cycloalkenyl, heterocycloalkyl, aryl and heteroaryl, wherein said alkyl, alkenyl, alkynyl, heteroalkyl, cycloalkyl, cycloalkenyl, heterocycloalkyl, aryl and heteroaryl are optionally substituted with one or more groups independently selected from alkyl, alkenyl, alkynyl, F, Cl, Br, I, CN, OR⁶, NR⁶R⁷, C(═O)R⁶, C(═O)OR⁶, OC(═O)R⁶, C(═O)NR⁶R⁷, (C₁-C₆ alkyl)amino, CH₃OCH₂O—, R⁶OC(O)CH═CH₂—, NR⁶SO₂R⁷, SR⁶ and SO₂R⁶,
or R⁶ and R⁷ together with the atom to which they are attached form a saturated or partially unsaturated heterocyclic ring, wherein said heterocyclic ring is optionally substituted with one or more groups independently selected from alkyl, alkenyl, alkynyl, F, Cl, Br, I, CN, OR⁶, NR⁶R⁷, C(═O)R⁶, C(═O)OR⁶, OC(═O)R⁶, C(═O)NR⁶R⁷, (C₁-C₆alkyl)amino, CH₃OCH₂O—, R⁶OC(═O)CH═CH₂—, NR⁶SO₂R⁷, SR⁶ and SO₂R⁶.

In some aspects, the aqueous formulations and lyophilized compositions described herein comprise a conjugate comprising a compound linked to a polypeptide, wherein the compound comprises a TLR modulator compound that has the structure of Formula X-8:

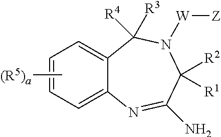

wherein
W is —C(O)—;
Z is H, alkyl, alkenyl, alkynyl, heteroalkyl, cycloalkyl, heterocycloalkyl, aryl, heteroaryl, OR⁶ or NR⁶R⁷, wherein the alkyl, alkenyl, alkynyl, heteroalkyl, cycloalkyl, heterocycloalkyl, aryl and heteroaryl are optionally substituted with one or more groups independently selected from alkyl, alkenyl, alkynyl. F, Cl, Br, I, CN, OR⁶, NR⁶R⁷, C(═O)R⁶, C(═O)OR⁶, OC(═O)R⁶, C(═O)NR⁶R⁷, (C₁-C₆ alkyl)amino, CH₃OCH₂O—, R⁶OCC═O)CH═CH₂—, NR⁶SO₂R⁷, SR⁶ and SO₂R⁶;
R¹, R², R³ and R⁴ are independently selected from H, alkyl, alkenyl, alkynyl, heteroalkyl, cycloalkyl, cycloalkenyl, heterocycloalkyl, aryl and heteroaryl, wherein said alkyl, alkenyl, alkynyl, heteroalkyl, cycloalkyl, cycloalkenyl, heterocycloalkyl, aryl, and heteroaryl are optionally substituted with one or more groups independently selected from alkyl, alkenyl, alkynyl, F, Cl, Br, I, CN, OR⁶, NR⁶R⁷, C(═O)R⁶, C(═O)OR⁶, OC(═O)R⁶, C(═O)NR⁶R⁷, (C₁-C₆ alkyl)amino, CH₃OCH₂O—, R⁶OC(C═O)CH═CH₂—, NR⁶SO₂R⁷, SR₆ and SO₂R⁶,
or R¹ and R² together with the atom to which they are attached form a saturated or partially unsaturated carbocyclic ring, wherein said carbocyclic ring is optionally substituted with one or more groups independently selected from alkyl, alkenyl, alkynyl, F, Cl, Br, I, CN, OR⁶, NR⁶R⁷, C(═O)R⁶, C(═O)OR⁶, OC(═O)R⁶, C(═O)NR⁶R⁷, (C₁-C₆ alkyl)amino, CH₃OCH₂O—, R⁶OC(═O)CH═CH₂—, NR⁶SO₂R⁷, SR⁶ and SO₂R⁶,
or R³ and R⁴ together are oxo;
R⁵ is H, F, Cl, Br, I, OMe, CH₃, CH₂F, CHF₂, CF₃ or CF₂CF₃;
R⁶ and R⁷ are independently selected from H, alkyl, alkenyl, alkynyl, heteroalkyl, cycloalkyl, cycloalkenyl, heterocycloalkyl, aryl, and heteroaryl, wherein said alkyl, alkenyl, alkynyl, heteroalkyl, cycloalkyl cycloalkenyl, heterocycloalkyl, aryl, and heteroaryl are optionally substituted with one or more groups independently selected from alkyl, alkenyl, alkynyl, F, Cl, Br, I, CN, OR⁶, NR⁶R⁷, C(═O)R⁶, C(═O)OR⁶, OC(═O)R⁶, C(═O)NR⁶R⁷, (C₁-C₆ alkyl)amino, CH₃OCH₂O—, R⁶OC(═O)CH═CH₂—, NR⁶SO₂R⁷, SR⁶ and SO₂R⁶;
or R⁶ and R⁷ together with the atom to which they are attached form a saturated or partially unsaturated heterocyclic ring, wherein said heterocyclic ring is optionally substituted with one or more groups independently selected from alkyl, alkenyl, alkynyl, F, Cl, Br, I, CN, OR⁶, NR⁶R⁷, C(═O)R⁶, C(═O)OR⁶, OC(═O)R⁶, C(═O)NR⁶R⁷, (C₁-C₆ alkyl)amino, CH₃OCH₂O—, R⁶OC(═O)CH═CH₂—, NR⁶SO₂R⁷, SR⁶ and SO₂R⁶; and
n is 0, 1, 2,3 or 4.

Compounds of Formula X-6, X-7, and X-8 are described, for example, in U.S. Publication No. US 2019/0016808 and US 2014/0088085.

Included in the present disclosure are salts, particularly pharmaceutically acceptable salts, of the compounds described herein. The compounds of the present disclosure that possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as bromide, chloride, or fluoride.

The compounds described herein may in some cases exist as diastereomers, enantiomers, or other stereoisomeric forms. The compounds presented herein include all diastereomeric, enantiomeric, and epimeric forms as well as the appropriate mixtures thereof. Separation of stereoisomers may be performed by chromatography or by forming diastereomers and separating by recrystallization, or chromatography, or any combination thereof. (Jean Jacques, Andre Collet, Samuel H. Wilen, “Enantiomers, Racemates and Resolutions”, John Wiley and Sons, Inc., 1981, herein incorporated by reference for this disclosure). Stereoisomers may also be obtained by stereoselective synthesis.

The compounds described herein may exist in amorphous forms or in crystalline forms (also known as polymorphs). In addition, the compounds described herein can exist in unsolvated as well as solvated forms with pharmaceutically acceptable solvents such as water, ethanol, and the like. The solvated forms of the compounds presented herein are also considered to be disclosed herein.

The present disclosure also includes metabolites and prodrugs of the compounds described herein. Metabolites of these compounds having the same type of activity are included in the scope of the present disclosure. The term “prodrug” is intended to encompass compounds which, under physiologic conditions, are converted into active compounds, e.g., benzazepine and benzazepine-like compounds as described herein, including but not limited to immune-stimulatory compounds or TLR8 agonists. One method for making a prodrug is to include one or more selected moieties which are hydrolyzed or otherwise cleaved under physiologic conditions to reveal the desired molecule. In other embodiments, the prodrug is converted by an enzymatic activity of the host animal such as specific target cells in the host animal. Prodrug forms of the herein described compounds, wherein the prodrug is metabolized in vivo to produce a compound described herein are included within the scope of the disclosure. In some cases, some of the herein-described compounds may be a prodrug for another derivative or active compound.

In certain embodiments, a benzazepine and benzazepine-like compound, such as an immune-stimulatory compound or a TLR8 agonist, is modified as a prodrug with a masking group, such that the compound has limited activity or is inactive until it reaches an environment where the masking group is removed to reveal the active compound.

Synthetic chemistry transformations and methodologies useful in synthesizing the compounds described herein are known in the art and include, for example, those described in R. Larock, Comprehensive Organic Transformations (1989); T. W. Greene and P. G. M. Wuts, Protective Groups in Organic Synthesis, 2d. Ed. (1991); L. Fieser and M. Fieser, Fieser and Fieser's Reagents for Organic Synthesis (1994); and L. Paquette, ed., Encyclopedia of Reagents for Organic Synthesis (1995).

### Exemplary Linkers

The conjugates include a linker(s) that attaches a polypeptide to at least one benzazepine or benzazepine-like compound, such as at least one immune-stimulatory compound, such as a myeloid cell agonist. A linker can be, for example, a cleavable or a non-cleavable linker. A conjugate can comprise multiple linkers. The linkers in a conjugate can be the same linkers or different linkers.

As will be appreciated by skilled artisans, a linker connects a benzazepine or benzazepine-like compound (e.g., an immune-stimulatory compound(s), such as a myeloid cell agonist) to the polypeptide (e.g., antibody) by forming a covalent linkage to the compound at one location and a covalent linkage to the polypeptide at another location. The covalent linkages can be formed by reaction between functional groups on the linker and functional groups on the immune-stimulatory compound and on the polypeptide. As used herein, the expression “linker” can include (i) unattached forms of the linker that can include a functional group capable of covalently attaching the linker to the compound and a functional group capable of covalently attached the linker to the polypeptide; (ii) partially attached forms of the linker that can include a functional group capable of covalently attaching the linker to the polypeptide and that can be covalently attached to a compound, or vice versa; and (iii) fully attached forms of the linker that can be covalently attached to both a compound and to a polypeptide. In some specific embodiments, the functional groups on a linker and covalent linkages formed between the linker and a polypeptide, such as an antibody, can be specifically illustrated as Rx and Rx′, respectively.

A linker can be short or long, flexible, rigid, cleavable, non-cleavable, hydrophilic, or hydrophobic, or a combination thereof. A linker can contain segments that have different characteristics, such as segments of flexibility or segments of rigidity, segments of hydrophilicity, and/or segments of hydrophobicity. A linker can contain multiple segments, such as one or more non-cleavable segments and one or more cleavable segments. A linker can comprise alkylene, alkenylene, alkynylene, polyether, polyester, polyamide, polyamino acid, peptide, polypeptide, cleavable peptide, and/or aminobenzylcarbamate groups.

In some embodiments, a linker can include a “non-cleavable” segment that is chemically stable in the blood stream and in intracellular environments. In some embodiments, a linker comprises a “cleavable” segment that includes one or more linkages that are not stable, such as linkages that are designed to cleave and/or immolate or otherwise breakdown specifically or non-specifically in the blood stream and/or inside cells (i.e., in an intracellular environment). Linkers comprise one or more cleavable segments, one or more non-cleavable segments, or a combination thereof.

A cleavable linker can be sensitive to (i.e., cleavable by) enzymes at a specific site. A cleavable linker can be cleaved by enzymes such as protesases. A cleavable linker can be a valine-citrulline peptide or a valine-alanine peptide. A valine-citrulline- or valine-alanine-containing linker can contain a pentafluorophenyl group. A valine-citrulline or valine-alanine-containing linker can contain a succimide group. A valine-citrulline- or valine-alanine-containing linker can contain a para aminobenzoic acid (PABA) group. A valine-citrulline- or valine-alanine-containing linker can contain a PABA group and a pentafluorophenyl group. A valine-citrulline- or valine-alanine-containing linker can contain a PABA group and a succinimide group. A valine-citrulline- or valine-alanine-containing linker can contain a PABA group and a succinimide group.

Cleavable linkers can be cleavable in vitro and in vivo. Cleavable linkers can include chemically or enzymatically unstable or degradable linkages. Cleavable linkers can rely on processes inside the cell to liberate an immune-stimulatory compound, such as reduction in the cytoplasm, exposure to acidic conditions in the lysosome, or cleavage by specific proteases or other enzymes within the cell. Cleavable linkers can incorporate one or more chemical bonds that are chemically or enzymatically cleavable while the remainder of the linker can be non-cleavable.

A linker can contain a chemically labile group such as hydrazone and/or disulfide group. Linkers comprising chemically labile groups can exploit differential properties between the plasma and some cytoplasmic compartments. The intracellular conditions that can facilitate compound release for hydrazine-containing linkers can be the acidic environment of endosomes and lysosomes, while disulfide-containing linkers can be reduced in the cytosol, which can contain high thiol concentrations, e.g., glutathione. The plasma stability of a linker containing a chemically labile group can be increased by introducing steric hindrance using substituents near the chemically labile group.

Acid-labile groups, such as hydrazone, can remain intact during systemic circulation in the blood's neutral pH environment (pH 7.3-7.5) and can undergo hydrolysis and can release an immune-stimulatory compound once the conjugate is internalized into mildly acidic endosomal (pH 5.0-6.5) and lysosomal (pH 4.5-5.0) compartments of the cell. This pH dependent release mechanism can be associated with nonspecific release of the immune-stimulatory compound. To increase the stability of the hydrazone group of the linker, the linker can be varied by chemical modification, e.g., substitution, allowing tuning to achieve more efficient release in the lysosome with a minimized loss in circulation.

Hydrazone-containing linkers can contain additional cleavage sites, such as additional acid-labile cleavage sites and/or enzymatically labile cleavage sites. Exemplary cleavable linkers including hydrazine moieties and disulfide moieties include, for example, the portions of the following structures:

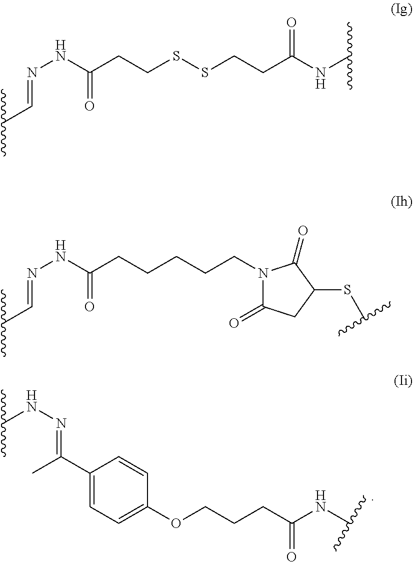 [SMILES: */C=N/NC(=O)CCSSCCC(=O)N* ; */C=N/NC(=O)CCCCCN1C(=O)CC(S*)C1=O ; *N/N=C(\C)c1ccc(OCCCC(=O)N*)cc1]

In certain linkers such as linker (Ig), the linker can comprise two cleavable groups- a disulfide and a hydrazone moiety. For such linkers, effective cleavage can require acidic pH or disulfide reduction and acidic pH. Linkers such as (Ih) and (Ii) can be effective with a single hydrazone cleavage site.

Other acid-labile groups that can be included in linkers include cis-aconityl-containing linkers. cis-Aconityl chemistry can use a carboxylic acid juxtaposed to an amide bond to accelerate amide hydrolysis under acidic conditions.

Cleavable linkers can also include a disulfide group. Disulfides can be thermodynamically stable at physiological pH and can be designed to release upon internalization inside cells, wherein the cytosol can provide a significantly more reducing environment compared to the extracellular environment. Scission of disulfide bonds can require the presence of a cytoplasmic thiol cofactor, such as (reduced) glutathione (GSH), such that disulfide-containing linkers can be reasonably stable in circulation, selectively releasing the myeloid cell agonist in the cytosol. The intracellular enzyme protein disulfide isomerase, or similar enzymes capable of cleaving disulfide bonds, can also contribute to the preferential cleavage of disulfide bonds inside cells. GSH can be present in cells in the concentration range of 0.5-10 mM compared with a significantly lower concentration of GSH or cysteine, the most abundant low-molecular weight thiol, in circulation at approximately 5 μM. Tumor cells, where irregular blood flow can lead to a hypoxic state, can result in enhanced activity of reductive enzymes and therefore even higher glutathione concentrations. The in vivo stability of a disulfide-containing linker can be enhanced by chemical modification of the linker, e.g., use of steric hindrance adjacent to the disulfide bond.

Exemplary cleavable linkers including disulfide moieties can include the following structures:

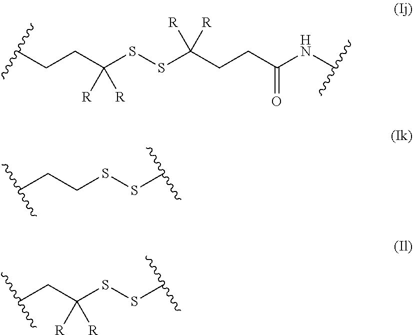

wherein R is independently selected at each occurrence from hydrogen or alkyl, for example. Increasing steric hindrance adjacent to the disulfide bond can increase the stability of the linker. Structures such as (Ij) and (Il) can show increased in vivo stability when one or more R groups is selected from a lower alkyl such as methyl.

Another type of cleavable linker is specifically cleaved by an enzyme. For example, the linker can be cleaved by a lysosomal enzyme. Such linkers can be peptide-based or can include peptidic regions that can act as substrates for enzymes. Peptide-based linkers can be more stable in plasma and extracellular milieu than chemically labile linkers.

Peptide bonds can have good serum stability, as lysosomal proteolytic enzymes can have very low activity in blood due to endogenous inhibitors and the unfavorably high pH value of blood compared to lysosomes. Release of a myeloid cell agonist from a conjugate can occur due to the action of lysosomal proteases, e.g., cathepsin and/or plasmin. These proteases can be present at elevated levels in certain tumor tissues. The linker can be cleavable by a lysosomal enzyme. The lysosomal enzyme can be, for example, cathepsin B, β-glucuronidase, or β-galactosidase.

In a linker, a cleavable peptide can be selected from tetrapeptides or dipeptides such as Val-Cit, Val-Ala, and Phe-Lys. Dipeptides can have lower hydrophobicity compared to longer peptides, depending on the composition of the peptide. A variety of dipeptide-based cleavable linkers can be used in the conjugates described herein.

In some embodiments, the cleavable linker comprises a cleavable peptide. In some embodiments, the cleavable peptide is a dipeptide, tripeptide, or tetrapeptide. In some embodiments, the cleavable peptide is Val-Cit; Cit-Val; Ala-Ala; Ala-Cit; Cit-Ala; Asn-Cit; Cit-Asn; Cit-Cit; Val-Glu; Glu-Val; Ser-Cit; Cit-Ser; Lys-Cit; Cit-Lys; Asp-Cit; Cit-Asp; Ala-Val; Val-Ala; Phe-Lys; Lys-Phe; Val-Lys; Lys-Val; Ala-Lys; Lys-Ala; Phe-Cit; Cit-Phe; Leu-Cit; Cit-Leu; Ile-Cit; Cit-Ile; Phe-Arg; Arg-Phe; Cit-Trp; Trp-Cit; Glu-Val-Cit; Ala-Ala-Asn; Glu-Glu-Val-Cit (SEQ ID NO: 72); Gly-Phe-Leu-Gly (SEQ ID NO: 73); Gly-Gly-Phe-Gly (SEQ ID NO: 74); or Ala-Leu-Ala-Leu (SEQ ID NO: 75).

In some embodiments, the cleavable linker is a structure of formula:

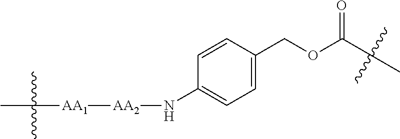

wherein -AA₁-AA₂- is the cleavable dipeptide and AA₁ and AA₂ are each an amino acid. In some embodiments, the cleavable dipeptide is Val-Cit.

Enzymatically cleavable linkers can include a self-immolative spacer to spatially separate the myeloid cell agonist from the site of enzymatic cleavage. The direct attachment of a myeloid cell agonist to a peptide linker can result in proteolytic release of an amino acid adduct of the compound (e.g., benzazepine or myeloid cell agonist), thereby impairing its activity. The use of a self-immolative spacer can allow for the elimination of the fully active, chemically unmodified benzazepine or myeloid cell agonist upon amide bond hydrolysis.

One self-immolative spacer can be a bifunctional para-aminobenzyl alcohol group, which can link to the peptide through the amino group, forming an amide bond, while amine containing benzazepines or myeloid cell agonists can be attached through carbamate functionalities to the benzylic hydroxyl group of the linker (to give a p-amidobenzylcarbamate, PABC). The resulting pro-benzazepine or pro-myeloid cell agonist can be activated upon protease-mediated cleavage, leading to a 1,6-elimination reaction releasing the unmodified benzazepine or myeloid cell agonist, carbon dioxide, and remnants of the linker group.

The enzymatically cleavable linker can be a β-glucuronic acid-based linker. Facile release of the myeloid cell agonist can be realized through cleavage of the β-glucuronide glycosidic bond by the lysosomal enzyme β-glucuronidase. This enzyme can be abundantly present within lysosomes and can be overexpressed in some tumor types, while the enzyme activity outside cells can be low. β-Glucuronic acid-based linkers can be used to circumvent the tendency of a conjugate to undergo aggregation due to the hydrophilic nature of β-glucuronides. In certain embodiments, β-glucuronic acid-based linkers can link the ASGR ligand and/or Fc domain to a hydrophobic myeloid cell agonist.

A variety of cleavable β-glucuronic acid-based linkers useful for linking drugs such as auristatins, camptothecin and doxorubicin analogues, CBI minor-groove binders, and psymberin to antibodies have been described. All of these β-glucuronic acid-based linkers may be used in the conjugates comprising a myeloid cell agonist described herein. In certain embodiments, the enzymatically cleavable linker is a β-galactoside-based linker. β-Galactoside is present abundantly within lysosomes, while the enzyme activity outside cells is low.

Additionally, cleavable linkers may comprise a phenol and connection through the phenolic oxygen. One such linker employs diamino-ethane unit in conjunction with traditional “PABO”-based self-immolative groups to deliver a phenol.

Benzazepines or myeloid cell agonists containing an aromatic or aliphatic hydroxyl group can be covalently bonded to a linker through the hydroxyl group using a methodology that relies on a methylene carbamate linkage, as described in WO 2015/095755.

Degradable linkages may be present in otherwise non-cleavable linkers. For example, polyethylene glycol (PEG) and related polymers can include cleavable groups in the polymer backbone. For example, a polyethylene glycol or polymer linker can include one or more cleavable groups such as a disulfide, a hydrazone or a dipeptide. Other degradable linkages that can be included in cleavable linkers include ester linkages formed by the reaction of PEG carboxylic acids or activated PEG carboxylic acids with alcohol groups on a myeloid cell agonist, wherein such ester groups can hydrolyze under physiological conditions to release the myeloid cell agonist. Hydrolytically degradable linkages can include, but are not limited to, carbonate linkages; imine linkages resulting from reaction of an amine and an aldehyde; phosphate ester linkages formed by reacting an alcohol with a phosphate group; acetal linkages that are the reaction product of an aldehyde and an alcohol; orthoester linkages that are the reaction product of a formate and an alcohol; and oligonucleotide linkages formed by a phosphoramidite group, including but not limited to, at the end of a polymer, and a 5′ hydroxyl group of an oligonucleotide.

In some embodiments, a cleavable linker is a (succinimidocaproyl)-(valine-citrulline)-(para-aminobenzyloxycarbonyl) group. In some embodiments, a cleavable linker comprises a lysine with an N-terminal amine acetylated, and a valine-citrulline cleavage site.

A non-cleavable linker can be protease insensitive. A non-cleavable linker can contain a succinimide group. A non-cleavable linker can be succinimidocaproyl spacer. A succinimidocaproyl spacer can comprise N-succinimidomethylcyclohexane-1-carboxylate. A succinimidocaproyl spacer can contain pentafluorophenyl group.

A non-cleavable linker can be a combination of a succinimidocaproyl group and one or more ethylene glycol units. A non-cleavable linker can be a succinimide-PEG4 linker. A non-cleavable linker can be a combination of a succinimidocaproyl linker containing a succinimide group and one or more ethylene glycol units. A non-cleavable linker can be a combination of a succinimidocaproyl group, a pentafluorophenyl group, and one or more ethylene glycol units. A non-cleavable linker can contain one or more succinimido groups linked to polyethylene glycol units in which the polyethylene glycol can allow for more linker flexibility or can be used lengthen the linker.

A linker can be polyvalent such that it covalently links more than one compound to a single site on the polypeptide, or monovalent such that it covalently links a single compound to a single site on the polypeptide.

Exemplary connector regions or connector segments include Fleximer® linker technology that has the potential to enable high-DAR conjugates with good physicochemical properties. The Fleximer® linker technology is based on incorporating drug molecules into a solubilizing poly-acetal backbone via a sequence of ester bonds. The methodology renders highly-loaded conjugates (DAR up to 20) whilst maintaining good physicochemical properties.

A connector region can comprise one or more non-cleavable spacers and/or one or more cleavable linkers. In some embodiments, a connector region comprises a cleavable linker comprising cleavable peptide, for example, a linker comprising structural formula (IVa), (IVb), (IVc), or (IVd):

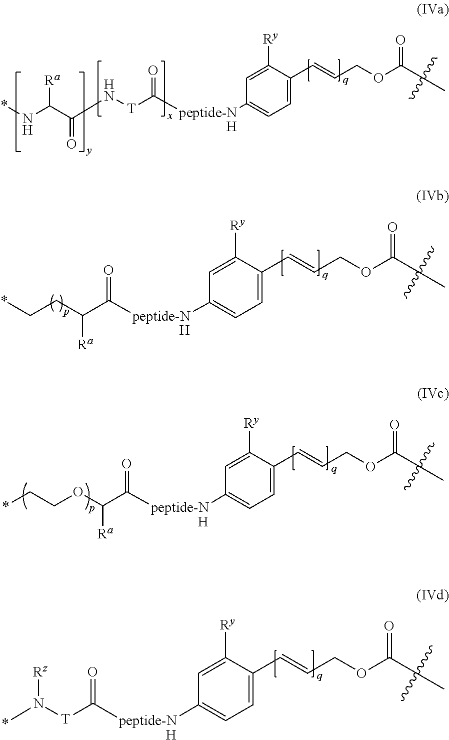

or a salt thereof, wherein:
peptide represents a cleavable peptide (illustrated N→C, wherein peptide includes the amino and carboxy “termini”) as described herein;
T represents a polymer comprising one or more ethylene glycol units or an alkylene chain, or combinations thereof;
R^{a} is selected from hydrogen, alkyl, sulfonate and methyl sulfonate;
R^{y} is hydrogen or C₁₋₄ alkyl-(O)ᵣ—(C₁₋₄ alkylene)ₛ-G¹ or C₁₋₄ alkyl-(N)—[(C₁₋₄ alkylene)-G¹]₂;
R^{z} is C₁₋₄ alkyl-(O)ᵣ—(C₁₋₄ alkylene)ₛ-G²;
G¹ is SO₃H, CO₂H, PEG 4-32, or sugar moiety;
G² is SO₃H, CO₂H, or PEG 4-32 moiety;
r is 0 or 1;
s is 0 or 1;
p is an integer ranging from 0 to 5;
q is 0 or 1;
x is 0 or 1;
y is 0 or 1;
[symbol] represents one point of attachment of the connector to the rest of the conjugate; and
* represents the point of attachment to another portion of the conjugate.

Exemplary embodiments of divalent connector regions or connector segments according to structural formula (IVa) that can be included in the conjugates described herein can include the structures illustrated below:

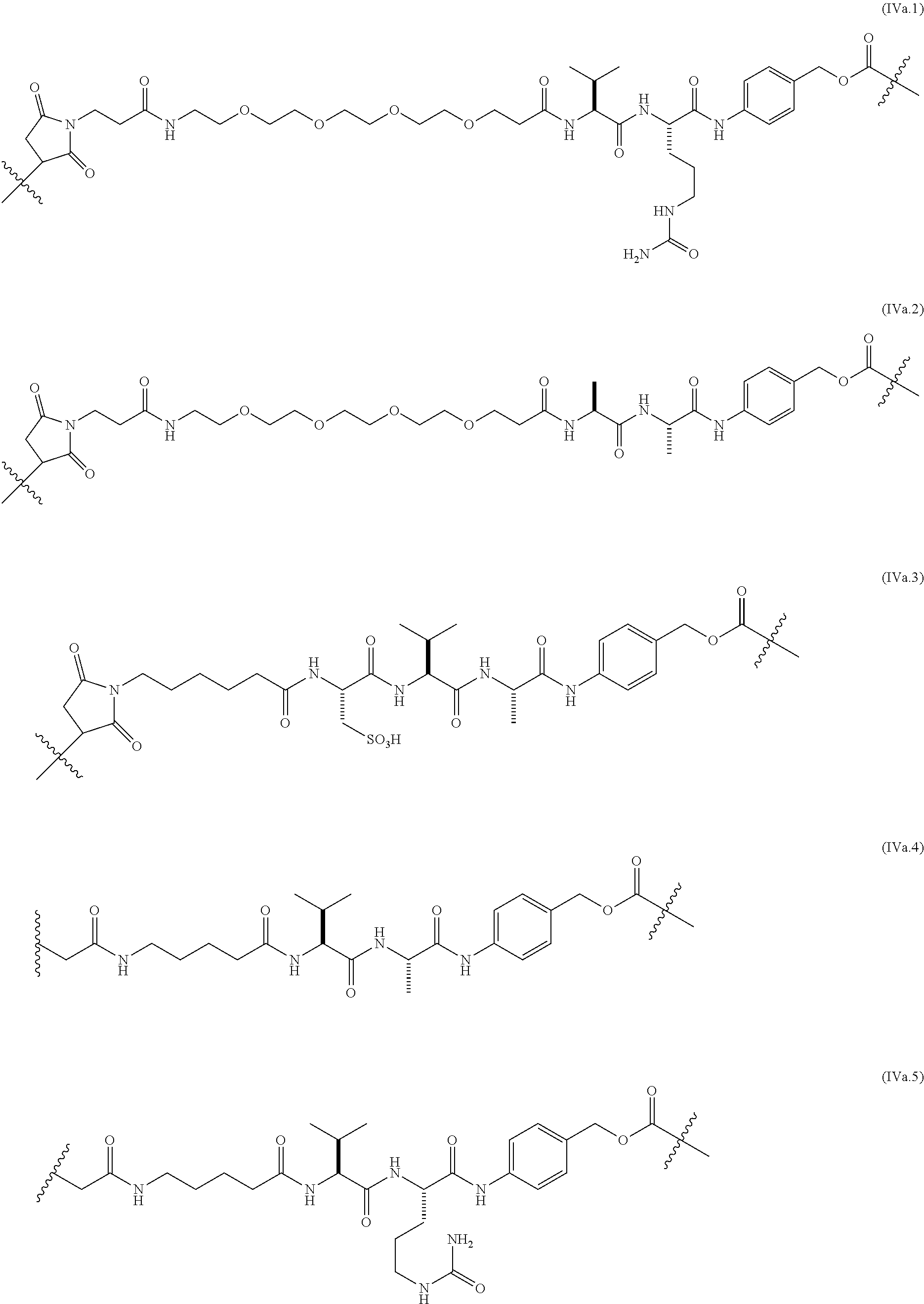 [SMILES: *C(=O)OCc1ccc(NC(=O)[C@H](CCCNC(N)=O)NC(=O)[C@@H](NC(=O)CCOCCOCCOCCOCCNC(=O)CCN2C(=O)CC(*)C2=O)C(C)C)cc1 ; *C(=O)OCc1ccc(NC(=O)[C@H](C)NC(=O)[C@H](C)NC(=O)CCOCCOCCOCCOCCNC(=O)CCN2C(=O)CC(*)C2=O)cc1 ; *C(=O)OCc1ccc(NC(=O)[C@H](C)NC(=O)[C@@H](NC(=O)[C@H](CS(=O)(=O)O)NC(=O)CCCCCN2C(=O)CC(*)C2=O)C(C)C)cc1 ; *CC(=O)NCCCCC(=O)N[C@H](C(=O)N[C@@H](C)C(=O)Nc1ccc(COC(*)=O)cc1)C(C)C ; *CC(=O)NCCCCC(=O)N[C@H](C(=O)N[C@@H](CCCNC(N)=O)C(=O)Nc1ccc(COC(*)=O)cc1)C(C)C]

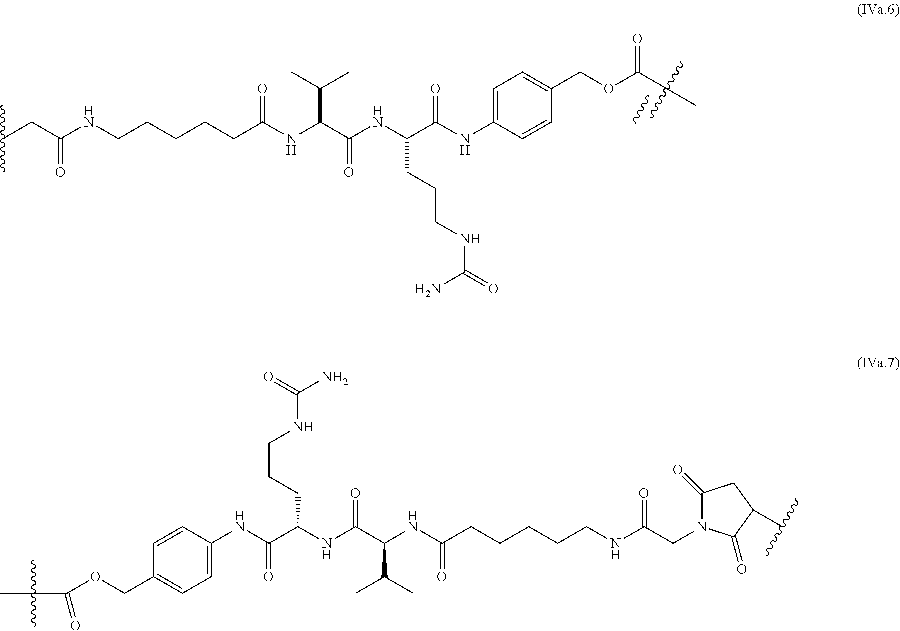

Exemplary embodiments of connector regions or connector segments according to structural formula (IVb), (IVc), or (IVd) that can be included in the conjugates described herein can include the linkers illustrated below.

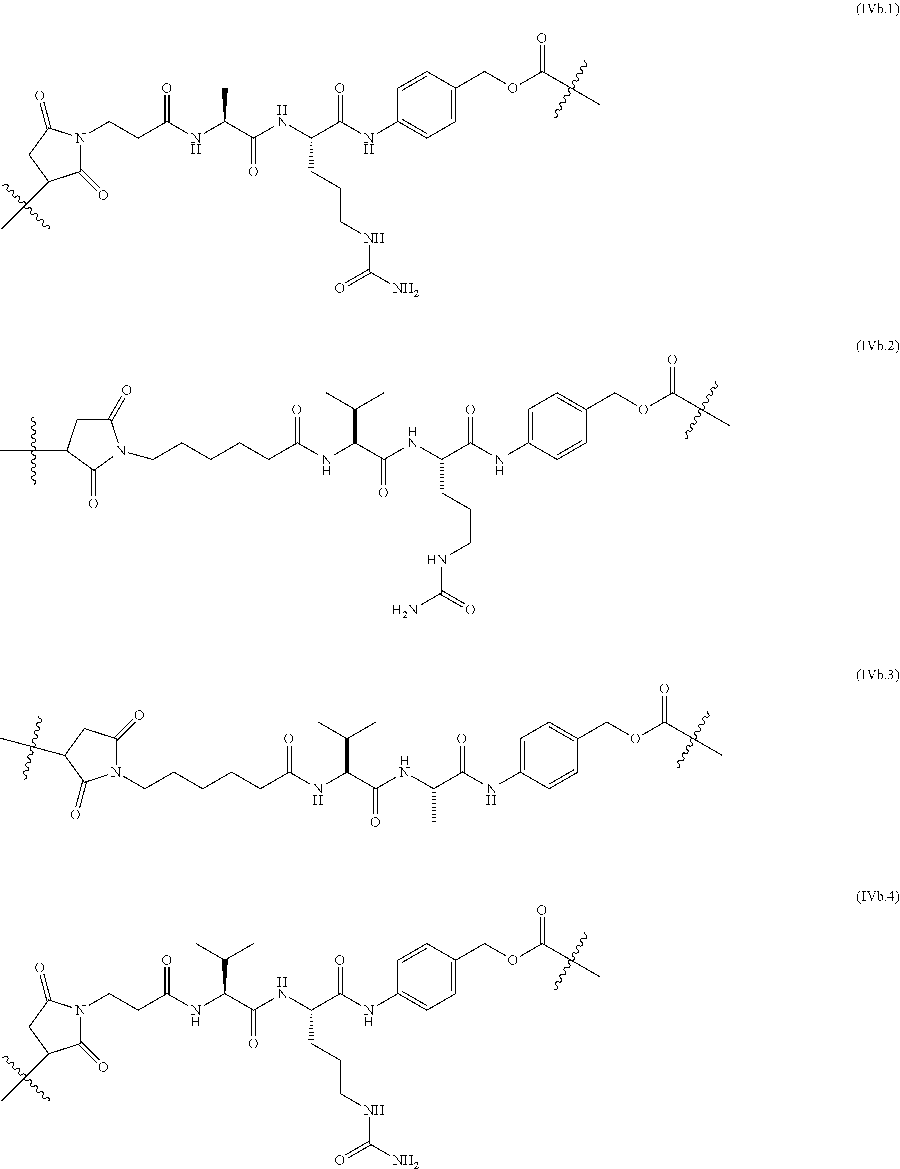 [SMILES: *C(=O)OCc1ccc(NC(=O)[C@H](CCCNC(N)=O)NC(=O)[C@H](C)NC(=O)CCN2C(=O)CC(*)C2=O)cc1 ; *C(=O)OCc1ccc(NC(=O)[C@H](CCCNC(N)=O)NC(=O)[C@@H](NC(=O)CCCCCN2C(=O)CC(*)C2=O)C(C)C)cc1 ; *C(=O)OCc1ccc(NC(=O)[C@H](C)NC(=O)[C@@H](NC(=O)CCCCCN2C(=O)CC(*)C2=O)C(C)C)cc1 ; *C(=O)OCc1ccc(NC(=O)[C@H](CCCNC(N)=O)NC(=O)[C@@H](NC(=O)CCN2C(=O)CC(*)C2=O)C(C)C)cc1]

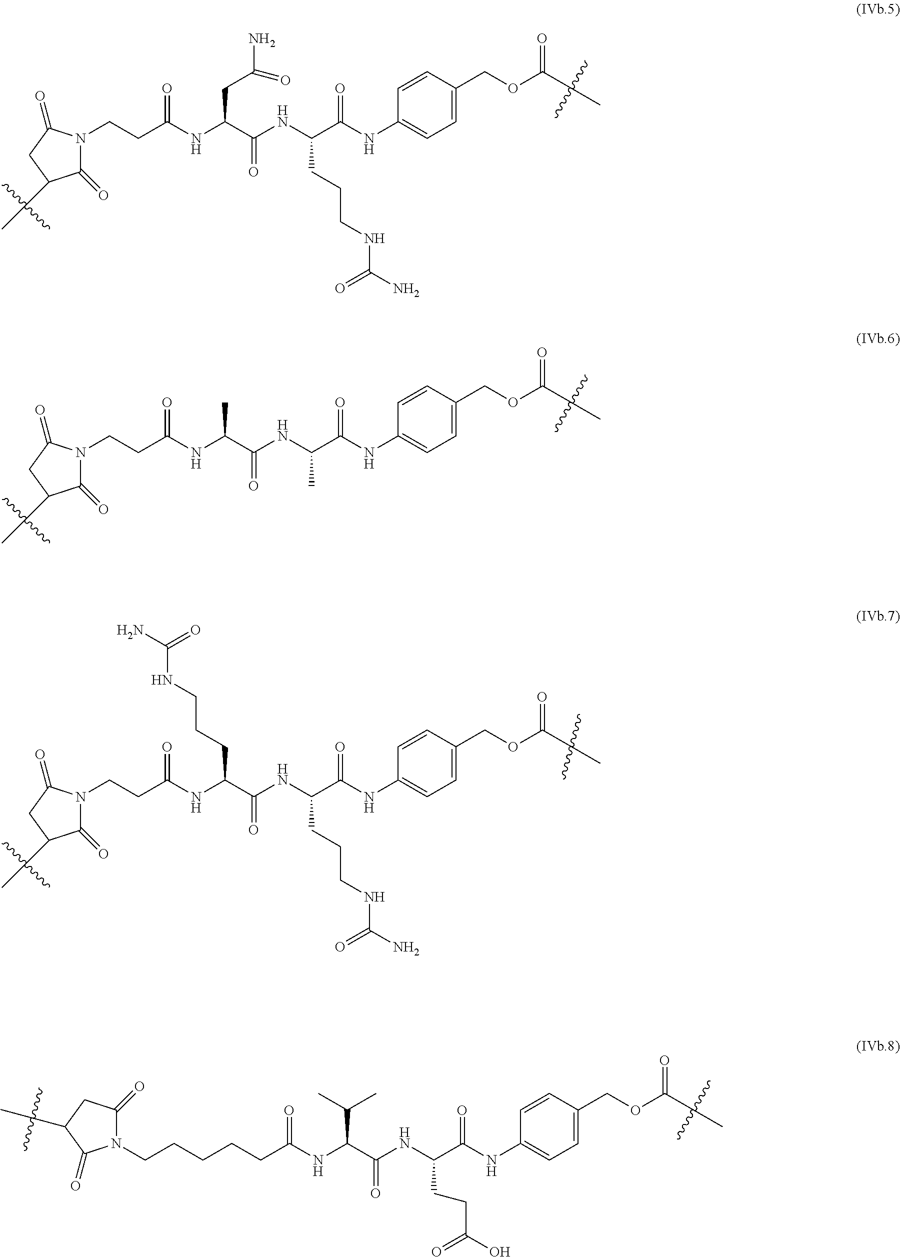 [SMILES: *C(=O)OCc1ccc(NC(=O)[C@H](CCCNC(N)=O)NC(=O)[C@H](CC(N)=O)NC(=O)CCN2C(=O)CC(*)C2=O)cc1 ; *C(=O)OCc1ccc(NC(=O)[C@H](C)NC(=O)[C@H](C)NC(=O)CCN2C(=O)CC(*)C2=O)cc1 ; *C(=O)OCc1ccc(NC(=O)[C@H](CCCNC(N)=O)NC(=O)[C@H](CCCNC(N)=O)NC(=O)CCN2C(=O)CC(*)C2=O)cc1 ; *C(=O)OCc1ccc(NC(=O)[C@H](CCC(=O)O)NC(=O)[C@@H](NC(=O)CCCCCN2C(=O)CC(*)C2=O)C(C)C)cc1]

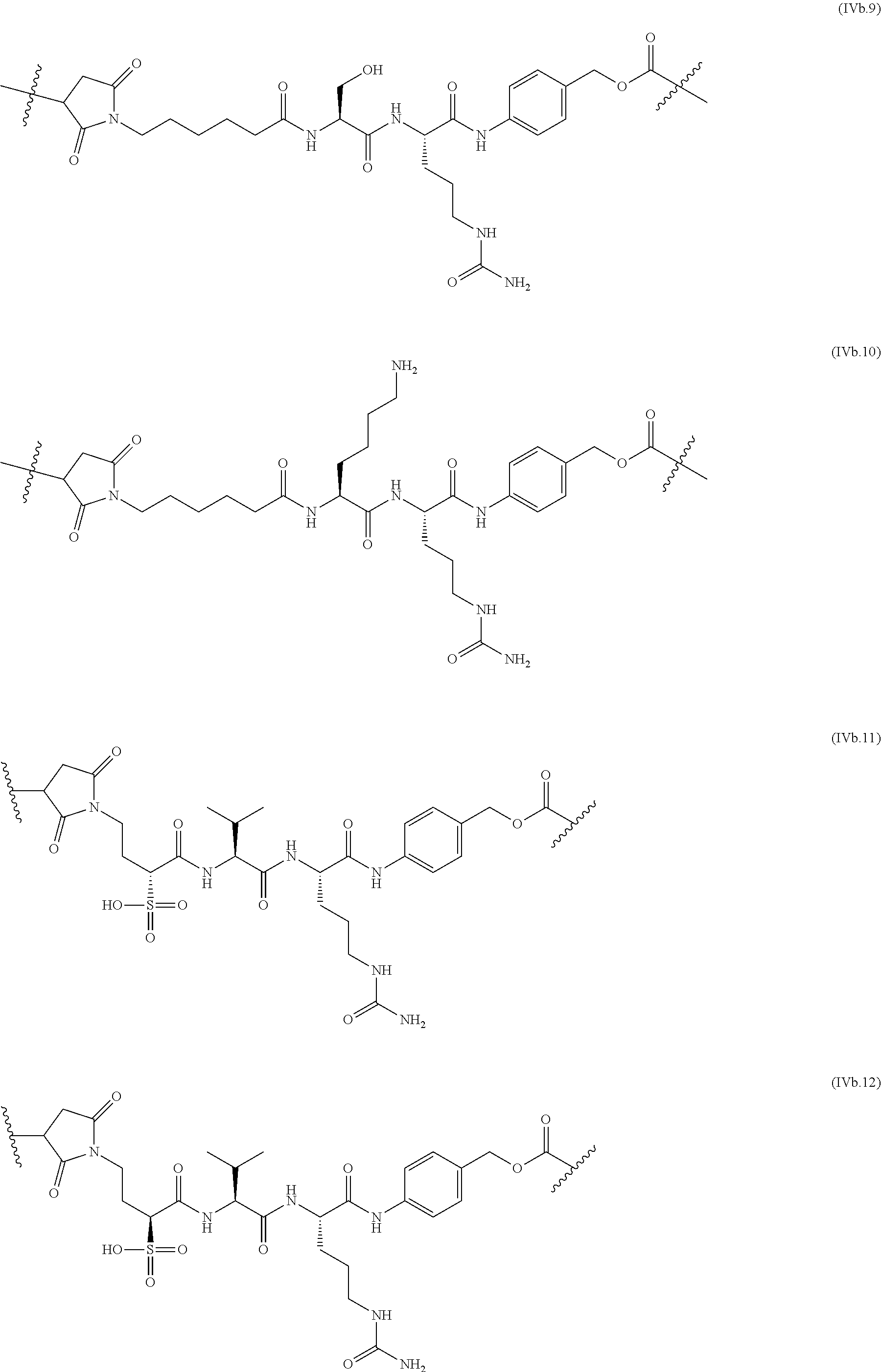 [SMILES: *C(=O)OCc1ccc(NC(=O)[C@H](CCCNC(N)=O)NC(=O)[C@H](CO)NC(=O)CCCCCN2C(=O)CC(*)C2=O)cc1 ; *C(=O)OCc1ccc(NC(=O)[C@H](CCCNC(N)=O)NC(=O)[C@H](CCCCN)NC(=O)CCCCCN2C(=O)CC(*)C2=O)cc1 ; *C(=O)OCc1ccc(NC(=O)[C@H](CCCNC(N)=O)NC(=O)[C@@H](NC(=O)[C@@H](CCN2C(=O)CC(*)C2=O)S(=O)(=O)O)C(C)C)cc1 ; *C(=O)OCc1ccc(NC(=O)[C@H](CCCNC(N)=O)NC(=O)[C@@H](NC(=O)[C@H](CCN2C(=O)CC(*)C2=O)S(=O)(=O)O)C(C)C)cc1]

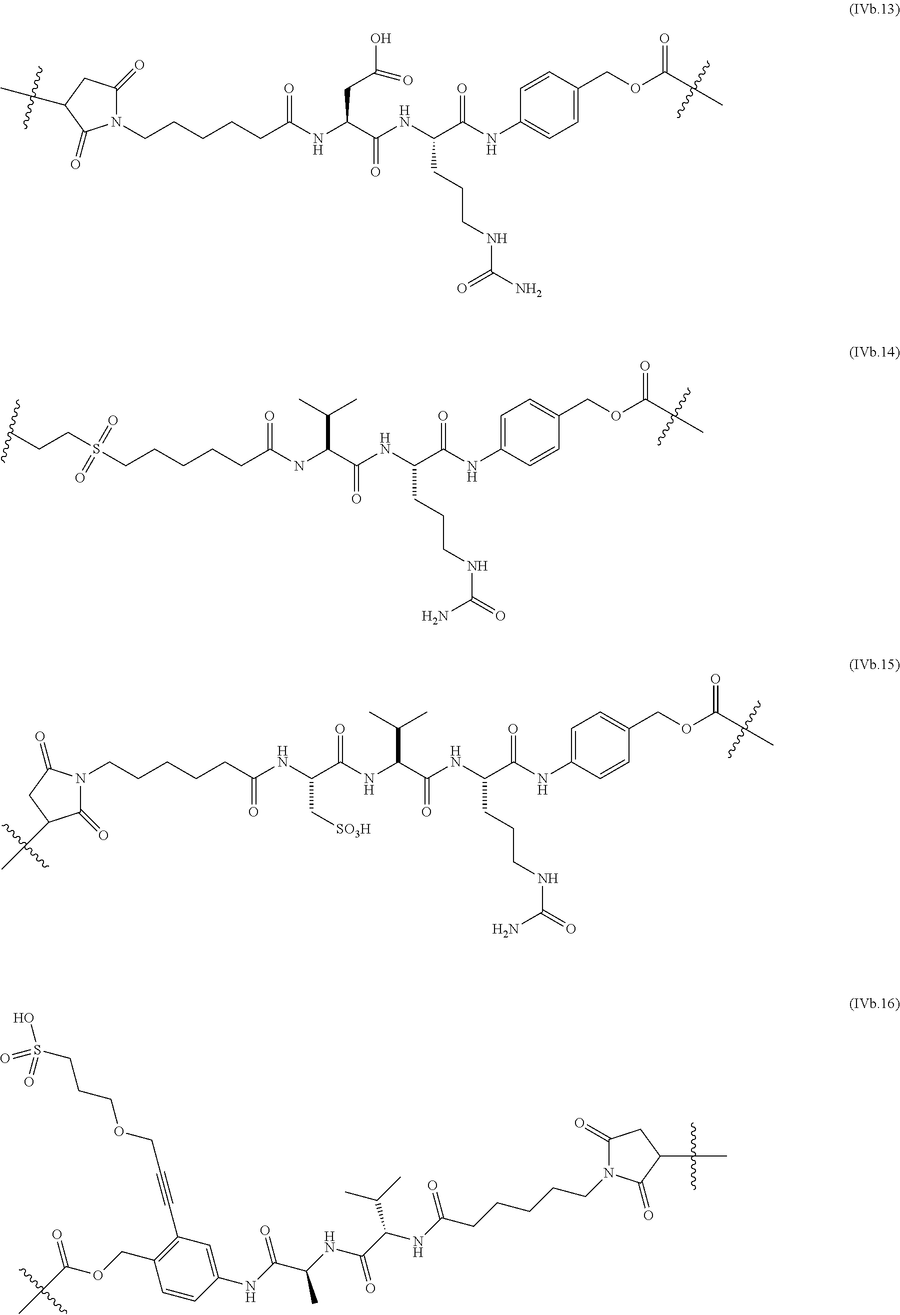 [SMILES: *C(=O)OCc1ccc(NC(=O)[C@H](CCCNC(N)=O)NC(=O)[C@H](CC(=O)O)NC(=O)CCCCCN2C(=O)CC(*)C2=O)cc1 ; *CCS(=O)(=O)CCCCCC(=O)N[C@H](C(=O)N[C@@H](CCCNC(N)=O)C(=O)Nc1ccc(COC(*)=O)cc1)C(C)C ; *C(=O)OCc1ccc(NC(=O)[C@H](CCCNC(N)=O)NC(=O)[C@@H](NC(=O)[C@H](CS(=O)(=O)O)NC(=O)CCCCCN2C(=O)CC(*)C2=O)C(C)C)cc1 ; *C(=O)OCc1ccc(NC(=O)[C@H](C)NC(=O)[C@@H](NC(=O)CCCCCN2C(=O)CC(*)C2=O)C(C)C)cc1C#CCOCCCS(=O)(=O)O]

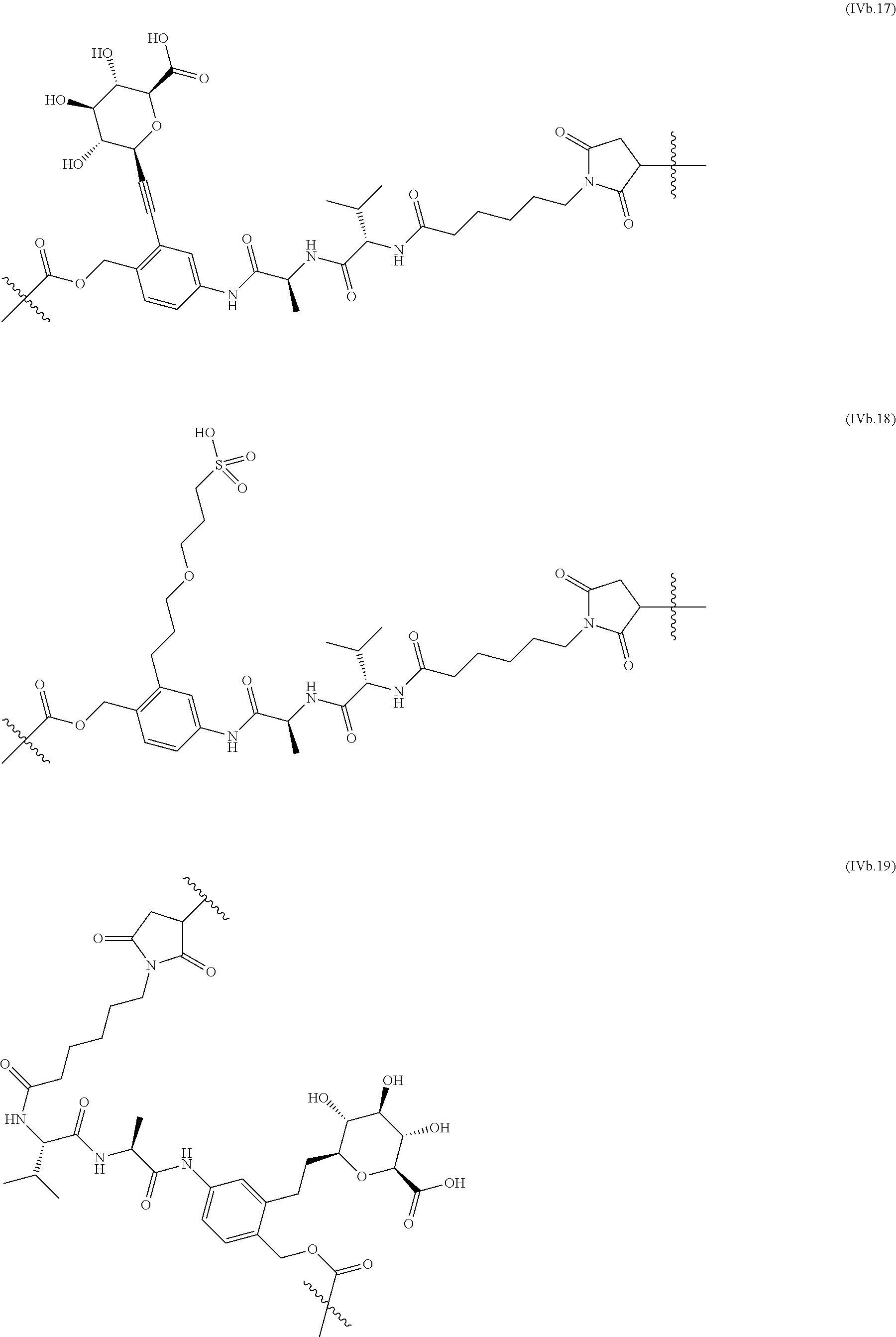 [SMILES: *C(=O)OCc1ccc(NC(=O)[C@H](C)NC(=O)[C@@H](NC(=O)CCCCCN2C(=O)CC(*)C2=O)C(C)C)cc1C#C[C@@H]1O[C@H](C(=O)O)[C@@H](O)[C@H](O)[C@H]1O ; *C(=O)OCc1ccc(NC(=O)[C@H](C)NC(=O)[C@@H](NC(=O)CCCCCN2C(=O)CC(*)C2=O)C(C)C)cc1CCCOCCCS(=O)(=O)O ; *C(=O)OCc1ccc(NC(=O)[C@H](C)NC(=O)[C@@H](NC(=O)CCCCCN2C(=O)CC(*)C2=O)C(C)C)cc1CC[C@@H]1O[C@H](C(=O)O)[C@@H](O)[C@H](O)[C@H]1O]

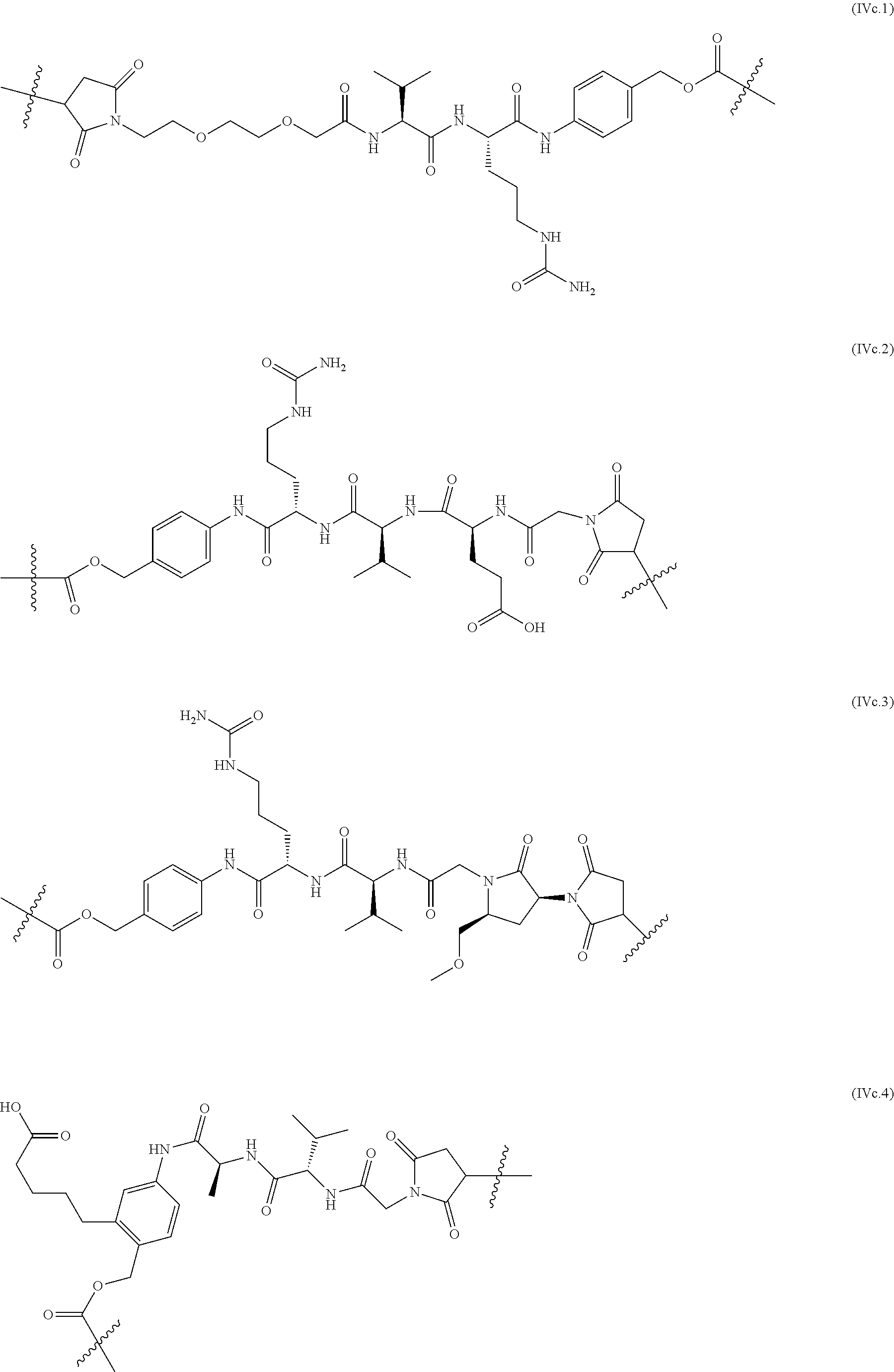 [SMILES: *C(=O)OCc1ccc(NC(=O)[C@H](CCCNC(N)=O)NC(=O)[C@@H](NC(=O)COCCOCCN2C(=O)CC(*)C2=O)C(C)C)cc1 ; *C(=O)OCc1ccc(NC(=O)[C@H](CCCNC(N)=O)NC(=O)[C@@H](NC(=O)[C@H](CCC(=O)O)NC(=O)CN2C(=O)CC(*)C2=O)C(C)C)cc1 ; *C(=O)OCc1ccc(NC(=O)[C@H](CCCNC(N)=O)NC(=O)[C@@H](NC(=O)CN2C(=O)[C@@H](N3C(=O)CC(*)C3=O)C[C@H]2COC)C(C)C)cc1 ; *C(=O)OCc1ccc(NC(=O)[C@H](C)NC(=O)[C@@H](NC(=O)CN2C(=O)CC(*)C2=O)C(C)C)cc1CCCCC(=O)O]

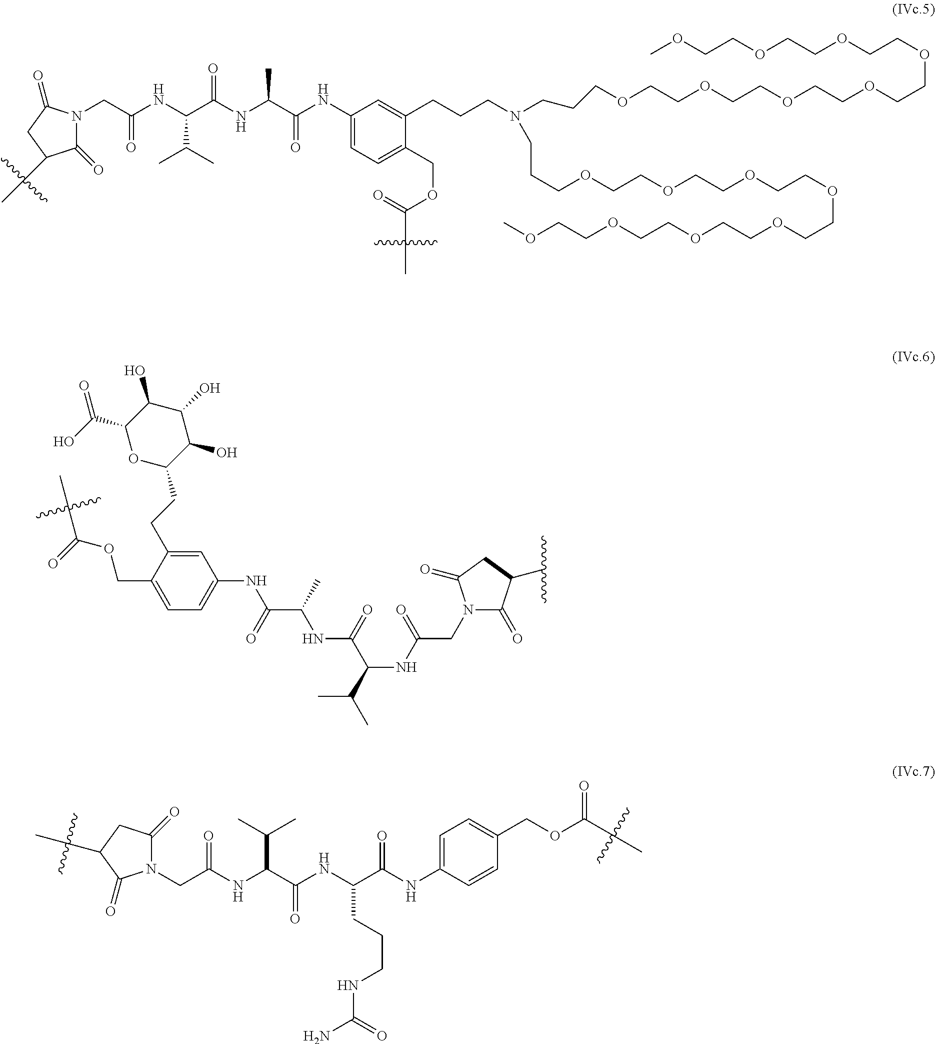 [SMILES: *C(=O)OCc1ccc(NC(=O)[C@H](C)NC(=O)[C@@H](NC(=O)CN2C(=O)CC(*)C2=O)C(C)C)cc1CCCN(CCCOCCOCCOCCOCCOCCOCCOCCOC)CCCOCCOCCOCCOCCOCCOCCOCCOC ; *C(=O)OCc1ccc(NC(=O)[C@H](C)NC(=O)[C@@H](NC(=O)CN2C(=O)C[C@@H](*)C2=O)C(C)C)cc1CC[C@@H]1O[C@H](C(=O)O)[C@@H](O)[C@H](O)[C@H]1O ; *C(=O)OCc1ccc(NC(=O)[C@H](CCCNC(N)=O)NC(=O)[C@@H](NC(=O)CN2C(=O)CC(*)C2=O)C(C)C)cc1]

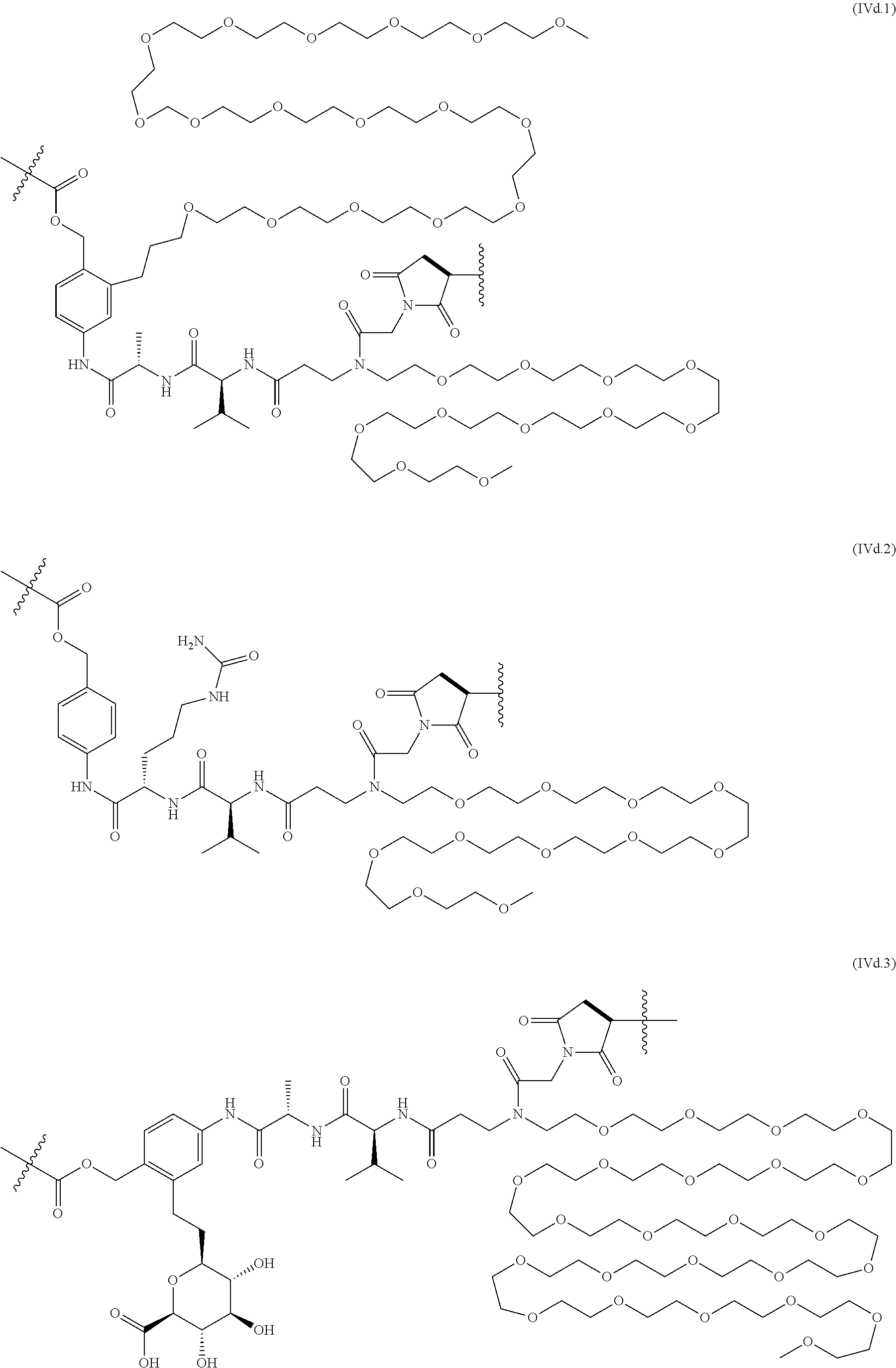

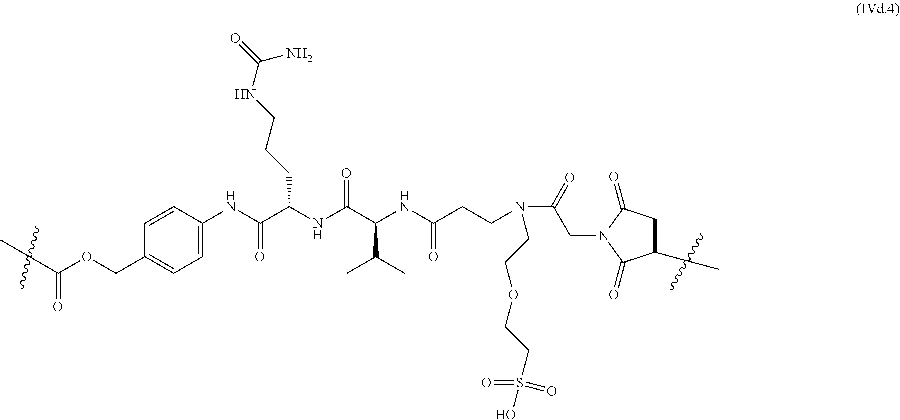 [SMILES: *C(=O)OCc1ccc(NC(=O)[C@H](CCCNC(N)=O)NC(=O)[C@@H](NC(=O)CCN(CCOCCS(=O)(=O)O)C(=O)CN2C(=O)C[C@@H](*)C2=O)C(C)C)cc1]

The cleavable linker can contain an enzymatically cleavable sugar moiety, for example, a linker comprising structural formula (Va), (Vb), (Vc), (Vd), or (Ve):

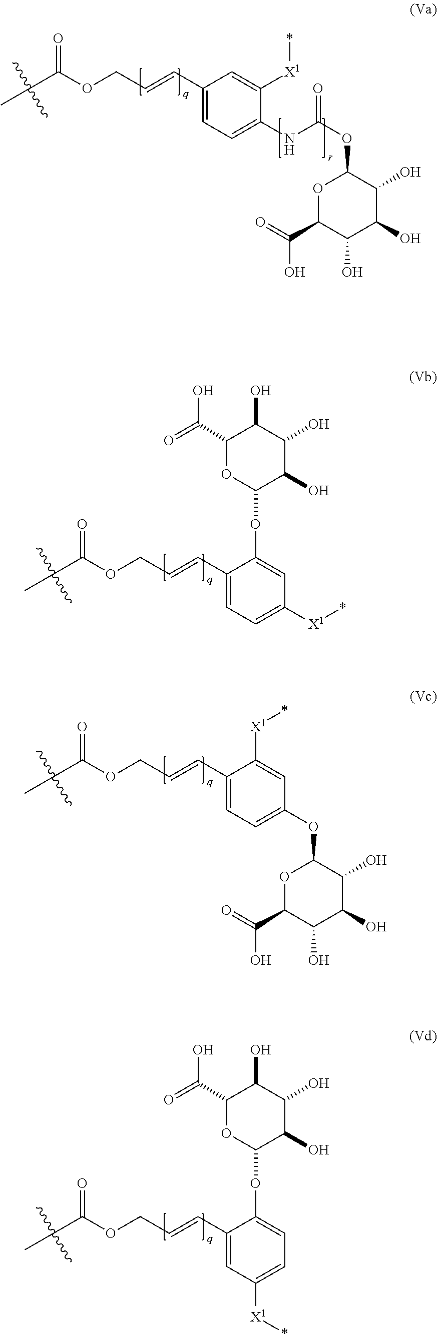

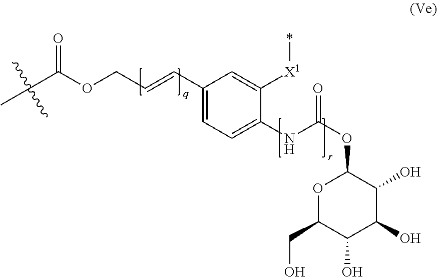

or a salt thereof, wherein: q is 0 or 1; r is 0 or 1; X¹ is CH₂, O or NH; [symbol] represents the point of attachment of the linker to the myeloid cell agonist; and *represents the point of attachment to the remainder of the conjugate.

Exemplary embodiments of connector regions or connector segments according to structural formula (Va) that may be included in the conjugates described herein can include the incorporated moieties from the structures illustrated below, where the skilled practitioner would understand that, when linked within the conjugate, the maleimide in each structure will be in its linked form, i.e., a succinimide moiety

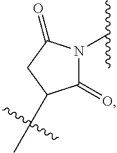 [SMILES: *C1CC(=O)N(*)C1=O]

—S—CH═CH₂ in each structure will be in its linked form i.e.,

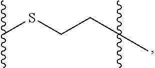 [SMILES: *CCS*]

and —SO₂—CH═CH₂ in will be in its linked form, i.e.,

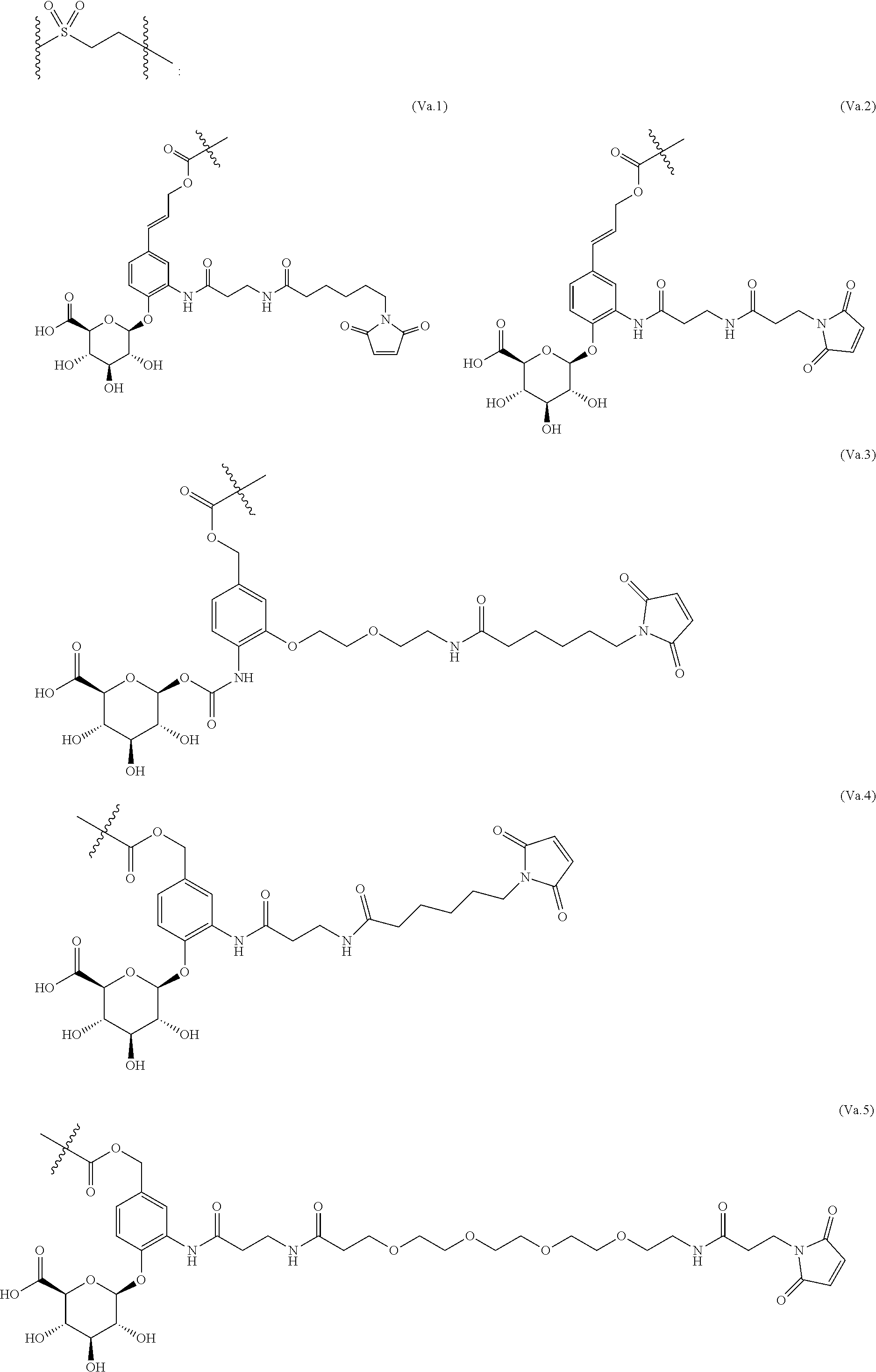

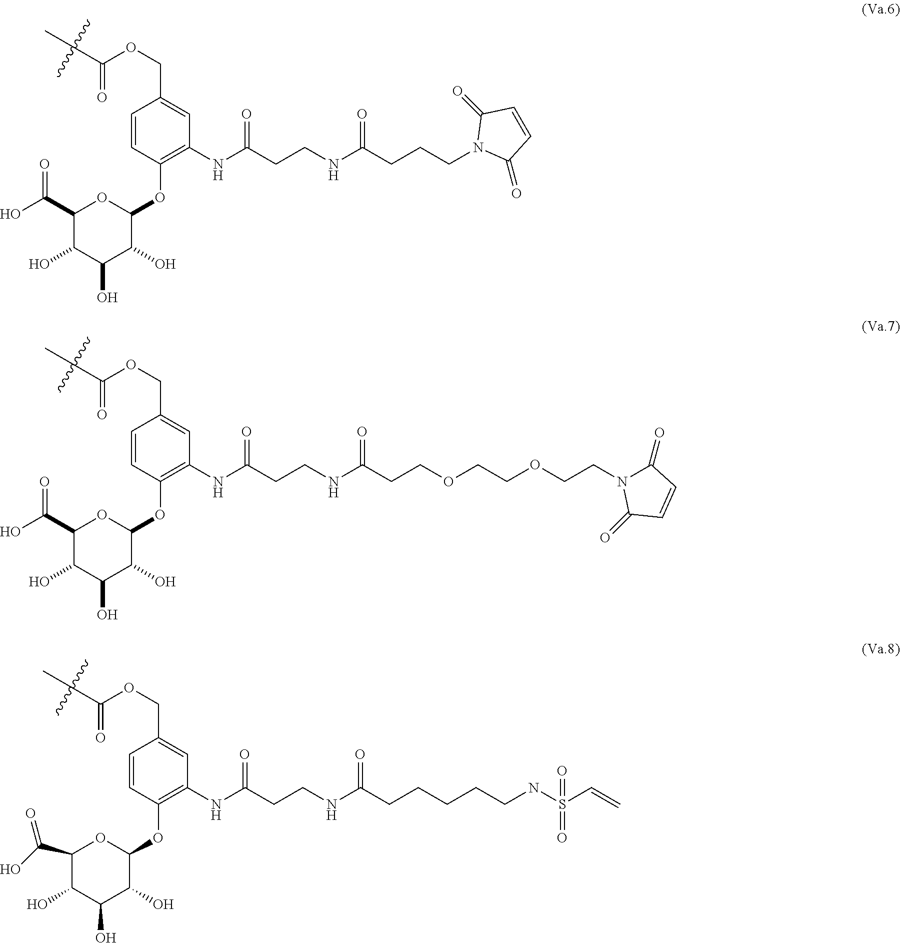 [SMILES: *C(=O)OCc1ccc(O[C@@H]2O[C@H](C(=O)O)[C@@H](O)[C@H](O)[C@H]2O)c(NC(=O)CCNC(=O)CCCN2C(=O)C=CC2=O)c1 ; *C(=O)OCc1ccc(O[C@@H]2O[C@H](C(=O)O)[C@@H](O)[C@H](O)[C@H]2O)c(NC(=O)CCNC(=O)CCOCCOCCN2C(=O)C=CC2=O)c1 ; *C(=O)OCc1ccc(O[C@@H]2O[C@H](C(=O)O)[C@@H](O)[C@H](O)[C@H]2O)c(NC(=O)CCNC(=O)CCCCCNS(=O)(=O)C=C)c1]

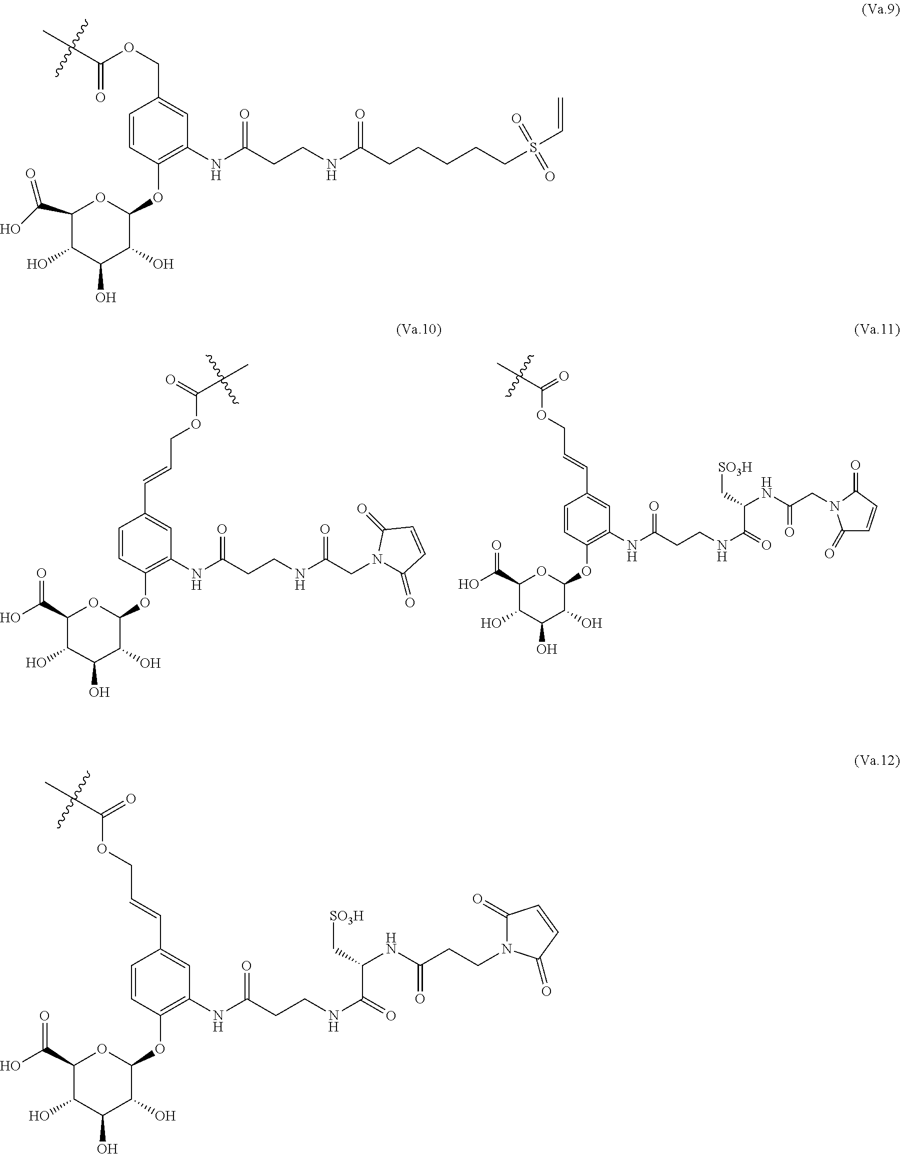 [SMILES: *C(=O)OCc1ccc(O[C@@H]2O[C@H](C(=O)O)[C@@H](O)[C@H](O)[C@H]2O)c(NC(=O)CCNC(=O)CCCCCS(=O)(=O)C=C)c1 ; *C(=O)OC/C=C/c1ccc(O[C@@H]2O[C@H](C(=O)O)[C@@H](O)[C@H](O)[C@H]2O)c(NC(=O)CCNC(=O)CN2C(=O)C=CC2=O)c1 ; *C(=O)OC/C=C/c1ccc(O[C@@H]2O[C@H](C(=O)O)[C@@H](O)[C@H](O)[C@H]2O)c(NC(=O)CCNC(=O)[C@H](CS(=O)(=O)O)NC(=O)CN2C(=O)C=CC2=O)c1 ; *C(=O)OC/C=C/c1ccc(O[C@@H]2O[C@H](C(=O)O)[C@@H](O)[C@H](O)[C@H]2O)c(NC(=O)CCNC(=O)[C@H](CS(=O)(=O)O)NC(=O)CCN2C(=O)C=CC2=O)c1]

Exemplary embodiments of connector regions or connector segments according to structural formula (Vb) that may be included in the conjugates described herein include the structures illustrated below, where the maleimide in each structure is replaced with a succinimide moiety

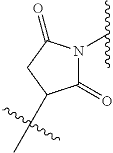 [SMILES: *C1CC(=O)N(*)C1=O]

in the conjugate:

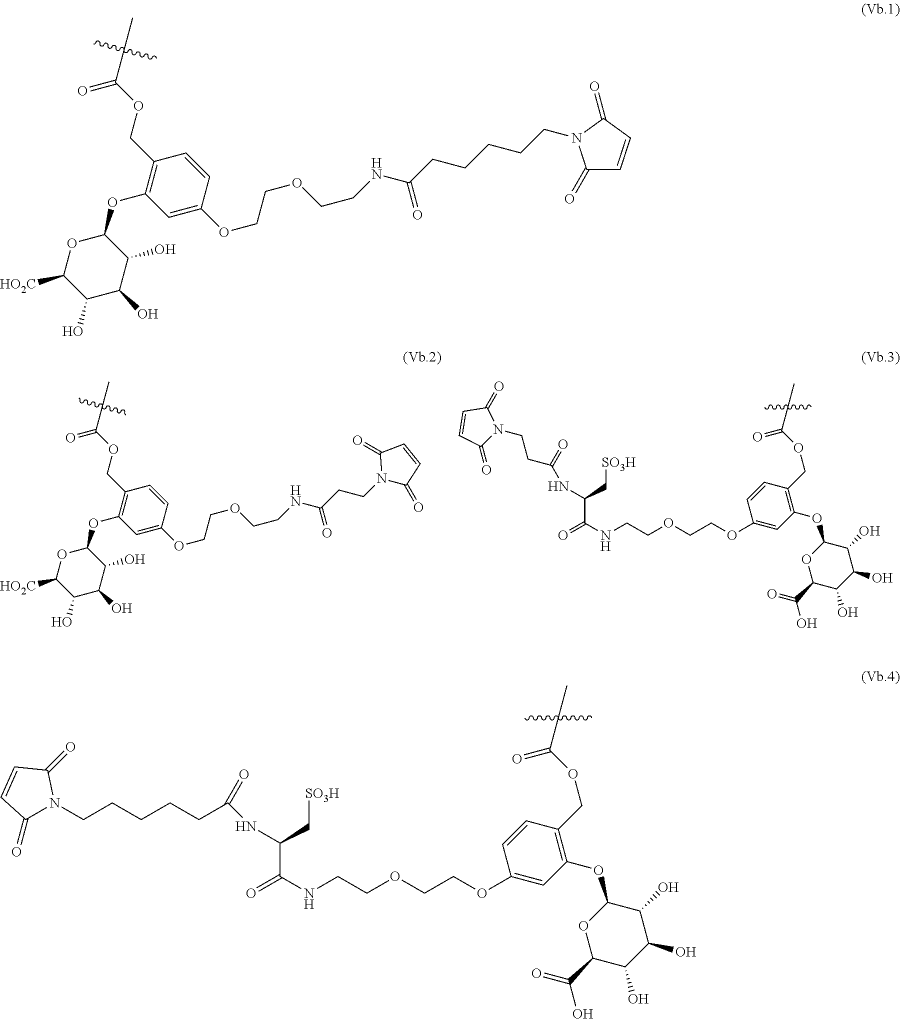 [SMILES: *C(=O)OCc1ccc(OCCOCCNC(=O)CCCCCN2C(=O)C=CC2=O)cc1O[C@@H]1O[C@H](C(=O)O)[C@@H](O)[C@H](O)[C@H]1O ; *C(=O)OCc1ccc(OCCOCCNC(=O)CCN2C(=O)C=CC2=O)cc1O[C@@H]1O[C@H](C(=O)O)[C@@H](O)[C@H](O)[C@H]1O ; *C(=O)OCc1ccc(OCCOCCNC(=O)[C@H](CS(=O)(=O)O)NC(=O)CCN2C(=O)C=CC2=O)cc1O[C@@H]1O[C@H](C(=O)O)[C@@H](O)[C@H](O)[C@H]1O ; *C(=O)OCc1ccc(OCCOCCNC(=O)[C@H](CS(=O)(=O)O)NC(=O)CCCCCN2C(=O)C=CC2=O)cc1O[C@@H]1O[C@H](C(=O)O)[C@@H](O)[C@H](O)[C@H]1O]

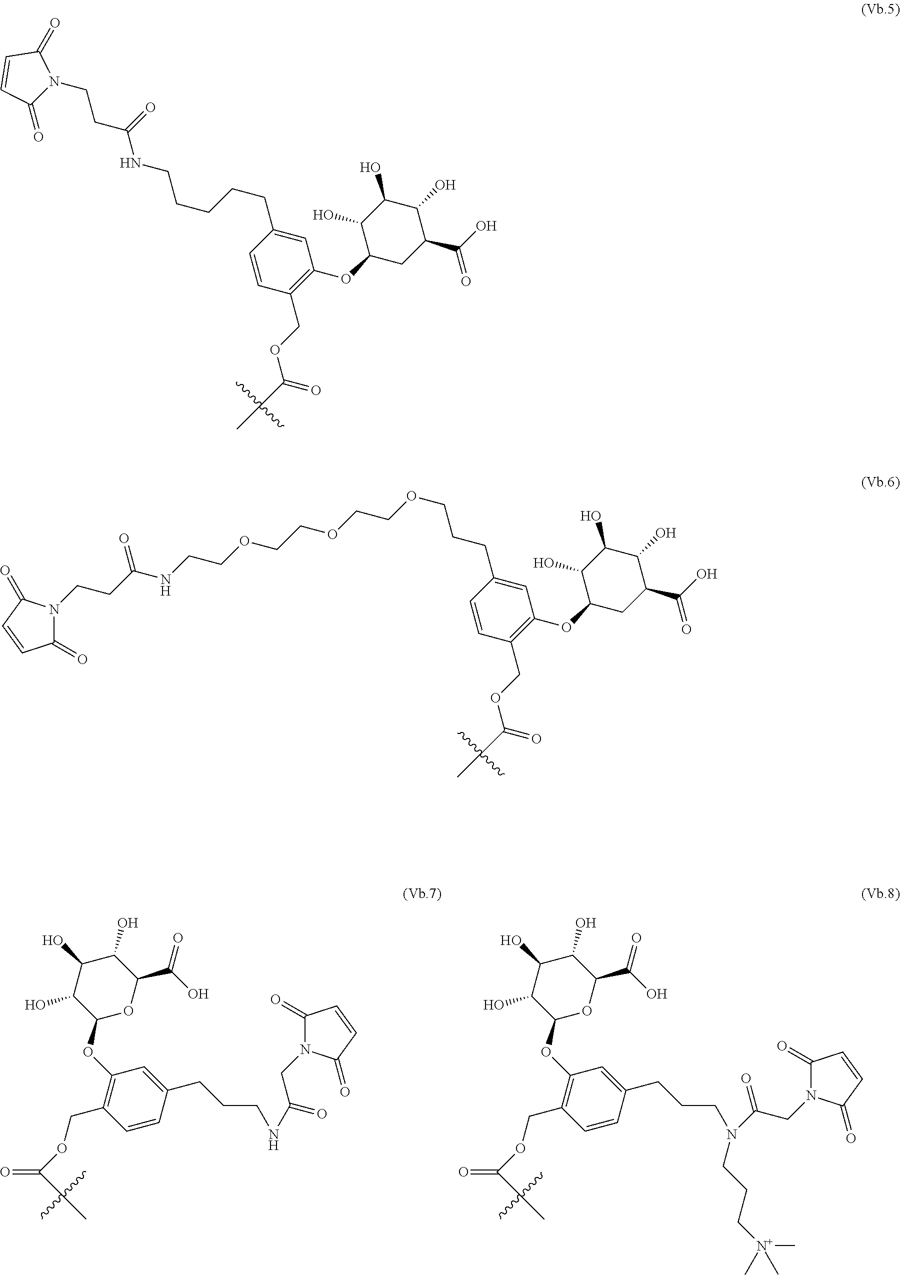 [SMILES: *C(=O)OCc1ccc(CCCCCNC(=O)CCN2C(=O)C=CC2=O)cc1O[C@@H]1C[C@H](C(=O)O)[C@@H](O)[C@H](O)[C@H]1O ; *C(=O)OCc1ccc(CCCOCCOCCOCCNC(=O)CCN2C(=O)C=CC2=O)cc1O[C@@H]1C[C@H](C(=O)O)[C@@H](O)[C@H](O)[C@H]1O ; *C(=O)OCc1ccc(CCCNC(=O)CN2C(=O)C=CC2=O)cc1O[C@@H]1O[C@H](C(=O)O)[C@@H](O)[C@H](O)[C@H]1O ; *C(=O)OCc1ccc(CCCN(CCC[N+](C)(C)C)C(=O)CN2C(=O)C=CC2=O)cc1O[C@@H]1O[C@H](C(=O)O)[C@@H](O)[C@H](O)[C@H]1O]

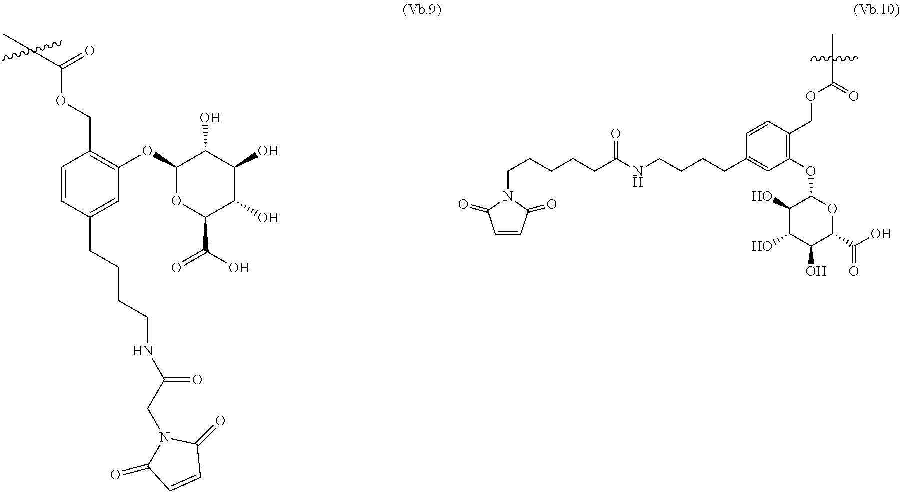 [SMILES: *C(=O)OCc1ccc(CCCCNC(=O)CN2C(=O)C=CC2=O)cc1O[C@@H]1O[C@H](C(=O)O)[C@@H](O)[C@H](O)[C@H]1O ; *C(=O)OCc1ccc(CCCCNC(=O)CCCCCN2C(=O)C=CC2=O)cc1O[C@@H]1O[C@H](C(=O)O)[C@@H](O)[C@H](O)[C@H]1O]

Exemplary embodiments of connector regions or connector segments according to structural formula (Vc) that may be included in the conjugates described herein include the linkers illustrated below, where the maleimide in each structure is replaced with a succinimide moiety

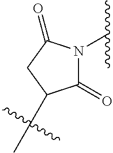 [SMILES: *C1CC(=O)N(*)C1=O]

in the conjugate:

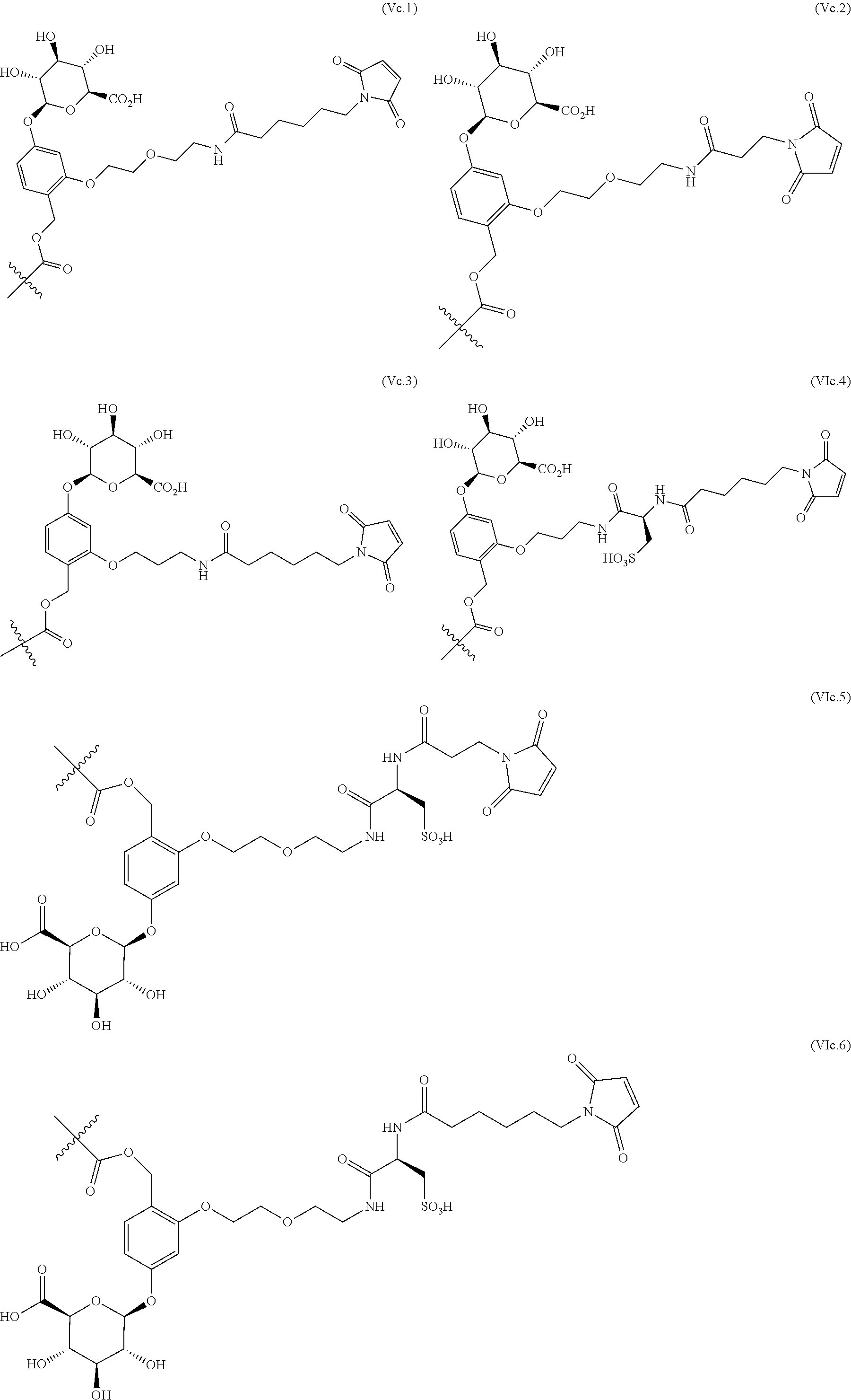

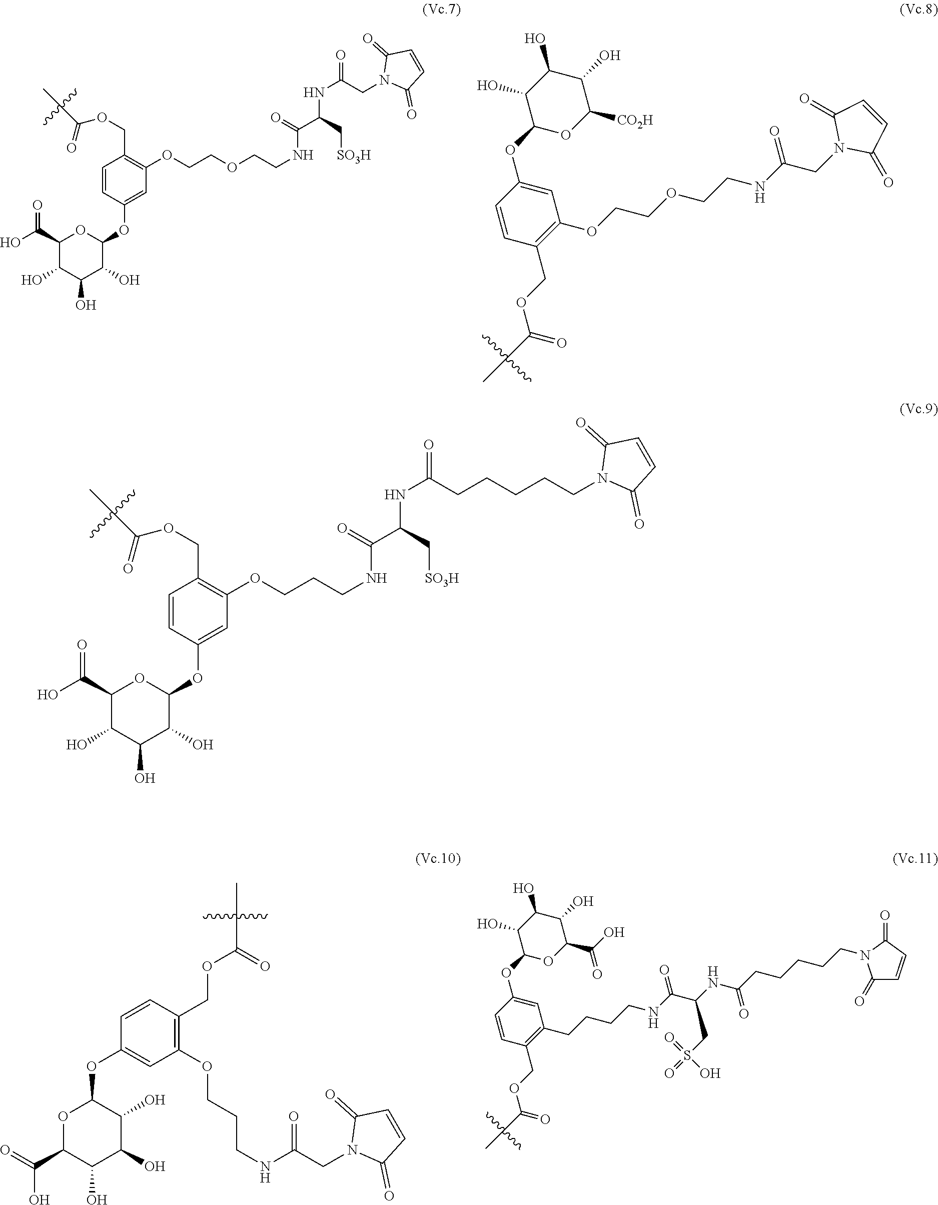

Exemplary embodiments of connector regions or connector segments according to structural formula (Vd) that may be included in the conjugates described herein include the structures illustrated below, where the maleimide in each structure is replaced with a succinimide moiety

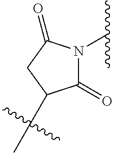 [SMILES: *C1CC(=O)N(*)C1=O]

in the conjugate:

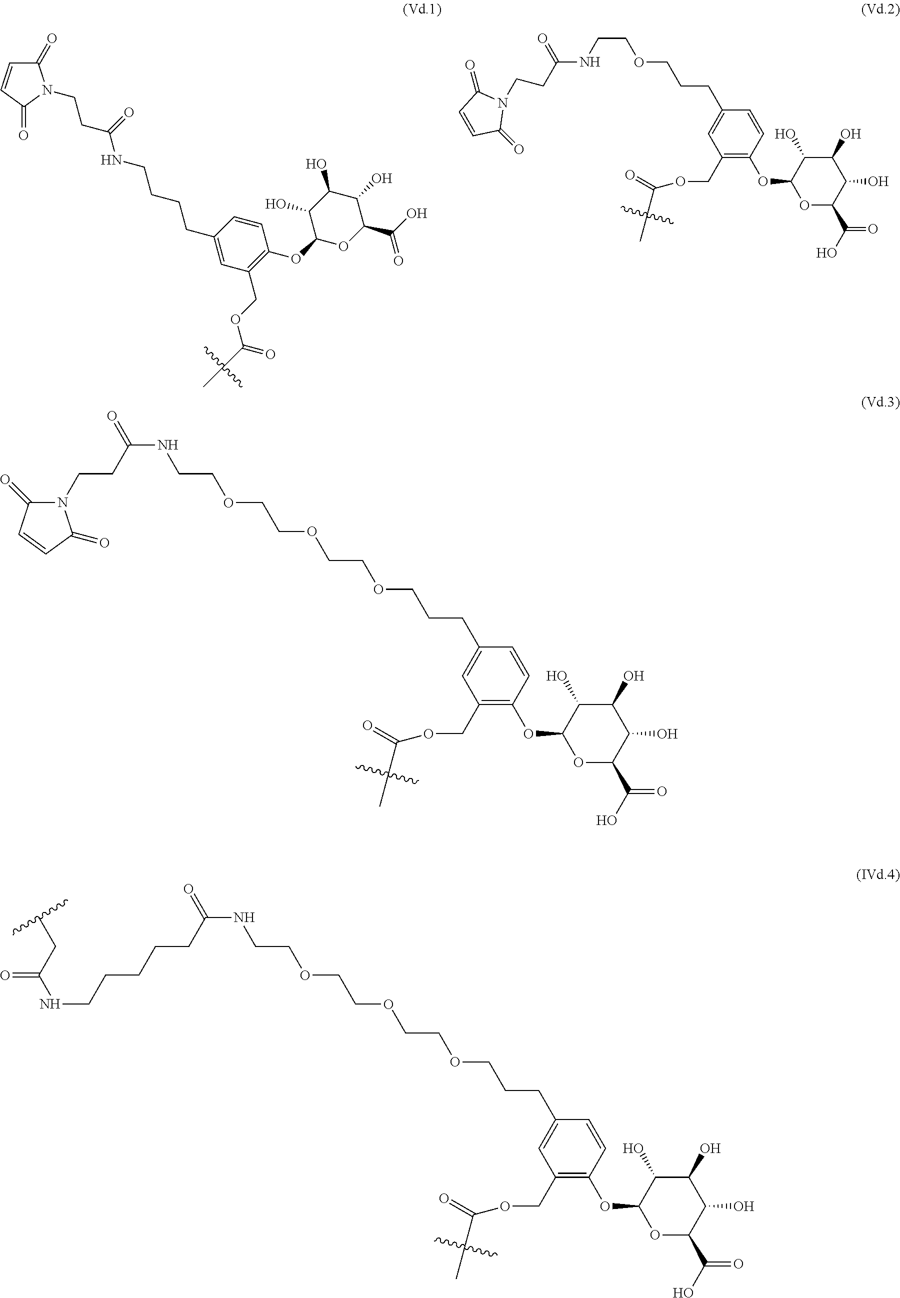 [SMILES: *C(=O)OCc1cc(CCCCNC(=O)CCN2C(=O)C=CC2=O)ccc1O[C@@H]1O[C@H](C(=O)O)[C@@H](O)[C@H](O)[C@H]1O ; *C(=O)OCc1cc(CCCOCCNC(=O)CCN2C(=O)C=CC2=O)ccc1O[C@@H]1O[C@H](C(=O)O)[C@@H](O)[C@H](O)[C@H]1O ; *C(=O)OCc1cc(CCCOCCOCCOCCNC(=O)CCN2C(=O)C=CC2=O)ccc1O[C@@H]1O[C@H](C(=O)O)[C@@H](O)[C@H](O)[C@H]1O ; *CC(=O)NCCCCCC(=O)NCCOCCOCCOCCCc1ccc(O[C@@H]2O[C@H](C(=O)O)[C@@H](O)[C@H](O)[C@H]2O)c(COC(*)=O)c1]

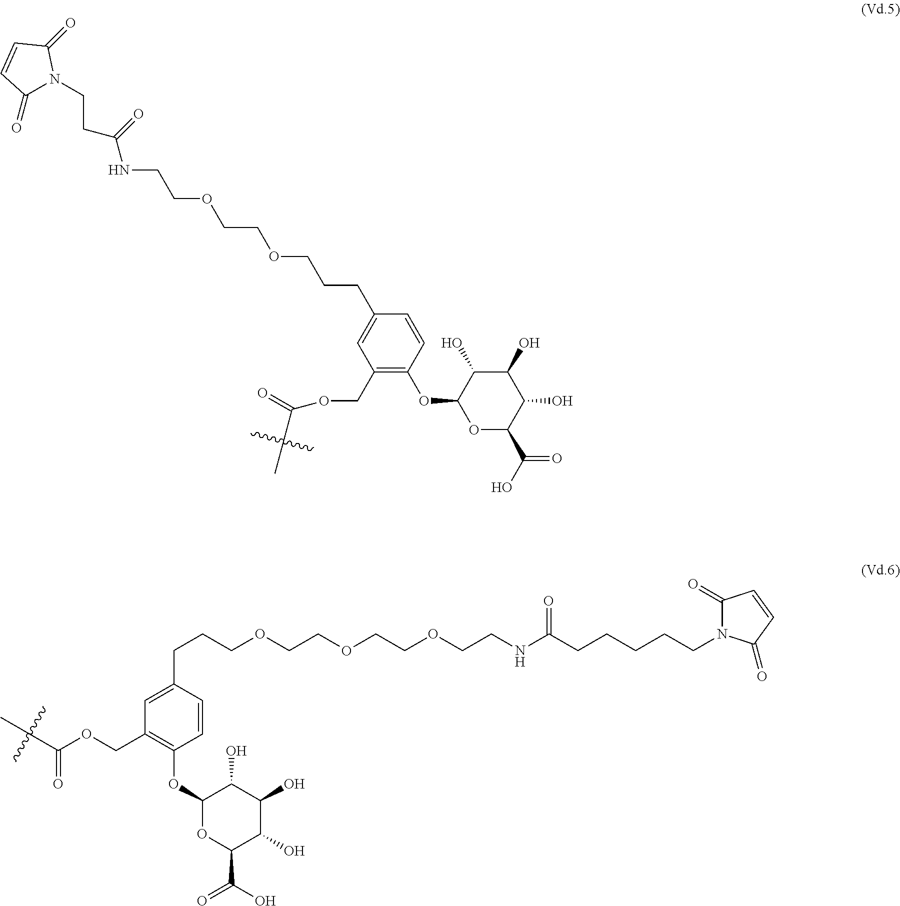 [SMILES: *C(=O)OCc1cc(CCCOCCOCCNC(=O)CCN2C(=O)C=CC2=O)ccc1O[C@@H]1O[C@H](C(=O)O)[C@@H](O)[C@H](O)[C@H]1O ; *C(=O)OCc1cc(CCCOCCOCCOCCNC(=O)CCCCCN2C(=O)C=CC2=O)ccc1O[C@@H]1O[C@H](C(=O)O)[C@@H](O)[C@H](O)[C@H]1O]

Exemplary embodiments of connector regions or connector segments according to structural formula (Ve) that may be included in the conjugates described herein include the structures illustrated below, where the maleimide in each structure is replaced with a succinimide moiety

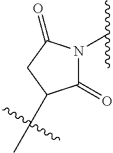 [SMILES: *C1CC(=O)N(*)C1=O]

in the conjugate.

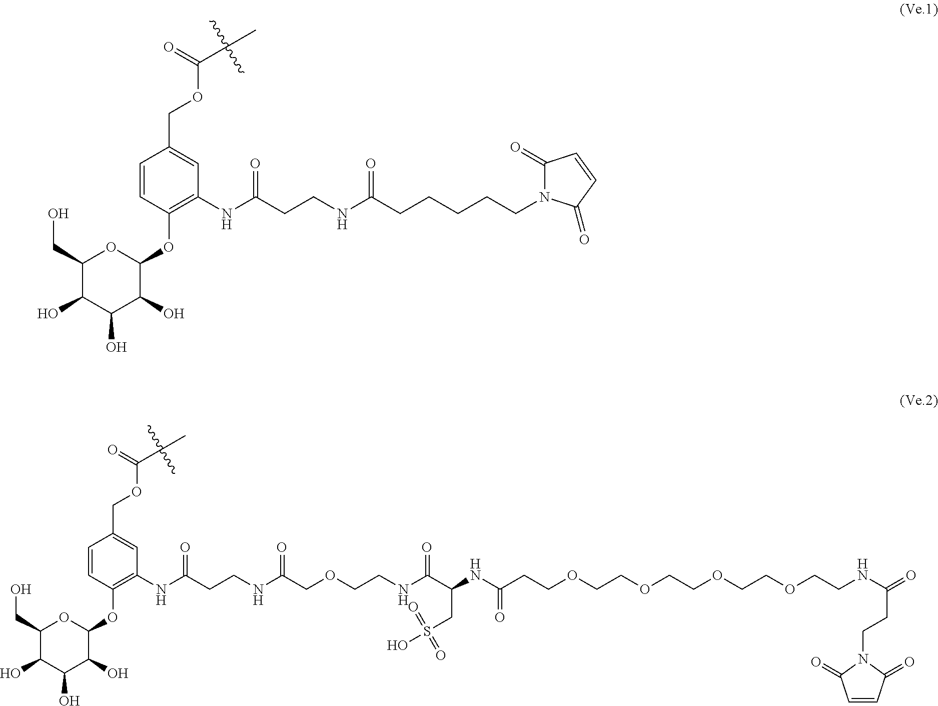 [SMILES: *C(=O)OCc1ccc(O[C@@H]2O[C@H](CO)[C@H](O)[C@H](O)[C@@H]2O)c(NC(=O)CCNC(=O)CCCCCN2C(=O)C=CC2=O)c1 ; *C(=O)OCc1ccc(O[C@@H]2O[C@H](CO)[C@H](O)[C@H](O)[C@@H]2O)c(NC(=O)CCNC(=O)COCCNC(=O)[C@H](CS(=O)(=O)O)NC(=O)CCOCCOCCOCCOCCNC(=O)CCN2C(=O)C=CC2=O)c1]

Although cleavable linkers can provide certain advantages, the connector regions in the conjugates described herein need not include cleavable linkers. For non-cleavable linkers, the compound or myeloid cell agonist release may not depend on the differential properties between the plasma and some cytoplasmic compartments.

The linker can be non-cleavable in vivo, for example, a linker according to the formulations below:

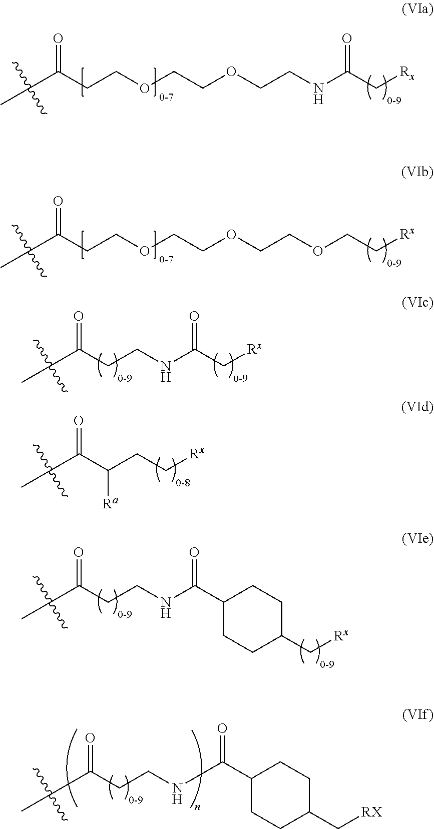

or salts thereof, wherein:
R^{a} is selected from hydrogen, alkyl, sulfonate and methyl sulfonate;
R^{x} is a moiety that covalently links the connector to the rest of the conjugate, such as a bond, a succinimide moiety, or a hydrolyzed succinimide moiety; and [symbol]
represents the point of attachment of the connector region or segment to the rest of the conjugate.

Exemplary embodiments of connector regions or connector segments according to structural formula (VIa)-(VId) that may be included in the conjugates described herein include the structures illustrated below, where the maleimide in each structure is replaced with a succinimide moiety

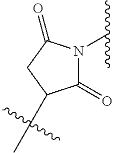 [SMILES: *C1CC(=O)N(*)C1=O]

in the conjugate and —SO₂—CH═CH₂ in each structure is replaced with

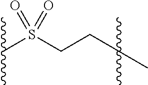 [SMILES: *CCS(*)(=O)=O]

in the conjugate:

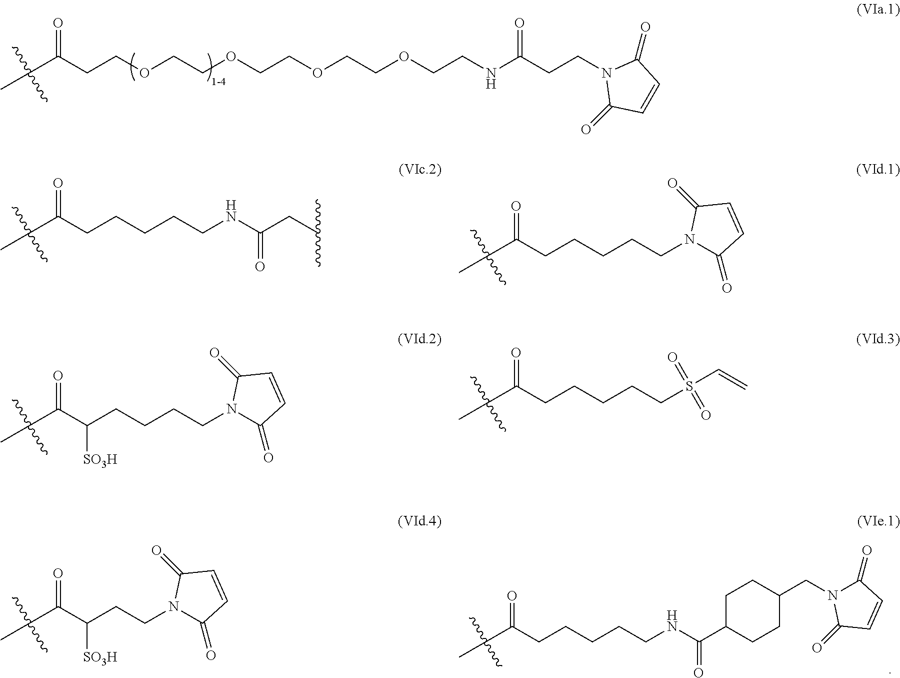

Attachment groups that are used to attach the connectors in a conjugate can be electrophilic in nature and include, for example, maleimide groups, activated disulfides, active esters such as NHS esters and HOBt esters, haloformates, acid halides, alkyl, and benzyl halides such as haloacetamides. There are also emerging technologies related to “self-stabilizing” maleimides and “bridging disulfides” that can be used in accordance with the disclosure.

Maleimide groups are frequently used in the preparation of conjugates because of their specificity for reacting with thiol groups of, for example, cysteine groups of the antibody of a conjugate. The reverse reaction leading to maleimide elimination from a thio-substituted succinimide may also take place. This reverse reaction is undesirable as the maleimide group may subsequently react with another available thiol group such as other proteins in the body having available cysteines. Accordingly, the reverse reaction can undermine the specificity of a conjugate. One method of preventing the reverse reaction is to incorporate a basic group into the linking group shown in the scheme above. Without wishing to be bound by theory, the presence of the basic group may increase the nucleophilicity of nearby water molecules to promote ring-opening hydrolysis of the succinimide group. The hydrolyzed form of the attachment group is resistant to deconjugation in the presence of plasma proteins. So-called “self-stabilizing” linkers provide conjugates with improved stability.

Examples of self-stabilizing linkers are provided in, e.g., U.S. Patent Publication Number 2013/0309256, the linkers of which are incorporated by reference herein. It will be understood that a self-stabilizing linker useful in conjunction with immune-stimulatory compounds may be equivalently described as unsubstituted maleimide-including linkers, thio-substituted succinimide-including linkers, or hydrolyzed, ring-opened thio-substituted succinimide-including linkers. In certain embodiments, a linker comprises a stabilizing linker moiety selected from:

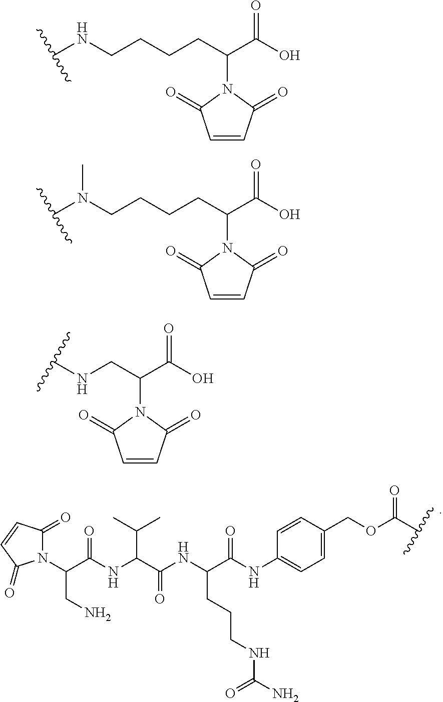

A method for bridging a pair of sulfhydryl groups derived from reduction of a native hinge disulfide bond has been disclosed and is depicted in the schematic below. An advantage of this methodology can be the ability to synthesize homogenous DAR4 conjugates by full reduction of IgGs (to give 4 pairs of sulfhydryls) followed by reaction with 4 equivalents of the alkylating agent. Similarly, as depicted below, a maleimide derivative that can bridge a pair of sulfhydryl groups has been developed.

The linker can contain the following structural formulas (VIIa), (VIIb), or (VIIc), where the maleimide in each structure is replaced with a succinimide moiety

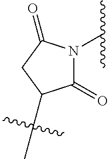 [SMILES: *C1CC(=O)N(*)C1=O]

in the conjugate:

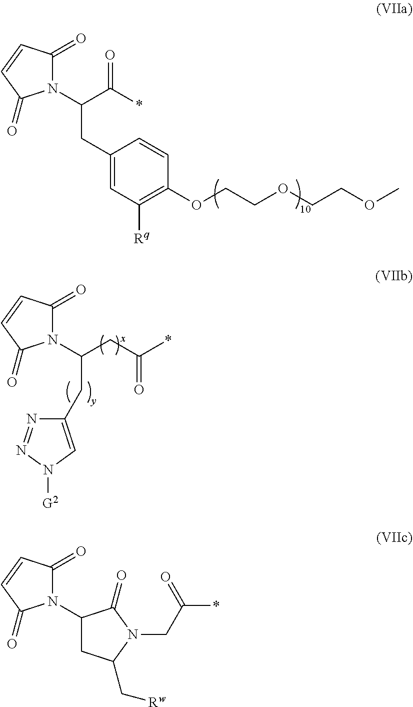

or salts thereof, wherein:
R^{q} is H or —O—(CH₂CH₂O)₁₁—CH₃;
x is 00r 1;
y is 00r 1;
G² is —CH₂CH₂CH₂SO₃H or —CH₂CH₂O—(CH₂CH₂O)₁₁—CH₃;
R^{w} is —O—CH₂CH₂SO₃H or —NH(CO)—CH₂CH₂O—(CH₂CH₂O)₁₂—CH₃; and
* represents the point of attachment to the remainder of the linker.

Exemplary embodiments of linkers that can be included in the conjugates described herein can include the structures illustrated below, where the maleimide in each structure is replaced with a succinimide moiety

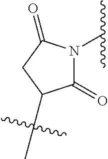 [SMILES: *C1CC(=O)N(*)C1=O]

in the conjugate:

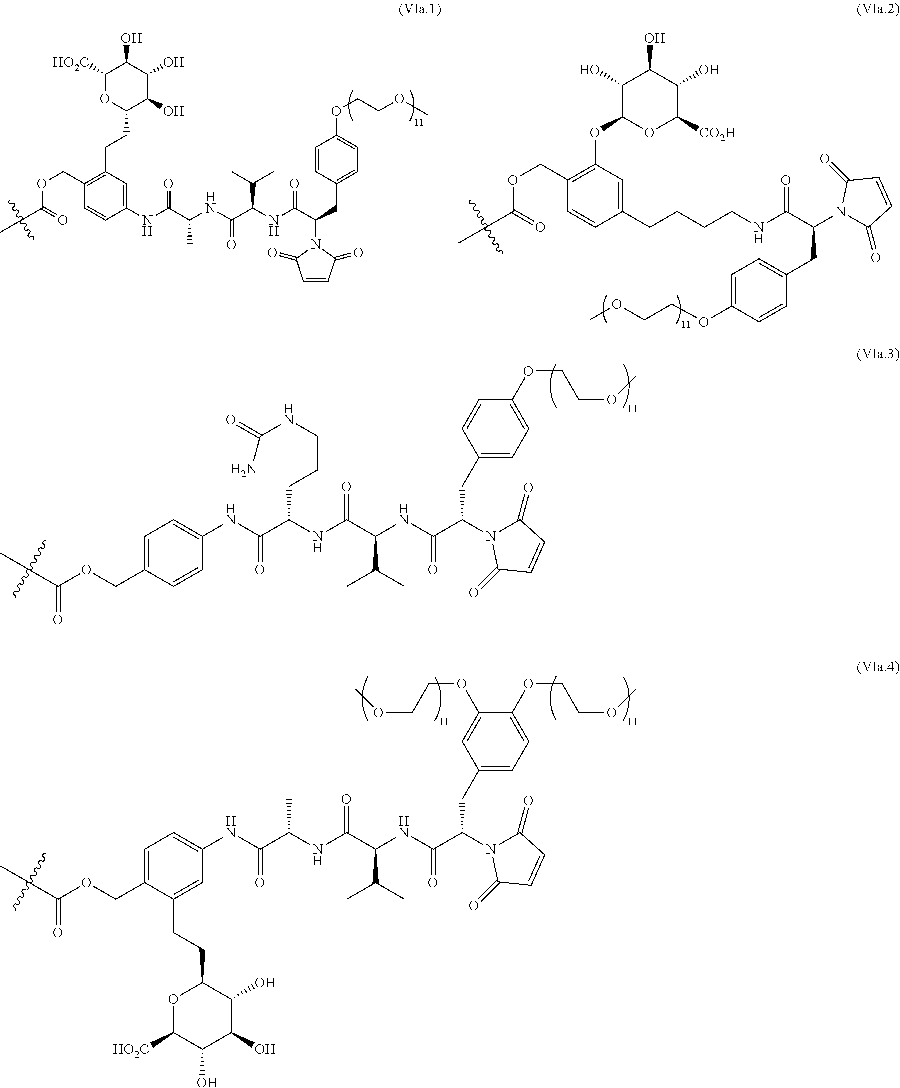

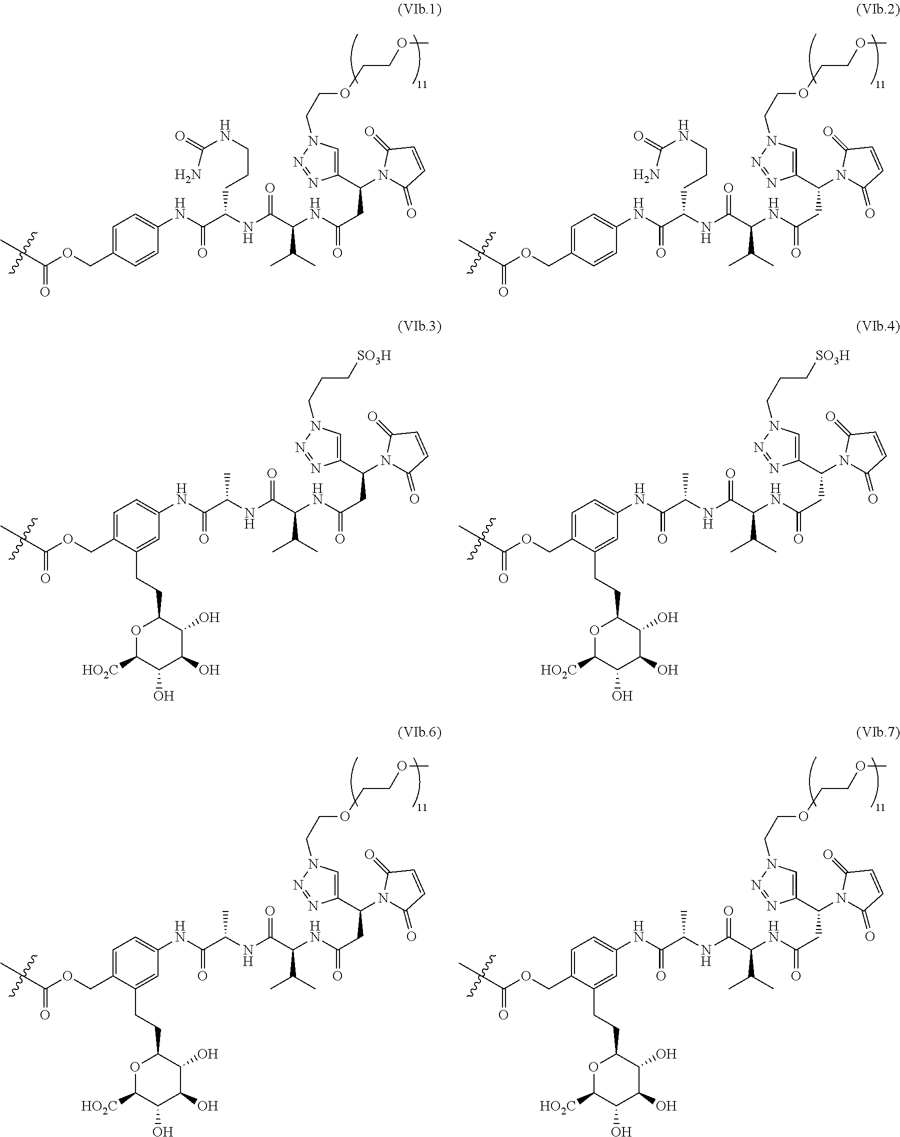

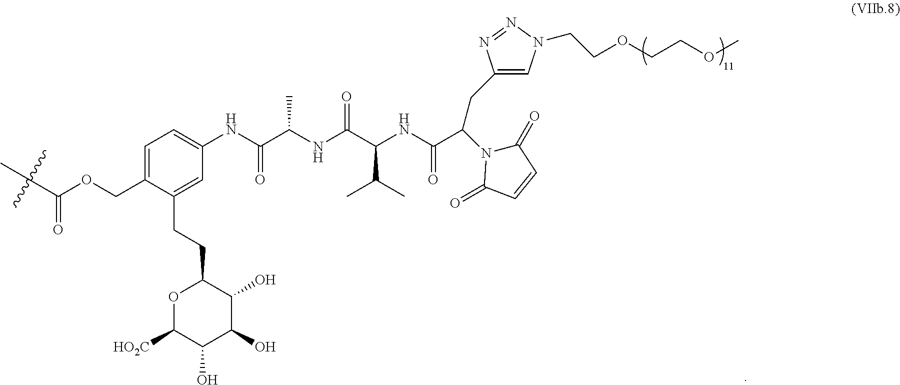

Exemplary embodiments of connector regions or connector segments according to structural formula (VIIc) that can be included in the conjugates described herein can include the structures illustrated below, where the maleimide in each structure is replaced with a succinimide moiety

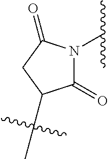 [SMILES: *C1CC(=O)N(*)C1=O]

in the conjugate:

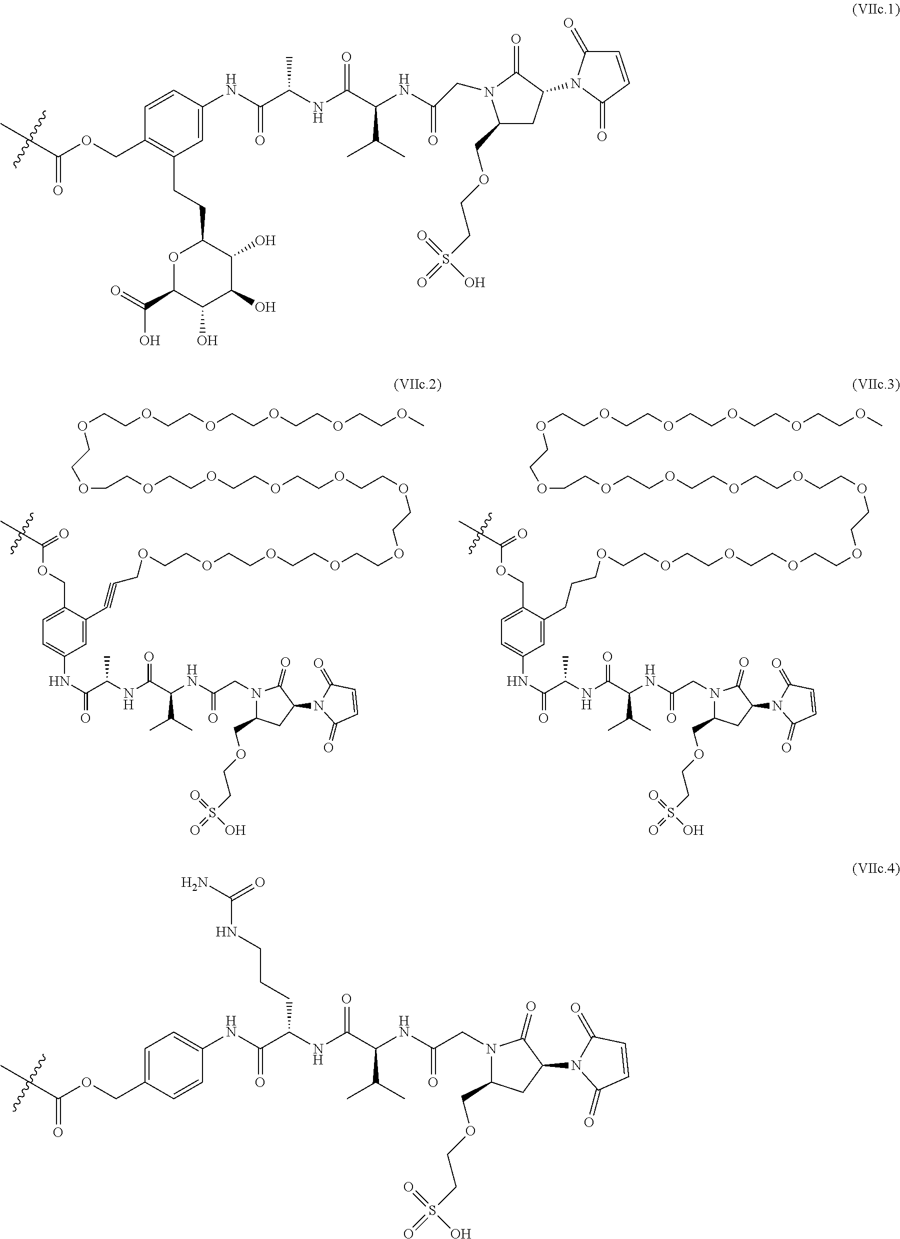

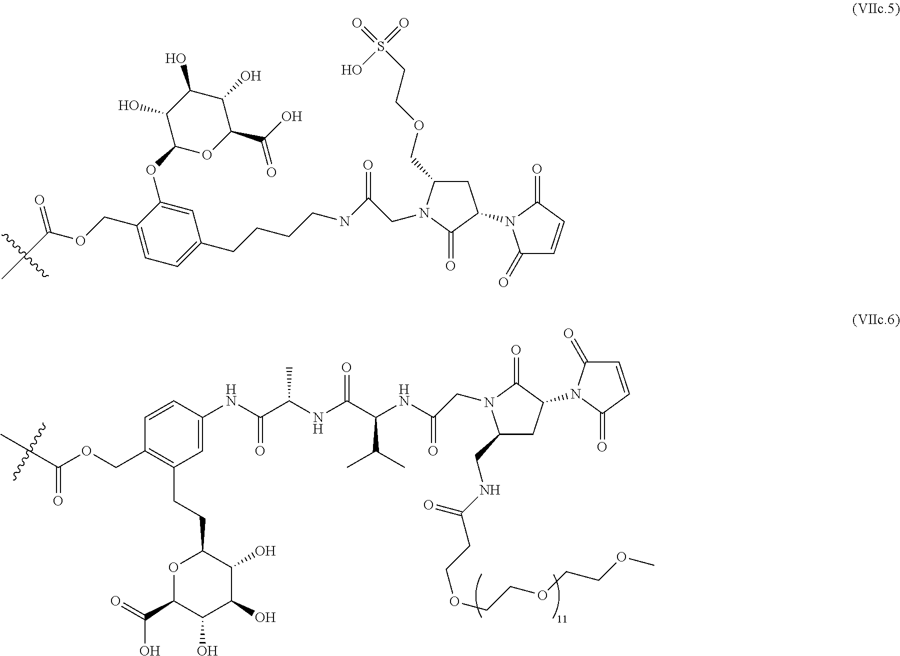

A linker can be attached to a polypeptide at any suitable position. Factors to be considered in selecting an attachment site include whether the linker is cleavable or non-cleavable, the reactive group of the linker for attachment to the polypeptide, the chemical nature of the compound and compatabiltity with reactive sites on the linker and the polypeptide, and the effect of the attachment site on functional activities of the polypeptide, such as functional activities of an Fc domain. A linker may be attached to a terminus of an amino acid sequence of polypeptide or can be attached to a side chain of an amino acid of a polypeptide, such as the side chain of a lysine, serine, threonine, cysteine, tyrosine, aspartic acid, glutamine, a non-natural amino acid residue, or glutamic acid residue. A linker may be bound to a terminus of an amino acid sequence of an Fc domain or Fc region of an antibody, or may be bound to a side chain of an amino acid of an Fc domain of an antibody, such as the side chain of a lysine, serine, threonine, cysteine, tyrosine, aspartic acid, glutamine, a non-natural amino acid residue, or glutamic acid residue.

In some embodiments, a linker is attached to a hinge cysteine of an antibody Fc domain. A linker can be attached to an antibody at a light chain constant domain lysine. A linker can be attached to an antibody at an engineered cysteine in the light chain. A linker can be attached to an antibody at an engineered light chain glutamine. A linker can be attached to an antibody at an unnatural amino acid engineered into the light chain. A linker can be attached to an antibody at a heavy chain constant domain lysine. A linker can be attached to an antibody at an engineered cysteine in the heavy chain. A linker can be attached to an antibody at an engineered heavy chain glutamine. A linker can be attached to an antibody at an unnatural amino acid engineered into the heavy chain. Amino acids can be engineered into an amino acid sequence of an antibody as described herein or as known to the skilled artisan and can be connected to a linker of a conjugate. Engineered amino acids can be added to a sequence of existing amino acids. Engineered amino acids can be substituted for one or more existing amino acids of a sequence of amino acids.

A linker can be attached to a polypeptide via a sulfhydryl group. A linker can be attached to an antibody via a primary amine. A linker can be a link created between an unnatural amino acid on an antibody reacting with oxime bond that was formed by modifying a ketone group with an alkoxyamine on an immune stimulatory compound.

Benzazepine and benzazepine-like compounds may be synthesized using techniques and synthetic methods known in the art, including those described, for example, in PCT Publication Nos. WO2018/170179, WO2017/202703, WO2017/202704, WO2016/096778, WO2017/216054, WO2017/046112, and US 2019/0016808. Compound-linker units and compound-linker-polypeptide conjugates can be synthesized using methods known in the art, including those described in described, for example, in PCT Publication Nos. WO2018/170179, WO2017/202703, WO2017/202704, WO2016/096778, WO2017/216054, WO2017/046112, and US 2019/0016808.

### Exemplary Pharmaceutical Formulations

Provided herein are aqueous formulations comprising a conjugate, wherein the conjugate comprises a benzazepine or benzazepine-like compound linked to a polypeptide. The present inventors discovered that the benzazepine compound of a conjugate comprising a benzazepine compound drug linked to a polypeptide (such as an antibody) may undergo a chemical transformation (e.g., deaminate) in aqueous formulations at neutral pH and at elevated temperature (e.g., about 25° C. or higher), while the linkage of the benzazepine compound to the polypeptide is unaffected (i.e., the DAR stays essentially the same since the compound is not released). The inventors surprisingly discovered that formulating the benzazepine conjugates at a pH below about 5.4 reduces, minimizes, or eliminates the chemical transformation of the drug (even at higher temperatures, like 25° C.). Accordingly, in various embodiments, aqueous formulations of conjugates comprising a benzazepine or a benzazepine-like compound linked to a polypeptide (e.g., antibody) are provided, wherein the aqueous formulations has a pH ranging from about 4.5 to about 5.2 (e.g., a pH of 4.5, 4.6, 4.7, 4.8. 4.9, 5.0, 5.1, 5.2, 5.3 or 5.4). In certain embodiments, an aqueous formulation of a benzazepine conjugate of this disclosure has a pH of 4.5. In other embodiments, an aqueous formulation of a benzazepine conjugate of this disclosure has a pH of 4.6. In further embodiments, an aqueous formulation of a benzazepine conjugate of this disclosure has a pH of 4.7. In still further embodiments, an aqueous formulation of a benzazepine conjugate of this disclosure has a pH of 4.8. In yet further embodiments, an aqueous formulation of a benzazepine conjugate of this disclosure has a pH of 4.9. In still other embodiments, an aqueous formulation of a benzazepine conjugate of this disclosure has a pH of 5.0. In yet other embodiments, an aqueous formulation of a benzazepine conjugate of this disclosure has a pH of 5.1. In more embodiments, an aqueous formulation of a benzazepine conjugate of this disclosure has a pH of 5.2. In still more embodiments, an aqueous formulation of a benzazepine conjugate of this disclosure has a pH of 5.3. In yet more embodiments, an aqueous formulation of a benzazepine conjugate of this disclosure has a pH of 5.4.

The aqueous formulations and lyophilized compositions provided herein may comprise one or more excipients, such as, for example, one or more buffering agents, one or more lyoprotectants, and the like, as described herein. In some embodiments, an aqueous formulation of a conjugate provided herein comprises at least one buffering agent. In some embodiments, an aqueous formulation of a conjugate provided herein does not comprise a buffering agent. In some such embodiments, the polypeptide portion of the conjugate may be buffering. Without intending to be bound by any particular theory, in some such embodiments, the polypeptide portion of the conjugate comprises sufficient weakly acidic and/or weakly basic amino acids, such as ionizable surface-exposed amino acids, to buffer the aqueous formulation without the addition of a buffering agent.

As used herein, the term “excipient” means a therapeutically inactive substance that may be included in a formulation of a therapeutic agent. Excipients can be included in a formulation for a wide variety of purposes including, for example, as a diluent, vehicle, buffering agent (also referred to as a buffer), stabilizer, tonicity agent, bulking agent, surfactant, cryoprotectant, lyoprotectant, anti-oxidant, metal ion source, chelating agent and/or preservative. Excipients include, for example, polyols such as sorbitol or mannitol; sugars such as sucrose, lactose or dextrose; polymers such as polyethylene glycol; salts such as NaCl, KCl or calcium phosphate, amino acids such as glycine, methionine or glutamic acid, surfactants, metal ions, buffer salts such as propionate, acetate or succinate, preservatives and polypeptides such as human serum albumin, as well as saline and water. Excipients are known in the art and are described in, for example, Wang W., Int. J. Pharm. 185:129-88 (1999) and Wang W., Int. J. Pharm. 203:1-60 (2000).

A “buffer” or “buffering agent” as used herein means an excipient that, in an aqueous solution, is resistant to changes in pH. A buffer is typically a weak acid or a weak base with its conjugate salt. Nonlimiting exemplary buffers include histidine, citrate, aspartate, acetate, phosphate, lactate, tromethamine, gluconate, glutamate, tartrate, succinate, malate, fumarate, and α-ketoglutarate.

Nonlimiting exemplary excipients also include sugars, such as sugar alcohols, reducing sugars, non-reducing sugars and sugar acids.

Sugar alcohols, also known as a polyols, polyhydric alcohols, or polyalcohols, are hydrogenated forms of carbohydrate having a carbonyl group reduced to a primary or secondary hydroxyl group. Polyols can be used as stabilizing excipients and/or isotonicity agents in both liquid and lyophilized formulations. Polyols can protect polypeptides from both physical and chemical degradation pathways. Preferentially excluded co-solvents increase the effective surface tension of solvent at the protein interface whereby the most energetically favorable structural conformations are those with the smallest surface areas. Specific examples of sugar alcohols include sorbitol, glycerol, mannitol, xylitol, maltitol, lactitol, erythritol and threitol.

Reducing sugars include, for example, sugars with a ketone or aldehyde group and contain a reactive hemiacetal group, which allows the sugar to act as a reducing agent. Specific examples of reducing sugars include fructose, glucose, glyceraldehyde, lactose, arabinose, mannose, xylose, ribose, rhamnose, galactose and maltose.

Non-reducing sugars contain an anomeric carbon that is an acetal and is not substantially reactive with amino acids or polypeptides to initiate a Maillard reaction. Sugars that reduce Fehling's solution or Tollen's reagent also are known as reducing sugars. Specific examples of non-reducing sugars include sucrose, trehalose, sorbose, sucralose, melezitose and raffinose.

Sugar acids include, for example, saccharic acids, gluconate and other polyhydroxy sugars and salts thereof.

Buffer excipients maintain the pH of liquid formulations through product shelf-life and maintain the pH of lyophilized formulations during the lyophilization process and upon reconstitution, for example.

Tonicity agents and/or stabilizers included in liquid formulations can be used, for example, to provide isotonicity, hypotonicity or hypertonicity to a formulation such that it is suitable for administration. Such excipients also can be used, for example, to facilitate maintenance of a polypeptides' structure and/or to minimize electrostatic, solution protein-protein interactions. Specific examples of tonicity agents and/or stabilizers include polyols, salts and/or amino acids. Tonicity agents and/or stabilizers included in lyophilized formulations can be used, for example, as a cryoprotectant to guard polypeptides from freezing stresses or as a lyoprotectant to stabilize polypeptides in the freeze-dried state. Specific examples of such cryo- and lyoprotectants include polyols, sugars and polymers.

The term “cryoprotectant” as used herein generally includes agents that provide stability to a therapeutic agent, such as a polypeptide-containing therapeutic agent, from freezing-induced stresses. Examples of cryoprotectants include, but are not limited to, polyols such as, for example, mannitol, and include saccharides such as, for example, sucrose, as well as surfactants such as, for example, polysorbate, poloxamer, polyethylene glycol, and the like. Cryoprotectants may also contribute to the tonicity of the formulations.

The term “lyoprotectant” as used herein generally includes agents that provide stability to a therapeutic agent, such as a polypeptide-containing therapeutic agent, from freeze drying-induced stress.

Bulking or caking agents are useful in lyophilized formulations to, for example, enhance product elegance and to prevent blowout. Bulking agents provide structural strength to the lyo cake and include, for example, mannitol and glycine.

Anti-oxidants are useful in liquid formulations to control protein oxidation and also can be used in lyophilized formulations to retard oxidation reactions.

Metal ions can be included in a liquid formulation, for example, as a co-factor and divalent cations such as calcium, zinc, manganese and magnesium can be utilized in suspension formulations as, for example, a stabilizer against isoaspartic acid formation as described herein. Chelating agents included in liquid formulations can be used, for example, to inhibit metal ion catalyzed reactions. With respect to lyophilized formulations, metal ions also can be included, for example, as a co-factor or as a stabilizer against isoaspartic acid formation as described herein. Although chelating agents are generally omitted from lyophilized formulations, they also can be included as desired to reduce catalytic reactions during the lyophilization process and upon reconstitution.

Preservatives included in liquid and/or lyophilized formulations can be used, for example, to protect against microbial growth and are particularly beneficial in multi-dose formulations. In lyophilized formulations, preservatives are generally included in the reconstitution diluent. Benzyl alcohol is a specific example of a preservative useful in a formulation of the invention.

As used herein, the term “surfactant” refers to a substance that functions to reduce the surface tension of a liquid in which it is dissolved. Surfactants can be included in a formulation for a variety of purposes including, for example, to prevent or control aggregation, particle formation and/or surface adsorption in liquid formulations or to prevent or control these phenomena during the lyophilization and/or reconstitution process in lyophilized formulations. Surfactants include, for example, amphipathic organic compounds that exhibit partial solubility in both organic solvents and aqueous solutions. General characteristics of surfactants include their ability to reduce the surface tension of water, reduce the interfacial tension between oil and water and also form micelles. Surfactants of the invention include non-ionic and ionic surfactants. Surfactants are well known in the art and can be found described in, for example, Randolph T. W. and Jones L. S., Surfactant-protein interactions. Pharm Biotechnol. 13:159-75 (2002).

Briefly, non-ionic surfactants include, for example, alkyl poly (ethylene oxide), alkyl polyglucosides such as octyl glucoside and decyl maltoside, fatty alcohols such as cetyl alcohol and oleyl alcohol, cocamide MEA, cocamide DEA, and cocamide TEA. Specific examples of non-ionic surfactants include the polysorbates including, for example, polysorbate 20, polysorbate 28, polysorbate 40, polysorbate 60, polysorbate 65, polysorbate 80, polysorbate 81, polysorbate 85 and the like; the poloxamers including, for example, poloxamer 188, also known as poloxalkol or poly(ethylene oxide)-poly(propylene oxide), poloxamer 407 or polyethylene-polypropylene glycol and the like, and polyethylene glycol (PEG). Polysorbate 20 is synonymous with TWEEN 20, sorbitan monolaurate and polyoxyethylenesorbitan monolaurate.

Ionic surfactants include, for example, anionic, cationic and zwitterionic surfactants. Anionic surfactants include, for example, sulfonate-based or carboxylate-based surfactants such as soaps, fatty acid salts, sodium dodecyl sulfate (SDS), ammonium lauryl sulfate and other alkyl sulfate salts. Cationic surfactants include, for example, quaternary ammonium-based surfactants such as cetyl trimethylammonium bromide (CTAB), other alkyltrimethylammonium salts, cetyl pyridinium chloride, polyethoxylated tallow amine (POEA) and benzalkonium chloride. Zwitterionic or amphoteric surfactants include, for example, dodecyl betaine, dodecyl dimethylamine oxide, cocamidopropyl betaine and coco ampho glycinate.

In some embodiments, an aqueous formulation of this disclosure comprises a conjugate comprising a benzazepine or benzazepine-like compound linked to a polypeptide, wherein the compound comprises the structure:

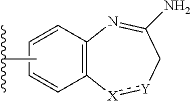

wherein [symbol] is a double bond or a single bond;

wherein when [symbol] is a double bond, X and Y are each CH; and

when [symbol] is a single bond, one of X and Y is CH₂ and the other is CH₂, O, or NH; and the structure is optionally substituted at any position other than the —NH₂. In some embodiments, the the pH of the formulation ranging from about 4.5 to about 5.2. In some embodiments, the pH of the formulation ranges from 4.4 to 5.4, 4.5 to 5.3, 4.6 to 5.2, 4.7 to 5.1, 4.8 to 5.1, 4.9 to 5.1, 4.4 to 5.0, 4.5 to 5.0, 4.6 to 5.0, 4.7 to 5.0, 4.8 to 5.0, or 4.9 to 5.0. In certain embodiments, the pH of the formulation of a benzazepine conjugate of this disclosure is 4.5. In other embodiments, the pH of the formulation of a benzazepine conjugate of this disclosure is 4.6. In further embodiments, the pH of the formulation of a benzazepine conjugate of this disclosure is 4.7. In still further embodiments, the pH of the formulation of a benzazepine conjugate of this disclosure is 4.8. In yet further embodiments, the pH of the formulation of a benzazepine conjugate of this disclosure is 4.9. In still other embodiments, the pH of the formulation of a benzazepine conjugate of this disclosure is 5.0. In yet other embodiments, the pH of the formulation of a benzazepine conjugate of this disclosure is 5.1. In more embodiments, the pH of the formulation of a benzazepine conjugate of this disclosure is 5.2. In still more embodiments, the pH of the formulation of a benzazepine conjugate of this disclosure is 5.3. In yet more embodiments, the pH of the formulation of a benzazepine conjugate of this disclosure is 5.4. In any of the aforementioned embodiments, the polypeptide is an antibody.

In further embodiments, an aqueous formulation of this disclosure comprises a conjugate represented by Formula (I):

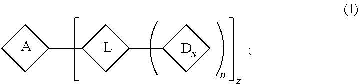

wherein A is a polypeptide; L is a linker; Dₓ is a benzazepine compound; n is selected from 1 to 20; and z is selected from 1 to 20.

In some embodiments of a Formula (I) conjugate formulation, n is 1. In some Formula (I) conjugate formulations, z ranges from 1 to 8, or ranges from about 2 to about 5, or is about 2, about 3, about 4, or about 5.

In any of the aforementioned formulations of Formula (I), L and Dₓ together have a structure selected from:

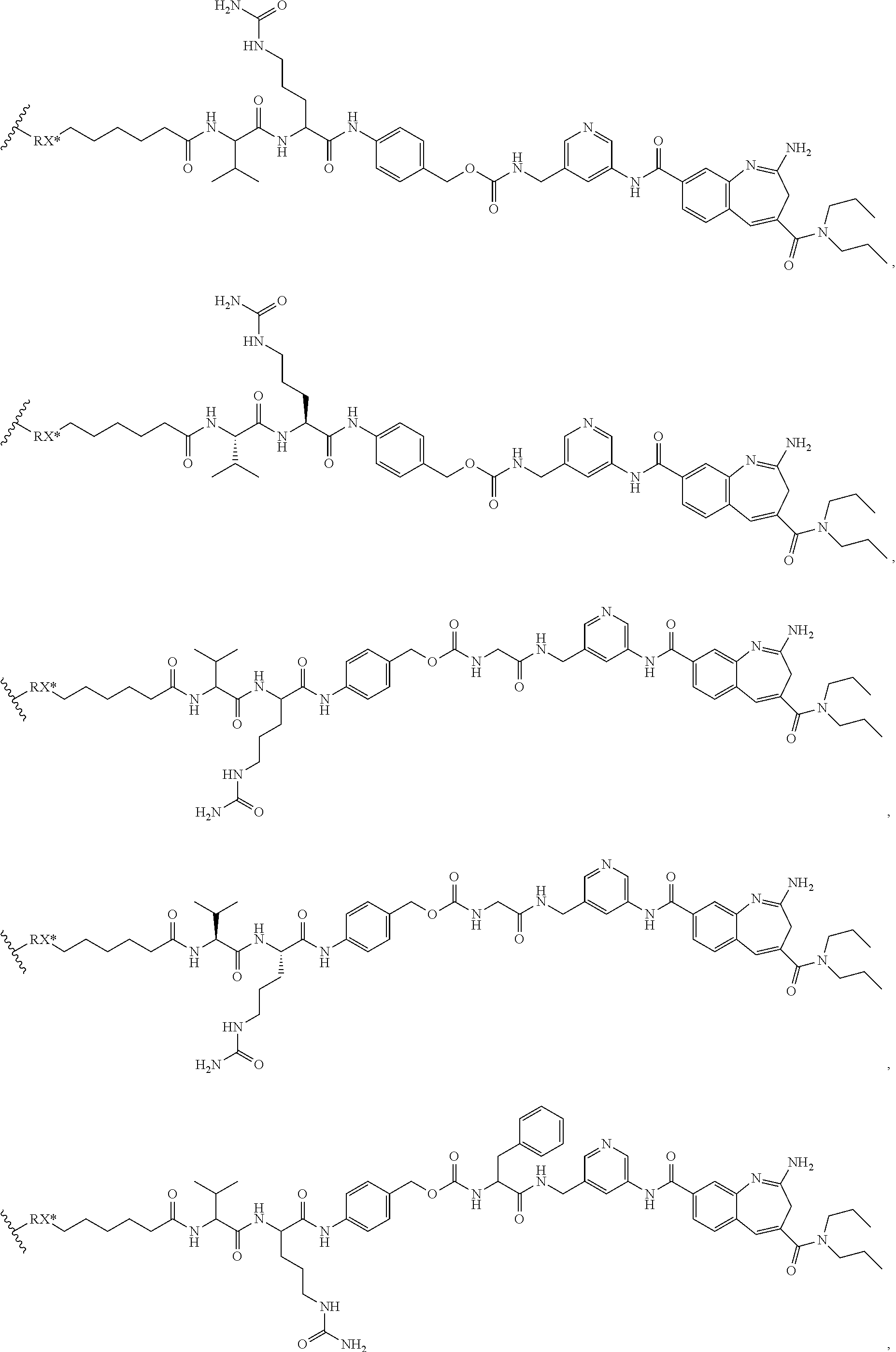

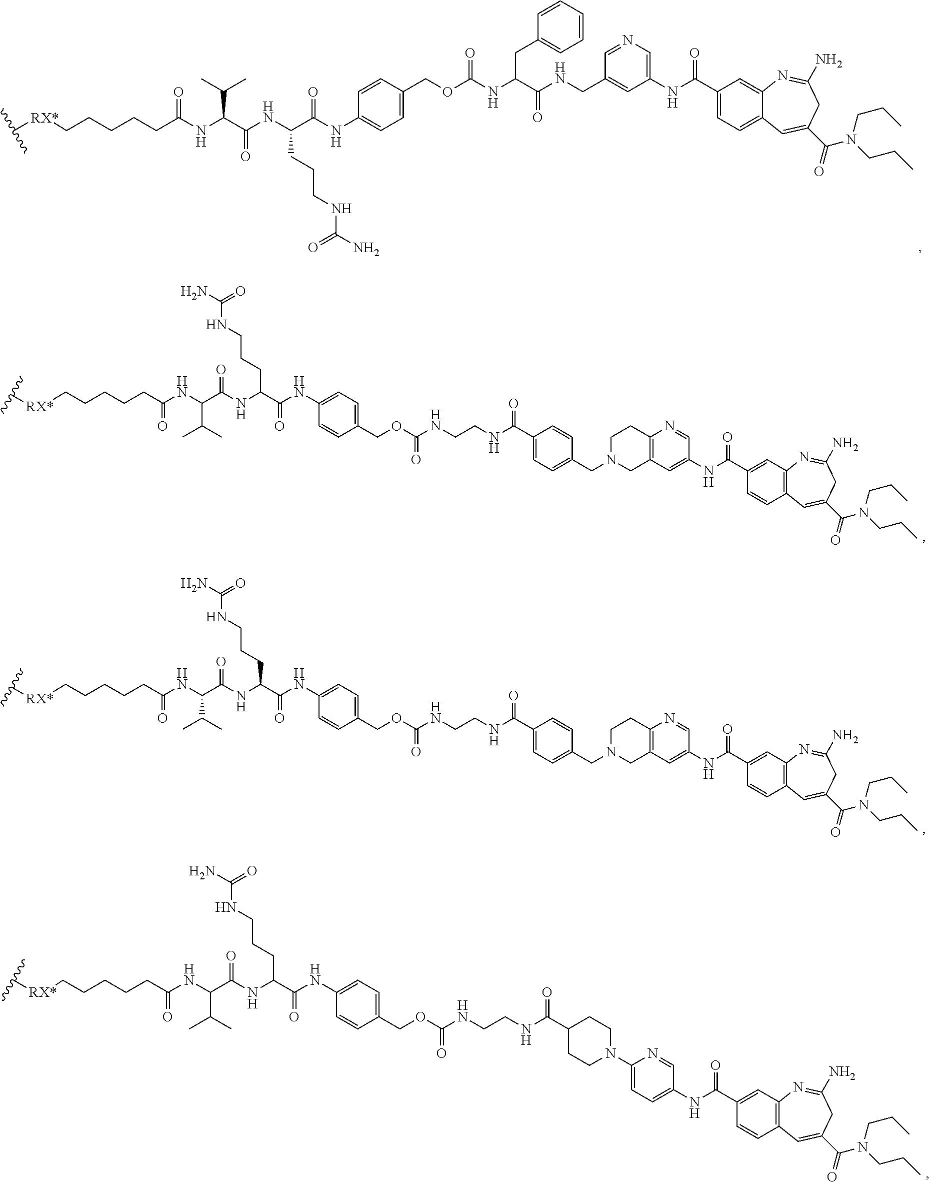

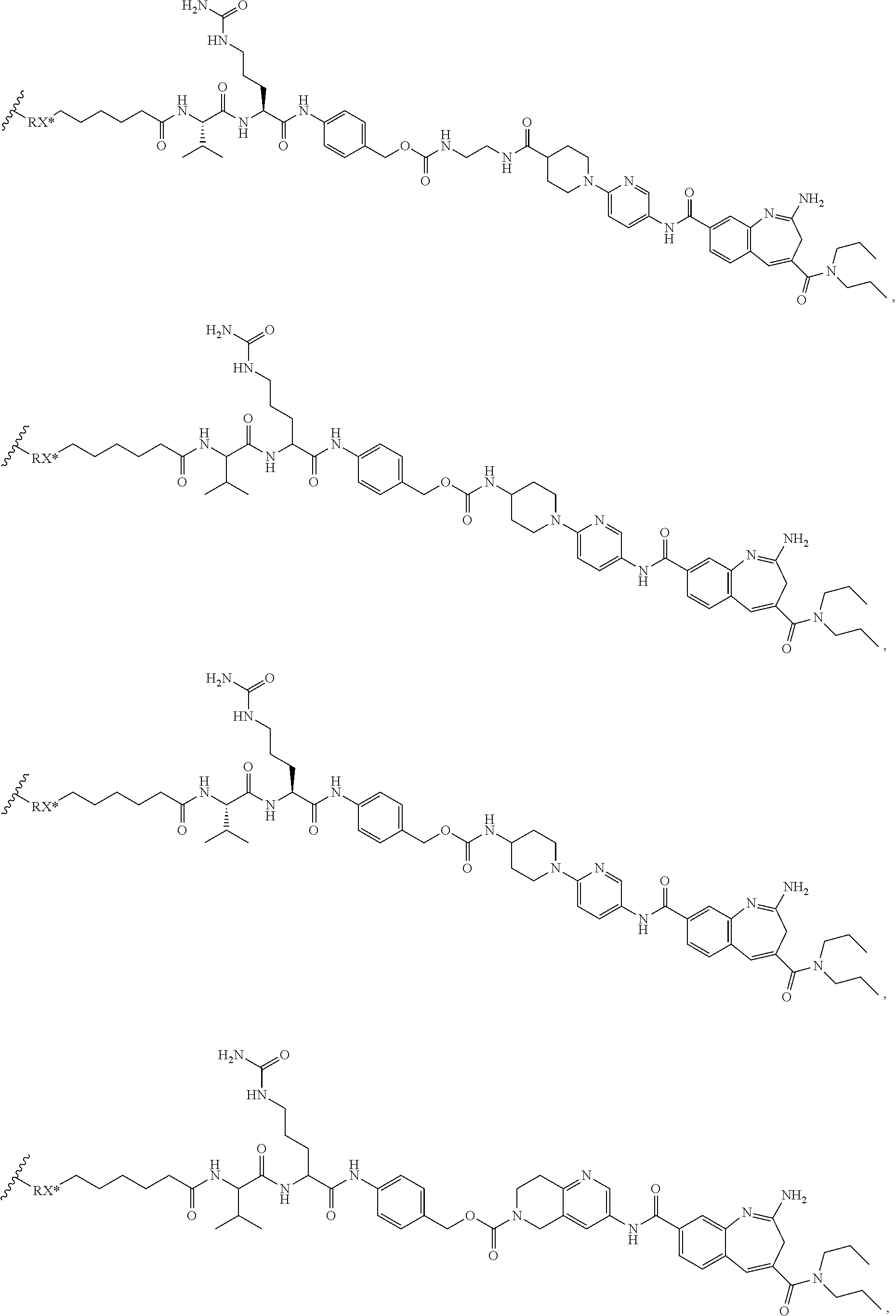

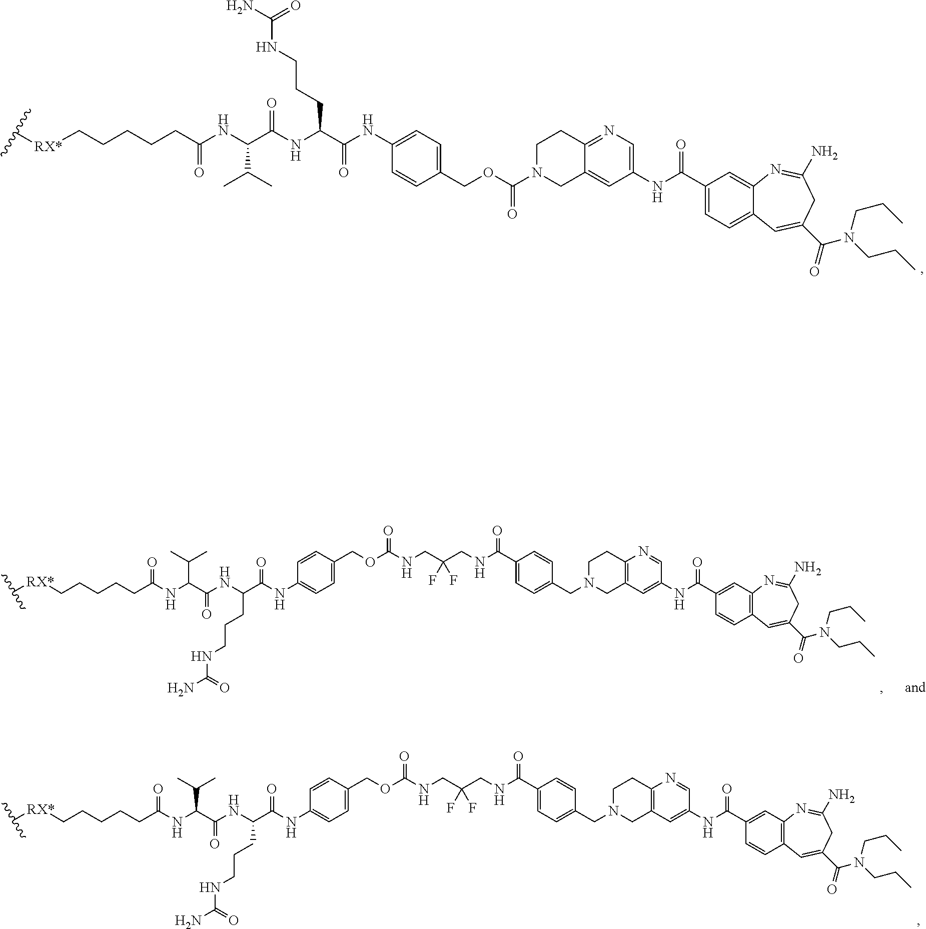

wherein the RX* is a bond, a succinimide moiety, or a hydrolyzed succinimide moiety bound to a residue of a polypeptide, such as an antibody, and wherein

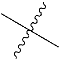

on RX* represents the point of attachment to the residue of the polypeptide. In certain formulations of Formula (I), L and Dx together have a structure of:

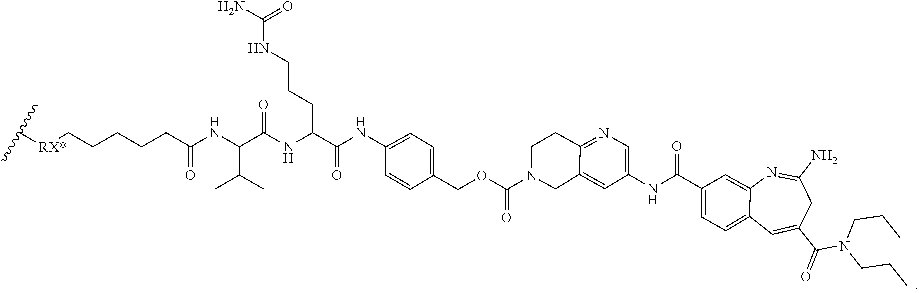

In further formulations of Formula (I), L and Dₓ together have a structure of:

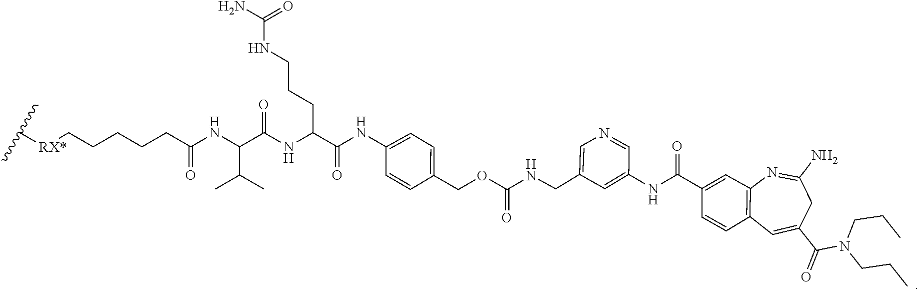

In still further formulations of Formula (I), L and Dx together have a structure of:

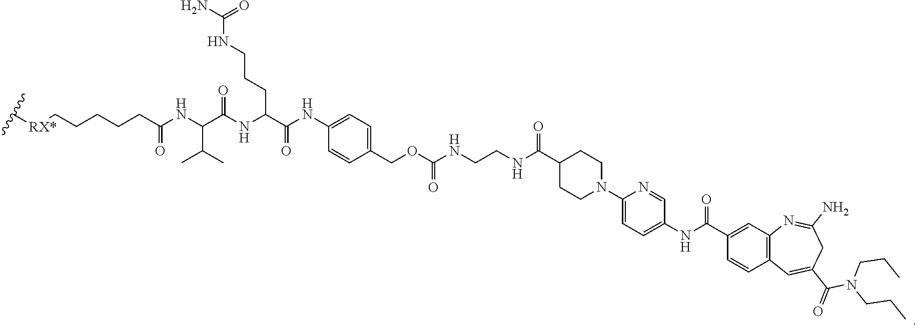

In any of the aforementioned formulation embodiments, RX* comprises a succinamide moiety and is bound to a cysteine residue of a polypeptide, such as an antibody. In some embodiments, RX* comprises a hydrolyzed succinamide moiety and is bound to a cysteine residue of a polypeptide.

In any of the aforementioned formulations of a conjugate comprising a benzazepine or benzazepine-like compound linked to a polypeptide or of a conjugate represented by Formula (I), the polypeptide is an antibody. In certain preferred embodiments, the antibody of the conjugate is specific for HER2, Nectin-4, mesothelin, or PSMA.

In some embodiments, the formulation comprises at least one buffer. In various embodiments, the buffer may be selected from histidine, citrate, aspartate, acetate, phosphate, lactate, tromethamine, gluconate, glutamate, tartrate, succinate, malic acid, fumarate, α-ketoglutarate, and combinations thereof. In some embodiments, the buffer is at least one buffer selected from histidine, citrate, aspartate, acetate, and combinations thereof. In some embodiments, the buffer is a combination of histidine and aspartate. In some embodiments, the total concentration of the buffer in the aqueous formulation ranges from about 10 mM to about 40 mM, such as from about 15 mM to about 30 mM, about 15 mM to about 25 mM, or about 20 mM. In any of the aforementioned formulation embodiments, the buffer comprises histidine and aspartate at a total concentration ranging from about 15 mM to about 25 mM, or ranging from 15 mM to 25 mM, or is about 20 mM, or is 20 mM.

In some embodiments, the aqueous formulation comprises at least one lyoprotectant. In some such embodiments, the at least one lyoprotectant is selected from sucrose, arginine, glycine, sorbitol, glycerol, trehalose, dextrose, alpha-cyclodextrin, hydroxypropyl beta-cyclodextrin, hydroxypropyl gamma-cyclodextrin, proline, methionine, albumin, mannitol, maltose, dextran, and combinations thereof. In some embodiments, the lyoprotectant is sucrose. In some embodiments, the total concentration of lyoprotectant in the aqueous formulation ranges from about 5% to about 12%, such as from about 6% to about 12%, about 6% to about 10%, about 6% to about 9%, about 7% to about 9%, or about 7% to about 8%. In any of the aforementioned formulation embodiments, the lyoprotectant comprises sucrose at a total concentration ranging from about 7% to about 8%, or ranging from 7% to 8%, or is about 8%, or is 8%.

In some embodiments, the aqueous formulation comprises at least one surfactant. Exemplary surfactants include polysorbate 80, polysorbate 20, poloxamer 88, and combinations thereof. In some embodiments, the aqueous formulation comprises polysorbate 80. In some embodiments, the total concentration of the at least one surfactant ranges from about 0.01% to about 0.1%, such as from about 0.01% to about 0.05%, about 0.01% to about 0.08%, about 0.01% to about 0.06%, about 0.01% to about 0.04%, about 0.01% to about 0.03%, or about 0.02%. In any of the aforementioned formulation embodiments, the at least one surfactant comprises polysorbate 80 at a total concentration ranging from about 0.01% to about 0.03%, or ranging from 0.01% to 0.03%, or is about 0.02%, or is 0.02%.

In some embodiments, the concentration of the conjugate in the aqueous formulation ranges from about 1 mg/mL to about 200 mg/mL, such as from about 10 mg/mL to about 160 mg/mL, about 20 mg/mL to about 140 mg/mL, about 30 mg/mL to about 120 mg/mL, about 40 mg/mL to about 110 mg/mL, about 50 mg/mL to about 100 mg/mL, about 60 mg/mL to about 95 mg/mL, about 70 mg/mL to about 90 mg/mL, or about 80 mg/mL. In any of the aforementioned formulation embodiments, the concentration of the conjugate in the aqueous formulation ranging from about 70 mg/mL to about 90 mg/mL, or ranging from 70 mg/mL to 90 mg/mL, or is about 80 mg/mL, or is 80 mg/mL.

In some embodiments, an aqueous formulation of this disclosure comprises:

(a) a conjugate at a total concentration ranging from about 50 mg/mL to about 100 mg/mL and represented by Formula (I):

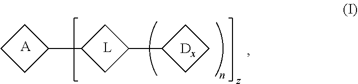

wherein A is an antibody; n is 1; z ranges from 2 to 8; and L is a linker and Dₓ is a benzazepine compound, wherein L and Dx together have a structure of:

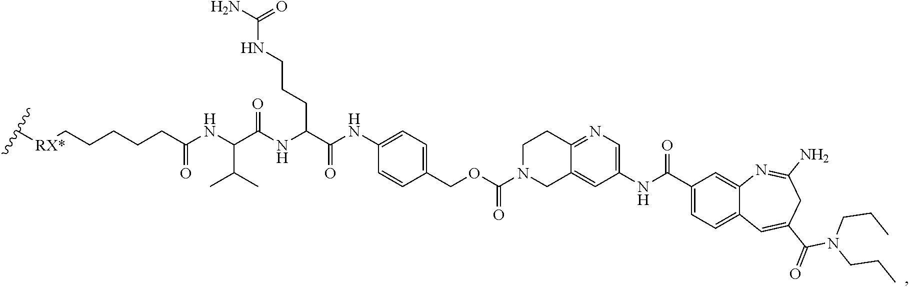

wherein RX* comprises a hydrolyzed succinamide moiety and is bound to a cysteine residue of the antibody;

(b) a buffer comprised of histidine and aspartate at a total concentration ranging from about 15 mM to about 25 mM;

(c) a lyoprotectant comprised of sucrose at a total concentration ranging from about 7% to about 8%; and

(d) a surfactant comprised of polysorbate 80 at a total concentration ranging from about 0.01% to about 0.03%.

In certain embodiments, the antibody of the conjugate is specific for HER2, Nectin-4, mesothelin, or PSMA.

In particular embodiments, an anti-HER2 antibody of a conjugate for use in formulations of this disclosure comprises heavy chain (HC)-CDR1, HC-CDR2, HC-CDR3, light chain (LC)-CDR1, LC-CDR2, and LC-CDR3 of SEQ ID NOS:1-6, respectively. In further embodiments, the anti-HER2 antibody of the conjugates for use in formulations of this disclosure comprises a heavy chain and light chain, wherein: (a) the heavy chain comprises HC-CDR1, HC-CDR2, and HC-CDR3 of SEQ ID NOS:1-3, respectively, and comprises a heavy chain variable region (V_{H}) having an amino acid sequence that has at least 90%, at least 91%, at least 92%, at least 93%, at least 94%, at least 95%, at least 96%, at least 97%, at least 98%, at least 99%, at least 99.5%, or 100% identity to the V_{H} amino acid sequence of SEQ ID NO:7; and (b) the light chain comprises LC-CDR1, LC-CDR2, and LC-CDR3 of SEQ ID NOS:4-6, respectively, and a light chain variable region (V_{L}) having an amino acid sequence that has at least 90%, at least 91%, at least 92%, at least 93%, at least 94%, at least 95%, at least 96%, at least 97%, at least 98%, at least 99%, at least 99.5%, or 100% identity to the V_{L} amino acid sequence of SEQ ID NO:8. In still further embodiments, the anti-HER2 antibody of the conjugates for use in formulations of this disclosure comprises a V_{H} comprising or consisting of the amino acid sequence of SEQ ID NO:7 and a V_{L} comprising or consisting of the amino acid sequence of SEQ ID NO:8. In yet further embodiments, the anti-HER2 antibody of the conjugates for use in formulations of this disclosure comprises a heavy chain and light chain, wherein: (a) the heavy chain comprises HC-CDR1, HC-CDR2, and HC-CDR3 of SEQ ID NOS:1-3, respectively, and comprises an amino acid sequence that has at least 90%, at least 91%, at least 92%, at least 93%, at least 94%, at least 95%, at least 96%, at least 97%, at least 98%, at least 99%, at least 99.5%, or 100% identity to the heavy chain amino acid sequence of SEQ ID NO:9; and (b) the light chain comprises LC-CDR1, LC-CDR2, and LC-CDR3 of SEQ ID NOS:4-6, respectively, and an amino acid sequence that has at least 90%, at least 91%, at least 92%, at least 93%, at least 94%, at least 95%, at least 96%, at least 97%, at least 98%, at least 99%, at least 99.5%, or 100% identity to the light chain amino acid sequence of SEQ ID NO:10. In more embodiments, the anti-HER2 antibody of the conjugate for use in formulations of this disclosure comprises a heavy chain comprising or consisting of the amino acid sequence of SEQ ID NO:9 and a light chain comprising or consisting of the amino acid sequence of SEQ ID NO:10.

In particular embodiments, an anti-Nectin-4 antibody of a conjugate for use in formulations of this disclosure comprises heavy chain (HC)-CDR1, HC-CDR2, and HC-CDR3 of SEQ ID NOS:11-13, respectively, and light chain (LC)-CDR1 of SEQ ID NO:14 or 15, LC-CDR2 of SEQ ID NO:16, and LC-CDR3 of SEQ ID NO:17. In further embodiments, the anti-Nectin-4 antibody of the conjugates for use in formulations of this disclosure comprises a heavy chain and light chain, wherein: (a) the heavy chain comprises HC-CDR1, HC-CDR2, and HC-CDR3 of SEQ ID NOS:11-13, respectively, and comprises a heavy chain variable region (V_{H}) having an amino acid sequence that has at least 90%, at least 91%, at least 92%, at least 93%, at least 94%, at least 95%, at least 96%, at least 97%, at least 98%, at least 99%, at least 99.5%, or 100% identity to the V_{H} amino acid sequence of SEQ ID NO:18; and (b) the light chain comprises LC-CDR1, LC-CDR2, and LC-CDR3 of SEQ ID NOS:14, 16 and 17, respectively, or 15, 16 and 17, respectively, and a light chain variable region (V_{L}) having an amino acid sequence that has at least 90%, at least 91%, at least 92%, at least 93%, at least 94%, at least 95%, at least 96%, at least 97%, at least 98%, at least 99%, at least 99.5%, or 100% identity to the VL amino acid sequence of SEQ ID NO:19 or 20. In still further embodiments, the anti-Nectin-4 antibody of the conjugates for use in formulations of this disclosure comprises a VH comprising or consisting of the amino acid sequence of SEQ ID NO:18 and a VL comprising or consisting of the amino acid sequence of SEQ ID NO:19 or 20. In yet further embodiments, the anti-Nectin-4 antibody of the conjugates for use in formulations of this disclosure comprises a heavy chain and light chain, wherein: (a) the heavy chain comprises HC-CDR1, HC-CDR2, and HC-CDR3 of SEQ ID NOS:11-13, respectively, and comprises an amino acid sequence that has at least 90%, at least 91%, at least 92%, at least 93%, at least 94%, at least 95%, at least 96%, at least 97%, at least 98%, at least 99%, at least 99.5%, or 100% identity to the heavy chain amino acid sequence of SEQ ID NO:21; and (b) the light chain comprises LC-CDR1, LC-CDR2, and LC-CDR3 of SEQ ID NOS:14, 16 and 17, respectively, or 15, 16 and 17, respectively, and an amino acid sequence that has at least 90%, at least 91%, at least 92%, at least 93%, at least 94%, at least 95%, at least 96%, at least 97%, at least 98%, at least 99%, at least 99.5%, or 100% identity to the light chain amino acid sequence of SEQ ID NO:22 or 23. In more embodiments, the anti-Nectin-4 antibody of the conjugate for use in formulations of this disclosure comprises a heavy chain comprising or consisting of the amino acid sequence of SEQ ID NO:21 and a light chain comprising or consisting of the amino acid sequence of SEQ ID NO:22 or 23.

In particular embodiments, an anti-ASGR1 antibody of a conjugate for use in formulations of this disclosure comprises heavy chain (HC)-CDR1 of SEQ ID NO:24 or 25, HC-CDR2 of SEQ ID NO:26, 27 or 28, and HC-CDR3 of SEQ ID NO:29 or 30, and light chain (LC)-CDR1 of SEQ ID NO:31 or 32, LC-CDR2 of SEQ ID NO:33, 34, 35 or 36, and LC-CDR3 of SEQ ID NO:37 or 38. In further embodiments, the anti-ASGR1 antibody of the conjugates for use in formulations of this disclosure comprises a heavy chain and light chain, wherein: (a) the heavy chain comprises HC-CDR1, HC-CDR2, and HC-CDR3 of SEQ ID NOS:24, 26, and 29, respectively, and comprises a heavy chain variable region (V_{H}) having an amino acid sequence that has at least 90%, at least 91%, at least 92%, at least 93%, at least 94%, at least 95%, at least 96%, at least 97%, at least 98%, at least 99%, at least 99.5%, or 100% identity to the V_{H} amino acid sequence of SEQ ID NO:39; and (b) the light chain comprises LC-CDR1, LC-CDR2, and LC-CDR3 of SEQ ID NOS:31, 33 and 37, respectively, or 31, 34 and 37, respectively, and a light chain variable region (V_{L}) having an amino acid sequence that has at least 90%, at least 91%, at least 92%, at least 93%, at least 94%, at least 95%, at least 96%, at least 97%, at least 98%, at least 99%, at least 99.5%, or 100% identity to the V_{L} amino acid sequence of SEQ ID NO:42 or 43. In further embodiments, the anti-ASGR1 antibody of the conjugates for use in formulations of this disclosure comprises a heavy chain and light chain, wherein: (a) the heavy chain comprises HC-CDR1, HC-CDR2, and HC-CDR3 of SEQ ID NOS:25, 27 and 30, respectively, or 25, 28 and 30, respectively, and comprises a heavy chain variable region (V_{H}) having an amino acid sequence that has at least 90%, at least 91%, at least 92%, at least 93%, at least 94%, at least 95%, at least 96%, at least 97%, at least 98%, at least 99%, at least 99.5%, or 100% identity to the VH amino acid sequence of SEQ ID NO:40 or 41; and (b) the light chain comprises LC-CDR1, LC-CDR2, and LC-CDR3 of SEQ ID NOS:32, 35 and 38, respectively, or 32, 36 and 38, respectively, and a light chain variable region (V_{L}) having an amino acid sequence that has at least 90%, at least 91%, at least 92%, at least 93%, at least 94%, at least 95%, at least 96%, at least 97%, at least 98%, at least 99%, at least 99.5%, or 100% identity to the VL amino acid sequence of SEQ ID NO:44 or 45. In still further embodiments, the anti-ASGR1 antibody of the conjugates for use in formulations of this disclosure comprises a V_{H} comprising or consisting of the amino acid sequence of SEQ ID NO:39 and a V_{L} comprising or consisting of the amino acid sequence of SEQ ID NO:42 or 43. In still further embodiments, the anti-ASGR1 antibody of the conjugates for use in formulations of this disclosure comprises a V_{H} comprising or consisting of the amino acid sequence of SEQ ID NO:40 or 41 and a V_{L} comprising or consisting of the amino acid sequence of SEQ ID NO:44 or 45. In yet further embodiments, the anti-ASGR1 antibody of the conjugates for use in formulations of this disclosure comprises a heavy chain and light chain, wherein: (a) the heavy chain comprises HC-CDR1, HC-CDR2, and HC-CDR3 of SEQ ID NOS:24, 26, and 29, respectively, and comprises an amino acid sequence that has at least 90%, at least 91%, at least 92%, at least 93%, at least 94%, at least 95%, at least 96%, at least 97%, at least 98%, at least 99%, at least 99.5%, or 100% identity to the heavy chain amino acid sequence of SEQ ID NO:46; and (b) the light chain comprises LC-CDR1, LC-CDR2, and LC-CDR3 of SEQ ID NOS:31, 33 and 37, respectively, or 31, 34 and 37, respectively, and an amino acid sequence that has at least 90%, at least 91%, at least 92%, at least 93%, at least 94%, at least 95%, at least 96%, at least 97%, at least 98%, at least 99%, at least 99.5%, or 100% identity to the light chain amino acid sequence of SEQ ID NO:49 or 50. In more embodiments, the anti-ASGR1 antibody of the conjugate for use in formulations of this disclosure comprises a heavy chain comprising or consisting of the amino acid sequence of SEQ ID NO:46 and a light chain comprising or consisting of the amino acid sequence of SEQ ID NO:49 or 50. In yet further embodiments, the anti-ASGR1 antibody of the conjugates for use in formulations of this disclosure comprises a heavy chain and light chain, wherein: (a) the heavy chain comprises HC-CDR1, HC-CDR2, and HC-CDR3 of SEQ ID NOS:25, 27, and 30, respectively, or 25, 28, and 30, respectively, and comprises an amino acid sequence that has at least 90%, at least 91%, at least 92%, at least 93%, at least 94%, at least 95%, at least 96%, at least 97%, at least 98%, at least 99%, at least 99.5%, or 100% identity to the heavy chain amino acid sequence of SEQ ID NO:47 or 48; and (b) the light chain comprises LC-CDR1, LC-CDR2, and LC-CDR3 of SEQ ID NOS:32, 35 and 38, respectively, or 32, 36 and 38, respectively, and an amino acid sequence that has at least 90%, at least 91%, at least 92%, at least 93%, at least 94%, at least 95%, at least 96%, at least 97%, at least 98%, at least 99%, at least 99.5%, or 100% identity to the light chain amino acid sequence of SEQ ID NO:51 or 52. In more embodiments, the anti-ASGR1 antibody of the conjugate for use in formulations of this disclosure comprises a heavy chain comprising or consisting of the amino acid sequence of SEQ ID NO:47 or 48 and a light chain comprising or consisting of the amino acid sequence of SEQ ID NO:51 or 52.

In particular embodiments, an anti-mesothelin antibody of a conjugate for use in formulations of this disclosure comprises heavy chain (HC)-CDR1, HC-CDR2, HC-CDR3, light chain (LC)-CDR1, LC-CDR2, and LC-CDR3 of SEQ ID NOS:53-58, respectively. In further embodiments, the anti-mesothelin antibody of the conjugates for use in formulations of this disclosure comprises a heavy chain and light chain, wherein: (a) the heavy chain comprises HC-CDR1, HC-CDR2, and HC-CDR3 of SEQ ID NOS:53-55, respectively, and comprises a heavy chain variable region (V_{H}) having an amino acid sequence that has at least 90%, at least 91%, at least 92%, at least 93%, at least 94%, at least 95%, at least 96%, at least 97%, at least 98%, at least 99%, at least 99.5%, or 100% identity to the VH amino acid sequence of SEQ ID NO:59; and (b) the light chain comprises LC-CDR1, LC-CDR2, and LC-CDR3 of SEQ ID NOS:56-58, respectively, and a light chain variable region (VL) having an amino acid sequence that has at least 90%, at least 91%, at least 92%, at least 93%, at least 94%, at least 95%, at least 96%, at least 97%, at least 98%, at least 99%, at least 99.5%, or 100% identity to the VL amino acid sequence of SEQ ID NO:60. In still further embodiments, the anti-mesothelin antibody of the conjugates for use in formulations of this disclosure comprises a V_{H} comprising or consisting of the amino acid sequence of SEQ ID NO:59 and a V_{L} comprising or consisting of the amino acid sequence of SEQ ID NO:60. In yet further embodiments, the anti-HER2 antibody of the conjugates for use in formulations of this disclosure comprises a heavy chain and light chain, wherein: (a) the heavy chain comprises HC-CDR1, HC-CDR2, and HC-CDR3 of SEQ ID NOS:53-55, respectively, and comprises an amino acid sequence that has at least 90%, at least 91%, at least 92%, at least 93%, at least 94%, at least 95%, at least 96%, at least 97%, at least 98%, at least 99%, at least 99.5%, or 100% identity to the heavy chain amino acid sequence of SEQ ID NO:70; and (b) the light chain comprises LC-CDR1, LC-CDR2, and LC-CDR3 of SEQ ID NOS:56-58, respectively, and an amino acid sequence that has at least 90%, at least 91%, at least 92%, at least 93%, at least 94%, at least 95%, at least 96%, at least 97%, at least 98%, at least 99%, at least 99.5%, or 100% identity to the light chain amino acid sequence of SEQ ID NO:71. In more embodiments, the anti-HER2 antibody of the conjugate for use in formulations of this disclosure comprises a heavy chain comprising or consisting of the amino acid sequence of SEQ ID NO:70 and a light chain comprising or consisting of the amino acid sequence of SEQ ID NO:71.

In various embodiments, lyophilized compositions comprising a conjugate of this disclosure are provided, wherein reconstitution of the lyophilized composition in water, and optionally with one or more of a buffer, a lyoprotectant and a surfactant, produces an aqueous formulation described herein. In some embodiments, a lyophilized composition is produced by lyophilizing an aqueous formulation provided herein.

Methods for formulation of the pharmaceutical compositions can include formulating any of the conjugates described as described herein to form an aqueous composition for parenteral administration, such as subcutaneous or intravenous administration. As discussed herein, the compositions described herein can be lyophilized or in powder form for reconstitution with a suitable vehicle, e.g., sterile pyrogen-free water, before use.

The pharmaceutical compositions and formulations can be sterilized. Sterilization can be accomplished by filtration through sterile filtration.

The conjugates can be formulated for administration in a unit dosage form in association with a pharmaceutically acceptable vehicle. Such vehicles can be inherently nontoxic, and non-therapeutic. A vehicle can be water, saline, Ringer's solution, dextrose solution, and 5% human serum albumin. Nonaqueous vehicles such as fixed oils and ethyl oleate can also be used. The vehicle can contain minor amounts of additives such as substances that enhance isotonicity and chemical stability (e.g., buffers and preservatives).

### Exemplary Therapeutic Applications

The aqueous formulations comprising a conjugate comprising a benzazepine or benzazepine-like compound linked to a polypeptide are useful for treating plurality of different subjects including, but not limited to, a mammal, human, non-human mammal, a domesticated animal (e.g., laboratory animals, household pets, or livestock), non-domesticated animal (e.g., wildlife), dog, cat, rodent, mouse, hamster, cow, bird, chicken, fish, pig, horse, goat, sheep, rabbit, and any combination thereof. In various embodiments, the subject is a human.

The disclosure provides an aqueous formulation or lyophilized composition of a conjugate of a benzazepine or benzazepine-like compound suitable for parenteral administration, such as subcutaneous or intravenous administration. In some embodiments, methods of treatment comprise subcutaneous administration, or intravenous administration by slow infusion.

The conjugates and pharmaceutical compositions thereof can be used in the methods described herein as a therapeutic, for example, as a treatment that can be administered in an effective regimen to a subject in need thereof to achieve a therapeutic effect, while alleviating, sparing, or avoiding toxicity(ies) associated with bolus repetitive intravenous administration of the conjugate. Toxicities that can be alleviated, spared, or avoided include anaphylaxis-like toxicity. A therapeutic effect can be obtained in a subject by reduction, suppression, remission, alleviation or eradication of a disease state, including, but not limited to, one or more symptoms thereof. A therapeutic effect in a subject having a disease or condition, or exhibiting an early symptom thereof or exhibiting or otherwise suspected of being in or approaching an early stage of a disease or condition, can be obtained by a reduction, a suppression, a prevention, a delay, a remission, an alleviation or an eradication of the condition or disease, or pre-condition or pre-disease state. In various embodiments, the effective regimen results in a Tmax of the conjugate of greater than 4 hours following each administration of the conjugate. In some embodiments, the effective regimen results in a Tmax greater than 6 hours, greater than 8 hours, greater than 10 hours, greater than 12 hours, or greater than 15 hours following each administration of the conjugate. In some embodiments, the conjugate is an immune-stimulatory conjugate.

In certain embodiments, the methods include administration of an immune-stimulatory conjugate, or a pharmaceutical composition thereof, to a subject in need thereof in an effective regimen to activate, stimulate or augment an immune response against a disease treatable with a TLR agonist (e.g., cancer, fibrosis, or a viral disease). The polypeptide of the conjugate recognizes an antigen associated with the disease or disease state.

In certain embodiments, the methods include administration of an immune-stimulatory conjugate to a subject in need thereof in an effective regimen to activate, stimulate or augment an immune response against cell of a disease of condition. In certain embodiments, the methods include administration of an immune-stimulatory conjugate to a subject in need thereof in an effective regimen to activate, stimulate or augment an immune response against cancer cells, where the cancer cells express a tumor antigen or a tumor associated antigen recognized by the polypeptide of the conjugate.

In certain embodiments, the methods include administration of an immune-stimulatory conjugate to a subject in need thereof in an effective regimen to activate, stimulate or augment an immune response against tumor cells of a solid tumor, such as a sarcoma, a carcinoma or lymphoma. In some such embodiments, the polypeptide of the conjugate recognizes an antigen on the target cells, such as tumor cells. In certain embodiments, the methods include administration of an immune-stimulatory conjugate to a subject in need thereof in an effective regimen to activate, stimulate or augment an immune response against tumor cells of a sarcoma.

In some such embodiments, the polypeptide of the conjugate recognizes an antigen on the sarcoma cells. In certain embodiments, the methods include administration of an immune-stimulatory conjugate to a subject in need thereof in an effective regimen to activate, stimulate or augment an immune response against tumor cells of a carcinoma. In some such embodiments, the polypeptide of the conjugate recognizes an antigen on the tumor cells. In certain embodiments, the methods include administration of an immune-stimulatory conjugate to a subject in need thereof in an effective regimen to activate, stimulate or augment an immune response against tumor cells of a lymphoma. In some such embodiments, the polypeptide of the conjugate recognizes an antigen on the tumor cells.

In certain embodiments, the methods include administration of an immune-stimulatory conjugate to a subject in need thereof in an effective regimen to activate, stimulate or augment an immune response against tumor cells of a solid tumor, such as brain, breast, lung, liver, kidney, pancreatic, colorectal, ovarian, head and neck, bone, skin, mesothelioma, bladder, stomach, prostate, thyroid, uterine or cervical/endometrial cells. In some such embodiments, the polypeptide of the conjugate recognizes an antigen on the tumor cells.

In certain embodiments, the cancer is a HER2 expressing cancer and the methods include administration of an immune-stimulatory conjugate to a subject in need thereof in an effective regimen to activate, stimulate or augment an immune response against cells of the HER2 expressing cancer. In some aspects, the HER2 expresssing cancer expresses HER2 at a level of 2+ or 3+ as determined by immunohistochemistry. In further embodiments, the cancer is a Nectin-4 expressing cancer and the methods include administration of an immune-stimulatory conjugate to a subject in need thereof in an effective regimen to activate, stimulate or augment an immune response against cells of the Nectin-4 expressing cancer. In still further embodiments, the cancer is a mesothelin expressing cancer and the methods include administration of an immune-stimulatory conjugate to a subject in need thereof in an effective regimen to activate, stimulate or augment an immune response against cells of the mesothelin expressing cancer. In yet further embodiments, the cancer is a PSMA expressing cancer and the methods include administration of an immune-stimulatory conjugate to a subject in need thereof in an effective regimen to activate, stimulate or augment an immune response against cells of the PSMA expressing cancer.

In some cases, treatment comprises reduced tumor growth. In some cases, treatment comprises tumor arrest.

In some embodiments, toxicities associated with intravenous administration of immune-stimulatory conjugates can be spared, alleviated, or avoided by administering the immune-stimulatory conjugates by subcutaneous or intravenous slow infusion administration. In some embodiments, the toxicities are anaphylaxis-like toxicities. Such toxicities can be associated with single or multiple intravenous administrations of an immune-stimulatory conjugate. As used herein, “alleviating” or “to alleviate” a toxicity refers to making the toxicity less severe. The terms “sparing” or “to spare” refer to significantly reducing the toxicity and to reduce harm to the subject. An anaphylaxis-like response refers to symptoms such as hypotension, airway constriction, hypothermia and/or vacular leak syndrome, in the absence of significant cytokine release. As used herein, an anaphylaxis-like response is other than classical anaphylaxis, resulting from an IgG or IgE response. In some embodiments, grade 1 or greater, grade 2 or greater, grade 3 or greater, or grade 4 or greater anaphylaxis-like adverse events associated with repetitive bolus intravenous administration of an immune-stimulatory conjugate are spared, alleviated, or avoided.

One of ordinary skill in the art would understand that the amount, duration and frequency of administration of an aqueous formulation of a conjugate described herein to a subject in need thereof depends on several factors including, for example but not limited to, the health of the subject, the specific disease or condition of the subject, the grade or level of a specific disease or condition of the subject, the additional therapeutics the subject is being or has been administered, and the like.

In some aspects of practicing the methods described herein, the conjugates are administered in an effective regimen of at least two or at least three cycles. Each cycle can optionally include a resting stage between cycles. Cycles of administration can be of any suitable length. In some embodiments, each cycle is a week (7 days), 10 days, every two weeks (14 days or biweekly), every three week (21 days) or every four weeks (28 days). In some embodiments, each cycle is a month. In some embodiments, at least two doses of the immune-stimulatory conjugate are administered more than 7 days apart, or more than 10 days apart. In some embodiments, at least one dose of the conjugate is administered more than 7 days, or more than 10 days, after the initial dose of the conjugate.

In certain embodiments, the total dose of a conjugate of this disclosure within a cycle is from about 0.1 mg/kg to about 10 mg/kg. In some embodiments, the total dose is from about 0.5 mg/kg to about 7.5 mg/kg. In some embodiments, the total dose is from about 0.5 mg/kg to about 5 mg/kg. In some embodiments, the total dose is from about 0.5 mg/kg to about 4 mg/kg. In some embodiments, the total dose is from about 0.5 mg/kg to about 3.5 mg/kg. In some embodiments, the total dose is from about 0.5 mg/kg to about 2 mg/kg. In certain preferred embodiments, the total dose of a conjugate of this disclosure within a cycle ranges from about 0.3 mg/kg to about 2.4 mg/kg, or from about 0.6 mg/kg to about 1.2 mg/kg, or is about 0.6 mg/kg.

Application of immune-stimulatory conjugates described herein shows substantial benefit in directing a subject's own immune response to cells of a particular site of disease or disorder, such as cells associated with the disease or disorder. Activating or stimulating an immune response directed to targeted cells facilitates the reduction, inhibition of proliferation, inhibition of growth, inhibition of progression, inhibition of metastasis or otherwise inhibition up to and including in some cases clearance of the targeted cells. Thus, in some cases a targeted immune response activation or stimulation leads to inhibition of disease progression, or alleviation of at least one symptom of a manifest disease in a patient, up to and in some cases including complete elimination of from one symptom to an entire disease state in a subject.

In some embodiments, B cells are deplated prior to administration of an immune-stimulatory conjugate. In some embodiments, an immune stimulatory conjugate is administered with a B-cell depleting agent. The B-cell depleting agent may be administered prior to, at the same time as, or after the immune stimulatory conjugate. The B-cell depleting agent may be administered, for example, within 14 days, within 7 days, within 1 day, within 24, 12, 6, 4, 3, 2, or 1 hour of the first administration of the immune-stimulatory conjugate. B-cell depleting agents include, but are not limited to, anti-CD20 antibodies, anti-CD19 antibodies, anti-CD22 antibodies, anti-BLyS antibodies, TACI-Ig, BR3-Fc, and anti-BR3 antibodies. Nonlimiting exemplary B-cell depleting agents include rituximab, ocrelizumab, ofatumumab, epratuzumab, MEDI-51 (anti-CD19 antibody), belimumab, BR3-Fc, AMG-623, and atacicept.

In some embodiments, the immune-stimulatory conjugate is administered with an agent that mitigates an anaphylactic-like toxicity. Nonlimiting exemplary agents that mitigate an anaphylactic-like toxicity include epinephrine, an antihistamine, a cortisone, and a beta-agonist. Administration may be, for example, within 1 hour or within minutes of administration of the immune-stimulatory conjugate.

Methods of administration as disclosed herein are consistent with the use of a broad range of conjugates comprising benzazepine and benzazepine-like compounds attached to polypeptides, such as antibodies. In particular, the methods disclosed herein are well suited for use with immune stimulatory conjugates, such as immune stimulatory conjugates that direct an immune response in a subject to a particular disorder or disease location, cell type or cell. Accordingly, practice of some methods herein comprises selection of a suitable subject such as a subject to be subjected to or undergoing a treatment with a conjugate that directs a benzazepine or benzazepine-like compound of the conjugate to a particular disorder or disease site, cell type or cell. Often, the subject is selected for practice of the method due to having at least one symptom of a disease or disorder, or projected to develop at least one symptom of a disease or disorder (such as a subject in remission and at risk for relapse), suitable for treatment by a conjugate as disclosed herein. Some diseases are selected not based upon or not based solely on disease type, but upon detection or presence of a suitable epitope on a tumor, cell type or particular cell that facilitates localization of an immune-stimulatory conjugate to the epitope.

### EXAMPLES

The following examples are included to further describe some embodiments of the present disclosure and should not be used to limit the scope of the disclosure. The examples are not intended to represent that the experiments below are all or the only experiments performed. Efforts have been made to ensure accuracy with respect to numbers used (for example, amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

While aspects of the present disclosure have been shown and described herein, it will be apparent to those skilled in the art that such aspects are provided by way of example only. Numerous variations, changes, and substitutions will now occur to those skilled in the art without departing from the disclosure. It should be understood that various alternatives to the aspects of the disclosure described herein may be employed in practicing the disclosure. It is intended that the following claims define the scope of the disclosure and that methods and structures within the scope of these claims and their equivalents be covered thereby.

### Example 1: Size-Exclusion Chromatography Analysis of Conjugates Formulations

The stability of conjugate formulations 1-5 from Table 1 was tested for appearance, pH, osmolality, monomer content, and concentration. The conjugate used in this example was an amino-benzazepine compound linker conjugated to a humanized anti-HER2 antibody. Conjugation was via the interchain disulfides and average drug load was from 3 to 5. See, e.g., U.S. Pat. No. 10,239,862. The compound linker prior to conjugation to the antibody has the following structure:

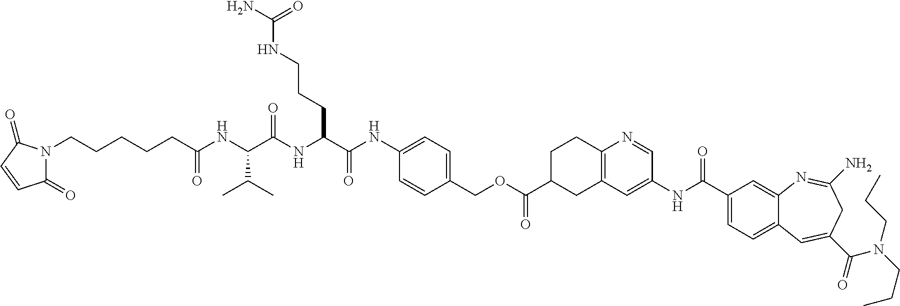 [SMILES: CCCN(CCC)C(=O)C1=Cc2ccc(C(=O)Nc3cnc4c(c3)CC(C(=O)OCc3ccc(NC(=O)[C@H](CCCNC(N)=O)NC(=O)[C@@H](NC(=O)CCCCCN5C(=O)C=CC5=O)C(C)C)cc3)CC4)cc2N=C(N)C1]

At the start of the study (time zero), conjugates in formulations 1-5 were slightly opalescent, colorless and free of visible particulates. After 2 and at 4 weeks of storage at 2-8° C., 25° C., and 40° C., conjugates in formulations 1-5 remained slightly opalescent, colorless, and free of visible matter. The pH, osmolality, and concentration measurements remained largely constant through the study.

TABLE 1

| Formulation | [Conjugate] | [Buffer] | Buffer | Buffer | |
|---|---|---|---|---|---|
| #¹ | (m/mL) | (mM) | Type | pH² | Sucrose |
| 1 | 10 | 20 | Histidine | 5.5 | 6% |
| 2 | 10 | 20 | Histidine | 6 | 6% |
| 3 | 10 | 20 | Histidine | 6.5 | 6% |
| 4 | 10 | 20 | Citrate | 5.5 | 6% |
| 5 | 10 | 20 | Citrate | 6 | 6% |
| 6 | 10 | 20 | Histidine and | 5.5 | 6% |
| | | | Aspartate | | |
| 7 | 10 | 20 | Succinate | 5 | 6% |
| 8 | 10 | 20 | Succinate | 4.5 | 6% |
| 9 | 10 | 20 | Acetate | 4.5 | 8% |
| 10 | 10 | 20 | Acetete | 5 | 8% |
| 11 | 10 | 20 | Histidine and | 4.5 | 8% |
| | | | Aspartate | | |
| 12 | 10 | 20 | Histidine and | 5 | 8% |
| | | | Aspartate | | |
| 13 | 10 | 10 | Histidine | 5.5 | 8% |
| 14 | 10 | 20 | Histidine | 5.5 | 8% |
| 15 | 10 | 30 | Histidine | 5.5 | 8% |
| 16 | 10 | 5 | Histidine | 5.5 | 8% |
| 17 | 10 | 5 | Histidine and | 5 | 8% |
| | | | Aspartate | | |
| 18 | 10 | 0 | None | Not | 8% |
| | | | (H2O) | tested | |
| 19 | 40 | 10 | Histidine | 5.5 | 8% |
| 20 | 40 | 20 | Histidine and | 4.5 | 8% |
| | | | Aspartate | | |
| 21 | 50 | 20 | Histidine and | 4.5 | 8% |
| | | | Aspartate | | |
| 22 | 60 | 20 | Histidine and | 4.5 | 8% |
| | | | Aspartate | | |
| 23 | 70 | 20 | Histidine and | 4.5 | 8% |
| | | | Aspartate | | |
| 24 | 80 | 20 | Histidine and | 4.5 | 8% |
| | | | Aspartate | | |
| 25 | 90 | 20 | Histidine and | 4.5 | 8% |
| | | | Aspartate | | |
| 26 | 110 | 20 | Histidine and | 4.5 | 8% |
| | | | Aspartate | | |
| 27 | 130 | 20 | Histidine and | 4.5 | 8% |
| | | | Aspartate | | |

¹All formulations contain 0.02% Polysorbate 80
²Buffer pH is the pH of the buffer prior to addition of the conjugate

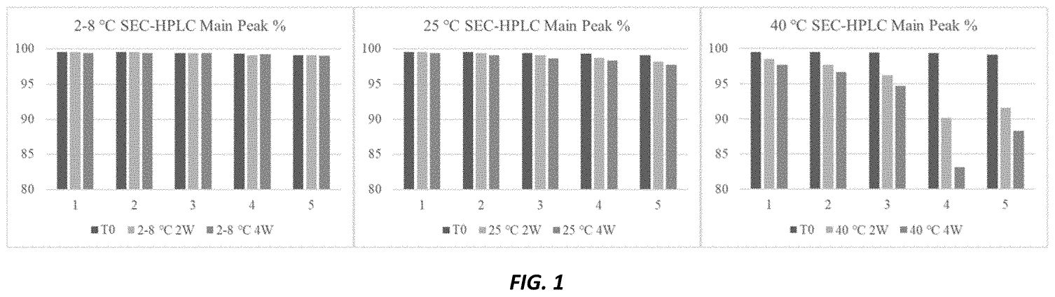

Monomer content was also monitored for conjugates in formulations 1-5 (each formulation having pH of 5.5, 6.0, or 6.5) by SEC-HPLC. At the 2- and 4-week mark, there was minimal change in monomer content when stored at 2-8° C. or 25° C. Only under the conditions of high thermal stress at 40° C., decreasing monomer content was first detected at 2 weeks and continued progression was observed at 4 weeks (FIG. 1). The decrease in monomer content correlated to an increase in high molecular weight (HMW) aggregate species formation, which were identified by size-exclusion chromatography (SEC-HPLC). The formation of HMW aggregates is a well-known pathway of protein degradation and its increase upon storage at 40° C. for ≥2 weeks was not unexpected for protein formulations.

The data from these SEC-HPLC studies appeared to indicate that the conjugate was stable at 25° C. for at least 4 weeks in formulations 1-5, and sufficiently stable for development purposes.

### Example 2: -Analysis of Conjugate Formulations by Hydrophobic Interaction Chromatography (HIC)

While an SEC column separates by size and gives one peak for a mAb dimer, a HIC column separates by hydrophobicity and resolves the different isomeric forms of a dimer. Consequently, a HIC profile analysis was performed to obtain additional information of the aggregate forms present (which are not provided by SEC). HIC analysis was carried out as described below. Briefly, 10 μL of a 6 mg/mL solution of a conjugate was injected into an HPLC system set-up with a TOSOH TSKgel Butyl-NPR™ hydrophobic interaction chromatography (HIC) column (2.5 μM particle size, 4.6 mm×35 mm). A mobile phase gradient was run from 100% mobile phase A (1.5 M ammonium sulfate, 25 mM sodium phosphate (pH 7)) to 100% mobile phase B (25% isopropanol in 25 mM sodium phosphate (pH 7)) over the course of 12 minutes, followed by a six-minute re-equilibration at 100% mobile phase A. The flow rate was 0.8 mL/min and the detector was set at 280 nm. Elution with a gradient of decreasing salt concentration resulted in the least conjugated (least hydrophobic) form eluting first and the most conjugated (most hydrophobic-drug) form eluting last. The percentage peak area from the HIC represents the relative proportion of a particular drug-loaded form. The weighted average drug-to antibody ratio (DAR) can be calculated using the peak percentage and drug load. Over a 2-week time period at 25° C. and 40° C., unexpected and dramatic changes were observed by HIC analysis for conjugates in formulations 1-5 from Table 1, which were formulations at pH 5.5, 6.0, or 6.5.

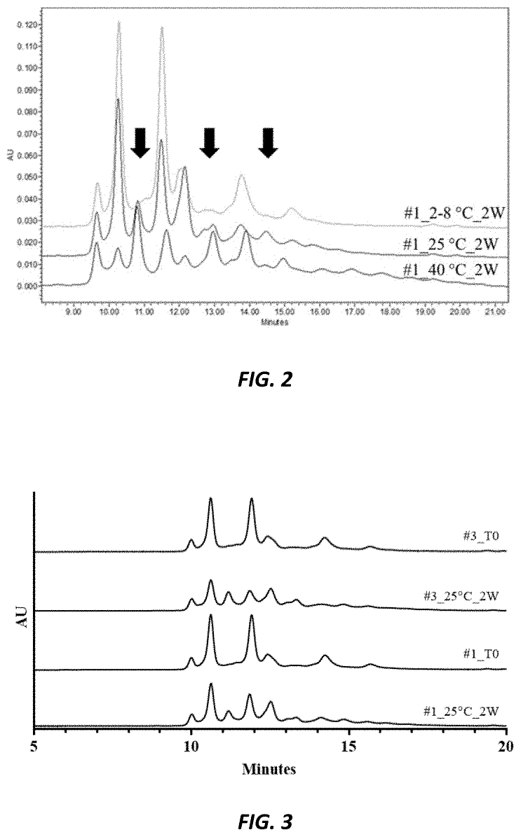

FIG. 2 shows a HIC profile of conjugate in formulation 1 after two weeks of storage at 2-8° C., 25° C., and 40° C. Similar results were observed for conjugates in formulations 2-5. (Data not shown). The arrows identify new peaks attributed to a change in the antibody drug conjugate, with the magnitude of changes increasing with temperature and duration of storage (i.e., when stored at 25° C. and 40° C. as compared to storage at 2-8° C.)

FIG. 3 shows the HIC profile for conjugates in formulations 1 (pH 5.5) and 3 (pH 6.5) from Table 1 at time zero and after storage at 25° C. for 2 weeks. An increase in new peak formation was observed for the antibody drug conjugate in both formulation 1 and formulation 3 at 2 weeks compared to their corresponding profiles at time zero. The extent of changes in the HIC profile was more significant in formulation 3, which indicated that higher pH may influence the emergence of new HIC peaks.

### Example 3: Effect of Lower pH Formulations on SEC Measurement and HIC Profile of the Conjugate

As described in Example 2, HIC analysis of conjugates in formulations 1-5 revealed that the pH of the formulation contributed (as did temperature) to the rate at which new peaks emerged in the HIC profile. This potential pH effect was further examined by preparing conjugates in formulations having a lower pH of 5.5, 5.0, or 4.5 (see formulations 6-19 from Table 1). Formulations 6-19 containing the conjugate from Example 1 were tested for appearance, pH, osmolality, concentration, and monomer content. At the start of the study (time zero), formulations 6-19 containing the conjugate were slightly opalescent, colorless, and free of visible particulates. At the final timepoint (1 week), all formulations of conjugate remained slightly opalescent, colorless, and free of visible matter when stored at 2-8° C., 25° C., or 40° C. The pH, osmolality, and concentration measurements remained largely constant through the study. SEC-HPLC measurement at one week showed there was minimal change in conjugate monomer content when stored at 2-8° C., 25° C., or 40° C. (Data not shown.)

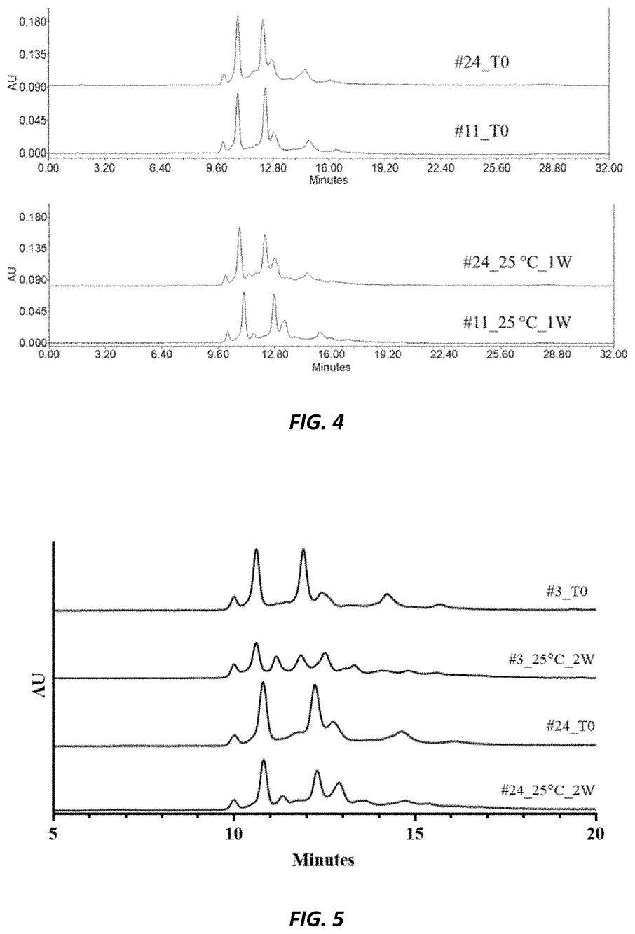

Formulations containing 20 mM histidine (His)/aspartic acid (Asp) buffer at pH 4.5 consistently displayed equivalent or superior behavior compared to the other formulations by all methods referenced, with one comparison shown in FIG. 4. Generally, increasing pH of the formulation resulted in an altered HIC profile of the conjugate over time at 25° C. and 40° C.

### Example 4: Effect of Conjugate Concentration on HIC Profile

Varying the concentration of the conjugate from Example 1 (50, 70, and 90 mg/ml) in formulation 11 from Table 1 (20 mM His/Asp, 8% sucrose, 0.02% polysorbate 80 (PS800, pH 4.5) was used to evaluate whether intermolecular reactions at higher conjugate concentrations were contributing to the altered HIC profile. Buffering by using acid or base and its conjugate salt and/or using titratable groups present on polypeptides themselves and other entities, especially at high concentrations, can be used to control the pH of a formulation. Solubility data showed the conjugate was soluble to at least 130 mg/mL (see, e.g., formulations 20-27 from Table 1). At high concentrations (>50 mg/ml), the buffering contribution of the protein (in this case, antibody) component of the conjugate became more evident, with the final pH of formulations 21, 23 and 25 reaching 4.7 to 5.0. Formulations 21, 23 and 25 were tested for appearance, pH, osmolality, concentration, monomer content and hydrophobicity profile. After 3 freeze/thaw cycles, there was no significant change in the HIC profiles of the samples. After storage at 25° C., the high conjugate concentration formulations showed similar physical and chemical stability when compared to the 10 mg/ml conjugate sample.

FIG. 4 shows HIC profiles for the conjugate of Example 1 in formulations 11 and 24 from Table 1, which have conjugate concentrations of 10 mg/ml and 80 mg/ml, respectively. After storage at 25° C. for 1 week, similar changes were observed at both concentrations when compared to their respective time zero profile. Without intending to be bound by any particular theory, these results indicate that the altered HIC profile may have resulted from an intramolecular reaction that was partially dependent on pH, as opposed to an intermolecular reaction that is generally dependent on conjugate concentration (which was not the case here as shown in FIG. 4).

FIG. 5 compares the HIC profiles of conjugate formulated at the highest and lowest pH examined in Table 1 at time zero and after storage at 25° C. for 2 weeks. Formulation 3 contains 10 mg/mL of the conjugate, 20 mM histidine, 6% sucrose, 0.02% PS80, pH 6.5. Formulation 24 contains 80 mg/mL ADC and 20 mM His/Asp, 8% sucrose, 0.02% PS80, pH 4.5. A reduction in new peak formation was observed at the lower pH (formulation 24) when the stressed (i.e., storage at 25° C. for 2 weeks) samples were compared to their corresponding profiles at time zero. This observation is independent of conjugate concentration; again, highlighting that the altered HIC profile of the conjugate may be result from a surprising intramolecular reaction partially dependent by pH, but not dependent on conjugate concentration.

### Example 5: Conjugation of Compound to Antibody is Stable in all Formulations

Formulations 1-24 containing the antibody-linker-compound conjugate were each monitored for changes in free linker-compound content by reversed phase high-performance liquid chromatography (RP-HPLC) across all timepoints and temperatures evaluated. Increases in the amount of free linker-compound would be an indication of an unstable conjugate structure. In these experiments, no significant change was observed in the free linker-compound content over time for any of the timepoints (data not shown), which indicated that the antibody-compound conjugate was stable under all tested conditions.

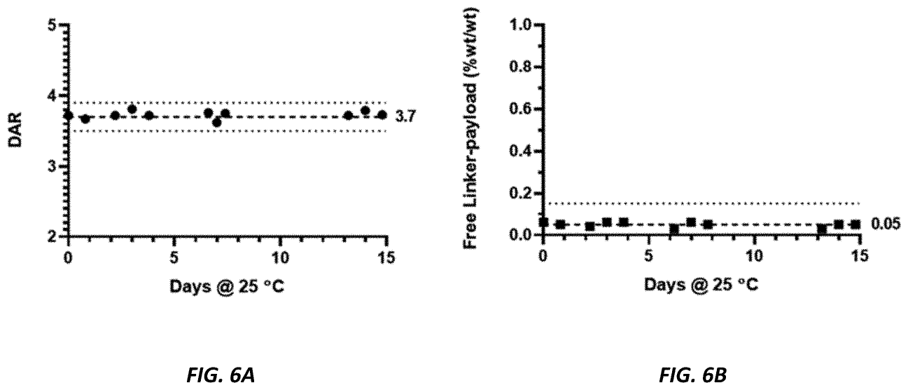

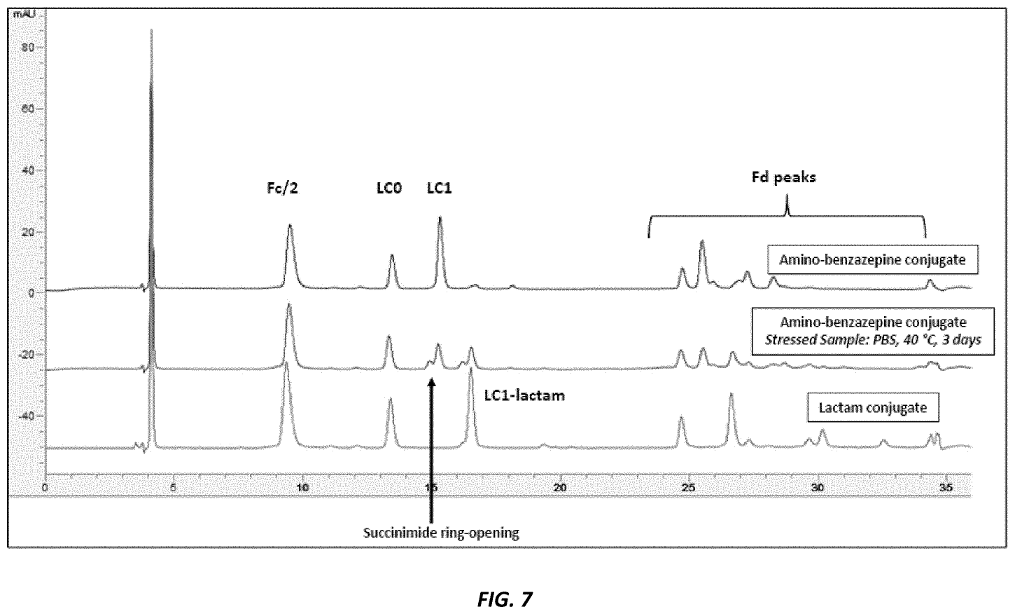

The further asses the stability of the linker-compound-antibody conjugate, the antibody conjugates were examined for any change in drug to antibody ratio (DAR). Briefly, the free-drug level for formulation 24 containg the conjugate (80 mg/mL conjugate and 20 mM His/Asp, 8% sucrose, 0.02% PS80, pH 4.5) stressed at 25° C. for 2 weeks. The DAR and the free-drug level under these conditions remain constant over time as shown in FIGS. 6A and 6B, respectively, with the dotted horizontal lines representing the typical analytical variability window expected for the assay and the dashed line representing the center point. Thus, the drug-antibody conjugates were stable.

### Example 6: Altered HIC Profile Result from Intramoleular Transformation of the Conjugated Compound

Since the conjugate DAR was stable and an increase in concentration of the conjugate showed no signs of intermolecular changes, the results from the HIC analysis indicated that a change to a component of the conjugate was occurring. The change appeared to be a chemical transformation of the conjugated drug that has an impact on the hydrophobicity of the conjugate, with the stressed samples (e.g., stored at higher temperature and formulated at higher pH) tending to be significantly more hydrophobic than the unstressed counterparts. Without wishing to be bound by theory, the systematic study of the stressed samples indicated that the amino-benzazepine compound portion of the conjugate might be undergoing a chemical transformation, such as being hydrolyzed to an inactive lactam compound as shown below:

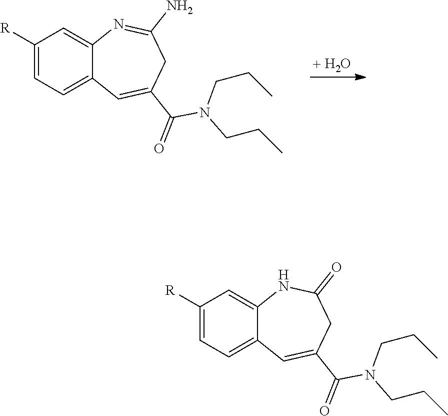

Surprisingly, the amino-benzazepine compound did not demonstrate a sensitivity toward hydrolysis during its preparation or as a free (unconjugated) drug.

To test this theory, a lactam compound-linker and conjugate comprising the lactam compound-linker were prepared to confirm that the altered HIC profile of the conjugate formulated at higher pH was resulting in a chemical transformation of the amino-benzazepine compound to its lactam form without affecting the drug conjugate DAR. The conjugates with the amino-benzazepine compound linker and the lactam compound linker were analyzed by reverse-phase liquid chromatography (RP-LC) to examine whether the chemical transformation to lactam led to the observed altered HIC profiles of the conjugates under stressed conditions.

For this analysis, the conjugate is first enzymatically cleaved below its IgG1 hinge and reduced to generate three fragments: Fc, light chain (LC), and Fd, which includes the heavy chain variable region and CH. While Fc was not expected to have any conjugation sites, LC and Fd had one and three sites, respectively. Accordingly, enzymatic cleavage was expected to produce seven different fragments: Fc, LC-0, LC-1, Fd-0, Fd-1, Fd-2, and Fd-3, where the number indicates the number of conjugated compound-linkers. The higher the number of conjugated compound-linkers on a fragment, the more hydrophobic it will be relative to its unmodified form. The hydrolyzed compound (lactam) is more hydrophobic than its unhydrolyzed form (amino-benzazepine).

The enzymatic cleavage was performed using FabRICATOR© (IdeS enzyme). Briefly, 25 μL FabRICATOR© (4 units/μL) was added to 100 μg conjugate and the digestion was allowed to proceed for 30 minutes at 37° C. The reaction was then cooled to room temperature, and an equal volume of 100 mM DTT was added to the digested mixture to obtain a final concentration of 50 mM DTT. The mixture was mixed gently, and the reaction was incubated for 2 hours at room temperature.

The digested and reduced mixture was then analyzed via RP-HPLC (column: Agilent, Zorbax 300SB-CN, 4.6 mm×250 mm, 5 μm particle size) using the following gradient:
1. Load per run: 15 μg conjugate, fragmented and reduced
2. Flow rate @ 0.750 mL/min, high pressure limit: 400 bar
3. Buffers:
   a. Eluent A: ddH₂O, 0.05% (v/v) trifluoroacetic acid (TFA)
   b. Eluent B: Acetonitrile (ACN), 0.05% (v/v) trifluoroacetic acid (TFA)
4. Column temperature: 75-80° C.
5. Elution:

| Time | Eluent B |
|---|---|
| (min) | (%) |
| 0.0 | 28 |
| 30.0 | 37 |
| 31.0 | 95 |
| 33.0 | 95 |
| 33.1 | 28 |
| 39.0 | 28 |

As shown in FIG. 6, the LC-1 (“L1”) fragment of the antibody conjugate comprising the benzazepine compound elutes earlier than the L1 fragment of the antibody comprising the lactam conjugate. A stressed sample of the benzazepine conjugate (40° C. in PBS for 3 days) shows a loss of the benzazepine-conjugated L1 fragment and appearance of a conjugated L1 fragment that elutes at the same time as the lactam-conjugated L1 fragment. In addition, an intermediate peak appears, which results from opening of the succinimide ring. These results indicate that the benzazepine drug conjugated to an antibody (but not as a free drug) is stable as a conjugate (DAR does not change), but the drug itself undergoes a detectable chemical transformation when formulated at a higher pH (above 5.4) and when stressed (stored at 25° C. or higher). Such chemical transformation can be minimized or eliminated under stress conditions (25° C. or higher) when the benzazepine-antibody conjugate is formulated at a lower pH, such as a pH ranging from 4.4 to 5.4.

### Example 7: Manufacturing Process

A process of manufacturing a formulation comprising a conjugate comprising an antibody and a benzazepine compound is described below.

Methods for synthesizing benzazepine conjugates are known in the art. See, e.g., U.S. Pat. No. 10,239,862. The pH of the quenched and filtered reaction mixture comprising the conjugate is adjusted from neutral pH (7.2-7.5) to pH 4.5 by addition of acetic acid, and is then subjected to Ultrafiltration/Diafiltration (UF/DF). UF/DF is performed to remove small molecule process-related impurities, exchange into the DF buffer (20 mM Histidine/Aspartate, pH 4.5), and to increase the conjugate concentration towards the target conjugate concentration. This pH adjustment step prior to UF/DF potentially increases the solubility of the quenched linker-drug making its removal more efficient during diafiltration, and keeps the compound at an optimal pH for stability during the remainder of the UF/DF process.

The 20 mM Histidine/Aspartate pH 4.5 DF buffer is prepared using weight-based measurements to achieve about a 9.2 mM L-histidine and 10.8 mM L-aspartic acid solution. The reaction mixture with the conjugate at 20 mg/ml is concentrated using ultrafiltration to 30-40 mg/mL, determined to be optimal for the subsequent diafiltration step. The concentrated mixture is then buffer exchanged using diafiltration against the DF without additional excipients over 12 diavolumes (DVs), which was empirically determined to result in adequate removal of linker-drug, related impurities and residual solvent. The process stream is subsequently concentrated to >95 mg/mL conjugate using a second ultrafiltration step. Then, a conditioning step is implemented to adjust the sample to its final formulation. To carry out conditioning, the conjugate concentration is measured, and diluted using concentrated stock solutions of sucrose and polysorbate 80 (PS80) in 20 mM Histidine/Aspartate, pH 4.5 buffer to achieve the final formulation containing the conjugate and 20 mM Histidine/Aspartate, 8% sucrose, 0.02% PS80. Further dilutions are carried out with 20 mM Histidine/Aspartate, 8% sucrose, 0.02% PS80, pH 4.5, if necessary, to achieve the target conjugate concentration.

Table of Certain Sequences

| SEQ ID | | |
|---|---|---|
| NO. | Description | Sequence |
| 1 | Anti-HER2 antibody heavy | GFTFTDYTMD |
| | chain (HC) CDR1 | |
| 2 | Anti-HER2 antibody HC | DVNPNSGGSIYNQRFKG |
| | CDR2 | |
| 3 | Anti-HER2 antibody HC | NLGPSFYFDY |
| | CDR3 | |
| 4 | Anti-HER2 antibody light | KASQDVSIGVA |
| | chain (LC) CDR1 | |
| 5 | Anti-HER2 antibody LC | SASYRYT |
| | CDR2 | |
| 6 | Anti-HER2 antibody LC | QQYYIYPYT |
| | CDR3 | |
| 7 | Anti-HER2 antibody heavy | EVQLVESGGGLVQPGGSLRLSCAASGFTFTDYTMDWVRQAPGK |
| | chain variable region (VH) | GLEWVADVNPNSGGSIYNQRFKGRFTLSVDRSKNTLYLQMNSLR |
| | | AEDTAVYYCARNLGPSFYFDYWGQGTLVTVSS |
| 8 | Anti-HER2 antibody light | DIQMTQSPSSLSASVGDRVTITCKASQDVSIGVAWYQQKPGKAPK |
| | chain variable region (VL) | LLIYSASYRYTGVPSRFSGSGSGTDFTLTISSLQPEDFATYYCQQYYIY |
| | | PYTFGQGTKVEIK |
| 9 | Anti-HER2 antibody heavy | EVQLVESGGGLVQPGGSLRLSCAASGFTFTDYTMDWVRQAPGK |
| | chain | GLEWVADVNPNSGGSIYNQRFKGRFTLSVDRSKNTLYLQMNSLR |
| | | AEDTAVYYCARNLGPSFYFDYWGQGTLVTVSSASTKGPSVFPLAP |
| | | SSKSTSGGTAALGCLVKDYFPEPVTVSWNSGALTSGVHTFPAVLQ |
| | | SSGLYSLSSVVTVPSSSLGTQTYICNVNHKPSNTKVDKKVEPKSCDK |
| | | THTCPPCPAPELLGGPSVFLFPPKPKDTLMISRTPEVTCVVVDVSHE |
| | | DPEVKFNWYVDGVEVHNAKTKPREEQYNSTYRVVSVLTVLHQD |
| | | WLNGKEYKCKVSNKALPAPIEKTISKAKGQPREPQVYTLPPSREEM |
| | | TKNQVSLTCLVKGFYPSDIAVEWESNGQPENNYKTTPPVLDSDGS |
| | | FFLYSKLTVDKSRWQQGNVFSCSVMHEALHNHYTQKSLSLSPG |
| 10 | Anti-HER2 antibody light | DIQMTQSPSSLSASVGDRVTITCKASQDVSIGVAWYQQKPGKAPK |
| | chain | LLIYSASYRYTGVPSRFSGSGSGTDFTLTISSLQPEDFATYYCQQYYIY |
| | | PYTFGQGTKVEIKRTVAAPSVFIFPPSDEQLKSGTASVVCLLNNFYP |
| | | REAKVQWKVDNALQSGNSQESVTEQDSKDSTYSLSSTLTLSKADY |
| | | EKHKVYACEVTHQGLSSPVTKSFNRGEC |
| 11 | Anti-Nectin-4 antibody heavy | NYDMS |
| | chain (HC) CDR1 | |
| 12 | Anti-Nectin-4 antibody HC | TISSGGSYTYYVDSVKG |
| | CDR2 | |
| 13 | Anti-Nectin-4 antibody HC | QELGSYYAMDY |
| | CDR3 | |
| 14 | Anti-Nectin-4 antibody light | RSSQSIVHSNANTYLE |
| | chain (LC) CDR1 v1 | |
| 15 | Anti-Nectin-4 antibody light | RSSQSIVHSNGNTYLE |
| | chain (LC) CDR1 v2 | |
| 16 | Anti-Nectin-4 antibody LC | KVSNRFS |
| | CDR2 | |
| 17 | Anti-Nectin-4 antibody LC | FQGSHVPYT |
| | CDR3 | |
| 18 | Anti-Nectin-4 antibody heavy | EVMLVESGGALVKPGGSLKLSCVASGFTFSNYDMSWVRQTPEKR |
| | chain variable region (VH) | LEWVATISSGGSYTYYVDSVKGRFTISRDNARNTLHLQMSSLRSKD |
| | | TAMYYCARQELGSYYAMDYWGQGTSVTVSS |
| 19 | Anti-Nectin-4 antibody light | DIVMTQTPLSLPVTPGEPASISCRSSQSIVHSNANTYLEWYLQKPG |
| | chain variable region (VL) v1 | QSPQLLIYKVSNRFSGVPDRFSGSGSGTDFTLKISRVEAEDVGVYYC |
| | | FQGSHVPYTFGGGTKVEIK |
| 20 | Anti-Nectin-4 antibody light | DVVMTQTPLSLPVTPGEPASISCRSSQSIVHSNGNTYLEWYLQKPG |
| | chain variable region (VL) v2 | QSPQLLIYKVSNRFSGVPDRFSGSGSGTDFTLKISRVEAEDVGVYYC |
| | | FQGSHVPYTFGGGTKVEIK |
| 21 | Anti-Nectin-4 antibody heavy | EVQLVESGGGLVQPGGSLRLSCAASGFTFSNYDMSWVRQAPGK |
| | chain | GLEWVATISSGGSYTYYVDSVKGRFTISRDNAKNSLYLQMNSLRAE |
| | | DTAVYYCARQELGSYYAMDYWGQGTTVTVSSASTKGPSVFPLAP |
| | | SSKSTSGGTAALGCLVKDYFPEPVTVSWNSGALTSGVHTFPAVLQ |
| | | SSGLYSLSSVVTVPSSSLGTQTYICNVNHKPSNTKVDKKVEPKSCDK |
| | | THTCPPCPAPELLGGPSVFLFPPKPKDTLMISRTPEVTCVVVDVSHE |
| | | DPEVKFNWYVDGVEVHNAKTKPREEQYNSTYRVVSVLTVLHQD |
| | | WLNGKEYKCKVSNKALPAPIEKTISKAKGQPREPQVYTLPPSREEM |
| | | TKNQVSLTCLVKGFYPSDIAVEWESNGQPENNYKTTPPVLDSDGS |
| | | FFLYSKLTVDKSRWQQGNVFSCSVMHEALHNHYTQKSLSLSPGK |
| 22 | Anti-Nectin-4 antibody light | DIVMTQTPLSLPVTPGEPASISCRSSQSIVHSNANTYLEWYLQKPG |
| | chain v1 | QSPQLLIYKVSNRFSGVPDRFSGSGSGTDFTLKISRVEAEDVGVYYC |
| | | FQGSHVPYTFGGGTKVEIKRTVAAPSVFIFPPSDEQLKSGTASVVCL |
| | | LNNFYPREAKVQWKVDNALQSGNSQESVTEQDSKDSTYSLSSTLT |
| | | LSKADYEKHKVYACEVTHQGLSSPVTKSFNRGEC |
| 23 | Anti-Nectin-4 antibody light | DVVMTQTPLSLPVTPGEPASISCRSSQSIVHSNGNTYLEWYLQKPG |
| | chain v2 | QSPQLLIYKVSNRFSGVPDRFSGSGSGTDFTLKISRVEAEDVGVYYC |
| | | FQGSHVPYTFGGGTKVEIKRTVAAPSVFIFPPSDEQLKSGTASVVCL |
| | | LNNFYPREAKVQWKVDNALQSGNSQESVTEQDSKDSTYSLSSTLT |
| | | LSKADYEKHKVYACEVTHQGLSSPVTKSFNRGEC |
| 24 | Anti-ASGR1 antibody heavy | SYTMH |
| | chain (HC) CDR1 K2E | |
| 25 | Anti-ASGR1 antibody heavy | GYYMH |
| | chain (HC) CDR1 G2D | |
| 26 | Anti-ASGR1 antibody HC | YISPSSGYTEYNQKFKD |
| | CDR2 K2E | |
| 27 | Anti-ASGR1 antibody HC | RINPNNGATNYNQNFKD |
| | CDR2 G2D | |
| 28 | Anti-ASGR1 antibody HC | RINPNNAATNYNQNFKD |
| | CDR2 G2.2D | |
| 29 | Anti-ASGR1 antibody HC | KFDY |
| | CDR3 K2E | |
| 30 | Anti-ASGR1 antibody HC | VNFYY |
| | CDR3 G2D | |
| 31 | Anti-ASGR1 antibody light | KASQDINSYLS |
| | chain (LC) CDR1 K2E | |
| 32 | Anti-ASGR1 antibody light | KASQVINSYLS |
| | chain (LC) CDR1 G2D | |
| 33 | Anti-ASGR1 antibody LC | RANRLVD |
| | CDR2 K2E | |
| 34 | Anti-ASGR1 antibody LC | RANRLVE |
| | CDR2 K2.1E | |
| 35 | Anti-ASGR1 antibody LC | RANTLVD |
| | CDR2 G2D | |
| 36 | Anti-ASGR1 antibody LC | RANTLVS |
| | CDR2 G2.1D | |
| 37 | Anti-ASGR1 antibody LC | LQYDEFPFT |
| | CDR3 K2E | |
| 38 | Anti-ASGR1 antibody LC | LQYAEFPYT |
| | CDR3 G2D | |
| 39 | Anti-ASGR1 antibody heavy | QVQLVQSGAEVKKPGASVKVSCKASGYTFTSYTMHWVRQAPGQ |
| | chain variable region (VH) | GLEWMGYISPSSGYTEYNQKFKDRVTMTRDTSTSTVYMELSSLRS |
| | K2E | EDTAVYYCARKFDYWGQGTTVTVSS |
| 40 | Anti-ASGR1 antibody heavy | QVQLVQSGAEVKKPGASVKVSCKASGYTFTGYYMHWVRQAPGQ |
| | chain variable region (VH) | RLEWMGRINPNNGATNYNQNFKDKASLTVDTSASTAYMELSSLR |
| | G2.42D | SEDTAVYYCTSVNFYYWGQGTTLTVSS |
| 41 | Anti-ASGR1 antibody heavy | QVQLVQSGAEVKKPGASVKVSCKASGYTFTGYYMHWVRQAPGQ |
| | chain variable region (VH) | RLEWMGRINPNNAATNYNQNFKDKASLTVDTSASTAYMELSSLR |
| | G2.35D | SEDTAVYYCTSVNFYYWGQGTTLTVSS |
| 42 | Anti-ASGR1 antibody light | DIQMTQSPSSLSASVGDRVTITCKASQDINSYLSWFQQKPGKAPK |
| | chain variable region (VL) | SLIYRANRLVDGVPSRFSGSGSGTDFTLTISSLQPEDFATYYCLQYDE |
| | K2E | FPFTFGQGTKLEIK |
| 43 | Anti-ASGR1 antibody light | DIQMTQSPSSLSASVGDRVTITCKASQDINSYLSWFQQKPGKAPK |
| | chain variable region (VL) | SLIYRANRLVEGVPSRFSGSGSGTDFTLTISSLQPEDFATYYCLQYDE |
| | K2.2E | FPFTFGQGTKLEIK |
| 44 | Anti-ASGR1 antibody light | DIQMTQSPSSLSASVGDRVTITCKASQVINSYLSWFQQKPGKAPKS |
| | chain variable region (VL) | LIYRANTLVDGVPSRFSGSGSGTDFTLTISSLQPEDFATYYCLQYAEF |
| | G2D | PYTFGGGTKVEIK |
| 45 | Anti-ASGR1 antibody light | DIQMTQSPSSLSASVGDRVTITCKASQVINSYLSWFQQKPGKAPKS |
| | chain variable region (VL) | LIYRANTLVSGVPSRFSGSGSGTDFTLTISSLQPEDFATYYCLQYAEF |
| | G2.1D | PYTFGGGTKVEIK |
| 46 | Anti-ASGR1 antibody heavy | QVQLVQSGAEVKKPGASVKVSCKASGYTFTSYTMHWVRQAPGQ |
| | chain K2E | GLEWMGYISPSSGYTEYNQKFKDRVTMTRDTSTSTVYMELSSLRS |
| | | EDTAVYYCARKFDYWGQGTTVTVSSASTKGPSVFPLAPSSKSTSG |
| | | GTAALGCLVKDYFPEPVTVSWNSGALTSGVHTFPAVLQSSGLYSLS |
| | | SVVTVPSSSLGTQTYICNVNHKPSNTKVDKKVEPKSCDKTHTCPPC |
| | | PAPELLGGPSVFLFPPKPKDTLMISRTPEVTCVVVDVSHEDPEVKF |
| | | NWYVDGVEVHNAKTKPREEQYNSTYRVVSVLTVLHQDWLNGKE |
| | | YKCKVSNKALPAPIEKTISKAKGQPREPQVYTLPPSREEMTKNQVS |
| | | LTCLVKGFYPSDIAVEWESNGQPENNYKTTPPVLDSDGSFFLYSKL |
| | | TVDKSRWQQGNVFSCSVMHEALHNHYTQKSLSLSPGK |
| 47 | Anti-ASGR1 antibody heavy | QVQLVQSGAEVKKPGASVKVSCKASGYTFTGYYMHWVRQAPGQ |
| | chain G2.42D | RLEWMGRINPNNGATNYNQNFKDKASLTVDTSASTAYMELSSLR |
| | | SEDTAVYYCTSVNFYYWGQGTTLTVSSASTKGPSVFPLAPSSKSTS |
| | | GGTAALGCLVKDYFPEPVTVSWNSGALTSGVHTFPAVLQSSGLYS |
| | | LSSVVTVPSSSLGTQTYICNVNHKPSNTKVDKKVEPKSCDKTHTCP |
| | | PCPAPELLGGPSVFLFPPKPKDTLMISRTPEVTCVVVDVSHEDPEV |
| | | KFNWYVDGVEVHNAKTKPREEQYNSTYRVVSVLTVLHQDWLNG |
| | | KEYKCKVSNKALPAPIEKTISKAKGQPREPQVYTLPPSREEMTKNQ |
| | | VSLTCLVKGFYPSDIAVEWESNGQPENNYKTTPPVLDSDGSFFLYS |
| | | KLTVDKSRWQQGNVFSCSVMHEALHNHYTQKSLSLSPGK |
| 48 | Anti-ASGR1 antibody heavy | QVQLVQSGAEVKKPGASVKVSCKASGYTFTGYYMHWVRQAPGQ |
| | chain G2.35D | RLEWMGRINPNNAATNYNQNFKDKASLTVDTSASTAYMELSSLR |
| | | SEDTAVYYCTSVNFYYWGQGTTLTVSSASTKGPSVFPLAPSSKSTS |
| | | GGTAALGCLVKDYFPEPVTVSWNSGALTSGVHTFPAVLQSSGLYS |
| | | LSSVVTVPSSSLGTQTYICNVNHKPSNTKVDKKVEPKSCDKTHTCP |
| | | PCPAPELLGGPSVFLFPPKPKDTLMISRTPEVTCVVVDVSHEDPEV |
| | | KFNWYVDGVEVHNAKTKPREEQYNSTYRVVSVLTVLHQDWLNG |
| | | KEYKCKVSNKALPAPIEKTISKAKGQPREPQVYTLPPSREEMTKNQ |
| | | VSLTCLVKGFYPSDIAVEWESNGQPENNYKTTPPVLDSDGSFFLYS |
| | | KLTVDKSRWQQGNVFSCSVMHEALHNHYTQKSLSLSPGK |
| 49 | Anti-ASGR1 antibody light | DIQMTQSPSSLSASVGDRVTITCKASQDINSYLSWFQQKPGKAPK |
| | chain K2E | SLIYRANRLVDGVPSRFSGSGSGTDFTLTISSLQPEDFATYYCLQYDE |
| | | FPFTFGQGTKLEIKRTVAAPSVFIFPPSDEQLKSGTASVVCLLNNFY |
| | | PREAKVQWKVDNALQSGNSQESVTEQDSKDSTYSLSSTLTLSKAD |
| | | YEKHKVYACEVTHQGLSSPVTKSFNRGEC |
| 50 | Anti-ASGR1 antibody light | DIQMTQSPSSLSASVGDRVTITCKASQDINSYLSWFQQKPGKAPK |
| | chain K2.2E | SLIYRANRLVEGVPSRFSGSGSGTDFTLTISSLQPEDFATYYCLQYDE |
| | | FPFTFGQGTKLEIKRTVAAPSVFIFPPSDEQLKSGTASVVCLLNNFY |
| | | PREAKVQWKVDNALQSGNSQESVTEQDSKDSTYSLSSTLTLSKAD |
| | | YEKHKVYACEVTHQGLSSPVTKSFNRGEC |
| 51 | Anti-ASGR1 antibody light | DIQMTQSPSSLSASVGDRVTITCKASQVINSYLSWFQQKPGKAPKS |
| | chain G2D | LIYRANTLVDGVPSRFSGSGSGTDFTLTISSLQPEDFATYYCLQYAEF |
| | | PYTFGGGTKVEIKRTVAAPSVFIFPPSDEQLKSGTASVVCLLNNFYP |
| | | REAKVQWKVDNALQSGNSQESVTEQDSKDSTYSLSSTLTLSKADY |
| | | EKHKVYACEVTHQGLSSPVTKSFNRGEC |
| 52 | Anti-ASGR1 antibody light | DIQMTQSPSSLSASVGDRVTITCKASQVINSYLSWFQQKPGKAPKS |
| | chain G2.1D | LIYRANTLVSGVPSRFSGSGSGTDFTLTISSLQPEDFATYYCLQYAEF |
| | | PYTFGGGTKVEIKRTVAAPSVFIFPPSDEQLKSGTASVVCLLNNFYP |
| | | REAKVQWKVDNALQSGNSQESVTEQDSKDSTYSLSSTLTLSKADY |
| | | EKHKVYACEVTHQGLSSPVTKSFNRGEC |
| 53 | Anti-Mesothelin antibody | GYTMN |
| | heavy chain (HC) CDR1 | |
| 54 | Anti-Mesothelin antibody HC | LITPYNAASSYNQKFRG |
| | CDR2 | |
| 55 | Anti-Mesothelin antibody HC | GGYDGRGFDY |
| | CDR3 | |
| 56 | Anti-Mesothelin antibody light | SASSSVSYMH |
| | chain (LC) CDR1 | |
| 57 | Anti-Mesothelin antibody LC | DTSKLAS |
| | CDR2 | |
| 58 | Anti-Mesothelin antibody LC | QQWSKHPLT |
| | CDR3 | |
| 59 | Anti-Mesothelin antibody | QVQLVQSGAEVKKPGSSVKVSCKASGGTFSGYTMNWVRQAPGQ |
| | heavy chain variable region | GLEWMGLITPYNAASSYNQKFRGRVTITADKSTSTAYMELSSLRSE |
| | (VH) | DTAVYYCARGGYDGRGFDYWGQGTTVTVSS |
| 60 | Anti-Mesothelin antibody light | DIQMTQSPSTLSASVGDRVTITCSASSSVSYMHWYQQKPGKAPKL |
| | chain variable region (VL) | LIYDTSKLASGVPSRFSGSGSGTEFTLTISSLQPDDFATYYCQQWSK |
| | | HPLTFGGGTKVEIK |
| 70 | Anti-Mesothelin antibody | QVQLVQSGAEVKKPGSSVKVSCKASGGTFSGYTMNWVRQAPGQ |
| | heavy chain | GLEWMGLITPYNAASSYNQKFRGRVTITADKSTSTAYMELSSLRSE |
| | | DTAVYYCARGGYDGRGFDYWGQGTTVTVSSASTKGPSVFPLAPS |
| | | SKSTSGGTAALGCLVKDYFPEPVTVSWNSGALTSGVHTFPAVLQS |
| | | SGLYSLSSVVTVPSSSLGTQTYICNVNHKPSNTKVDKKVEPKSCDKT |
| | | HTCPPCPAPELLGGPSVFLFPPKPKDTLMISRTPEVTCVVVDVSHE |
| | | DPEVKFNWYVDGVEVHNAKTKPREEQYNSTYRVVSVLTVLHQD |
| | | WLNGKEYKCKVSNKALPAPIEKTISKAKGQPREPQVYTLPPSREEM |
| | | TKNQVSLTCLVKGFYPSDIAVEWESNGQPENNYKTTPPVLDSDGS |
| | | FFLYSKLTVDKSRWQQGNVFSCSVMHEALHNHYTQKSLSLSPGK |
| 71 | Anti-Mesothelin antibody light | DIQMTQSPSTLSASVGDRVTITCSASSSVSYMHWYQQKPGKAPKL |
| | chain | LIYDTSKLASGVPSRFSGSGSGTEFTLTISSLQPDDFATYYCQQWSK |
| | | HPLTFGGGTKVEIKRTVAAPSVFIFPPSDEQLKSGTASVVCLLNNFY |
| | | PREAKVQWKVDNALQSGNSQESVTEQDSKDSTYSLSSTLTLSKAD |
| | | YEKHKVYACEVTHQGLSSPVTKSFNRGEC |
| 72 | Exemplary cleavable linker | EEVX |
| 73 | Exemplary cleavable linker | GFLG |
| 74 | Exemplary cleavable linker | GGFG |
| 75 | Exemplary cleavable linker | ALAL |

1. An aqueous formulation comprising a conjugate comprising a compound linked to a polypeptide, wherein the compound comprises the structure: 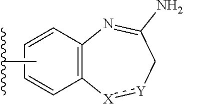
wherein [symbol] is a double bond or a single bond;
wherein when [symbol] is a double bond, X and Y are each CH; and
when [symbol] is a single bond, one of X and Y is CH₂ and the other is CH₂, O, or NH; and
the structure is optionally substituted at any position other than the —NH₂;
wherein the pH of the formulation ranges from about 4.5 to about 5.2; and
wherein the polypeptide is an antibody that binds to a tumor associated antigen selected from HER2, Nectin-4, ASGR1, mesothelin, PSMA, rsPSMA, TROP2, LIV-1, MUC16, CEACAM1, CEACAM3, CEACAM4, CEACAM5, CEACAM6, CEACAM7, CEACAM8, CEACAM16, CEACAM18, CEACAM19, CEACAM20, CEACAM21, URLC10, NY-ESO-1, GAA, OFA, cyclin B1, WT-1, CEF, VEGRR1, VEGFR2, TTK, MUC1, HPV16E7, CEA IMA910, KOC1, SL-701, MART-1, gp100, tyrosinase, GSK2302050A, survivin, MAGE-3.1, MAGE-10.A2, OVA BiP, gp209-2M, melan-A, NA17.A2, KOC1, CO16, DEPDC1, MPHOSPH1, MAGE12, ONT-10, GD2L, GD3L, GSK2302032A, URLC10, CDCA1, TF, PSA, MUC-2, TERT, HPV16, HPV18, STF-II, G17DT, ICT-107, Dex2, hTERT, PAP, tyrosinase related peptide 2 (TRP2), and LRRC15.

2-11. (canceled)

12. The aqueous formulation of claim 1, wherein the pH of the formulation ranges from 4.7 to 5.0.

13. (canceled)

14. The aqueous formulation of claim 1, wherein the formulation further comprises at least one buffering agent selected from histidine, citrate, aspartate, acetate, phosphate, lactate, tromethamine, gluconate, glutamate, tartrate, succinate, malic acid, fumarate, α-ketoglutarate, and combinations thereof.

15. The aqueous formulation of claim 14, wherein the at least one buffering agent comprises histidine and aspartate.

16. The aqueous formulation of claim 15, wherein the total concentration of histidine and aspartate ranges from about 15 mM to about 25 mM.

17. The aqueous formulation of claim 14, wherein the aqueous formulation further comprises at least one lyoprotectant selected from sucrose, arginine, glycine, sorbitol, glycerol, trehalose, dextrose, alpha-cyclodextrin, hydroxypropyl beta-cyclodextrin, hydroxypropyl gamma-cyclodextrin, proline, methionine, albumin, mannitol, maltose, dextran, and combinations thereof.

18. The aqueous formulation of claim 17, wherein the at least one lyoprotectant comprises sucrose.

19. The aqueous formulation of claim 18, wherein the concentration of the sucrose is about 7% to about 9%.

20. The aqueous formulation of claim 17, wherein the aqueous formulation further comprises at least one surfactant.

21. The aqueous formulation of claim 20, wherein the at least one surfactant is selected from polysorbate 80, polysorbate 20, poloxamer 88, and combinations thereof.

22. The aqueous formulation of claim 21, wherein the at least one surfactant comprises polysorbate 80.

23. The aqueous formulation of claim 20, wherein the concentration of the polysorbate 80 is about 0.01% to about 0.05%.

24. The aqueous formulation of claim 1, wherein the concentration of the conjugate is about 70 mg/mL to about 100 mg/mL.

25-30. (canceled)

31. The aqueous formulation of claim 1, wherein the antibody is an anti-HER2 antibody.

32-36. (canceled)

37. The aqueous formulation of claim 1, wherein the antibody is an anti-Nectin-4 antibody.

38-42. (canceled)

43. The aqueous formulation of claim 1, wherein the antibody is an anti-ASGR1 antibody.

44-53. (canceled)

54. The aqueous formulation of claim 1, wherein the antibody is an anti-mesothelin antibody.

55-61. (canceled)

62. The aqueous formulation of claim 1, wherein the compound is a TLR8 agonist.

63. (canceled)

64. The aqueous formulation of claim 1, wherein the conjugate is represented by Formula (I): 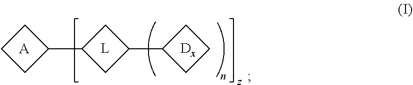
wherein:
A is the polypeptide,
L is a linker;
Dₓ is the compound;
n is selected from 1 to 20; and
z is selected from 1 to 20.

65-69. (canceled)

70. The aqueous formulation of claim 64, wherein L and Dx together have a structure selected from: 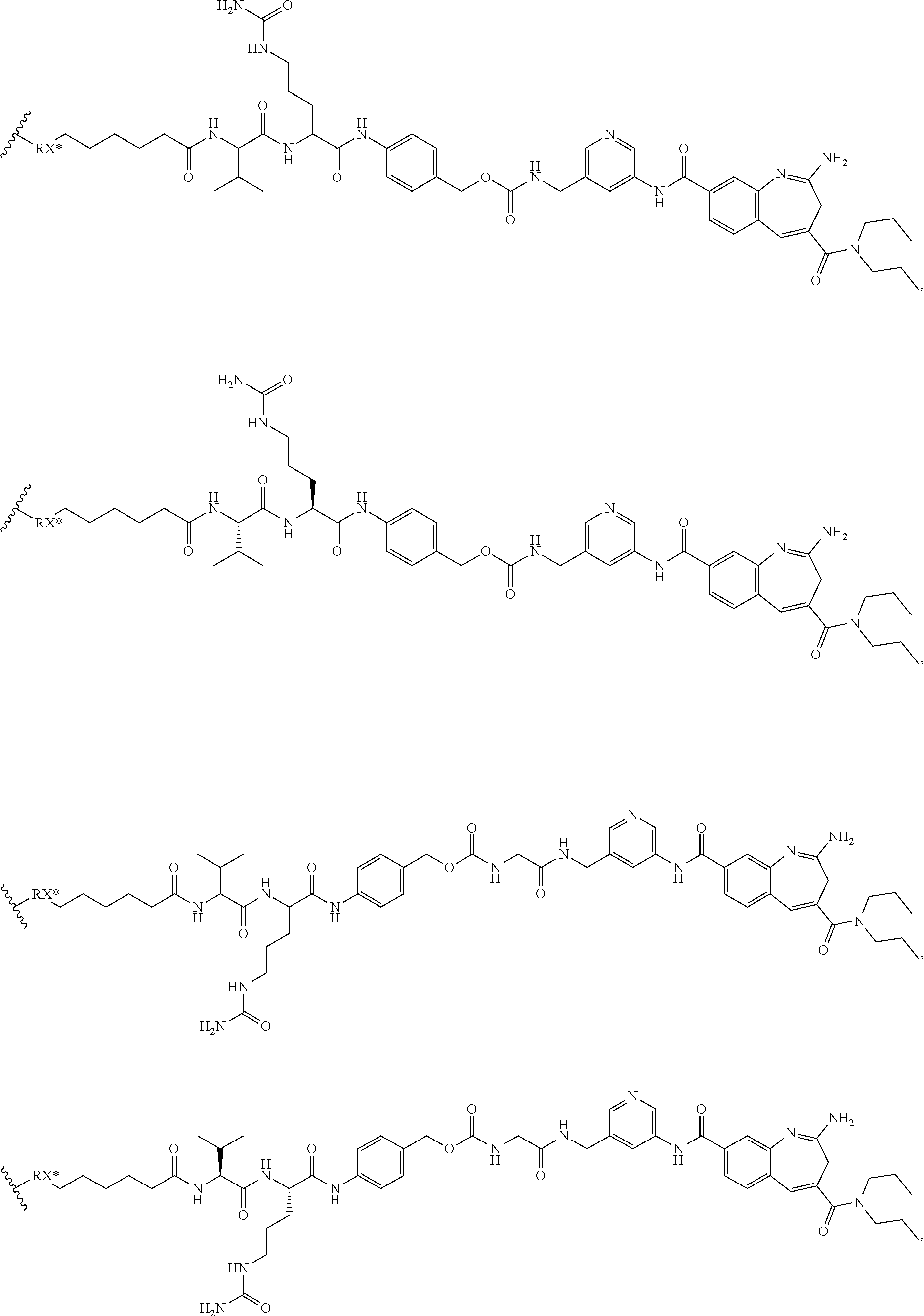 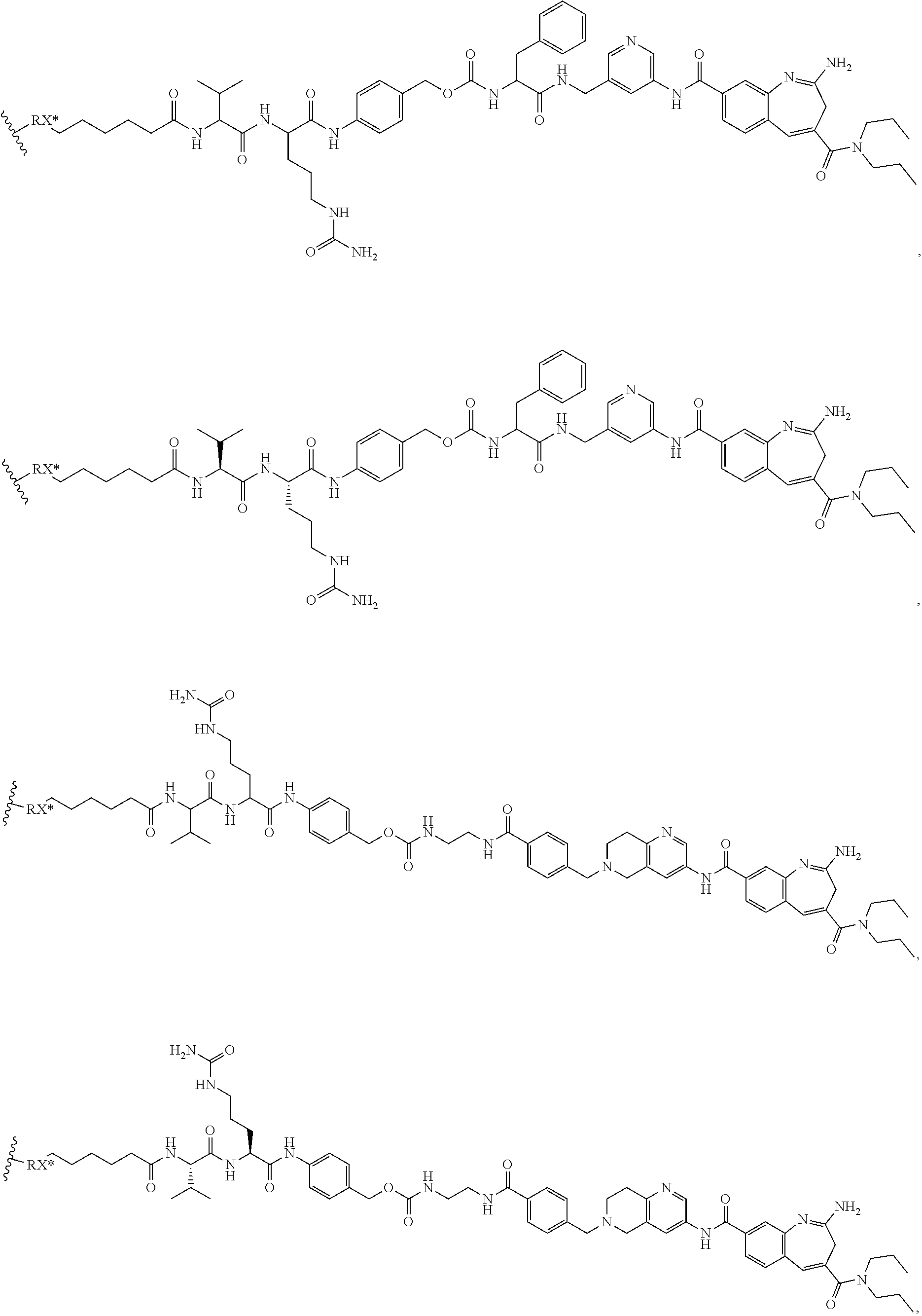 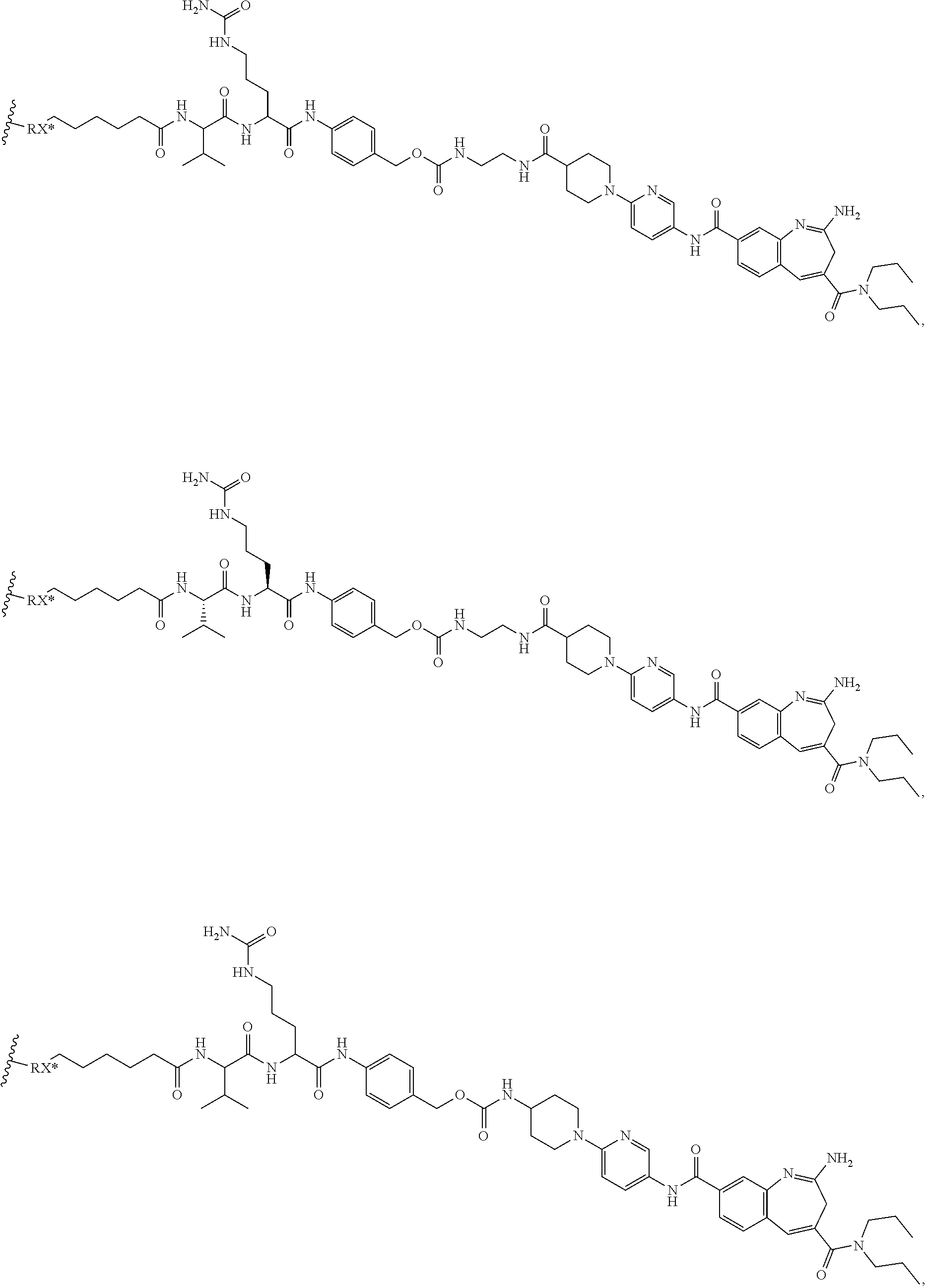 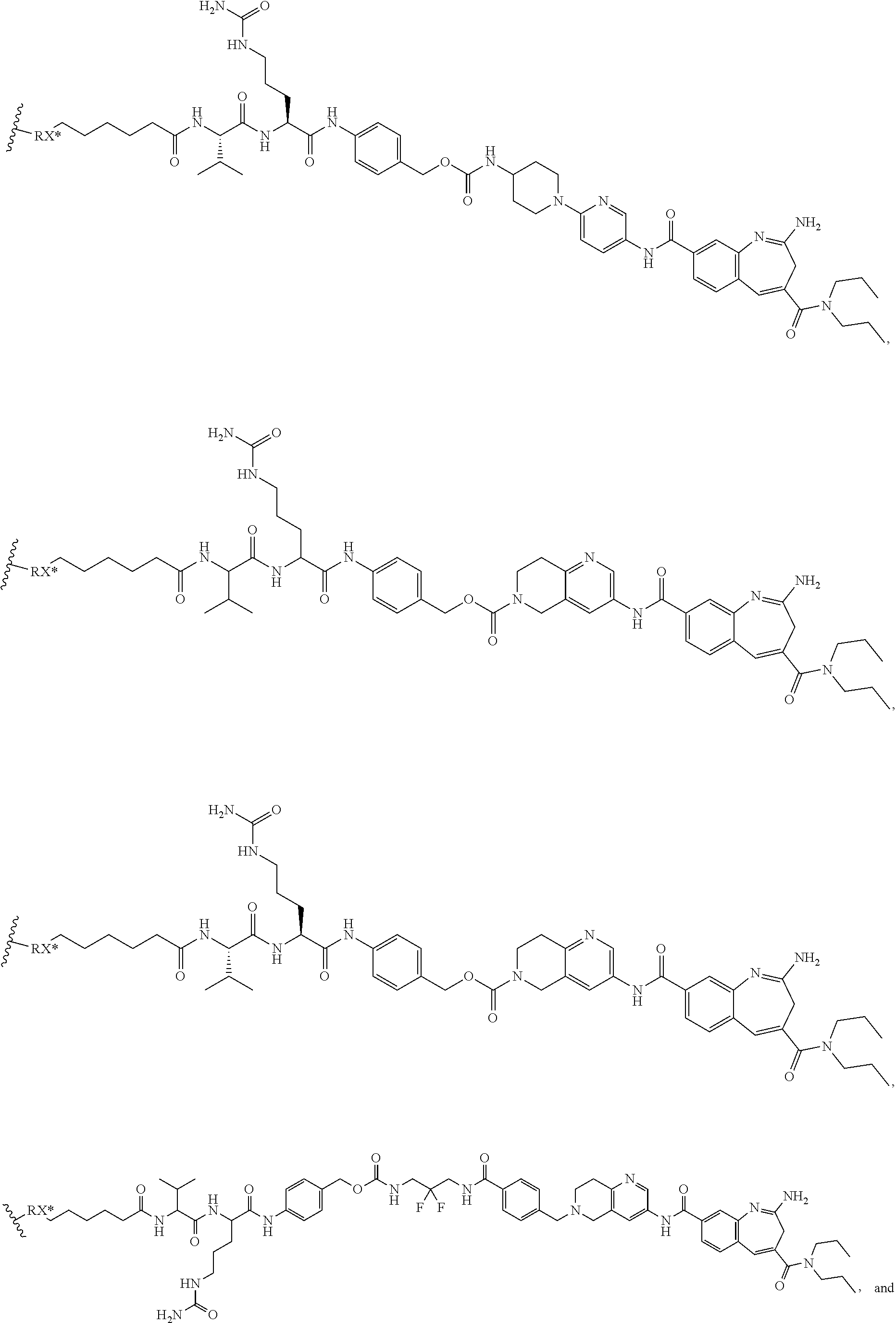 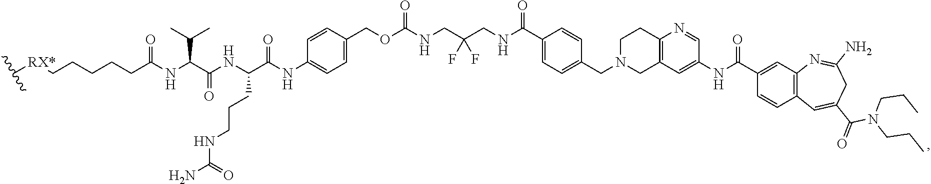
and salts thereof,
wherein the RX* is a bond, a succinimide moiety, or a hydrolyzed succinimide moiety bound to a residue of a polypeptide,
wherein 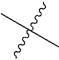
on RX* represents the point of attachment to the residue of the polypeptide.

71. (canceled)

72. (canceled)

73. A lyophilized composition comprising a conjugate comprising a compound linked to a polypeptide, wherein the compound comprises the structure: 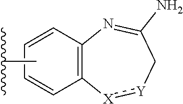
wherein [symbol] is a double bond or a single bond;
wherein when [symbol] is a double bond, X and Y are each CH; and
when [symbol] is a single bond, one of X and Y is CH₂ and the other is CH₂, O, or NH; and
the structure is optionally substituted at any position other than the —NH₂;
wherein upon reconstitution of the lyophilized composition in water to form an aqueous formulation, the pH of the aqueous formulation ranges from about 4.5 to about 5.2; and
wherein the polypeptide is an antibody that binds to a tumor associated antigen selected from HER2, Nectin-4, ASGR1, mesothelin, PSMA, rsPSMA, TROP2, LIV-1, MUC16, CEACAM1, CEACAM3, CEACAM4, CEACAM5, CEACAM6, CEACAM7, CEACAM8, CEACAM16, CEACAM18, CEACAM19, CEACAM20, CEACAM21, URLC10, NY-ESO-1, GAA, OFA, cycline B1, WT-1, CEF, VEGRR1, VEGFR2, TTK, MUC1, HPV16E7, CEA IMA910, KOC1, SL-701, MART-1, gp100, tyrosinase, GSK2302050A, survivin, MAGE-3.1, MAGE-10.A2, OVA BiP, gp209-2M, melan-A, NA17.A2, KOC1, CO16, DEPDC1, MPHOSPH1, MAGE12, ONT-10, GD2L, GD3L, GSK2302032A, URLC10, CDCA1, TF, PSA, MUC-2, TERT, HPV16, HPV18, STF-II, G17DT, ICT-107, Dex2, hTERT, PAP, tyrosinase related peptide 2 (TRP2), and LRRC15.

74-148. (canceled)

149. A method of treating a cancer in a subject, comprising administering to a subject in need thereof a therapeutically effective amount of the aqueous formulation of claim 1.

150-267. (canceled)


SEQUENCE LISTING (75 sequences)

SEQ ID NO: 1 - protein, 10 aa, Artificial sequence
  Anti-HER2 antibody heavy chain (HC) CDR1
GFTFTDYTMD

SEQ ID NO: 2 - protein, 17 aa, Artificial sequence
  Anti-HER2 antibody HC CDR2
DVNPNSGGSIYNQRFKG

SEQ ID NO: 3 - protein, 10 aa, Artificial sequence
  Anti-HER2 antibody HC CDR3
NLGPSFYFDY

SEQ ID NO: 4 - protein, 11 aa, Artificial sequence
  Anti-HER2 antibody light chain (LC) CDR1
KASQDVSIGVA

SEQ ID NO: 5 - protein, 7 aa, Artificial sequence
  Anti-HER2 antibody LC CDR2
SASYRYT

SEQ ID NO: 6 - protein, 9 aa, Artificial sequence
  Anti-HER2 antibody LC CDR3
QQYYIYPYT

SEQ ID NO: 7 - protein, 119 aa, Artificial sequence [sequence omitted]

SEQ ID NO: 8 - protein, 107 aa, Artificial sequence [sequence omitted]

SEQ ID NO: 9 - protein, 448 aa, Artificial sequence
  Anti-HER2 antibody heavy chain
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 214 aa, Artificial sequence
  Anti-HER2 antibody light chain
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 5 aa, Artificial sequence
  Anti-Nectin-4 antibody heavy chain (HC) CDR1
NYDMS

SEQ ID NO: 12 - protein, 17 aa, Artificial sequence
  Anti-Nectin-4 antibody HC CDR2
TISSGGSYTYYVDSVKG

SEQ ID NO: 13 - protein, 11 aa, Artificial sequence
  Anti-Nectin-4 antibody HC CDR3
QELGSYYAMDY

SEQ ID NO: 14 - protein, 16 aa, Artificial sequence
  Anti-Nectin-4 antibody light chain (LC) CDR1 v1
RSSQSIVHSNANTYLE

SEQ ID NO: 15 - protein, 16 aa, Artificial sequence
  Anti-Nectin-4 antibody light chain (LC) CDR1 v2
RSSQSIVHSNGNTYLE

SEQ ID NO: 16 - protein, 7 aa, Artificial sequence
  Anti-Nectin-4 antibody LC CDR2
KVSNRFS

SEQ ID NO: 17 - protein, 9 aa, Artificial sequence
  Anti-Nectin-4 antibody LC CDR3
FQGSHVPYT

SEQ ID NO: 18 - protein, 120 aa, Artificial sequence
  Anti-Nectin-4 antibody heavy chain variable region (VH)
EVMLVESGGALVKPGGSLKLSCVASGFTFSNYDMSWVRQTPEKRLEWVATISSGGSYTYYVDSVKGRFTISRDNARNTLHLQMSSLRSKDTAMYYCARQELGSYYAMDYWGQGTSVTVSS

SEQ ID NO: 19 - protein, 112 aa, Artificial sequence
  Anti-Nectin-4 antibody light chain variable region (VL) v1
DIVMTQTPLSLPVTPGEPASISCRSSQSIVHSNANTYLEWYLQKPGQSPQLLIYKVSNRFSGVPDRFSGSGSGTDFTLKISRVEAEDVGVYYCFQGSHVPYTFGGGTKVEIK

SEQ ID NO: 20 - protein, 112 aa, Artificial sequence
  Anti-Nectin-4 antibody light chain variable region (VL) v2
DVVMTQTPLSLPVTPGEPASISCRSSQSIVHSNGNTYLEWYLQKPGQSPQLLIYKVSNRFSGVPDRFSGSGSGTDFTLKISRVEAEDVGVYYCFQGSHVPYTFGGGTKVEIK

SEQ ID NO: 21 - protein, 450 aa, Artificial sequence
  Anti-Nectin-4 antibody heavy chain
EVQLVESGGGLVQPGGSLRLSCAASGFTFSNYDMSWVRQAPGKGLEWVATISSGGSYTYYVDSVKGRFTISRDNAKNSLYLQMNSLRAEDTAVYYCARQELGSYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 219 aa, Artificial sequence
  Anti-Nectin-4 antibody light chain v1
DIVMTQTPLSLPVTPGEPASISCRSSQSIVHSNANTYLEWYLQKPGQSPQLLIYKVSNRFSGVPDRFSGSGSGTDFTLKISRVEAEDVGVYYCFQGSHVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 219 aa, Artificial sequence
  Anti-Nectin-4 antibody light chain v2
DVVMTQTPLSLPVTPGEPASISCRSSQSIVHSNGNTYLEWYLQKPGQSPQLLIYKVSNRFSGVPDRFSGSGSGTDFTLKISRVEAEDVGVYYCFQGSHVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 5 aa, Artificial sequence
  Anti-ASGR1 antibody heavy chain (HC) CDR1 K2E
SYTMH

SEQ ID NO: 25 - protein, 5 aa, Artificial sequence
  Anti-ASGR1 antibody heavy chain (HC) CDR1 G2D
GYYMH

SEQ ID NO: 26 - protein, 17 aa, Artificial sequence
  Anti-ASGR1 antibody HC CDR2 K2E
YISPSSGYTEYNQKFKD

SEQ ID NO: 27 - protein, 17 aa, Artificial sequence
  Anti-ASGR1 antibody HC CDR2 G2D
RINPNNGATNYNQNFKD

SEQ ID NO: 28 - protein, 17 aa, Artificial sequence
  Anti-ASGR1 antibody HC CDR2 G2.2D
RINPNNAATNYNQNFKD

SEQ ID NO: 29 - protein, 4 aa, Artificial sequence
  Anti-ASGR1 antibody HC CDR3 K2E
KFDY

SEQ ID NO: 30 - protein, 5 aa, Artificial sequence
  Anti-ASGR1 antibody HC CDR3 G2D
VNFYY

SEQ ID NO: 31 - protein, 11 aa, Artificial sequence
  Anti-ASGR1 antibody light chain (LC) CDR1 K2E
KASQDINSYLS

SEQ ID NO: 32 - protein, 11 aa, Artificial sequence
  Anti-ASGR1 antibody light chain (LC) CDR1 G2D
KASQVINSYLS

SEQ ID NO: 33 - protein, 7 aa, Artificial sequence
  Anti-ASGR1 antibody LC CDR2 K2E
RANRLVD

SEQ ID NO: 34 - protein, 7 aa, Artificial sequence
  Anti-ASGR1 antibody LC CDR2 K2.1E
RANRLVE

SEQ ID NO: 35 - protein, 7 aa, Artificial sequence
  Anti-ASGR1 antibody LC CDR2 G2D
RANTLVD

SEQ ID NO: 36 - protein, 7 aa, Artificial sequence
  Anti-ASGR1 antibody LC CDR2 G2.1D
RANTLVS

SEQ ID NO: 37 - protein, 9 aa, Artificial sequence
  Anti-ASGR1 antibody LC CDR3 K2E
LQYDEFPFT

SEQ ID NO: 38 - protein, 9 aa, Artificial sequence
  Anti-ASGR1 antibody LC CDR3 G2D
LQYAEFPYT

SEQ ID NO: 39 - protein, 113 aa, Artificial sequence
  Anti-ASGR1 antibody heavy chain variable region (VH) K2E
QVQLVQSGAEVKKPGASVKVSCKASGYTFTSYTMHWVRQAPGQGLEWMGYISPSSGYTEYNQKFKDRVTMTRDTSTSTVYMELSSLRSEDTAVYYCARKFDYWGQGTTVTVSS

SEQ ID NO: 40 - protein, 114 aa, Artificial sequence
  Anti-ASGR1 antibody heavy chain variable region (VH) G2.42D
QVQLVQSGAEVKKPGASVKVSCKASGYTFTGYYMHWVRQAPGQRLEWMGRINPNNGATNYNQNFKDKASLTVDTSASTAYMELSSLRSEDTAVYYCTSVNFYYWGQGTTLTVSS

SEQ ID NO: 41 - protein, 114 aa, Artificial sequence
  Anti-ASGR1 antibody heavy chain variable region (VH) G2.35D
QVQLVQSGAEVKKPGASVKVSCKASGYTFTGYYMHWVRQAPGQRLEWMGRINPNNAATNYNQNFKDKASLTVDTSASTAYMELSSLRSEDTAVYYCTSVNFYYWGQGTTLTVSS

SEQ ID NO: 42 - protein, 107 aa, Artificial sequence
  Anti-ASGR1 antibody light chain variable region (VL) K2E
DIQMTQSPSSLSASVGDRVTITCKASQDINSYLSWFQQKPGKAPKSLIYRANRLVDGVPSRFSGSGSGTDFTLTISSLQPEDFATYYCLQYDEFPFTFGQGTKLEIK

SEQ ID NO: 43 - protein, 107 aa, Artificial sequence
  Anti-ASGR1 antibody light chain variable region (VL) K2.2E
DIQMTQSPSSLSASVGDRVTITCKASQDINSYLSWFQQKPGKAPKSLIYRANRLVEGVPSRFSGSGSGTDFTLTISSLQPEDFATYYCLQYDEFPFTFGQGTKLEIK

SEQ ID NO: 44 - protein, 107 aa, Artificial sequence
  Anti-ASGR1 antibody light chain variable region (VL) G2D
DIQMTQSPSSLSASVGDRVTITCKASQVINSYLSWFQQKPGKAPKSLIYRANTLVDGVPSRFSGSGSGTDFTLTISSLQPEDFATYYCLQYAEFPYTFGGGTKVEIK

SEQ ID NO: 45 - protein, 107 aa, Artificial sequence
  Anti-ASGR1 antibody light chain variable region (VL) G2.1D
DIQMTQSPSSLSASVGDRVTITCKASQVINSYLSWFQQKPGKAPKSLIYRANTLVSGVPSRFSGSGSGTDFTLTISSLQPEDFATYYCLQYAEFPYTFGGGTKVEIK

SEQ ID NO: 46 - protein, 443 aa, Artificial sequence
  Anti-ASGR1 antibody heavy chain K2E
QVQLVQSGAEVKKPGASVKVSCKASGYTFTSYTMHWVRQAPGQGLEWMGYISPSSGYTEYNQKFKDRVTMTRDTSTSTVYMELSSLRSEDTAVYYCARKFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 444 aa, Artificial sequence
  Anti-ASGR1 antibody heavy chain G2.42D
QVQLVQSGAEVKKPGASVKVSCKASGYTFTGYYMHWVRQAPGQRLEWMGRINPNNGATNYNQNFKDKASLTVDTSASTAYMELSSLRSEDTAVYYCTSVNFYYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 444 aa, Artificial sequence
  Anti-ASGR1 antibody heavy chain G2.35D
QVQLVQSGAEVKKPGASVKVSCKASGYTFTGYYMHWVRQAPGQRLEWMGRINPNNAATNYNQNFKDKASLTVDTSASTAYMELSSLRSEDTAVYYCTSVNFYYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 214 aa, Artificial sequence
  Anti-ASGR1 antibody light chain K2E
DIQMTQSPSSLSASVGDRVTITCKASQDINSYLSWFQQKPGKAPKSLIYRANRLVDGVPSRFSGSGSGTDFTLTISSLQPEDFATYYCLQYDEFPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 214 aa, Artificial sequence
  Anti-ASGR1 antibody light chain K2.2E
DIQMTQSPSSLSASVGDRVTITCKASQDINSYLSWFQQKPGKAPKSLIYRANRLVEGVPSRFSGSGSGTDFTLTISSLQPEDFATYYCLQYDEFPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 214 aa, Artificial sequence
  Anti-ASGR1 antibody light chain G2D
DIQMTQSPSSLSASVGDRVTITCKASQVINSYLSWFQQKPGKAPKSLIYRANTLVDGVPSRFSGSGSGTDFTLTISSLQPEDFATYYCLQYAEFPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 214 aa, Artificial sequence
  Anti-ASGR1 antibody light chain G2.1D
DIQMTQSPSSLSASVGDRVTITCKASQVINSYLSWFQQKPGKAPKSLIYRANTLVSGVPSRFSGSGSGTDFTLTISSLQPEDFATYYCLQYAEFPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 5 aa, Artificial sequence
  Anti-Mesothelin antibody heavy chain (HC) CDR1
GYTMN

SEQ ID NO: 54 - protein, 17 aa, Artificial sequence
  Anti-Mesothelin antibody HC CDR2
LITPYNAASSYNQKFRG

SEQ ID NO: 55 - protein, 10 aa, Artificial sequence
  Anti-Mesothelin antibody HC CDR3
GGYDGRGFDY

SEQ ID NO: 56 - protein, 10 aa, Artificial sequence
  Anti-Mesothelin antibody light chain (LC) CDR1
SASSSVSYMH

SEQ ID NO: 57 - protein, 7 aa, Artificial sequence
  Anti-Mesothelin antibody LC CDR2
DTSKLAS

SEQ ID NO: 58 - protein, 9 aa, Artificial sequence
  Anti-Mesothelin antibody LC CDR3
QQWSKHPLT

SEQ ID NO: 59 - protein, 119 aa, Artificial sequence
  Anti-Mesothelin antibody heavy chain variable region (VH)
QVQLVQSGAEVKKPGSSVKVSCKASGGTFSGYTMNWVRQAPGQGLEWMGLITPYNAASSYNQKFRGRVTITADKSTSTAYMELSSLRSEDTAVYYCARGGYDGRGFDYWGQGTTVTVSS

SEQ ID NO: 60 - protein, 106 aa, Artificial sequence
  Anti-Mesothelin antibody light chain variable region (VL)
DIQMTQSPSTLSASVGDRVTITCSASSSVSYMHWYQQKPGKAPKLLIYDTSKLASGVPSRFSGSGSGTEFTLTISSLQPDDFATYYCQQWSKHPLTFGGGTKVEIK

SEQ ID NO: 61-69 - skipped (9 entries)

SEQ ID NO: 70 - protein, 449 aa, Artificial sequence
  Anti-Mesothelin antibody heavy chain
QVQLVQSGAEVKKPGSSVKVSCKASGGTFSGYTMNWVRQAPGQGLEWMGLITPYNAASSYNQKFRGRVTITADKSTSTAYMELSSLRSEDTAVYYCARGGYDGRGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 213 aa, Artificial sequence
  Anti-Mesothelin antibody light chain
DIQMTQSPSTLSASVGDRVTITCSASSSVSYMHWYQQKPGKAPKLLIYDTSKLASGVPSRFSGSGSGTEFTLTISSLQPDDFATYYCQQWSKHPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - protein, 4 aa, Artificial sequence
  Exemplary cleavable linker
  MISC_FEATURE (4)..(4): X = Cit
EEVX

SEQ ID NO: 73 - protein, 4 aa, Artificial sequence
  Exemplary cleavable linker
GFLG

SEQ ID NO: 74 - protein, 4 aa, Artificial sequence
  Exemplary cleavable linker
GGFG

SEQ ID NO: 75 - protein, 4 aa, Artificial sequence
  Exemplary cleavable linker
ALAL